(19) United States
(12) Patent Application Publication
(10) Pub. No.: US 2009/0305279 A1
(43) Pub. Date: Dec. 10, 2009

(54) METHOD OF DIAGNOSIS

(75) Inventors: Mark William James Ferguson, Manchester (GB); Hugh Gerald Laverty, Manchester (GB); Nicholas Occleston, Manchester (GB); Sharon O'Kane, Manchester (GB); Darren Hodgson, Manchester (GB); Neil French, Manchester (GB); Claire Cridland, Manchester (GB); Philip Roby, Manchester (GB); Ardeshir Bayat, Manchester (GB)

Correspondence Address: BAKER & DANIELS LLP, 300 NORTH MERIDIAN STREET, SUITE 2700, INDIANAPOLIS, IN, 46204, US

(73) Assignee: Renovo Limited, Manchester (GB)

(21) Appl. No.: 12/439,278

(22) PCT Filed: Aug. 28, 2007
(86) PCT No.: PCT/GB2007/003221

§ 371 Date: Feb. 27, 2009

(30) Foreign Application Priority Data
Aug. 31, 2006 (GB) 0617116.9

(51) Int. Cl. C12Q 1/68; C40B 40/06; C40B 40/10

(52) U.S. Cl. 435/6; 506/16; 506/18

(57) ABSTRACT

Provided are methods, kits and arrays for use in determining whether a scar of interest is keloid or non-keloid in nature. These determine keloid or non-keloid nature based on comparison of gene expression in the scar of interest with expression in a control sample. If expression of at least one gene, selected from the group of genes set out in Table 1, is decreased in a sample representative of gene expression in the scar of interest compared to expression of the same gene (or genes) in the control sample this indicates that the scar of interest comprises a keloid.

The invention relates to a method of diagnosing a scar of interest as keloid or non-keloid. The invention also provides kits and oligonucleotide arrays suitable for use in the diagnosis of a scar of interest as keloid or non-keloid.

Keloids (also referred to as keloid scars) are pathological scars produced by an aberrant and over-exuberant wound healing response. Keloids comprise raised scars that spread beyond the margins of an original wound and invade the normal skin surrounding the wound site. Keloids continue to grow over time, and do not regress spontaneously.

Keloids occur with equal frequency in men and women. The incidence of keloid formation is increased in those aged between 10 and 30 years. Keloids may arise as a result of a wide range of injuries, including piercing, surgery, vaccination, tattoos, bites, blunt trauma and burns.

Keloids may have a “domed”, nodular or ridged appearance. Keloids may have a colour similar to that of the surrounding unwounded skin, but are frequently somewhat darker, with a red, purple or brown appearance. Such colour mismatches may increase the visual prominence of keloids. The tendency for hyperpigmentation in keloids is increased on their exposure to solar ultraviolet radiation.

A keloid lesion may be considered to be made up of a number of different portions that may each exhibit quite different biological activity from one another. The central part of a mature keloid lesion (the intra-lesional portion) is largely acellular, while the peripheral part of the lesion (the peri-lesional portion) is relatively more cellular and is the site of increased angiogenic activity. This increase in new blood vessel formation has been linked with the outward growth of the lesion.

Although they represent examples of pathological scarring, keloids are primarily composed of the same cell types and extracellular matrix components that are found in undamaged skin and normal dermal scars. However, the relative abundance and arrangement of these cell types and extracellular matrix components differ from those found in either unwounded skin or normal dermal scars.

The major constituent of keloids is the extracellular matrix component collagen I. Fibroblasts derived from keloids exhibit up to a twenty-fold higher expression of collagen I in vitro, as compared to normal dermal fibroblasts. Similarly, cultured keloid fibroblasts also express elevated levels of elastin and proteoglycans, and it is believed that this increase in extracellular matrix deposition may play a role in keloid development and maintenance.

Collagen I present in keloids is arranged primarily in the form of thick “whorls”, which may be differentiated from the arrangement found in unwounded skin (a so-called “basket weave” of fibrils) and in normal scars (which contain collagen fibres that are thinner than those found in keloids and are arranged approximately parallel to one another). The frequent presence of thickened hyalinized collagen within keloids has led to this form of collagen being termed “keloidal collagen”.

Keloids contain fewer macrophages than do normal scars, but contain abundant eosinophils, mast cells, plasma cells and lymphocytes.

Keloids are seldom a direct cause of pain, but may give rise to discomfort, tenderness, irritation or itching during their formation or growth. Keloids may also impair mechanical function through their size or their increased stiffness compared to unwounded skin. This impairment may be particularly noticeable in the case of keloids located near a joint. Furthermore, it is well recognised that keloids, and in particular large or noticeably disfiguring examples, can cause psychological distress to those afflicted.

A further highly damaging property of keloids is their propensity to recur, particularly following surgical excision. Recurrence of keloids under such circumstances is normally also associated with further expansion of the lesion, and keloids may expand more aggressively following an earlier excision.

Treatment options for hypertrophic scars are similar to those for keloids with the exception that surgical excision is an acceptable and often more favourable approach.

It will, be appreciated that in the case of keloids, it will generally be preferred to avoid surgical intervention when possible. Given their high incidence of recurrence, and the fact that such recurrence is exacerbated by surgical intervention, it is important to be able to accurately diagnose keloids in order that suitable treatment regimes may be employed. Current treatment regimes for keloids include corticosteroid injections, cryotherapy, radiation therapy, silicone gel dressings and intra-lesional injection of agents intended to reduce the size of keloid scarring.

In present practice diagnosis of keloids is undertaken on the basis of the appearance of the scar. However, the accurate diagnosis of keloids is hampered by the fact that keloid morphology may be very similar to that of other pathological scars. The appearance of keloids and hypertrophic scars may be particularly similar. Hypertrophic scars resemble keloid scars in that they are also raised above the skin level. However, hypertrophic scars differ from keloids in that they remain within the boundaries of the original lesion, and may regress spontaneously several months after the initial injury. The visual similarity between keloid and hypertrophic scars means that diagnosis of a raised scar between these two distinct conditions is often confusing and cannot be accurately undertaken without long-term monitoring. There is a need for rapid and accurate means by which scars of interest may be diagnosed to indicate whether they are keloid in nature, or whether they belong to other pathological or excessive scarring types, such as hypertrophic scars.

Raised scars may often be assumed to be associated with keloid disease, and in the case of black patients an elevated scar will often be diagnosed by default as keloidal by many physicians (Rosenborough et al, 2004. J. Natl. Med. Assoc. 96, 108). This tendency can lead to the mis-identification of non-keloids scars (such as hypertrophic scars or very bad non-pathological scars) as keloids. It will be appreciated that this potential mis-diagnosis can result in inappropriate scar management decisions and can block the use of elective/scar revision surgery as a viable therapeutic approach in the case of such mis-diagnosed scars.

It is known that keloids are the only pathological dermal scars that grow beyond the boundaries of the original injury, as noted above. This property can provide a basis on which differential diagnosis between keloid and hypertrophic scars may be undertaken, although such diagnosis requires a very long time, given the need for prolonged observation of the scar to be diagnosed.

Other attempts to provide a basis on which tissues may be diagnosed as keloid or non-keloid have utilised histological assessments. Among the histological features suggested as providing suitable basis for diagnosis of keloids is the presence of so called “keloidal collagen”, a thickened hyalinized form of collagen, although this is not found in all keloid samples. Further features that may allow for the differentiation of keloids from other pathological scars (such as hypertrophic scars) are the presence of a non-flattened epidermis in keloids, non-fibrotic papillary dermis, the presence of a “tongue-like” advancing edge that surrounds keloid lesions (located below the normal-appearing epidermis and papillary dermis), presence of a horizontal cellular fibrous band located in the upper reticular dermis, and the presence of a prominent fascia-like band.

However, these histological cues for the diagnosis of keloids are also unsatisfactory. Not all of the features suggested as diagnostic markers are found in all keloid tissues, and similarly some of the suggested markers may also be found in non-keloid tissues.

Furthermore, the use of histological means for diagnosis of keloids requires considerable time to be expended in the preparation and analysis of histological samples, as well as the need for the application of skill and judgement on the part of the person undertaking such analysis.

Rapid and accurate methods and kits for the diagnosis of keloid scars will enable diagnosis to be undertaken with greater confidence. This will facilitate taking of correct decisions regarding the clinical treatment of skin lesions, and will be advantageous in treatment of both keloid and non-keloid lesions. In the case of tissues diagnosed as keloids it will be possible to avoid treatments that may otherwise exacerbate keloid recurrence and expansion, while such considerations will not be inappropriately applied in the treatment of non-keloid tissues. Furthermore, it is likely that early accurate diagnosis may have major benefits in relation to the success of palliative care regimes treatments, since many available treatments are believed to be more effective on less-mature scars.

The ability to differentiate between keloid-forming and non-keloid-forming patients may thus provide great advantages in terms of limiting surgery, and hence the risk of keloid formation, amongst those prone to keloid development, since it is generally considered that the prevention of keloid formation is of paramount importance in the management of keloid-forming patients, and avoidance of non-essential cosmetic surgery is recommended for these individuals.

In the light of the above it will be appreciated that there exists a well recognised need for the provision of new and alternative methods and kits for the diagnosis of keloids. Such methods and kits may preferably be suited to the safe and reliable diagnosis of keloids.

It is an aim of certain embodiments of the invention to provide novel methods and markers for the diagnosis of keloids. It is another aim of certain embodiments of the present invention to provide methods of diagnosis that allow a greater degree of certainty in diagnosis of a scar of interest as keloid or non-keloid than may be achieved by the prior art. It is another aim of certain embodiments of the invention to provide methods of diagnosis that allow greater speed of diagnosis, to determine whether a scar of interest is keloid or non-keloid, than do the methods of the prior art. It is still another aim of certain embodiments of the invention to provide methods for the diagnosis of a scar of interest as keloid or non-keloid that do not require the taking of large biopsies in order for a diagnosis to be made. It is still another aim of certain embodiments of the invention to provide methods, allowing the diagnosis of a scar of interest as keloid or non-keloid, that do not involve procedures that are likely to cause the recurrence and/or exacerbation of keloid formation.

According to a first aspect of the present invention there is provided a method for diagnosing a scar of interest as keloid or non-keloid, the method comprising:

comparing expression in a sample representative of gene expression in the scar of interest of at least one gene, selected from the group of genes set out in Table 1, with expression of the said at least one gene in a comparator tissue;
wherein decreased expression of said at least one gene in the scar of interest compared to expression of said at least one gene in the comparator tissue indicates that the scar of interest comprises a keloid.

In a second aspect of the invention there is provided a kit for diagnosing a scar of interest as keloid or non-keloid, the kit comprising:

i) at least one probe capable of binding specifically to a target molecule representative of expression in the scar of interest of at least one gene selected from the group set out in Table 1; and
ii) reference material able to indicate the level of expression of said at least one gene in comparator tissue.

It is preferred that the methods and kits of the invention to be used for in vitro diagnosis of a scar of interest as keloid or non-keloid.

Although the methods and kits of the invention are most suitable for use in diagnosis of human scars as keloid or non-keloid, they may also be useful in diagnosing similar conditions in non-human animals, such as “proud flesh” in horses.

The present invention is based on the identification by the inventors of a number of genes the decreased expression of which is diagnostic of keloid tissue. The inventors have found that comparison of the expression of one or more of these genes in a scar of interest with the expression occurring in a comparator tissue allows an accurate and rapid diagnosis as to whether the tissue is keloid or non-keloid. Identity of the scar of interest as keloid is indicated by a decrease in gene expression as compared to expression in the comparator tissue sample, whereas unchanged or increased expression in the scar of interest as compared to the comparator indicates that the scar of interest is non-keloid tissue.

The finding that decreased expression of the genes identified in Table 1 (i.e. the group comprising Gene Identification No. 1 to Gene Identification No. 590) may be used to diagnose whether a scar of interest is keloid or non-keloid is surprising, since although the expression of certain genes (such as those encoding VEGF, IGF1 and PAI1) has been linked to keloid tissue, the genes set out in Table 1 had never previously been identified as being associated with keloids, let alone as diagnostic of keloid scars.

In practicing the invention (whether by use of the methods, kits or arrays of the invention), expression of a selected gene (or genes) in a sample representative of gene expression in the scar of interest is compared with expression of the same gene (or genes) in a suitable comparator tissue. This comparison of expression of the selected gene (or genes) enables diagnosis of the scar of interest as keloid or non-keloid. If there is decreased expression of the selected gene (or genes) in the sample representative of gene expression in the scar of interest, as compared to in the comparator sample, then this indicates that the scar of interest comprises keloid tissue. If, on the other hand, there is no decrease in expression of the selected gene (or genes) in the sample representative of expression in the scar of interest (or, indeed, if there is an increase in expression of these genes), this indicates that the scar of interest does not comprise keloid tissue.

In general expression of selected genes in the scar of interest will be investigated by analysis of target molecules representative of gene expression. Suitable investigation may involve the analysis for presence or absence of such target molecules in a sample (qualitative analysis of gene expression, as discussed further elsewhere in the specification), or analysis of the relative abundance of target molecules in a sample (which may provide quantitative information as to gene expression, as considered in more detail elsewhere in the specification).

Gene expression in the comparator tissue may be represented by tissues or tissue extracts containing suitable target molecules, or may alternatively be represented by data setting out details of the gene expression levels in the comparator. The identification, isolation and analysis of suitable target molecules is discussed further elsewhere in the specification, as is the provision of information representative of gene expression in comparator tissue samples.

A comparator tissue, for the purposes of the present disclosure, is a tissue with which gene expression in a scar of interest can be compared, in order to allow diagnosis of the scar of interest as keloid or non-keloid. Specifically, the expression in the scar of interest of at least one gene set out in Table 1 is compared with expression of the same gene (or genes) in the comparator tissue.

A number of different tissue types may serve as suitable comparator tissues for use in accordance with the present invention. Suitable comparator tissues include normal skin. For the present purposes normal skin may be considered to be skin other than in a keloid scar, and preferably to be unscarred and unwounded skin.

Alternatively a comparator tissue suitable for use in accordance with the present invention may be tissue from a known keloid. For instance a suitable comparator tissue for use in accordance with the invention may comprise tissue from the skin adjacent to a known keloid (also referred to in the present specification as “extra-keloid comparator tissue”). Alternatively, a suitable comparator tissue may comprise tissue from the area at the periphery of a known keloid (also referred to herein as “peri-keloid comparator tissue”). In a further alternative, a suitable comparator tissue may comprise tissue from the interior part of a known keloid (also referred to as “intra-keloid comparator tissue”).

A “comparator sample” for the purposes of the present invention comprises any sample (such as a tissue extract, or the like, as considered elsewhere in the specification) that provides information as to the expression of a selected gene in the comparator tissue from which the comparator sample is derived.

Although the inventors have found that any of the genes represented by the group of genes set out in Table 1 may be used in accordance with the present invention, the inventors have further found that certain subsets of these genes have particular diagnostic value. These subsets are identified and considered in more detail below.

The inventors have noted that expression of certain genes set out in Table 1 varies between different areas of a keloid lesion. This information may be used to further refine diagnosis in accordance with the present invention (whether by methods, kits, or arrays of the invention).

The inventors have also found that preferred genes that may be investigated in the methods and kits of the invention may be selected with reference to their biological function.

A sample of interest, representative of a scar of interest to be diagnosed, may be further characterised with reference to the location from which it is derived in the scar. The inventors have found that characterisation of a sample of interest on this basis improves the efficacy of diagnoses undertaken in accordance with the invention. Samples of interest may be characterised as peri-lesional (which is to say samples taken from the periphery of a lesion comprising a scar of interest) and intra-lesional (those samples taken from the interior of a lesion comprising a scar of interest).

Genes from Table 1 that may be used in the diagnosis of peri-lesional samples of interest are set out in Table 2. It is a preferred embodiment that diagnosis in accordance with the invention (whether using the methods, kits or arrays of the invention) may be performed on the basis of comparison of one or more of the genes set out in Table 2.

Genes from Table 1 that may be used in the diagnosis of intra-lesional samples of interest are set out in Table 20. It is a preferred embodiment that diagnosis in accordance with the invention (whether using the methods, kits or arrays of the invention) may be performed on the basis of comparison of one or more of the genes set out in Table 20.

As set out above, comparator tissues suitable for use in diagnosis in accordance with the invention may also be characterised with reference to their source, as normal skin comparators; extra-keloid comparators; peri-keloid comparators; or intra-keloid comparators. The inventors have found that diagnosis in accordance with the invention may be improved by comparison of gene expression in a sample of interest characterised with reference to their location in a scar of interest, with gene expression of a comparator characterised in the manner set out above.

Thus it may be preferred that gene expression in a peri-lesional sample of interest is compared with gene expression in a normal skin comparator. Examples of suitable genes, expression of which may be compared between such samples in order to provide a diagnosis, are set out in Table 3. These genes may be further characterised with reference to their biological function. Accordingly those genes set out in Table 4 represent genes associated with cell motility, whereas those set out in Table 5 are associated with cell adhesion, the genes set out in Table 6 are associated with inflammation, and the genes set out in Table 7 are associated with the development of new blood vessels (particularly with angiogenesis).

Alternatively or additionally, it may be preferred that gene expression in a peri-lesional sample of interest is compared with gene expression in an extra-keloid comparator. Examples of suitable genes, expression of which may be compared between such samples in order to provide a diagnosis, are set out in Table 8. These genes may be further characterised with reference to their biological function. Accordingly those genes set out in Table 9 represent genes associated with cell motility, whereas those set out in Table 10 are associated with cell adhesion, the genes set out in Table 11 are associated with inflammation, and the genes set out in Table 12 are associated with the development of new blood vessels (particularly with angiogenesis).

Alternatively or additionally, it may be preferred that gene expression in a peri-lesional sample of interest is compared with gene expression in a peri-keloid comparator. Examples of suitable genes, expression of which may be compared between such samples in order to provide a diagnosis, are set out in Table 13. It will be appreciated that diagnosis on the basis of such comparisons will involve the gene expression in a tissue of interest and a comparator that are at different time-points in the healing process. Information regarding the time-points to be used is provided in Table 13. The genes set out in Table 13 may also be further characterised with reference to their biological function. Accordingly those genes set out in Table 14 are associated with cell adhesion, the genes set out in Table 15 are associated with inflammation, and the genes set out in Table 16 are associated with the development of new blood vessels (particularly with angiogenesis).

Alternatively or additionally, it may be preferred that gene expression in a peri-lesional sample of interest is compared with gene expression in an intra-keloid comparator. Examples of suitable genes, expression of which may be compared between such samples in order to provide a diagnosis, are set out in Table 17. These genes may be further characterised with reference to their biological function. Accordingly those genes set out in Table 18 represent genes associated with cell motility, whereas those set out in Table 19 are associated with inflammation.

Alternatively or additionally, it may be preferred that gene expression in an intra-lesional sample of interest is compared with gene expression in a normal skin comparator. Examples of suitable genes, expression of which may be compared between such samples in order to provide a diagnosis, are set out in Table 21.

Alternatively or additionally, it may be preferred that gene expression in an intra-lesional sample of interest is compared with gene expression in an extra-keloid comparator. Examples of suitable genes, expression of which may be compared between such samples in order to provide a diagnosis, are set out in Table 22. These genes may be further characterised with reference to their biological function. Accordingly those genes set out in Table 23 represent genes associated with cell motility.

Alternatively or additionally, it may be preferred that gene expression in an intra-lesional sample of interest is compared with gene expression in a peri-keloid comparator. Examples of suitable genes, expression of which may be compared between such samples in order to provide a diagnosis, are set out in Table 24. These genes may be further characterised with reference to their biological function. Accordingly those genes set out in Table 25 are associated with cell adhesion.

Alternatively or additionally, it may be preferred that gene expression in an intra-lesional sample of interest is compared with gene expression in an intra-keloid comparator. Examples of suitable genes, expression of which may be compared between such samples in order to provide a diagnosis, are set out in Table 26. It will be appreciated that diagnosis on the basis of such comparisons will involve the gene expression in a tissue of interest and a comparator that are at different time-points in the healing process. Information regarding the time-points to be used is provided in Table 26. The genes set out in Table 26 may also be further characterised with reference to their biological function. Accordingly those genes set out in Table 27 are associated with inflammation, and the genes set out in Table 28 are associated with the development of new blood vessels (particularly with angiogenesis).

It may be preferred that diagnosis in accordance with the present invention, whether carried out using the methods, kits or arrays of the invention, utilise comparison of one or more gene selected independently from one or more of the Tables 2 to 28.

A skilled person wishing to undertake a diagnosis in accordance with the invention may consider the nature of a sample that is available from a scar of interest, consider the nature of a comparator sample that is available, and thereby select appropriate genes expression of which may be compared with reference to the considerations set out above.

It is particularly preferred that the methods, kits or arrays of the invention may involve the comparison of genes selected from two or more of Tables 2 to 28. For example, preferred methods, kits or arrays may involve the comparison of at least one gene selected from each of two of Tables 2 to 28, more preferred methods, kits or arrays may involve comparison of at least one gene selected from each of three of Tables 2 to 28, even more preferred methods, kits or arrays may involve comparison of at least one gene selected from each of four of Tables 2 to 28, and most preferred methods, kits or arrays may involve comparison of at least one gene selected from each of five or more of Tables 2 to 28.

Diagnosis of a scar of interest as keloid or non-keloid in accordance with the present invention may be effected by comparing the expression in a sample representative of gene expression in the scar of interest with expression in a comparator sample of one gene selected from Table 1, however, it is preferred to utilise multiple genes from Table 1. Thus it may be preferred that diagnosis in accordance with the present invention may be effected by comparing the expression of up to five genes selected from Table 1. It is particularly preferred that diagnosis in accordance with the present invention is effected by comparing the expression of 5, 6, 7, 8, 9 or 10 genes selected from Table 1. Diagnosis may be effected by comparing expression of up to 20 or 50 genes selected from Table 1. Diagnosis in accordance with the present invention may be effected by comparing the expression of up to 100, 200, 300, 400 or even up to 500, genes selected from Table 1. Indeed it may in certain cases be preferred that diagnosis of a scar of interest as keloid or non-keloid in accordance with the present invention is effected by comparing the expression of 500 or more genes selected from Table 1. If so desired, a diagnosis can be made using any or all of the 590 genes represented in Table 1.

A scar of interest in the context of the present invention may be any scar for which it is desired to diagnose whether the scar comprises keloid or non-keloid tissue. It will be appreciated that dermal scars constitute preferred examples of suitable scars of interest. The ability to distinguish between keloids and other forms of severe or pathological scarring, such as hypertrophic scars, is of notable value. Such differentiation may allow the selection of clinical treatment that is appropriate to the type of scarring diagnosed. Accordingly, the use of the methods and kits of the present invention in effecting diagnosis of excessive or pathological dermal scars represents a particularly preferred example of their use.

A scar of interest may preferably be derived from an individual believed to be at elevated risk of keloid formation. Examples of such individuals include patients with a history of keloid formation, individuals of the African Continental Ancestry Group or individuals of the Asian Continental Ancestry Group.

Suitable scars of interest may be derived from individuals who have suffered injury to the skin. In particular these may include individuals suffering injury at a site where there is an elevated risk of keloid formation. Examples of such sites may typically include areas of high skin tension, such as the chest, back, shoulders, or neck. However, relevant sites may also include areas, such as the earlobes, that are common sites of keloid formation, although not subject to high skin tension.

Diagnosis using the methods, kits, and arrays of the invention may be useful in diagnosing scars of interest from patients who have experienced skin wounding, as well as in diagnosing scars of interest from patients who have experienced skin trauma.

For the purposes of the present invention “skin wounding” may be considered to comprise conditions or clinical situations in which partial or total penetration of the skin occurs, and also those in which partial or total destruction of one or more layers of the skin occurs. For example, wounds may include puncture wounds, incisional wounds, excisional wounds and partial or full thickness skin grafts (including both donor and recipient sites). Such wounds may be associated with surgical procedures or accidental injuries. Wounds may also include burn or scald injuries, resulting from exposure of the skin to substances at high or low temperatures sufficient to cause damage to the skin. Chemical “burns”, such as those caused by exposure of the skin to acid or alkali, and cosmetic procedures such as dermabrasion or exfoliation (included so-called “chemical peels” and “laser peels”) may also give rise to tissues for which it is wished to produce a diagnosis in accordance with the present invention.

For the purposes of the present invention “skin trauma” may be taken as referring to injuries that damage, but do not penetrate, the skin. Illustrative examples of injuries that may be considered as skin trauma include crush injuries to the skin, as well other “blunt” injuries.

Although the preceding paragraphs provide examples of individuals, or of scars of interest, that may particularly benefit from diagnosis in accordance with the present invention it will be appreciated that the methods, kits and arrays of the invention may be beneficially used in diagnosis of any scar of interest, particularly those that may be believed to be keloid scars. Generally tissues that are believed possibly to be keloid scars will be those that display one or more characteristics selected from the following group: an elevated profile compared to the surrounding skin; a lesion growing beyond its original boundaries; a lesion at the site of an earlier skin wound or trauma; hypo- or hyper-pigmentation compared to the surrounding skin.

Samples representative of gene expression in a scar of interest that may be used in accordance with the present invention encompass any sample that may provide information as to genes being expressed by the scar of interest.

Any suitable sample derived from the scar of interest may be used. Preferred sample include biopsies and, if available, samples of wound tissue, wound fluid, wound aspirates or wound exudates. Preferably such biopsies may be of a sort selected to reduce the level of injury inflicted to the patient, and thereby limit damage to those found to reduce the risk of (further) keloid formation. Such techniques may, for example, make use of needle biopsies in order to reduce the level of injury occurring. Any of the sample types discussed above may be used in diagnosis, in accordance with the invention, of the scar of interest from which the sample in question is derived.

Suitable samples may include tissue sections such as histological or frozen sections. Methods by which such sections may be prepared in such a way as to be able to provide information representative of gene expression in the scar of interest from which the section is derived will be well known to those skilled in the art, and should be selected with reference to the technique that it is intended to use when investigating gene expression.

Although the use of samples comprising a portion of the scar of interest is contemplated, it may generally be preferred that the sample representative of gene expression comprise a suitable extract taken from the scar of interest, said extract being capable of investigation to provide information regarding gene expression in the scar of interest. Suitable protocols which may be used for the production of tissue extracts capable of providing information regarding gene expression in a scar of interest will be well known to those skilled in the art. Preferred protocols may be selected with reference to the manner in which gene expression is to be investigated. Illustrative examples of protocols that may be used to produce tissue extracts representative of gene expression in a scar of interest are discussed below.

Suitable comparator samples, for use in accordance with methods, kits or arrays of the invention, may be selected with reference to the scar of interest in respect of which diagnosis is to be performed. Preferably the comparator tissue will be as well matched as possible to the scar of interest (matching may consider tissue type, tissue site, etc.). Sources and examples of suitable comparator samples will be apparent to those skilled in the art and include those derived from individuals that are not subject to keloid formation, as well as samples from keloid formers selected with reference to their location relative to a known keloid (i.e. non-keloid tissue, extra-keloid tissue, peri-lesional tissue, or intra-lesional tissue). It will be recognised that the skin constitutes a preferred source of comparator samples.

Suitable comparator samples may include portions of non-keloid tissues or organs including target molecules representative of gene expression (in which case the tissue should be preserved in such a manner that information regarding the expression of genes in the tissue may be extracted from the tissue, for example by analysis of the target molecules). Alternatively, suitable comparator samples may comprise tissue extracts incorporating extracted and/or isolated target molecules (such as mRNA or cDNA) that are representative of gene expression in the comparator sample. Relevant information regarding gene expression in comparator samples may also be provided in the form of data derived from such samples, as considered elsewhere in the specification.

Comparator samples from which information relating to the expression of selected genes may be derived include tissue samples and tissue extracts as considered herein with reference to samples derived from the scar of interest. For example, such information may be derived directly from a tissue or organ sample constituting the comparator sample, or from an extract capable of providing information regarding gene expression in the selected control sample. The expression of the selected gene, or genes, (selected from the group of genes set out in Table 1) in comparator samples of this type may be investigated using the methods described herein in connection with the investigation of gene expression in the scar of interest.

Although tissue or organ samples constituting comparator samples, or extracts from such samples, may be used directly as the source of information regarding gene expression in the comparator sample (as discussed elsewhere in the specification), it will generally be preferred that information regarding the expression of the selected gene (or genes) in the comparator sample be provided in the form of reference data. Such reference data may be provided in the form of tables indicative of gene expression in the chosen comparator tissue. Alternatively, the reference data may be supplied in the form of computer software containing retrievable information indicative of gene expression in the chosen comparator tissue. The reference data may, for example, be provided in the form of an algorithm enabling comparison of expression of at least one selected gene (or genes) in the scar of interest with expression of the same gene (or genes) in the comparator tissue sample.

In a preferred embodiment of the invention, a diagnosis as to whether the scar of interest is keloid or non-keloid may be delivered automatically on inputting results representative of expression of selected genes in the scar of interest into a predictive algorithm that has been trained upon data representative of gene expression in a suitable comparator sample. Well-established and commonly used classification systems include, but are not limited to, K-Nearest Neighbours, Centroid Classification, Linear Discriminant Analysis, Neural Networks and Support Vector Machines available, for example, in the Partek Genomics Suite software package (Partek Inc.).

A suitable sample representative of gene expression in a scar of interest or comparator sample may provide qualitatitive and/or quantitative information regarding gene expression. For the purpose of the present invention qualitative information regarding gene expression is to be considered to be information that provides identification as to genes expressed in a scar of interest or comparator sample, without providing information as to the relative amounts of expression (save as to whether a particular gene is, or is not, expressed). It will be appreciated that in some situations qualitative information may allow a sufficient comparison between expression in the scar of interest and the comparator sample to allow a diagnosis as to whether the scar of interest is keloid or non-keloid.

Qualitative information may be particularly suitable for diagnoses that are based on decreased expression of genes of Table 1 that are normally expressed in comparator samples, but are not expressed at all in keloids. In such cases the lack of expression of the gene the scar of interest will be sufficient to indicate an elevated risk of keloid formation. Examples include those genes identified by Gene Identification Numbers 2, 3 and 4 and it may be a preferred embodiment of the invention to investigate expression of these genes. The inventors have found that these genes may be used as the basis of a diagnosis when their expression is compared (quantitatively or qualitatively) between an intra-lesional scar of interest and a normal skin comparator.

It will, however, generally be preferred to use a sample capable of providing quantitative information regarding gene expression in the scar of interest or comparator sample. Such information allows ready comparison between the levels of expression in the scar of interest and the levels of expression in the comparator sample. For the purposes of the present invention quantitative information relating to gene expression may be taken to refer to either absolute or relative quantification. Methods by which absolute or relative quantitation may be achieved are discussed further below.

Samples representative of gene expression in the scar of interest or comparator sample will generally contain target molecules that are directly or indirectly representative of gene expression. Suitable samples may be provided in the form of tissue samples containing such target molecules, or, preferably as tissue extracts. A tissue extract representative of gene expression in a scar of interest will generally contain isolated target molecules that are representative of gene expression in the tissue from which the extract is obtained.

Suitable techniques by which tissue samples or tissue extracts may be obtained and prepared in order that they may provide information as to gene expression may be selected with reference to the type of target molecule that is to be employed. Examples of appropriate techniques that may be used will be readily apparent to the skilled person, however guidance as to suitable techniques is also provided elsewhere in the specification.

It will be appreciated that protein target molecules represent target molecules that are particularly amenable to direct detection. Such direct detection may provide qualitative or quantitative information as to the amount of the protein present in the scar of interest or comparator sample, thereby allowing comparison of expression.

In a preferred instance, the amount of certain target proteins present in a sample may also be assessed with reference to the biological activity of the target in the sample. Assessment and comparison of expression in this manner is particularly suitable in the case of protein targets having enzyme activity. Examples of genes set out in Table 1 having enzyme activity, and so particularly suitable for investigation in this manner, include those identified by Gene Identification Numbers 8, 22, 24, 29, 44, 46, 54, 56, 60, 69, 70, 75, 93, 94, 97, 102, 123, 133, 138, 147, 148, 150, 152, 159, 167, 170, 186, 194, 195, 209, 216, 221, 228, 232, 234, 239, 243, 262, 268, 289, 291, 293, 304, 306, 323, 324, 326, 357, 358, 359, 361, 366, 382, 385, 395, 398, 400, 402, 412, 419, 420, 437, 440, 446, 452, 456, 459, 460, 466, 467, 469, 472, 485, 486, 502, 505, 514, 516, 517, 534, 540, 558, 563, 569, 575, 581, 582 and 587. Enzyme activity of protein targets may, for example, be investigated by analysing breakdown of labelled enzyme substrate, and the amount of enzyme activity thereby correlated with gene expression occurring in the scar of interest or comparator sample. Merely by way of example, those enzymes identified by Gene Identification Nos. 56, 366, 412 and 581 all possess proteolytic activity, and it would therefore be possible to assess the presence or absence of these enzymes with reference to their ability to proteolytically degrade their substrates.

The presence or absence of target molecules in a tissue sample or extract will generally be detected using suitable probe molecules (although there may be some instances, such as those discussed above, where presence or absence of a target molecule may be determined directly without the need for a probe). Such detection will provide information as to gene expression, and thereby allow comparison between gene expression occurring in the scar of interest and expression occurring in the comparator sample. Diagnosis in accordance with the invention may be carried out based on such comparisons.

Probes will generally be capable of binding specifically to target molecules directly or indirectly representative of gene expression in the scar of interest or comparator sample. Binding of such probes may then be assessed and correlated with gene expression to allow an effective diagnostic comparison between gene expression in the scar of interest and in the comparator. Suitable probes that may be used in the methods, kits and arrays of the invention are discussed elsewhere in the specification.

Target molecules suitable for use in the methods, kits and arrays of the invention are molecules representative of gene expression either directly or indirectly, as considered in greater detail below. Target molecules may include mRNA gene transcripts, as well as natural and artificial products of such transcripts (e.g. proteins or cDNA respectively). It will be appreciated that samples for use in accordance with the present invention should be processed in a manner selected with reference to the nature of the target molecule that is to be used. Suitable protocols for processing of tissues to yield samples containing usable target molecules are discussed further below.

Suitable target molecules may comprise the direct products of gene expression. Such direct products of gene expression may, for example, comprise one or more gene transcripts representative of gene expression. The use of mRNA gene transcripts as target molecules allowing comparison of gene expression in the scar of interest with expression in the comparator sample is a preferred embodiment of the invention.

Alternatively, a sample representative of gene expression in the scar of interest or comparator sample may comprise target molecules that are indirectly representative of gene expression. Examples of such targets indirectly representative of gene expression may include natural products (such as proteins) that are produced on translation of a gene transcript, as well as artificial products generated from gene transcripts. Preferred examples of artificial target molecules generated from gene transcripts include cDNA and cRNA, either of which may be generated using well known protocols or commercially available kits or reagents.

For example, in a preferred embodiment, RNA representative of gene expression in a scar of interest or comparator sample may be isolated through a process of lysing cells taken from a suitable sample (which may be achieved using a commercially available lysis buffer such as that produced by Qiagen Ltd.) followed by centrifugation of the lysate using a commercially available nucleic acid separation column (such as the RNeasy midi spin column produced by Qiagen Ltd). Other methods for RNA extraction include variations on the phenol and guanidine isothiocyanate method of Chomczynski, P. and Sacchi, N. (1987) Analytical Biochemistry 162, 156. “Single Step Method of RNA Isolation by Acid Guanidinium Thiocyanate-Phenol-Chloroform Extraction.” RNA obtained in this manner may constitute a suitable target molecule itself, or may serve as a template for the production of target molecules representative of gene expression.

It may be preferred that RNA derived from a scar of interest or comparator sample may be used as substrate for cDNA synthesis, for example using the Superscript System (Invitrogen Corp.). The resulting cDNA may then be converted to biotinylated cRNA (for instance using the BioArray RNA Transcript labelling kit from Enzo Life Sciences Inc.) and this cRNA purified from the reaction mixture (for instance using an RNeasy mini kit from Qiagen Ltd).

In the case of protein target molecules, gene expression may be assessed with reference to the total amount of the protein target present. Suitable techniques for the measurement of the amount of a protein target present in a sample representative of gene expression in a scar of interest or comparator sample include, but are not limited to, aptamers and antibody-based techniques, such as radio-immunoassays (RIAs), enzyme-linked immunoassays (ELISAs) and Western blotting, immuno-PCR and multiplex approaches such as those using beads or microspheres (for example xMap technology from Luminex Inc), (Bloom and Dean (2003) Biomarkers in Clinical Drug Development; Crowther (1995) Elisa Theory and Practice (Humana Press); Singh et al (1993) Diagnostics in the year 2000: Antibody, Biosensor and nucleic acid Technologies (Van Nostrand Reinhold, New York); Niemeyer C M, Adler M, Wacker R. Immuno-PCR: high sensitivity detection of proteins by nucleic acid amplification. Trends Biotechnol. 2005 April; 23(4):208-16; Abreu I, Laroche P, Bastos A, Issert V, Cruz M, Nero P, Fonseca J E, Branco J, Machado Caetano J A. Multiplexed immunoassay for detection of rheumatoid factors by FIDISTM technology. Ann N Y Acad Sci. 2005 June; 1050:357-63).

The disclosures of the documents set out in the preceding paragraphs are incorporated by reference, insofar as they describe methods that may useful to the skilled person in practising the present invention.

In the event that expression of one or more genes from Table 1 in a comparator sample is to be investigated via processing of a tissue or organ sample constituting the comparator sample, or by processing of a tissue extract representative of gene expression in the comparator sample, for example to isolate suitable target molecules, it is preferred that such processing is conducted using the same methods used to process the sample from the scar of interest. Such parallel processing of samples from both the scar of interest and comparator tissue allows a greater degree of confidence that comparisons of gene expression in these tissues will be normalised relative to one another (since any artifacts associated with the selected method by which tissue is processed and gene expression investigated will be applied to both the scar of interest and comparator samples).

Furthermore, the parallel processing of the comparator sample in this manner provides an “internal control” that will allow the practitioner to confirm that processing has occurred successfully. Since the practitioner will be aware that the selected one or more genes from Table 1 that have been selected for comparison of expression are normally expressed by comparator tissues, the practitioner will be able to discount any instances of processing (for investigation of gene expression) which give rise to assays indicating that expression of these internal controls cannot be detected (since these results will likely be as a result of a processing error leading to artificially low readings). Such results may otherwise give rise to an incorrect assessment that the scar of interest comprises keloid tissue (since the same artificial decrease in assessed expression would be noted in respect of the selected gene or genes from Table 1).

Samples representative of gene expression in a scar of interest, or a comparator tissue, may be manipulated prior to effecting comparison of gene expression. Such manipulation may, for example, be designed to make comparison of expression easier, or to increase the information made available by the comparison. Examples of suitable ways in which such samples may be manipulated are considered below.

Preferably the methods or kits of the invention will provide means by which the expression data relating to the scar of interest and comparator tissue may be “normalised” with respect to one another. Normalisation ensures that comparisons being made are “like for like”, and suitable parameters for use in normalisation are well known to those skilled in the art. Purely by way of illustration, normalisation may be effected with reference to cell numbers in the samples to be compared; and/or total protein content of samples to be compared; and/or total nucleic acid content of samples to be compared; and/or expression level of one or more genes the expression of which does not change between keloid and non-keloid tissues. Alternatively or additionally, a suitable control may involve assessing expression of one or more genes known to be expressed in keloids. Detection of the expression of such genes (in combination with the reduced expression of one or more of the genes set out in Table 1) will provide a suitable control against which gene expression can be referenced. Suitable examples of such genes are considered elsewhere in the specification.

The inventors have found that preferred samples representative of gene expression for use in accordance with the present invention are those samples comprising nucleic acid target molecules representative of gene expression. For the purposes of the present invention a nucleic acid target is a nucleic acid the presence or absence of which is to be detected, or the amount of which present is to be quantified. Such detection or quantification will allow a diagnostic comparison of expression to be effected. A target nucleic acid may preferably have a sequence that is complementary to the nucleic acid sequence of a corresponding probe directed to the target. A nucleic acid target in accordance with the present invention may encompass both a specific subsequence of a larger nucleic acid to which a probe is directed or, alternatively, the overall sequence (e.g. complete mRNA transcript) whose expression level it is desired to detect. Suitable nucleic acid targets may include both RNAs and DNAs, and encompass both naturally occurring and artificial nucleic acids.

It will be understood that target nucleic acids suitable for use in accordance with the invention need not comprise “full length” nucleic acids (e.g. full length gene transcripts), but need merely comprise a sufficient length to allow specific binding of probe molecules.

It will be understood that “nucleic acids” or “nucleic acid molecules” for the purposes of the present invention refer to a deoxyribonucleotide or ribonucleotide polymers in either single- or double-stranded form. Furthermore, unless the context requires otherwise, these terms should be taken to encompass known analogues of natural nucleotides that can function in a similar manner to naturally occurring nucleotides.

mRNA constitutes a preferred form of target molecule that may be used in the methods and kits of the invention. mRNA gene transcripts are directly representative of gene expression in the scar of interest or comparator sample.

It will be recognised that mRNA, representative of gene expression, may be found directly in a scar of interest or comparator sample, without the need for mRNA extraction or purification. For example, mRNA present in, and representative of gene expression in, a scar of interest or comparator sample may be investigated using appropriately fixed sections or biopsies of such a tissue. The use of samples of this kind may provide benefits in terms of the rapidity with which comparisons of expression can be made, as well as the relatively cheap and simple tissue processing that may be used to produce the sample. In situ hybridisation techniques represent preferred methods by which gene expression may be investigated and compared in tissues samples of this kind. Techniques, for the processing of scars of interest that maintain the availability of RNA representative of gene expression in the scar of interest or comparator sample are well known to those of skill in the art.

However, techniques by which mRNAs representative of gene expression in a scar of interest or comparator sample may be extracted and collected are well known to those skilled in the art, and the inventors have found that such techniques may be advantageously employed in accordance with the present invention. Samples comprising extracted mRNA from a scar of interest or comparator sample may be preferred for use in the methods and kits of the invention, since such extracts tend to be more readily investigated than is the case for samples comprising the original tissues. For example, suitable target molecules allowing for comparison of gene expression may comprise the total RNA isolated from a sample of the scar of interest, or a sample of comparator tissue.

Furthermore, extracted RNA may be readily amplified to produce an enlarged mRNA sample capable of yielding increased information on gene expression in the scar of interest or comparator sample. Suitable examples of techniques for the extraction and amplification of mRNA populations are well known, and are considered in more detail below.

By way of example, methods of isolation and purification of nucleic acids to produce nucleic acid targets suitable for use in accordance with the invention are described in detail in Chapter 3 of Laboratory Techniques in Biochemistry and Molecular Biology:

Hybridization With Nucleic Acid Probes, Part I. Theory and Nucleic Acid Preparation, P. Tijssen, ed. Elsevier, N.Y. (1993).

In a preferred method, the total nucleic acid may be isolated from a given sample using, for example, an acid guanidinium-phenol-chloroform extraction method.

In the event that it is desired to amplify the nucleic acid targets prior to investigation and comparison of gene expression it may be preferred to use a method that maintains or controls for the relative frequencies of the amplified nucleic acids in the scar of interest or control tissue from which the sample is derived.

Suitable methods of “quantitative” amplification are well known to those of skill in the art. One well known example, quantitative PCR involves simultaneously co-amplifying a control sequence whose quantities are known to be unchanged between comparator samples and those from the scar of interest. This provides an internal standard that may be used to calibrate the PCR reaction.

In addition to the methods outlined above, the skilled person will appreciate that any technology coupling the amplification of gene-transcript specific product to the generation of a signal may also be suitable for quantitation. A preferred example employs convenient improvements to the polymerase chain reaction (U.S. Pat. No. 4,683,195 and 4683202) that have rendered it suitable for the exact quantitation of specific mRNA transcripts by incorporating an initial reverse transcription of mRNA to cDNA. Further key improvements enable the measurement of accumulating PCR products in real-time as the reaction progresses. Examples of suitable technologies using fluorescent resonance energy transfer to generate a quantitative gene-specific signal include Taqman (U.S. Pat. No. 5,210,015 and 5487972), molecular beacons (WO-95/13399) and scorpions (US2005/0164219). The parallel quantitation of multiple transcripts is possible via the use of different fluorescent moieties for each gene target.

Other suitable amplification methods include, but are not limited to Nucleic acid sequence based amplification (NASBA) (Saad F. UPM3: review of a new molecular diagnostic urine test for prostate cancer. Can J Urol. 2005 February; 12 Suppl 1:40-3); Rolling Circle Amplification (RCA) (Gomez K F, Lane J, Cunnick G, Grimshaw D, Jiang W G, Mansel R E. From PCR to RCA: a surgical trainee's guide to the techniques of genetic amplification. Eur J Surg Oncol. 2002 August; 28(5):554-9); Branched Chain Nucleic Acids (BCNA) (Andras S C, Power J B, Cocking E C, Davey M R. Strategies for signal amplification in nucleic acid detection. Mol Biotechnol. 2001 September; 19(1):29-44); the invader assay (de Arruda M, Lyamichev V I, Eis P S, Iszczyszyn W, Kwiatkowski R W, Law S M, Olson M C, Rasmussen E B. Invader technology for DNA and RNA analysis: principles and applications. Expert Rev Mol Diagn. 2002 September; 2(5):487-96); ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the mRNA transcripts from a tissue representative of gene expression in a scar of interest or comparator sample may be reverse transcribed with a reverse transcriptase and a promoter consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 (1992) provide a protocol that uses two rounds of amplification via in vitro transcription to achieve greater than 10⁶ fold amplification of the original starting material, thereby permitting expression monitoring even when only a small sample of the scar of interest is available.

It will be appreciated by one of skill in the art that the direct transcription method described above leads to the production of antisense RNA (aRNA) targets. In such cases probes, such as oligonucleotide probes, to be used to investigate and compare gene expression should be chosen to be complementary to sequences or sub-sequences of the antisense nucleic acids.

The skilled person will further appreciate that artificial nucleic acid molecules may also be used in the comparison of gene expression. Examples of artificial target molecules suitable for use in accordance with the present invention include cDNAs made by reverse transcription of mRNA or second strand cDNA or RNA (cRNA) transcribed from a double stranded cDNA intermediate. Methods for the production of cDNAs and cRNAs are well documented in the art, and will be known to the skilled person, and indeed kits and reagents suitable for their production are readily commercially available.

For the purposes of the present invention, a sample that is “representative” of gene expression in a scar of interest is to be considered to encompass any sample providing information as to the expression of genes in the scar of interest. For example, a representative sample may provide information as to all the genes expressed in the scar of interest, and preferably the relative levels of expression of said genes.

In a preferred embodiment, a representative sample is one in which the concentration of target molecules is proportional to the concentration of mRNA gene transcripts of the gene (or genes) expression of which, in the scar of interest, is to be compared to comparators. While it is preferred that the proportionality be relatively strict (e.g., a doubling in the number of mRNA gene transcript occurring in the scar of interest leading to a doubling in the number of corresponding target molecules present in the sample), the skilled person will appreciate that the proportionality can be more relaxed and even non-linear. For example, an assay where a five fold difference in concentration of the mRNA gene transcripts in the scar of interest results in a three to six fold difference in the concentration of target molecules in the representative sample is sufficient for most purposes.

In the event that more precise quantification is required, serial dilutions of “standard” target molecules can be used to prepare calibration curves according to methods well known to those skilled in the art. More preferably quantitation of target molecules will be relative and normalised with respect to each other and/or “housekeeping” genes whose expression levels are not increased in keloid forming as compared to non-keloid forming tissues. Examples of such genes include exportin 7 (XPO7), Cleavage and Polyadenylation Specific Factor 4, 30 kDa (CPSF4), F-box only protein 7 (FBXO7), ADP-ribosylation factor 1 (ARF1), signal sequence receptor, beta (SSR2) and methionine-tRNA synthetase (MARS).

It will, of course, be appreciated that in the case of a qualitative sample or samples (where simple detection of the presence or absence of gene expression is desired) no such elaborate control or calibration is required.

Although it may be preferred in many instances that the representative sample provides information as to all genes expressed in the scar of interest or comparator sample, a suitable representative sample may alternatively provide information relating to the expression of only a sub-set of the total number of genes undergoing expression.

In many cases it may be preferred to assess the degree of gene expression in a scar of interest or comparator sample using probe molecules capable of indicating the presence of target molecules (representative of one or more of the genes set out in Table 1) in the relevant sample.

The use of target molecules and probes in methods, kits or assays in accordance with the present invention may confer increased sensitivity on the methods of the invention. This may lead to an increased ability to discriminate between otherwise small differences between expression in the scar of interest and expression in the comparator sample. This will have appreciable benefits on diagnosis in accordance with the invention.

Generally, suitable probes for use in the present invention will bind to their target molecules, and thereby allow detection of the target molecule (this detection being indicative of expression of the gene selected from Table 1 represented by the target molecule).

It may be preferred that probes for use in accordance with the invention allow replication of the target molecules (suitably in combination with the probe molecule). Replication in this manner produces a greater number of target molecules, and thus allows further binding of the labelled probe. In turn, the increased amount of labelled probe thus bound amplifies the detectable signal indicative of gene expression.

Probes for use in the methods and kits of the invention may be selected with reference to the product (direct or indirect) of gene expression to be investigated. Examples of suitable probes include oligonucleotide probes, antibodies, aptamers, and binding proteins or small molecules having suitable specificity.

Oligonucleotide probes constitute preferred probes suitable for use in accordance with the methods and kits of the invention. The generation of suitable oligonucleotide probes is well known to those skilled in the art (Oligonucleotide synthesis: Methods and Applications, Piet Herdewijn (ed) Humana Press (2004).). Oligonucleotide and modified oligonucleotides are commercially available from numerous companies.

An oligonucleotide is a single-stranded nucleic acid ranging in length from 2 to about 500 nucleotide bases, preferably from about 5 to about 50 nucleotides, more preferably from about 10 to about 40 nucleotides and most preferably from about 15 to about 40 nucleotides in length. Suitable hybridization methods, conditions, times, fluid volumes, and suitable methods by which hybridisation of oligonucleotide probes may be detected are as described elsewhere in the present specification.

For the purposes of the present invention an oligonucleotide probe may be taken to comprise an oligonucleotide capable of hybridising specifically to a target nucleic acid of complementary sequence through one or more types of chemical bond. Such binding may usually occur through complementary base pairing, and usually through hydrogen bond formation. Suitable oligonucleotide probes may include natural (ie., A, G, C, or T) or modified bases (7-deazaguanosine, inosine, etc.). In addition, a linkage other than a phosphodiester bond may be used to join the bases in an oligonucleotide probe, so long as this variation does not interfere with hybridisation of the oligonucleotide probe to its target. Thus, oligonucleotide probes suitable for use in the methods and kits of the invention may be peptide nucleic acids in which the constituent bases are joined by peptide bonds rather than phosphodiester linkages.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Considerations for the design and selection of probes suitable for use with antisense nucleic acid targets (aRNA) have been discussed above. In the case that the nucleic acid targets comprise sense nucleic acids, suitable oligonucleotide probes may be selected to be complementary to sequences or sub-sequences of the sense nucleic acids. In the case of nucleic acid targets that are double stranded, suitable probes may be of either sense as the nucleic acid targets will provide both sense and antisense strands.

Antibodies suitable for use in the methods or kits of the invention may be used to detect target molecules, such as proteins, that represent gene expression in a scar of interest.

Antibodies that may be used to investigate gene expression in accordance with the methods and kits of the present invention include monoclonal antibodies and polyclonal antibodies, as well as fragments of such antibodies, including, but not limited to, Fab or F(ab′)hd 2, and Fv fragments.

Methods suitable for the generation and/or identification of antibodies capable of binding specifically to a given target are well known to those skilled in the art. In general suitable antibodies may be generated by the use of the isolated target as an immunogen. This immunogen is administered to a mammalian organism, such as, but not limited to, a rat, rabbit, goat or mouse, and antibodies elicited as part of the immune response. Generally antibodies will be used in the context of the methods and kits of the invention to bind to protein products of gene expression. Suitable immunogens may include the full-length protein to be investigated, or an antigenic peptide fragment thereof.

Monoclonal antibodies can be produced by hybridomas, immortalized cell lines capable of secreting a specific monoclonal antibody. The immortalized cell lines can be created in vitro by fusing two different cell types, usually lymphocytes, one of which is a tumour cell.

Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA).

Aptamers may be used to bind both nucleic acid and non-nucleic acid targets. Accordingly aptamers are suitable probes for use in the investigation of gene expression products including RNA, DNA and small molecules or proteins. Preferably aptamers may be used to investigate gene expression products having a molecular weight of between 100 and 10,000 Da. ssDNA aptamers may be preferred for use in the investigation of gene expression products comprising DNA.

Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

The use of aptamers for investigation of gene expression in accordance with the methods and kits of the invention may be advantageous, since aptamers have relatively stable shelf lives. Aptamers suitable for use in the methods and/or kits of the invention may preferably be stabilized by chemical modifications (for example 2′-NH₂ and 2′-F modifications).

Photoaptamers are a subclass of aptamers incorporating at least one bromo-deoxyuridine (BrdU) in place of a thymidine (T) nucleotide. The presence of the BrdU enables photoaptamers to form a specific covalent crosslink with their target ligands when exposed to ultraviolet light. Because crosslinking requires both affinity-based binding and close proximity between a BrdU (at a specific location in the photoaptamer) and an amino acid (at a specific location in the target ligand), photoaptamers may be preferred for use in the methods and kits of the invention when increased specificity of binding with a gene expression product is required.

Suitable methods by which gene expression may be compared in accordance with the present invention may be selected in the light of the considerations referred to in the preceding pages.

In general methods for analysis may be selected based on the nature of a target molecule to be investigated, and suitable selection criteria may distinguish between nucleic acid and protein target molecules.

However, as set out above, it may generally be preferred to investigate and compare gene expression using oligonucleotide probes capable of binding to nucleic acid target molecules.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies previously described herein, and technologies where quantitation of amplified species occurs subsequent to amplification, such as array technologies.

Array technologies involve the hybridisation of samples, representative of gene expression within the scar of interest or comparator sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonucleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane. More details of suitable conditions in which hybridisation may be effected are provided below, as are examples of techniques by which hybridisation may be detected.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a scar of interest may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those set out in Table 1. Accordingly it will be recognised that such oligonucleotide arrays may be particularly preferred in embodiments of the methods or kits of the invention where it is desired to compare expression of more than one gene selected from Table 1 in order to effect a diagnosis.

In a preferred embodiment investigation of gene expression using oligonucleotide arrays may be effected by hybridisation of oligonucleotide probes and nucleic acid targets at low stringency followed by at least one wash at higher stringency. Low stringency conditions suitable for use in accordance with these embodiments may comprise a reaction temperature of about 20° C. to about 50° C. (more preferably about 30° C. to about 40° C., and most preferably about 37° C.) and 6×SSPE-T buffer (or lower). Suitable hybridisation protocols may include subsequent washes at progressively increasing stringency until a desired level of hybridisation specificity is reached. Hybridisation stringency may also be varied by electronic means, for example as provided by Nanogen Inc. (Sosnowski R, Heller M J, Tu E, Forster A H, Radtkey R. Active microelectronic array system for DNA hybridization, genotyping and pharmacogenomic applications. Psychiatr Genet. 2002 December; 12(4):181-92).

Suitable techniques for the detection of hybridisation between oligonucleotide probes and nucleic acid targets are considered further below.

The identity of selected oligonucleotide probes incorporated in arrays may be altered to allow more detailed selection of the genes, the expression of which is to be compared. For example arrays suitable for use in the methods or kits of the invention may comprise one or more oligonucleotide probes selected with reference to the differential expression of selected genes from Tables 1 to 28 as considered previously.

Alternatively, assessment of gene expression in a scar of interest or comparator sample based on levels of nucleic acids sequences (such as mRNA or DNA) in a sample representative of gene expression in the scar of interest or comparator may be undertaken using other suitable techniques that will be apparent to the skilled person. For example, northern blotting provides a sensitive method by which levels of mRNA representative of gene expression in a scar of interest or comparator sample may be assessed.

Other suitable methodologies that may be used in the comparison of nucleic acid targets representative of gene expression include, but are not limited to, nucleic acid sequence based amplification (NASBA); rolling circle DNA amplification (RCA); branched chain nucleic acid and invader assays; the use of aptamers, antibodies or antibody derivatives (Singh et al, 1993; Boeckh and Boivin 1998; Bloom and Dean, 2003; Jain, 2004; Millar and Moore, 2004; Olson, 2004; Yang and Rothman, 2004).

As described previously, gene expression in a scar of interest or comparator sample may alternatively be investigated using samples comprising proteins representative of gene expression. Suitable techniques by which such protein samples may be investigated to assess gene expression include, but are not limited to, aptamer detection; mass spectrometry; nuclear magnetic resonance (NMR); antibody-based methods such as immuno-PCR and multiplex approaches such as those using arrays, beads or microspheres (for example xMap technology from Luminex Inc), ELISA, RIA and Western blotting; and other methods well known to those skilled in the art (Bloom and Dean (2003) Biomarkers in Clinical Drug Development; Crowther (1995) Elisa Theory and Practice (Humana Press); Singh et al (1993) Diagnostics in the year 2000: Antibody, Biosensor and nucleic acid Technologies (Van Nostrand Reinhold, New York); Niemeyer C M, Adler M, Wacker R. Immuno-PCR: high sensitivity detection of proteins by nucleic acid amplification. Trends Biotechnol. 2005 April; 23(4):208-16; Abreu I, Laroche P, Bastos A, Issert V, Cruz M, Nero P, Fonseca J E, Branco J, Machado Caetano J A. Multiplexed immunoassay for detection of rheumatoid factors by FIDISTM technology. Ann N Y Acad Sci. 2005 June; 1050:357-63).

For instance, expression of proteins having enzymatic activity may be investigated and compared using assays based around activity of the protein in question. Enzymatic protein extracts (here constituting samples representative of gene expression in the scar of interest or comparator sample) may, for example, be incubated with samples comprising known quantities of the appropriately labelled substrate. The amount of enzymatic activity, and hence an indication of the level of gene expression in the scar of interest or comparator sample, may be determined by the amount of substrate converted by the enzyme.

Detection of probe or target molecules can be facilitated by coupling (i.e., physical linking) of such molecules to a detectable moiety. Alternatively suitable probe or target molecules may be synthesised such that they incorporate detectable moieties. Techniques that may be used in the coupling or incorporation of detectable moieties in probe or target molecules suitable for use in the method, kits or arrays of the invention are considered below.

Examples of detectable moieties that may be used in the labelling of probes or targets suitable for use in accordance with the invention include any composition detectable by spectroscopic, photochemical, biochemical, immunochemical, electrical, optical or chemical means. Suitable detectable moieties include various enzymes, prosthetic groups, fluorescent materials, luminescent materials, bioluminescent materials, radioactive materials and colorimetric materials. These detectable moieties are suitable for incorporation in all types of probes or targets that may be used in the methods or kits of the invention unless indicated to the contrary.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, phycoerythrin, texas red, rhodamine, green fluorescent protein, and the like; an example of a luminescent material includes luminol; examples of bioluminescent materials include luciferase, luciferin, and aequorin; examples of suitable radioactive material include ¹²⁵I, ¹³¹I, ³⁵S, ³H, ¹⁴C, or ³²P; examples of suitable colorimetric materials include colloidal gold or coloured glass or plastic (e.g., polystyrene, polypropylene, latex, etc.) beads.

Means of detecting such labels are well known to the skilled person. For example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the coloured label.

In a preferred embodiment of the invention fluorescently labelled probes or targets may be scanned and fluorescence detected using a laser confocal scanner.

In the case of labelled nucleic acid probes or targets suitable labelling may take place before, during, or after hybridisation. In a preferred embodiment, nucleic acid probes or targets for use in the methods or kits of the invention are labelled before hybridisation. Fluorescence labels are particularly preferred and, where used, quantification of the hybridisation of the nucleic acid probes to their nucleic acid targets is by quantification of fluorescence from the hybridised fluorescently labelled nucleic acid. More preferably quantitation may be from a fluorescently labelled reagent that binds a hapten incorporated into the nucleic acid.

In a preferred embodiment of the invention analysis of hybridisation may be achieved using suitable analysis software, such as the Microarray Analysis Suite (Affymetrix Inc.) and diagnosis automated by use of classification software (for example Partek Genomics Suite from Partek Inc).

Effective quantification may be achieved using a fluorescence microscope which can be equipped with an automated stage to permit automatic scanning of the array, and which can be equipped with a data acquisition system for the automated measurement, recording and subsequent processing of the fluorescence intensity information. Suitable arrangements for such automation are conventional and well known to those skilled in the art.

In a preferred embodiment, the hybridised nucleic acids are detected by detecting one or more detectable moieties attached to the nucleic acids. The detectable moieties may be incorporated by any of a number of means well known to those of skill in the art.

However, in a preferred embodiment, such moieties are simultaneously incorporated during an amplification step in the preparation of the sample nucleic acids (probes or targets). Thus, for example, polymerase chain reaction (PCR) using primers or nucleotides labelled with a detectable moiety will provide an amplification product labelled with said moiety. In a preferred embodiment, transcription amplification using a fluorescently labelled nucleotide (e.g. fluorescein-labelled UTP and/or CTP) incorporates the label into the transcribed nucleic acids.

Alternatively, a suitable detectable moiety may be added directly to the original nucleic acid sample (e.g., mRNA, polyA mRNA, cDNA, etc. from the scar of interest) or to an amplification product after amplification of the original nucleic acid is completed. Means of attaching labels such as fluorescent labels to nucleic acids are well known to those skilled in the art and include, for example nick translation or end-labelling (e.g. with a labeled RNA) by kinasing of the nucleic acid and subsequent attachment (ligation) of a nucleic acid linker joining the sample nucleic acid to a label (such as a suitable fluorophore).

As set out previously, in addition to the methods and kits described above, the invention also provides a kit for diagnosing a scar of interest as keloid or non-keloid, the kit comprising:

i) at least one probe capable of binding specifically to a target molecule representative of expression in a scar of interest of at least one gene selected from the group set out in Table 1; and
ii) reference material able to indicate the level of expression of said at least one gene in a comparator sample.

Preferably kits in accordance with this aspect of the invention may further comprise assay control material able to indicate that an assay has been performed correctly. Suitably such assay control material may include target molecules representative of expression of genes the expression of which does not vary between keloid and non-keloid tissues. Suitable examples of such housekeeping genes are considered elsewhere in the specification, and target molecules representative of expression of any of these genes may be advantageously provided in the kits of the invention. The provision of housekeeping genes of this sort in known quantities may provide a “standard” against which assay results may be normalised.

It may be preferred that a kit according to the present invention further comprises material (such as target molecules) representative of one or more genes whose expression is increased in keloid tissue. The provision of such genes may increase the ability to discriminate a biologically meaningful result from a change in the absolute input material or a change in the efficiency of any assay process. For example, lysyl oxidase displays a 3-fold higher expression in keloid tissue than in non-keloid tissue. Lysyl oxidase is a key enzyme involved in collagen cross-linking and has previously been shown to be highly expressed in fibrotic tissue.

Kits of the invention may further comprise materials for the preparation of a population of target molecules representative of gene expression in a scar of interest (or in a comparator tissue). Such materials may be suitable for the preparation of a population of nucleic acid target molecules. Alternatively such materials may be suitable for the preparation of a population of protein target molecules. It may be preferred that the kits comprise materials for the preparation of a population of labelled target molecules representative of gene expression in a scar of interest or comparator tissue.

It is also preferred that kits of the present invention may further comprise an algorithm or reference data/material able to indicate that the level of expression of said at least one gene, selected from the group set out in Table 1, in the scar of interest is diagnostic that the scar of interest is keloid tissue.

The algorithm may be provided in the form of a mathematical model of the difference in gene expression of said at least one gene, selected from the group set out in Table 1, between comparator data and data from scars of interest (such as known patient data). This mathematical model may then be deployed on gene expression data of said at least one gene, selected from the group set out in Table 1, from a new patient sample. The output thus generated will thus provide a diagnosis as to whether a scar of interest comprises keloid or non-keloid tissue.

Probes for inclusion in kits in accordance with this second aspect of the invention may be selected using the same criteria as for the first aspect of the invention. Suitable probes may be selected from the group comprising oligonucleotide probes, antibodies, aptamers and specific binding proteins.

Kits in accordance with the present invention may preferably comprise probes capable of binding specifically to target molecules representative of expression of up to five genes selected from the group set out in Table 1 (i.e. target molecules representative of the expression of up to five genes selected from Table 1). It is particularly preferred that kits of the invention comprise probes capable of binding 5, 6, 7, 8, 9 or 10 such target molecules. Kits may include probes capable of binding to up to 20 or up to 50 genes selected from those set out in Table 1. Suitable kits may comprise probes capable of binding to up to 100, 200, 300, 400 or 500 such target molecules. Indeed, kits of the invention may comprise probes capable of binding specifically to 500 or more target molecules, and may even comprise probes capable of binding specifically to targets representative of expression of all 590 of the genes set out in Table 1.

A kit of the invention will comprise probes capable of binding to target molecules representative of expression of at least one gene selected from Table 1, and/or probes capable of binding to target molecules representative of expression of at least one gene selected from Table 2, and/or probes capable of binding to target molecules representative of expression of at least one gene selected from Table 3, and/or probes capable of binding to target molecules representative of expression of at least one gene selected from Table 8, and/or probes capable of binding to target molecules representative of expression of at least one gene selected from Table 13, and/or probes capable of binding to target molecules representative of expression of at least one gene selected from Table 17, and/or probes capable of binding to target molecules representative of expression of at least one gene selected from Table 20, and/or probes capable of binding to target molecules representative of expression of at least one gene selected from Table 22, and/or probes capable of binding to target molecules representative of expression of at least one gene selected from Table 24, and/or probes capable of binding to target molecules representative of expression of at least one gene selected from Table 26.

Kits of the invention may include probes capable of binding to target molecules representative of gene expression of any of the genes set out in any one of (or any combination of) Tables 2 to 28.

The probes provided in the kits of the invention may preferably be labelled probes. Labelled probes may comprise any detectable moiety considered in connection with the first aspect of the invention. Preferred labelled probes may be chosen from the group comprising haptens, fluorescently labelled probes, radioactively labelled probes and enzymatically labelled probes.

The reference material provided in kits of the invention may comprise a library of nucleic acid targets representative of expression in an appropriate comparator sample of one or more genes selected from the group of genes set out in Table 1.

In a preferred embodiment the reference material may comprise recorded information regarding the level of expression of one or more genes selected from the group of genes set out in Table 1 in keloid and non-keloid tissue.

In a most preferred example the reference data may be used to create an algorithm which may deliver a diagnosis based upon the level of expression of one or more genes selected from the group of genes set out in Table 1.

Oligonucleotide probes provided in kits of the invention, may preferably be provided in the form of an oligonucleotide array as considered elsewhere in the specification.

It will be appreciated from the preceding pages that the use of oligonucleotide arrays is particularly useful in effecting a diagnosis in accordance with the present invention as to whether a scar of interest is keloid or non-keloid tissue.

Accordingly, in a third aspect of the invention there is provided an array of oligonucleotide probes, characterised in that at least 7.0% of the oligonucleotides probes present in the array are representative of genes selected from the group of genes set out in Table 1.

The invention also provides an array comprising immobilized antibody probes capable of binding specifically to molecules representative of expression of one or more of the group of genes set out in Table 1. Furthermore, the invention also provides an array comprising a nylon substrate to which are adhered nucleic acid probes representative of genes selected from the group of genes set out in Table 1. The nucleic acid probes may preferably be cDNA molecules.

Although a planar array surface is preferred, the array may be fabricated on a surface of virtually any shape or even a multiplicity of surfaces. In a further example a suitable array may be fabricated on the surface of a library of addressable beads, in which each bead displays a known nucleic acid sequence. Alternatively, a suitable array may be fabricated on the surface of a nylon substrate, typically a woven or non-woven nylon membrane.

It will be appreciated that arrays in accordance with the present invention can be used to compare the expression of a large number of genes set out in Table 1 simultaneously (and indeed to compare simultaneous expression of such genes), and that this gives rise to significant advantages in reduced labour, cost and time. Furthermore, the comparison of expression levels of multiple genes allows a greater degree of confidence in diagnoses that may be effected in accordance with the invention.

An array in accordance with the present invention may comprise up to five probes specific for genes selected from the group set out in Table 1. Preferably an array may comprise 5, 6, 7, 8, 9 or 10 probes specific for genes selected from the group set out in Table 1. Arrays may comprise probes specific for up to 20 or up to 50 genes selected from the group set out in Table 1. Suitable arrays may comprise up to 100, up to 200, up to 300, up to 400 or up to 500 probes specific genes selected from the group set out in Table 1. Indeed, suitable arrays may comprise probes specific for 500 or more of the genes set out in Table 1, and may even comprise probes specific for all 590 genes set out in Table 1. It will be appreciated that each of the probes should be specific for a different selected gene, and that more than one copy of each probe may be provided.

Arrays of the invention may comprise probes capable of binding to a target representative of expression of at least one gene selected from the group set out in Table 2, and/or probes capable of binding to a target representative of expression of at least one gene selected from the group set out in Table 3, and/or probes capable of binding to a target representative of expression of at least one gene selected from the group set out in Table 8, and/or probes capable of binding to a target representative of expression of at least one gene selected from the group set out in Table 13, and/or probes capable of binding to a target representative of expression of at least one gene selected from the group set out in Table 17, and/or probes capable of binding to a target representative of expression of at least one gene selected from the group set out in Table 20, and/or probes capable of binding to a target representative of expression of at least one gene selected from the group set out in Table 22, and/or probes capable of binding to a target representative of expression of at least one gene selected from the group set out in Table 24, and/or probes capable of binding to a target representative of expression of at least one gene selected from the group set out in Table 26.

An array according to the present invention may comprise probes capable of binding to targets representative of the expression of one or more genes selected from any one of, or any combination of, Tables 1 to 28.

It is preferred that an array according to the present invention may further comprise one or more genes whose expression is increased in keloid tissue. The provision of such genes may increase the ability to discriminate a biologically meaningful result from a change in the absolute input material or a change in the efficiency of any assay process. For example, lysyl oxidase displays a 3-fold higher expression in keloid tissue. Lysyl oxidase is a key enzyme involved in collagen cross-linking and has previously been shown to be highly expressed in fibrotic tissue.

The methods, kits and arrays of the invention may also make use of one or more “housekeeping genes” to provide a control by which the efficiency of any assay may be assessed. These housekeeping genes may be provided in the kits of the invention, or on the arrays of the invention. Suitable housekeeping genes will be those that are either invariant between keloid and non-keloid tissue or show no association with keloid formation. Examples of genes that display invariant expression in both keloid and non-keloid (comparator) biopsy samples include exportin 7 (XPO7), Cleavage and Polyadenylation Specific Factor 4, 30 kDa (CPSF4), F-box only protein 7 (FBXO7), ADP-ribosylation factor 1 (ARF1), signal sequence receptor, beta (SSR2) and methionine-tRNA synthetase (MARS).

Oligonucleotide arrays in accordance with the invention may be synthesized by any suitable technique known in the art. A preferred technique that may be used in the synthesis of such arrays is light-directed very large scaled immobilized polymer synthesis (VLSIPS), which has previously been described in a number of publications (Lipshutz R J, Fodor S P, Gingeras T R, Lockhart D J. High density synthetic oligonucleotide arrays. Nat Genet. 1999 January; 21(1 Suppl):20-4; Jacobs J W, Fodor S P. Combinatorial chemistry—applications of light-directed chemical synthesis. Trends Biotechnol. 1994 January; 12(1):19-26).

An oligonucleotide array in accordance with the invention may allow comparison of hybridisation, and thereby gene expression, to be carried out in extremely small fluid volumes (e.g., 250 μl or less, more preferably 100 μl or less, and most preferably 10 μl or less). This confers a number of advantages. In small volumes, hybridization may proceed very rapidly. In addition, hybridization conditions are extremely uniform throughout the sample, and the hybridization format is amenable to automated processing.

The skilled person will appreciate that diagnosis in accordance with the present invention (whether carried out using the methods, kits or arrays of the invention) may be useful in assessing the efficacy of a treatment employed to alleviate or cure keloid scarring. A keloid in which a treatment is producing a beneficial effect may be identified by virtue of its ability to alleviate the reduction of expression observed in respect of the genes set out in any of Tables 1 to 28.

A treatment that renders expression of one or more genes selected from Table 1 within a treated keloid more similar to the expression of said gene (or genes) found in a normal skin comparator should be viewed as having a beneficial effect on the keloid being treated. In the event that expression in a treated keloid is not rendered more similar to the expression found in a normal skin comparator, the treatment may be viewed as not beneficial to the keloid scar in question. In such cases it may be wished to adopt an alternative treatment strategy, and optionally to subsequently assess the effectiveness of the alternative strategy in the same manner.

### Table Legends

Genes the expression of which may be investigated in accordance with the present invention are set out in the accompanying Tables. These Tables provide, in respect of each gene, a Gene Identification Number; a Public Identifier and Data Source (by which the skilled person may identify the gene in question and obtain further information regarding its sequence); the Gene Name; a Probe ID (setting out details of at least one probe that may be used to investigate expression of the gene in question); details of tissues that may be used in comparing expression of the gene in question; as well as details of the Fold Change in expression and P value derived from comparisons conducted as described in the Experimental Results section.

Table 1: Genes that diagnose a keloid scar. All genes are highly statistically significant with p-values less than 0.01.

Table 2: Genes that may be used in the diagnosis of a peri-lesional sample of a scar of interest as a keloid or non-keloid scar.

Table 3: Genes the expression of which may be compared between a peri-lesional sample from a scar of interest and a normal skin comparator to diagnose the scar of interest as a keloid or non-keloid scar.

Table 4: Genes the expression of which may be compared between a peri-lesional sample from a scar of interest and a normal skin comparator to diagnose the scar of interest as a keloid or non-keloid scar. Genes identified in this table encode proteins with cell motility functionality in accordance with the Gene Ontology classification (GO:0006928).

Table 5: Genes the expression of which may be compared between a peri-lesional sample from a scar of interest and a normal skin comparator to diagnose the scar of interest as a keloid or non-keloid scar. Genes identified in this table encode proteins with cell adhesion functionality in accordance with the Gene Ontology classification (GO:0007155).

Table 6: Genes the expression of which may be compared between a peri-lesional sample from a scar of interest and a normal skin comparator to diagnose the scar of interest as a keloid or non-keloid scar. Genes identified in this table encode proteins with inflammation functionality in accordance with the Gene Ontology classification (GO:0006954).

Table 7: Genes the expression of which may be compared between a peri-lesional sample from a scar of interest and a normal skin comparator to diagnose the scar of interest as a keloid or non-keloid scar. Genes identified in this table encode proteins with angiogenesis functionality in accordance with the Gene Ontology classification (GO:0001525).

Table 8: Genes the expression of which may be compared between a peri-lesional sample from a scar of interest and an extra-keloid comparator to diagnose the scar of interest as a keloid or non-keloid scar.

Table 9: Genes the expression of which may be compared between a peri-lesional sample from a scar of interest and an extra-keloid comparator to diagnose the scar of interest as a keloid or non-keloid scar. Genes identified in this table encode proteins with cell motility functionality in accordance with the Gene Ontology classification (GO:0006928). Table 10: Genes the expression of which may be compared between a peri-lesional sample from a scar of interest and an extra-keloid comparator to diagnose the scar of interest as a keloid or non-keloid scar. Genes identified in this table encode proteins with cell adhesion functionality in accordance with the Gene Ontology classification (GO:0007155).

Table 11: Genes the expression of which may be compared between a peri-lesional sample from a scar of interest and an extra-keloid comparator to diagnose the scar of interest as a keloid or non-keloid scar. Genes identified in this table encode proteins with inflammation functionality in accordance with the Gene Ontology classification (GO:0006954).

Table 12: Genes the expression of which may be compared between a peri-lesional sample from a scar of interest and an extra-keloid comparator to diagnose the scar of interest as a keloid or non-keloid scar. Genes identified in this table encode proteins with angiogenesis functionality in accordance with the Gene Ontology classification (GO:0001525).

Table 13: Genes the expression of which may be compared between a peri-lesional sample from a scar of interest and a peri-keloid comparator to diagnose the scar of interest as a keloid or non-keloid scar.

Table 14: Genes the expression of which may be compared between a peri-lesional sample from a scar of interest and a peri-keloid comparator to diagnose the scar of interest as a keloid or non-keloid scar. Genes identified in this table encode proteins with cell adhesion functionality in accordance with the Gene Ontology classification (GO:0007155).

Table 15: Genes the expression of which may be compared between a peri-lesional sample from a scar of interest and a peri-keloid comparator to diagnose the scar of interest as a keloid or non-keloid scar. Genes identified in this table encode proteins with inflammation functionality in accordance with the Gene Ontology classification (GO:0006954).

Table 16: Genes the expression of which may be compared between a peri-lesional sample from a scar of interest and a peri-keloid comparator to diagnose the scar of interest as a keloid or non-keloid scar. Genes identified in this table encode proteins with angiogenesis functionality in accordance with the Gene Ontology classification (GO:0001525).

Table 17: Genes the expression of which may be compared between a peri-lesional sample from a scar of interest and an intra-keloid comparator to diagnose the scar of interest as a keloid or non-keloid scar.

Table 18: Genes the expression of which may be compared between a peri-lesional sample from a scar of interest and an intra-keloid comparator to diagnose the scar of interest as a keloid or non-keloid scar. Genes identified in this table encode proteins with cell motility functionality in accordance with the Gene Ontology classification (GO:0006928).

Table 19: Genes the expression of which may be compared between a peri-lesional sample from a scar of interest and an intra-keloid comparator to diagnose the scar of interest as a keloid or non-keloid scar. Genes identified in this table encode proteins with inflammation functionality in accordance with the Gene Ontology classification (GO:0006954).

Table 20: Genes that may be used in the diagnosis of an intra-lesional sample of a scar of interest as a keloid or non-keloid scar.

Table 21: Genes the expression of which may be compared between an intra-lesional sample from a scar of interest and a normal skin comparator to diagnose the scar of interest as a keloid or non-keloid scar.

Table 22: Genes the expression of which may be compared between an intra-lesional sample from a scar of interest and an extra-keloid comparator to diagnose the scar of interest as a keloid or non-keloid scar.

Table 23: Genes the expression of which may be compared between an intra-lesional sample from a scar of interest and an extra-keloid comparator to diagnose the scar of interest as a keloid or non-keloid scar. Genes identified in this table encode proteins with cell motility functionality in accordance with the Gene Ontology classification (GO:0006928).

Table 24: Genes the expression of which may be compared between an intra-lesional sample from a scar of interest and a peri-keloid comparator to diagnose the scar of interest as a keloid or non-keloid scar.

Table 25: Genes the expression of which may be compared between an intra-lesional sample from a scar of interest and a peri-keloid comparator to diagnose the scar of interest as a keloid or non-keloid scar. Genes identified in this table encode proteins with cell adhesion functionality in accordance with the Gene Ontology classification (GO:00071 55).

Table 26: Genes the expression of which may be compared between an intra-lesional sample from a scar of interest and an intra-keloid comparator to diagnose the scar of interest as a keloid or non-keloid scar.

Table 27: Genes the expression of which may be compared between an intra-lesional sample from a scar of interest and an intra-keloid comparator to diagnose the scar of interest as a keloid or non-keloid scar. Genes identified in this table encode proteins with inflammation functionality in accordance with the Gene Ontology classification (GO:0006954).

Table 28: Genes the expression of which may be compared between an intra-lesional sample from a scar of interest and an intra-keloid comparator to diagnose the scar of interest as a keloid or non-keloid scar. Genes identified in this table encode proteins with angiogenesis functionality in accordance with the Gene Ontology classification (GO:0001 525).

The invention will now be further described with reference to the following Experimental Results.

### EXPERIMENTAL RESULTS

The suitability of the genes set out in Table 1 for use in the diagnosis of scars of interest as keloid or non-keloid is illustrated by the following study. In this study expression of the genes set out in Table 1 was compared between samples taken from known keloid tissues and suitably matched comparator tissues.

### 1.1 Diagnosis of Keloid Tissue.

Twenty patients of the African Continental Ancestry Group who had keloids that had been established for at least one year provided keloid samples for use in the present study. Only keloids for which a full medical history could be established were included. The age of the scar, a thorough review of the scar history and examination by a clinician, ensured that the scar had been correctly diagnosed as keloidal and not hypertrophic.

Three African Continental Ancestry Group subjects with no history of keloid formation provided control comparator tissue (“normal comparator”) for use in the study described herein.

### 1.2 Tissue Collection.

Keloids were sampled using ellipsoid excisions perpendicular to the keloid margin and the resulting biopsies were sectioned to provide samples comprising skin surrounding the keloid lesion (extra-keloid tissue), the peripheral portion of the keloid lesion (peri-lesional tissue), or the interior part of the keloid lesion (intra-lesional tissue). Since these tissues were selected from stringently diagnosed examples of keloids they provided a suitable experimental example to test the diagnostic capacity of the genes set out in Table 1.

Extra-keloid tissue collected in these procedures was used as a comparator tissue (extra-keloid comparator) for use in the following studies. Skin tissue from non-keloid forming individuals was also biopsied in a similar manner to provide relevant non-keloid comparator tissues.

Once collected, the biopsy sections were immersed in RNA Later solution (Ambion) and stored at −80° C. until later analysis of gene expression.

### 1.3 Preparation of Samples Representative of Gene Expression in Tissue.

Peri-lesional, intra-lesional and extra-lesional samples from keloid formers and skin samples from non-keloid formers were disrupted using a Diax (G-10) homogeniser in the presence of proprietary Qiagen lysis buffer, and the lysate produced then incubated with proteinase K at 55° C. for 20 minutes.

Following incubation the mixture was separated by centrifugation, and RNA present purified using a RNeasy midi spin column (Qiagen Ltd).

### 1.4 Production of Nucleic Acid Targets.

10 μg total RNA was used as substrate for cDNA synthesis using the Superscript System (Invitrogen Corp.). The resulting cDNA was then converted to biotinylated cRNA target molecules using the BioArray RNA Transcript labelling Kit (Enzo Life Sciences Inc.). The cRNA target molecules were subsequently purified from the reaction mixture using a RNeasy mini kit (Qiagen Ltd). 20 μg cRNA was fragmented for array hybridisation.

### 1.5 Comparison of Gene Expression.

Fragmented cRNA target molecules representative of gene expression in peri- and intra-lesional keloid tissues and in extra-keloid and non-keloid comparator tissues were hybridised to oligonucleotide arrays comprising oligonucleotide probes representing the genes set out in Table 1. Standard Affymetrix protocols (Affymetrix Inc) were used to effect hybridisation. The hybridised arrays were stained with streptavidin-phycoerythrin and then scanned using a laser confocal scanner to generate fluorescence intensities.

All arrays were normalised to a target intensity of 1000, and signal values and detection P-values were calculated using the Microarray Analysis Suite version 5.0 software. Data sets passing quality control were imported into the Spotfire analysis suite for comparison of expression with that in comparator tissues.

Signal values were transformed to log 2 scale and t-tests comparing the gene expression in samples representative of keloids with expression in comparators were performed on the log 2 transformed data. Mean signal values were calculated for each sample group and fold changes were calculated from these mean values.

### 1.6 Results.

T-tests comparing expression of the genes set out in Table 1 in keloid tissues (peri- and intra-lesional tissues) with expression of the same genes in comparator tissues all had a t-test p-value of less than 0.01. This confirms that the expression of each and all of the genes set out in Table 1 are highly significantly decreased in keloid tissue as opposed to comparators.

These results clearly illustrate that decreased expression in a sample from a scar of interest of one or more genes from the group set out in Table 1, as compared to expression of the same gene or genes in a comparator sample, provides a clear diagnosis that the scar of interest is a keloid tissue.

TABLE 1
Diagnostic Down

| Gene ID | Public Identifier | Data Source | Gene Name | Probe_ID | Comparison | Fold Change | P value |
|---|---|---|---|---|---|---|---|
| 1 | 4250 | Entrez Gene | secretoglobin, family 2A, member 2 | 36329_at | Day 0 Intra/Day 0 Control | 0.009 | 7.1701E−04 |
| | | | | 36329_at | Day 3 Intra/Day 3 Control | 0.017 | 3.3179E−04 |
| | | | | 36329_at | Day 7 Intra/Day 7 Control | 0.021 | 2.8985E−03 |
| | | | | 36329_at | Day 3 Intra/Day 3 Extra | 0.023 | 2.7212E−03 |
| | | | | 36329_at | Day 3 Peri/Day 3 Control | 0.131 | 2.0793E−03 |
| 2 | 5304 | Entrez Gene | prolactin-induced protein | 41094_at | Day 0 Intra/Day 0 Control | 0.032 | 5.1676E−04 |
| | | | | 41094_at | Day 7 Intra/Day 7 Control | 0.076 | 9.5561E−03 |
| | | | | 41094_at | Day 3 Intra/Day 3 Control | 0.094 | 1.9791E−04 |
| 3 | 10647 | Entrez Gene | secretoglobin, family 1D, member 2 | 32880_at | Day 0 Intra/Day 0 Control | 0.033 | 4.4000E−05 |
| | | | | 32880_at | Day 7 Intra/Day 7 Control | 0.065 | 7.5456E−03 |
| | | | | 32880_at | Day 3 Intra/Day 3 Control | 0.075 | 8.2511E−04 |
| | | | | 32880_at | Day 3 Intra/Day 3 Extra | 0.102 | 5.4085E−03 |
| | | | | 32880_at | Day 3 Peri/Day 3 Control | 0.211 | 2.5582E−03 |
| 4 | HG1763-HT178 | The Institute for | — | 325_s_at | Day 3 Intra/Day 3 Extra | 0.038 | 6.4653E−03 |
| | | Genomic Research | | 325_s_at | Day 7 Intra/Day 7 Control | 0.060 | 1.7598E−03 |
| | | | | 325_s_at | Day 3 Intra/Day 3 Control | 0.073 | 2.6304E−04 |
| | | | | 325_s_at | Day 0 Intra/Day 0 Control | 0.091 | 6.8631E−04 |
| 5 | 2167 | Entrez Gene | fatty acid binding protein 4, adipocyte | 38430_at | Day 7 Intra/Day 7 Extra | 0.053 | 1.3371E−03 |
| | | | | 38430_at | Day 7 Intra/Day 7 Control | 0.075 | 4.2884E−03 |
| | | | | 38430_at | Day 0 Peri/Day 0 Control | 0.219 | 8.7014E−03 |
| 6 | 3283 | Entrez Gene | hydroxy-delta-5-steroid dehydrogenase, 3 beta- and steroid delta-isomerase 1 | 35721_at | Day 0 Intra/Day 0 Peri | 0.062 | 4.4497E−03 |
| 7 | 6288 | Entrez Gene | serum amyloid A1 | 33272_at | Day 3 Intra/Day 3 Extra | 0.069 | 6.8147E−03 |
| | | | | 33272_at | Day 7 Intra/Day 7 Extra | 0.117 | 4.3324E−03 |
| 8 | 9415 | Entrez Gene | fatty acid desaturase 2 | 32190_at | Day 0 Intra/Day 0 Peri | 0.081 | 5.6381E−04 |
| | | | | 32190_at | Day 7 Intra/Day 7 Peri | 0.201 | 7.6424E−03 |
| | | | | 32190_at | Day 3 Peri/Day 0 Peri | 0.365 | 8.5757E−03 |
| 9 | 247 | Entrez Gene | arachidonate 15-lipoxygenase, second type | 37430_at | Day 0 Intra/Day 0 Peri | 0.096 | 4.5492E−03 |
| 10 | 1690 | Entrez Gene | coagulation factor C homolog, cochlin (Limulus polyphemus) | 34190_at | Day 3 Intra/Day 3 Extra | 0.129 | 1.6089E−03 |
| 11 | 553168 | Entrez Gene | chromosome 1 open reading frame 68 | 31326_at | Day 7 Peri/Day 7 Control | 0.138 | 7.6909E−03 |
| | | | | 31326_at | Day 3 Peri/Day 3 Control | 0.214 | 6.7718E−03 |
| | | | | 31326_at | Day 7 Peri/Day 0 Peri | 0.229 | 1.6500E−05 |
| | | | | 31326 at | Day 7 Peri/Day 7 Extra | 0.288 | 7.6740E−03 |
| | | | | 31326_at | Day 3 Peri/Day 0 Peri | 0.487 | 3.0725E−03 |
| 12 | HG371-HT2638 | The Institute for | — | 700_s_at | Day 0 Intra/Day 0 Peri | 0.143 | 4.4849E−03 |
| | | Genomic Research | | | | | |
| 13 | 125 | Entrez Gene | alcohol dehydrogenase IB (class I), beta polypeptide | 35730_at | Day 3 Intra/Day 0 Intra | 0.162 | 9.2302E−03 |
| | | | | 35730_at | Day 0 Peri/Day 0 Intra | 0.252 | 5.3255E−03 |
| 14 | 3205 | Entrez Gene | homeo box A9 | 37809_at | Day 3 Peri/Day 3 Control | 0.180 | 5.0202E−03 |
| | | | | 37809_at | Day 7 Peri/Day 7 Extra | 0.237 | 8.7426E−03 |
| | | | | 37809_at | Day 3 Peri/Day 3 Extra | 0.314 | 9.1991E−03 |
| 15 | 7136 | Entrez Gene | troponin I type 2 (skeletal, fast) | 38457_at | Day 0 Peri/Day 0 Control | 0.185 | 5.1425E−04 |
| 16 | L09190 | GenBank | Full-length cDNA clone CS0DI027YJ05 of Placenta Cot | 37635_at | Day 3 Intra/Day 3 Peri | 0.206 | 2.8404E−03 |
| | | | 25-normalized at Homo sapiens (human) | | | | |
| 17 | 6210 | Entrez Gene | ribosomal protein S15a | 34316_at | Day 3 Peri/Day 3 Control | 0.241 | 3.8100E−05 |
| | | | | 34316_at | Day 7 Peri/Day 7 Control | 0.432 | 2.3230E−04 |
| | | | | 34316_at | Day 3 Peri/Day 3 Extra | 0.476 | 1.6785E−04 |
| 18 | 7123 | Entrez Gene | C-type lectin domain family 3, member B | 36569_at | Day 3 Intra/Day 0 Intra | 0.243 | 7.7786E−03 |
| | | | | 36569_at | Day 3 Peri/Day 0 Peri | 0.356 | 1.8400E−05 |
| | | | | 36569_at | Day 7 Peri/Day 0 Peri | 0.428 | 5.8746E−04 |
| 19 | 10351 | Entrez Gene | ATP-binding cassette, sub-family A (ABC1), member 8 | 35717_at | Day 3 Intra/Day 0 Intra | 0.279 | 3.1166E−03 |
| | | | | 35717_at | Day 3 Intra/Day 3 Peri | 0.305 | 5.8783E−04 |
| 20 | AB011538 | GenBank | CDNA clone IMAGE: 5922621 | 35324_at | Day 3 Intra/Day 0 Intra | 0.280 | 3.8006E−03 |
| | | | | 35324_at | Day 3 Peri/Day 0 Peri | 0.447 | 1.3446E−04 |
| | | | | 35324_at | Day 7 Peri/Day 0 Peri | 0.623 | 4.2479E−03 |
| 21 | 3131 | Entrez Gene | Hepatic leukemia factor | 38627_at | Day 3 Intra/Day 0 Intra | 0.289 | 1.1838E−03 |
| | | | | 38627_at | Day 7 Intra/Day 0 Intra | 0.325 | 6.7101E−03 |
| | | | | 38627_at | Day 0 Peri/Day 0 Intra | 0.556 | 5.7849E−03 |
| 22 | M57951 | GenBank | UDP glucuronosyltransferase 1 family, polypeptide A10 | 32392_s_at | Day 3 Peri/Day 3 Extra | 0.320 | 3.1014E−03 |
| | | | /// UDP glucuronosyltransferase 1 family, polypeptide | | | | |
| | | | A8 /// UDP glucuronosyltransferase 1 fami | | | | |
| 23 | 4211 | Entrez Gene | Meis1, myeloid ecotropic viral integration site 1 homolog (mouse) | 40763_at | Day 3 Intra/Day 0 Intra | 0.324 | 4.5206E−03 |
| 24 | 2053 | Entrez Gene | epoxide hydrolase 2, cytoplasmic | 41473_at | Day 3 Intra/Day 0 Intra | 0.327 | 3.5922E−03 |
| | | | | 41473_at | Day 3 Peri/Day 0 Peri | 0.521 | 6.6949E−03 |
| 25 | 4253 | Entrez Gene | CTAGE family, member 5 | 41615_at | Day 3 Intra/Day 0 Intra | 0.330 | 3.8948E−03 |
| 26 | 5271 | Entrez Gene | serpin peptidase inhibitor, clade B (ovalbumin), member 8 | 36312_at | Day 7 Peri/Day 7 Extra | 0.331 | 4.7432E−04 |
| | | | | 36312_at | Day 7 Intra/Day 7 Extra | 0.482 | 4.6083E−03 |
| 27 | 3119 | Entrez Gene | major histocompatibility complex, class II, DQ beta 1 | 36876_f_at | Day 0 Intra/Day 0 Control | 0.350 | 9.9683E−03 |
| 28 | 10781 | Entrez Gene | zinc finger protein 266 | 41621_i_at | Day 0 Peri/Day 0 Control | 0.350 | 5.4116E−04 |
| 29 | 4128 | Entrez Gene | monoamine oxidase A | 41772_at | Day 3 Peri/Day 3 Extra | 0.351 | 3.2109E−03 |
| | | | | 41771_g_at | Day 3 Peri/Day 3 Control | 0.464 | 2.4030E−03 |
| | | | | 41772_at | Day 7 Peri/Day 7 Extra | 0.614 | 6.2007E−03 |
| 30 | 3885 | Entrez Gene | keratin, hair, acidic, 4 | 34012_at | Day 0 Intra/Day 0 Peri | 0.352 | 4.5590E−03 |
| 31 | 8483 | Entrez Gene | cartilage intermediate layer protein, nucleotide | 34985_at | Day 3 Peri/Day 0 Peri | 0.354 | 4.8214E−04 |
| | | | pyrophosphohydrolase | 34985_at | Day 7 Peri/Day 0 Peri | 0.381 | 2.9109E−03 |
| 32 | 11075 | Entrez Gene | stathmin-like 2 | 38800_at | Day 3 Intra/Day 0 Intra | 0.356 | 5.6882E−03 |
| 33 | 3075 | Entrez Gene | complement factor H | 32250_at | Day 7 Intra/Day 0 Intra | 0.356 | 2.4648E−03 |
| 34 | 10458 | Entrez Gene | BAl1-associated protein 2 | 37761_at | Day 7 Peri/Day 0 Peri | 0.358 | 3.2292E−04 |
| | | | | 37760_at | Day 7 Intra/Day 3 Intra | 0.799 | 8.1747E−03 |
| 35 | 9086 | Entrez Gene | eukaryotic translation initiation factor 1A, Y-linked | 40097_at | Day 3 Intra/Day 0 Intra | 0.358 | 8.6154E−03 |
| 36 | 2273 | Entrez Gene | four and a half LIM domains 1 | 32542_at | Day 3 Intra/Day 0 Intra | 0.367 | 5.7500E−03 |
| | | | | 32542_at | Day 3 Peri/Day 0 Peri | 0.598 | 1.0604E−03 |
| | | | | 32542_at | Day 7 Peri/Day 0 Peri | 0.643 | 1.4304E−03 |
| 37 | 23016 | Entrez Gene | exosome component 7 | 33294_at | Day 0 Peri/Day 0 Control | 0.369 | 2.9368E−04 |
| | | | | 33294_at | Day 3 Peri/Day 3 Control | 0.453 | 3.7416E−04 |
| 38 | 23352 | Entrez Gene | retinoblastoma-associated factor 600 | 33860_at | Day 0 Peri/Day 0 Control | 0.369 | 5.2550E−04 |
| | | | | 33860_at | Day 3 Peri/Day 3 Control | 0.374 | 1.4026E−03 |
| | | | | 33860_at | Day 7 Peri/Day 7 Control | 0.452 | 4.0374E−03 |
| | | | | 33860_at | Day 3 Peri/Day 3 Extra | 0.549 | 6.8632E−03 |
| | | | | 33860_at | Day 7 Peri/Day 7 Extra | 0.579 | 3.6353E−03 |
| 39 | 7138 | Entrez Gene | troponin T type 1 (skeletal, slow) | 36113_s_at | Day 7 Peri/Day 7 Control | 0.371 | 6.4628E−03 |
| | | | | 36113_s_at | Day 3 Peri/Day 3 Control | 0.452 | 4.3070E−03 |
| 40 | 4223 | Entrez Gene | mesenchyme homeo box 2 (growth arrest-specific | 40398_s_at | Day 3 Intra/Day 0 Intra | 0.376 | 7.2046E−03 |
| | | | homeo box) | | | | |
| 41 | 3400 | Entrez Gene | Inhibitor of DNA binding 4, dominant negative helix-loop | 41536_at | Day 3 Intra/Day 0 Intra | 0.381 | 8.0900E−05 |
| | | | helix protein | 41536_at | Day 7 Intra/Day 0 Intra | 0.410 | 2.1354E−03 |
| | | | | 41536_at | Day 3 Intra/Day 3 Control | 0.488 | 2.0560E−03 |
| 42 | 10521 | Entrez Gene | DEAD (Asp-Glu-Ala-Asp) box polypeptide 17 | 35363_at | Day 0 Peri/Day 0 Control | 0.383 | 2.2472E−03 |
| | | | | 35363_at | Day 7 Peri/Day 7 Extra | 0.577 | 6.8649E−03 |
| 43 | 8710 | Entrez Gene | serpin peptidase inhibitor, clade B (ovalbumin), member 7 | 35577_at | Day 0 Peri/Day 0 Control | 0.385 | 4.8105E−04 |
| | | | | 35577_at | Day 0 Intra/Day 0 Control | 0.489 | 6.7433E−03 |
| | | | | 35577_at | Day 3 Intra/Day 3 Control | 0.566 | 9.3574E−03 |
| 44 | 2194 | Entrez Gene | fatty acid synthase | 38429_at | Day 0 Intra/Day 0 Peri | 0.386 | 9.3714E−03 |
| | | | | 38429_at | Day 3 Peri/Day 0 Peri | 0.512 | 8.4627E−03 |
| 45 | 3202 | Entrez Gene | homeo box A5 | 873_at | Day 3 Peri/Day 3 Extra | 0.387 | 6.1920E−03 |
| 46 | 54 | Entrez Gene | acid phosphatase 5, tartrate resistant | 677_s_at | Day 7 Peri/Day 7 Control | 0.389 | 2.8442E−04 |
| | | | | 677_s_at | Day 7 Peri/Day 7 Extra | 0.672 | 8.0184E−03 |
| 47 | 406 | Entrez Gene | aryl hydrocarbon receptor nuclear translocator-like | 36896_s_at | Day 3 Intra/Day 3 Control | 0.391 | 9.1974E−03 |
| 48 | 9452 | Entrez Gene | Integral membrane protein 2A | 40775_at | Day 3 Intra/Day 0 Intra | 0.392 | 3.3491E−03 |
| | | | | 40775_at | Day 7 Intra/Day 0 Intra | 0.400 | 2.0273E−03 |
| 49 | 63928 | Entrez Gene | hepatocellular carcinoma antigen gene 520 | 33007_at | Day 3 Peri/Day 3 Control | 0.394 | 7.4079E−03 |
| | | | | 33007_at | Day 3 Intra/Day 3 Control | 0.466 | 7.7206E−03 |
| 50 | 5055 | Entrez Gene | serpin peptidase inhibitor, clade B (ovalbumin), member 2 | 37185_at | Day 0 Peri/Day 0 Control | 0.399 | 5.5226E−03 |
| 51 | 5354 | Entrez Gene | proteolipid protein 1 (Pelizaeus-Merzbacher disease, | 41158_at | Day 3 Intra/Day 0 Intra | 0.403 | 1.1357E−04 |
| | | | spastic paraplegia 2, uncomplicated) | 41158_at | Day 7 Intra/Day 0 Intra | 0.503 | 1.7500E−03 |
| | | | | 41158_at | Day 0 Peri/Day 0 Control | 0.544 | 4.8737E−03 |
| | | | | 41158_at | Day 3 Peri/Day 0 Peri | 0.715 | 7.5707E−03 |
| 52 | 642 | Entrez Gene | bleomycin hydrolase | 394_at | Day 3 Peri/Day 3 Control | 0.404 | 6.8138E−03 |
| 53 | 10443 | Entrez Gene | Hypothetical gene CG012 | 1532_g_at | Day 0 Peri/Day 0 Control | 0.406 | 9.9594E−04 |
| | | | | 1532_g_at | Day 3 Peri/Day 3 Control | 0.567 | 4.1852E−03 |
| | | | | 1532_g_at | Day 0 Peri/Day 0 Intra | 0.660 | 5.2680E−03 |
| 54 | 224 | Entrez Gene | aldehyde dehydrogenase 3 family, member A2 | 40409_at | Day 3 Intra/Day 3 Control | 0.410 | 5.0007E−03 |
| | | | | 40409_at | Day 3 Peri/Day 3 Control | 0.445 | 7.4233E−03 |
| | | | | 40409_at | Day 3 Intra/Day 0 Intra | 0.466 | 8.5061E−04 |
| 55 | 90634 | Entrez Gene | hypothetical gene CG018 | 1527_s_at | Day 0 Peri/Day 0 Control | 0.410 | 2.0450E−03 |
| | | | | 34239_at | Day 3 Intra/Day 3 Control | 0.472 | 5.9625E−03 |
| | | | | 1527_s_at | Day 3 Peri/Day 3 Control | 0.515 | 2.5140E−03 |
| 56 | 1675 | Entrez Gene | D component of complement (adipsin) | 40282_s_at | Day 7 Peri/Day 0 Peri | 0.410 | 3.2238E−03 |
| 57 | 23242 | Entrez Gene | cordon-bleu homolog (mouse) | 35669_at | Day 3 Intra/Day 0 Intra | 0.414 | 5.3389E−03 |
| | | | | 35669_at | Day 3 Intra/Day 3 Control | 0.417 | 3.3977E−03 |
| | | | | 35669_at | Day 3 Intra/Day 3 Extra | 0.522 | 4.0595E−04 |
| | | | | 35669_at | Day 7 Peri/Day 0 Peri | 0.543 | 1.1342E−03 |
| | | | | 35669_at | Day 3 Peri/Day 0 Peri | 0.585 | 1.2820E−03 |
| 58 | 10158 | Entrez Gene | PDZK1 interacting protein 1 | 31610_at | Day 3 Peri/Day 3 Extra | 0.421 | 4.1131E−03 |
| 59 | 10001 | Entrez Gene | mediator of RNA polymerase II transcription, subunit 6 homolog (yeast) | 35430_at | Day 3 Peri/Day 3 Control | 0.427 | 4.0549E−03 |
| 60 | 97 | Entrez Gene | acylphosphatase 1, erythrocyte (common) type | 33334_at | Day 0 Peri/Day 0 Control | 0.427 | 3.3326E−03 |
| 61 | M11119 | GenBank | — | 38850_at | Day 3 Intra/Day 0 Intra | 0.428 | 8.8745E−03 |
| 62 | 9687 | Entrez Gene | GREB1 protein | 38875_r_at | Day 7 Intra/Day 0 Intra | 0.428 | 1.0961E−04 |
| 63 | 51097 | Entrez Gene | saccharopine dehydrogenase (putative) | 34863_s_at | Day 3 Peri/Day 3 Control | 0.430 | 9.0435E−03 |
| | | | | 34862_at | Day 7 Intra/Day 0 Intra | 0.680 | 6.5823E−03 |
| 64 | 2217 | Entrez Gene | Fc fragment of IgG, receptor, transporter, alpha | 31431_at | Day 7 Peri/Day 7 Control | 0.430 | 7.9132E−04 |
| 65 | 1983 | Entrez Gene | eukaryotic translation initiation factor 5 | 167_at | Day 3 Peri/Day 3 Control | 0.431 | 3.2376E−04 |
| | | | | 167_at | Day 7 Peri/Day 7 Extra | 0.519 | 7.0199E−03 |
| 66 | 51601 | Entrez Gene | lipoyltransferase 1 | 37441_at | Day 0 Peri/Day 0 Intra | 0.432 | 3.4529E−03 |
| 67 | 9145 | Entrez Gene | synaptogyrin 1 | 35354_at | Day 7 Intra/Day 0 Intra | 0.432 | 1.9735E−03 |
| | | | | 35354_at | Day 3 Intra/Day 0 Intra | 0.469 | 5.7597E−03 |
| 68 | 1117 | Entrez Gene | chitinase 3-like 2 | 31891_at | Day 0 Intra/Day 0 Peri | 0.432 | 5.3219E−04 |
| 69 | 13 | Entrez Gene | arylacetamide deacetylase (esterase) | 36512_at | Day 3 Intra/Day 3 Extra | 0.434 | 1.5905E−04 |
| 70 | 2618 | Entrez Gene | phosphoribosylglycinamide formyltransferase, | 38384_at | Day 3 Peri/Day 3 Extra | 0.436 | 8.9104E−03 |
| | | | phosphoribosylglycinamide synthetase, | | | | |
| | | | phosphoribosylaminoimidazole synthetase | | | | |
| 71 | HG3570-HT377 | The Institute for | — | 936_s_at | Day 3 Peri/Day 3 Control | 0.437 | 1.3929E−04 |
| | | Genomic Research | | 936_s_at | Day 7 Peri/Day 7 Control | 0.554 | 6.0851E−03 |
| | | | | 936_s_at | Day 7 Peri/Day 0 Peri | 0.679 | 6.6192E−03 |
| 72 | 53335 | Entrez Gene | B-cell CLL/lymphoma 11A (zinc finger protein) | 41356_at | Day 3 Peri/Day 3 Extra | 0.438 | 5.3163E−03 |
| 73 | 57798 | Entrez Gene | GATA zinc finger domain containing 1 | 34195_at | Day 3 Intra/Day 3 Peri | 0.441 | 6.2766E−04 |
| 74 | 8906 | Entrez Gene | adaptor-related protein complex 1, gamma 2 subunit | 38798_s_at | Day 0 Peri/Day 0 Control | 0.441 | 6.9369E−03 |
| | | | | 38798_s_at | Day 3 Peri/Day 3 Control | 0.480 | 6.4090E−03 |
| 75 | 26 | Entrez Gene | amiloride binding protein 1 (amine oxidase (copper- | 37186_s_at | Day 3 Intra/Day 3 Extra | 0.443 | 5.1668E−03 |
| | | | containing)) | | | | |
| 76 | AL080082 | GenBank | MRNA; cDNA DKFZp564G1162 (from clone | 35581_at | Day 3 Intra/Day 0 Intra | 0.444 | 9.2254E−03 |
| | | | DKFZp564G1162) | | | | |
| 77 | 9524 | Entrez Gene | glycoprotein, synaptic 2 | 38966_at | Day 7 Peri/Day 7 Control | 0.444 | 3.9851E−03 |
| | | | | 38966_at | Day 3 Peri/Day 3 Control | 0.530 | 1.4148E−03 |
| | | | | 38966_at | Day 7 Peri/Day 0 Peri | 0.614 | 2.1060E−03 |
| 78 | 91137 | Entrez Gene | hypothetical protein BC017169 | 37178_at | Day 3 Peri/Day 3 Control | 0.449 | 3.9314E−03 |
| 79 | 56288 | Entrez Gene | par-3 partitioning defective 3 homolog (C. elegans) | 40973_at | Day 0 Peri/Day 0 Control | 0.451 | 2.5568E−03 |
| | | | | 40973_at | Day 3 Intra/Day 3 Control | 0.533 | 5.0405E−03 |
| 80 | 26154 | Entrez Gene | ATP-binding cassette, sub-family A (ABC1), member 12 | 31754_at | Day 3 Peri/Day 3 Extra | 0.453 | 4.6700E−03 |
| | | | | 31754_at | Day 3 Intra/Day 3 Extra | 0.557 | 5.7474E−03 |
| 81 | 260294 | Entrez Gene | Williams Beuren syndrome chromosome region 20C | 41258_at | Day 0 Peri/Day 0 Control | 0.453 | 3.7268E−03 |
| 82 | 1410 | Entrez Gene | crystallin, alpha B | 32242_at | Day 3 Peri/Day 3 Extra | 0.454 | 8.8596E−03 |
| | | | | 32243_g_at | Day 3 Intra/Day 3 Extra | 0.468 | 5.9480E−03 |
| 83 | 131544 | Entrez Gene | hypothetical protein DKFZp667G2110 | 41274_at | Day 0 Peri/Day 0 Control | 0.454 | 4.4963E−03 |
| | | | | 41274_at | Days 3 Peri/Day 3 Control | 0.589 | 7.6530E−03 |
| 84 | 10733 | Entrez Gene | polo-like kinase 4 (Drosophila) | 975_at | Day 7 Intra/Day 7 Extra | 0.455 | 5.4588E−03 |
| | | | | 975_at | Day 7 Peri/Day 7 Extra | 0.459 | 4.3469E−03 |
| 85 | 8848 | Entrez Gene | TSC22 domain family, member 1 | 39032_at | Day 3 Intra/Day 0 Intra | 0.455 | 1.8273E−03 |
| 86 | 10181 | Entrez Gene | RNA binding motif protein 5 | 1556_at | Day 0 Peri/Day 0 Control | 0.460 | 1.4047E−03 |
| 87 | 5264 | Entrez Gene | phytanoyl-CoA hydroxylase (Refsum disease) | 32724_at | Day 3 Intra/Day 0 Intra | 0.465 | 4.1244E−03 |
| 88 | 10529 | Entrez Gene | nebulette | 34873_at | Day 3 Intra/Day 3 Control | 0.466 | 5.5136E−03 |
| 89 | 60481 | Entrez Gene | ELOVL family member 5, elongation of long chain fatty | 33821_at | Day 0 Intra/Day 0 Peri | 0.468 | 8.7794E−03 |
| | | | acids (FEN1/Elo2, SUR4/Elo3-like, yeast) | | | | |
| 90 | 6924 | Entrez Gene | Transcription elongation factor B (SIII), polypeptide 3 | 32049_f_at | Day 0 Peri/Day 0 Intra | 0.469 | 3.1906E−03 |
| | | | (110 kDa, elongin A) | | | | |
| 91 | 10248 | Entrez Gene | processing of precursor 7, ribonuclease P subunit (S. cerevisiae) | 32213_at | Day 7 Intra/Day 3 Intra | 0.470 | 9.0083E−03 |
| 92 | 1545 | Entrez Gene | cytochrome P450, family 1, subfamily B, polypeptide 1 | 40071_at | Day 7 Intra/Day 7 Peri | 0.471 | 9.8480E−03 |
| 93 | 1152 | Entrez Gene | creatine kinase, brain | 40863_r_at | Day 7 Intra/Day 7 Control | 0.472 | 4.0142E−03 |
| | | | | 40863_r_at | Day 7 Peri/Day 0 Peri | 0.507 | 3.2271E−04 |
| 94 | 10450 | Entrez Gene | peptidylprolyl isomerase E (cyclophilin E) | 34365_at | Day 3 Peri/Day 3 Control | 0.473 | 5.7231E−03 |
| | | | | 34365_at | Day 3 Intra/Day 3 Control | 0.518 | 4.1270E−03 |
| | | | | 34365_at | Day 7 Peri/Day 0 Peri | 0.638 | 2.8273E−03 |
| 95 | HG1112-HT111 | The Institute for | — | 1839_at | Day 3 Peri/Day 3 Extra | 0.473 | 1.6274E−03 |
| | | Genomic Research | | 1839_at | Day 3 Peri/Day 3 Control | 0.555 | 9.8116E−03 |
| 96 | 10090 | Entrez Gene | uronyl-2-sulfotransferase | 41859_at | Day 3 Intra/Day 0 Intra | 0.474 | 1.8927E−03 |
| | | | | 41859_at | Day 7 Intra/Day 0 Intra | 0.521 | 1.4198E−04 |
| 97 | 4129 | Entrez Gene | monoamine oxidase B | 37628_at | Day 3 Peri/Day 3 Extra | 0.475 | 8.4099E−04 |
| 98 | 352961 | Entrez Gene | MHC class I mRNA fragment 3.8-1 | 34934_at | Day 3 Intra/Day 0 Intra | 0.479 | 6.5297E−03 |
| 99 | 10314 | Entrez Gene | LanC lantibiotic synthetase component C-like 1 | 39441_at | Day 0 Peri/Day 0 Control | 0.482 | 2.7594E−03 |
| | | | (bacterial) | 39441_at | Day 0 Intra/Day 0 Control | 0.488 | 6.0119E−04 |
| 100 | 10628 | Entrez Gene | thioredoxin interacting protein | 31508_at | Day 3 Intra/Day 0 Intra | 0.482 | 9.0159E−03 |
| 101 | 8532 | Entrez Gene | carboxypeptidase Z | 37248_at | Day 7 Peri/Day 0 Peri | 0.483 | 2.2125E−04 |
| | | | | 37248_at | Day 3 Peri/Day 0 Peri | 0.522 | 7.1260E−04 |
| 102 | 6038 | Entrez Gene | ribonuclease, RNase A family, 4 | 32664_at | Day 3 Intra/Day 0 Intra | 0.483 | 2.7472E−03 |
| | | | | 32664_at | Day 7 Intra/Day 0 Intra | 0.569 | 4.9291E−03 |
| | | | | 32664_at | Day 3 Peri/Day 0 Peri | 0.636 | 5.8524E−04 |
| | | | | 32664_at | Day 7 Peri/Day 0 Peri | 0.691 | 3.6796E−03 |
| 103 | 10924 | Entrez Gene | sphingomyelin phosphodiesterase, acid-like 3A | 39950_at | Day 3 Peri/Day 3 Control | 0.483 | 8.6125E−03 |
| 104 | 6414 | Entrez Gene | selenoprotein P, plasma, 1 | 34363_at | Day 3 Intra/Day 0 Intra | 0.488 | 6.0330E−03 |
| 105 | 9987 | Entrez Gene | Heterogeneous nuclear ribonucleoproteln D-like | 32393_s_at | Day 3 Intra/Day 3 Peri | 0.489 | 1.9407E−03 |
| 106 | 8634 | Entrez Gene | RNA terminal phosphate cyclase domain 1 | 35195_at | Day 7 Peri/Day 7 Extra | 0.489 | 1.6326E−03 |
| 107 | 4163 | Entrez Gene | mutated in colorectal cancers | 35561_at | Day 0 Intra/Day 0 Peri | 0.489 | 6.2800E−05 |
| | | | | 35561_at | Day 7 Peri/Day 0 Peri | 0.606 | 7.2171E−03 |
| 108 | 7262 | Entrez Gene | pleckstrin homology-like domain, family A, member 2 | 31888_s_at | Day 3 Peri/Day 3 Extra | 0.490 | 4.0424E−03 |
| 109 | 26039 | Entrez Gene | synovial sarcoma translocation gene on chromosome | 31903_at | Day 0 Peri/Day 0 Control | 0.493 | 1.3274E−03 |
| | | | 18-like 1 | | | | |
| 110 | 1525 | Entrez Gene | coxsackie virus and adenovirus receptor | 37534_at | Day 3 Peri/Day 3 Extra | 0.493 | 7.3178E−03 |
| 111 | 2947 | Entrez Gene | glutathione S-transferase M3 (brain) | 1120_at | Day 7 Intra/Day 0 Intra | 0.494 | 7.1503E−03 |
| 112 | 11072 | Entrez Gene | dual specificity phosphatase 14 | 38272_at | Day 7 Peri/Day 7 Extra | 0.494 | 1.4839E−03 |
| 113 | 6490 | Entrez Gene | silver homolog (mouse) | 38327_at | Day 3 Peri/Day 3 Extra | 0.497 | 4.4983E−04 |
| | | | | 38327_at | Day 3 Peri/Day 3 Control | 0.512 | 5.3933E−04 |
| | | | | 38327_at | Day 3 Peri/Day 0 Peri | 0.710 | 9.2062E−03 |
| 114 | 8864 | Entrez Gene | period homolog 2 (Drosophila) | 35008_at | Day 3 Intra/Day 0 Intra | 0.497 | 4.3990E−03 |
| 115 | 4176 | Entrez Gene | MCM7 minichromosome maintenance deficient 7 (S. cerevisiae) | 947_at | Day 0 Peri/Day 0 Control | 0.498 | 7.8436E−04 |
| 116 | 2197 | Entrez Gene | Finkel-Biskis-Reilly murine sarcoma virus (FBR-MuSV) | 31955_at | Day 7 Peri/Day 7 Control | 0.498 | 8.3003E−03 |
| | | | ubiquitously expressed (fox derived); ribosomal protein | | | | |
| | | | S30 | | | | |
| 117 | 104 | Entrez Gene | adenosine deaminase, RNA-specific, B1 (RED1 | 38748_at | Day 3 Intra/Day 0 Intra | 0.499 | 8.5077E−04 |
| | | | homolog rat) | 38748_at | Day 7 Intra/Day 0 Intra | 0.541 | 1.9004E−03 |
| 118 | 4898 | Entrez Gene | nardilysin (N-arginine dibasic convertase) | 34787_at | Day 3 Peri/Day 3 Control | 0.499 | 2.6275E−03 |
| 119 | 6662 | Entrez Gene | SRY (sex determining region Y)-box 9 (campomelic | 33436_at | Day 3 Peri/Day 3 Extra | 0.500 | 5.3219E−03 |
| | | | dysplasia, autosomal sex-reversal) | 33436_at | Day 0 Intra/Day 0 Peri | 0.521 | 8.5777E−03 |
| 120 | 2114 | Entrez Gene | v-ets erythroblastosis virus E26 oncogene homolog 2 | 38739_at | Day 3 Peri/Day 3 Extra | 0.501 | 4.4662E−03 |
| | | | (avian) | | | | |
| 121 | 1158 | Entrez Gene | creatine kinase, muscle | 32486_at | Day 0 Peri/Day 0 Intra | 0.501 | 6.1425E−03 |
| 122 | 9831 | Entrez Gene | zinc finger protein 623 | 39954_r_at | Day 3 Intra/Day 0 Intra | 0.505 | 3.3609E−03 |
| 123 | 1573 | Entrez Gene | cytochrome P450, family 2, subfamily J, polypeptide 2 | 501_g_at | Day 3 Peri/Day 3 Control | 0.505 | 2.0051E−03 |
| | | | | 501_g_at | Day 3 Intra/Day 3 Control | 0.529 | 4.2791E−03 |
| 124 | 22982 | Entrez Gene | KIAA0934 | 33408_at | Day 3 Intra/Day 0 Intra | 0.506 | 3.1621E−03 |
| 125 | 51710 | Entrez Gene | zinc finger protein 44 (KOX7) | 35409_r_at | Day 3 Intra/Day 3 Peri | 0.507 | 5.1572E−03 |
| 126 | 2533 | Entrez Gene | FYN binding protein (FYB-120/130) | 41819_at | Day 3 Intra/Day 3 Peri | 0.507 | 3.0115E−03 |
| 127 | 9857 | Entrez Gene | centrosome-associated protein 350 | 33805_at | Day 3 Intra/Day 3 Peri | 0.508 | 4.3400E−05 |
| 128 | 9891 | Entrez Gene | NUAK family, SNF1-like kinase, 1 | 33787_at | Day 3 Intra/Day 0 Intra | 0.508 | 1.4528E−03 |
| 129 | 9833 | Entrez Gene | maternal embryonic leucine zipper kinase | 38847_at | Day 7 Peri/Day 7 Extra | 0.510 | 9.6404E−03 |
| 130 | U18300 | GenBank | damage-specific DNA binding protein 2, 48 kDa /// LIM | 1243_at | Day 3 Peri/Day 3 Control | 0.511 | 4.6848E−04 |
| | | | homeobox 3 | 1243_at | Day 0 Peri/Day 0 Control | 0.556 | 6.7443E−03 |
| | | | | 1243_at | Day 7 Peri/Day 0 Peri | 0.749 | 8.6022E−03 |
| 131 | 23051 | Entrez Gene | zinc fingers and homeoboxes 3 | 40461_at | Day 3 Intra/Day 0 Intra | 0.513 | 6.7006E−04 |
| | | | | 40461_at | Day 7 Intra/Day 0 Intra | 0.609 | 6.3269E−03 |
| | | | | 40461_at | Day 3 Peri/Day 0 Peri | 0.738 | 3.2244E−03 |
| 132 | 51706 | Entrez Gene | cytochrome b5 reductase 1 | 35329_at | Day 3 Peri/Day 3 Control | 0.513 | 1.8894E−03 |
| 133 | 3422 | Entrez Gene | isopentenyl-diphosphate delta isomerase 1 | 36985_at | Day 7 Intra/Day 7 Extra | 0.515 | 5.4407E−03 |
| 134 | 7728 | Entrez Gene | zinc finger protein 175 | 37500_at | Day 7 Intra/Day 7 Peri | 0.515 | 4.1955E−03 |
| 135 | 36 | Entrez Gene | acyl-Coenzyme A dehydrogenase, short/branched chain | 40673_at | Day 3 Intra/Day 3 Control | 0.515 | 1.3679E−03 |
| 136 | 2013 | Entrez Gene | epithelial membrane protein 2 | 39631_at | Day 3 Peri/Day 3 Extra | 0.517 | 3.1103E−03 |
| | | | | 39631_at | Day 7 Peri/Day 0 Peri | 0.629 | 5.1967E−04 |
| 137 | 81563 | Entrez Gene | chromosome 1 open reading frame 21 | 41679_at | Day 7 Peri/Day 0 Peri | 0.517 | 12344E−03 |
| | | | | 41679_at | Day 3 Peri/Day 0 Peri | 0.607 | 8.6951E−03 |
| 138 | 1638 | Entrez Gene | dopachrome tautomerase (dopachrome delta- | 41182_at | Day 7 Peri/Day 0 Peri | 0.518 | 2.7300E−05 |
| | | | isomerase, tyrosine-related protein 2) | 41182_at | Day 3 Peri/Day 0 Peri | 0.596 | 1.9071E−04 |
| 139 | 10965 | Entrez Gene | acyl-CoA thioesterase 2 | 36625_at | Day 7 Intra/Day 7 Control | 0.518 | 7.3351E−04 |
| 140 | 10284 | Entrez Gene | Sin3-associated polypeptide, 18 kDa | 41277_at | Day 3 Intra/Day 0 Intra | 0.520 | 2.4318E−03 |
| 141 | 57613 | Entrez Gene | KIAA1467 protein | 41826_at | Day 0 Peri/Day 0 Control | 0.520 | 5.9359E−03 |
| 142 | 2647 | Entrez Gene | biogenesis of lysosome-related organelles complex-1, | 39133_at | Day 7 Peri/Day 7 Control | 0.520 | 1.5729E−03 |
| | | | subunit 1 | | | | |
| 143 | 7073 | Entrez Gene | TIA1 cytotoxic granule-associated RNA binding protein- | 41761_at | Day 3 Intra/Day 0 Intra | 0.521 | 4.5836E−03 |
| | | | like 1 | | | | |
| 144 | AF096870 | GenBank | tripartite motif-containing 16 /// similar to tripartite motif- | 38881_i_at | Day 3 Peri/Day 3 Extra | 0.521 | 1.0549E−04 |
| | | | containing 16; estrogen-responsive B box protein | | | | |
| 145 | 8853 | Entrez Gene | development and differentiation enhancing factor 2 | 39410_at | Day 7 Intra/Day 0 Intra | 0.521 | 2.9990E−03 |
| 146 | 4968 | Entrez Gene | 8-oxoguanine DNA glycosylase | 38335_at | Day 3 Intra/Day 0 Intra | 0.522 | 7.5868E−04 |
| | | | | 38335_at | Day 7 Intra/Day 0 Intra | 0.626 | 6.6349E−03 |
| | | | | 38335_at | Day 0 Peri/Day 0 Intra | 0.759 | 8.2179E−03 |
| 147 | 2110 | Entrez Gene | electron-transferring-flavoprotein dehydrogenase | 33494_at | Day 7 Intra/Day 7 Extra | 0.523 | 1.1689E−03 |
| 148 | 4713 | Entrez Gene | NADH dehydrogenase (ubiquinone) 1 beta subcomplex, | 35773_i_at | Day 7 Peri/Day 7 Control | 0.525 | 1.8840E−03 |
| | | | 7, 18 kDa | 35773_i_at Day | 7 Peri/Day 0 Peri | 0.748 | 5.4735E−03 |
| | | | | 35774_r_at | Day 7 Peri/Day 3 Peri | 0.791 | 1.2591E−03 |
| 149 | 1396 | Entrez Gene | cysteine-rich protein 1 (intestinal) | 33232_at | Day 7 Peri/Day 0 Peri | 0.526 | 3.2907E−04 |
| | | | | 33232_at | Day 3 Peri/Day 0 Peri | 0.596 | 1.5417E−03 |
| 150 | 771 | Entrez Gene | carbonic anhydrase XII | 36454_at | Day 0 Peri/Day 0 Control | 0.526 | 3.3461E−03 |
| | | | | 35275_at | Day 3 Peri/Day 3 Extra | 0.650 | 2.8445E−03 |
| 151 | 55187 | Entrez Gene | vacuolar protein sorting 13D (yeast) | 32743_at | Day 3 Intra/Day 0 Intra | 0.527 | 2.5585E−03 |
| | | | | 32743_at | Day 0 Peri/Day 0 Control | 0.619 | 1.5268E−03 |
| | | | | 32743_at | Day 3 Peri/Day 0 Peri | 0.772 | 6.0079E−03 |
| 152 | 498 | Entrez Gene | ATP synthase, H+ transporting, mitochondrial F1 | 40096_at | Day 3 Peri/Day 3 Control | 0.527 | 6.5302E−03 |
| | | | complex, alpha subunit, isoform 1, cardiac muscle | 40096_at | Day 0 Peri/Day 0 Control | 0.563 | 2.0338E−04 |
| 153 | 9140 | Entrez Gene | ATG12 autophagy related 12 homolog (S. cerevisiae) | 32720_at | Day 3 Intra/Day 3 Peri | 0.530 | 2.7861E−04 |
| 154 | 242 | Entrez Gene | arachidonate 12-lipoxygenase, 12R type | 33029_at | Day 7 Peri/Day 7 Extra | 0.530 | 9.3336E−03 |
| 155 | 23506 | Entrez Gene | KIAA0240 | 38892_at | Day 3 Intra/Day 0 Intra | 0.531 | 5.3954E−03 |
| 156 | 5985 | Entrez Gene | replication factor C (activator 1) 5, 36.5 kDa | 653_at | Day 0 Peri/Day 0 Control | 0.533 | 6.1850E−03 |
| 157 | 26610 | Entrez Gene | elongation protein 4 homolog (S. cerevisiae) | 38347_at | Day 0 Peri/Day 0 Intra | 0.533 | 6.3391E−03 |
| 158 | 6595 | Entrez Gene | SWI/SNF related, matrix associated, actin dependent | 40961_at | Day 3 Intra/Day 0 Intra | 0.533 | 6.2437E−03 |
| | | | regulator of chromatin, subfamily a, member 2 | | | | |
| 159 | 5110 | Entrez Gene | Protein-L-isoaspartate (D-aspartate) O- | 37737_at | Day 7 Intra/Day 7 Extra | 0.534 | 2.8151E−03 |
| | | | methyltransferase | 37738_g_at | Day 3 Peri/Day 3 Control | 0.678 | 3.6789E−03 |
| 160 | 113146 | Entrez Gene | chromosome 14 open reading frame 78 | 36497_at | Day 3 Peri/Day 3 Control | 0.535 | 3.4244E−03 |
| | | | | 36497_at | Day 7 Peri/Day 0 Peri | 0.545 | 2.9700E−05 |
| | | | | 36497_at | Day 3 Peri/Day 0 Peri | 0.577 | 3.7100E−05 |
| 161 | 201229 | Entrez Gene | hypothetical protein LOC201229 | 39557_at | Day 3 Intra/Day 0 Intra | 0.535 | 2.3943E−03 |
| 162 | 22909 | Entrez Gene | KIAA1018 protein | 36458_at | Day 3 Intra/Day 0 Intra | 0.536 | 6.1234E−03 |
| 163 | 51099 | Entrez Gene | Abhydrolase domain containing 5 | 33309_at | Day 3 Intra/Day 3 Peri | 0.536 | 9.8735E−03 |
| 164 | 114882 | Entrez Gene | oxysterol binding protein-like 8 | 41438_at | Day 3 Intra/Day 3 Peri | 0.536 | 3.0621E−03 |
| 165 | 10124 | Entrez Gene | ADP-ribosylation factor-like 4 | 33796_at | Day 3 Peri/Day 3 Control | 0.537 | 6.8833E−03 |
| 166 | 10653 | Entrez Gene | serine peptidase inhibitor, Kunliz type, 2 | 34348_at | Day 3 Peri/Day 3 Control | 0.537 | 9.4449E−03 |
| | | | | 34348_at | Day 0 Peri/Day 0 Control | 0.573 | 7.9326E−03 |
| | | | | 34348_at | Day 7 Intra/Day 7 Extra | 0.699 | 5.3002E−03 |
| 167 | 10434 | Entrez Gene | lysophospholipase I | 39396_at | Day 3 Peri/Day 3 Control | 0.541 | 7.9687E−03 |
| 168 | 399563 | Entrez Gene | hypothetical protein FLJ43806 | 37151_at | Day 7 Intra/Day 0 Intra | 0.542 | 7.7014E−03 |
| 169 | 123 | Entrez Gene | adipose differentiation-related protein | 34378_at | Day 7 Peri/Day 7 Extra | 0.542 | 1.2411E−03 |
| 170 | 80308 | Entrez Gene | Fad1, flavin adenine dinucleotide synthetase, homolog | 39074_at | Day 3 Peri/Day 3 Control | 0.544 | 3.5087E−04 |
| | | | (yeast) | | | | |
| 171 | 1662 | Entrez Gene | DEAD (Asp-Glu-Ala-Asp) box polypeptide 10 | 831_at | Day 7 Intra/Day 7 Extra | 0.544 | 5.2360E−03 |
| 172 | 5130 | Entrez Gene | phosphate cytidylyltransferase 1, choline, alpha | 39285_at | Day 0 Intra/Day 0 Peri | 0.545 | 2.3355E−03 |
| 173 | 23345 | Entrez Gene | spectrin repeat containing, nuclear envelope 1 | 38113_at | Day 0 Peri/Day 0 Control | 0.546 | 1.8607E−03 |
| 174 | 26235 | Entrez Gene | F-box and leucine-rich repeat protein 4 | 39987_at | Day 7 Intra/Day 7 Peri | 0.546 | 3.2958E−03 |
| 175 | 80208 | Entrez Gene | hypothetical protein FLJ21439 | 40615_at | Day 0 Peri/Day 0 Control | 0.546 | 5.1650E−03 |
| 176 | 202 | Entrez Gene | absent in melanoma 1 | 32112_s_at | Day 3 Peri/Day 3 Extra | 0.547 | 1.9891E−03 |
| | | | | 32112_s_at | Day 3 Intra/Day 3 Extra | 0.617 | 9.6499E−03 |
| 177 | 9774 | Entrez Gene | BCL2-associated transcription factor 1 | 38050_at | Day 7 Intra/Day 7 Extra | 0.547 | 4.7061E−03 |
| 178 | 5268 | Entrez Gene | serpin peptidase inhibitor, clade B (ovalbumin), member 5 | 862_at | Day 3 Peri/Day 3 Extra | 0.548 | 8.5245E−03 |
| | | | | 862_at | Day 7 Peri/Day 7 Extra | 0.576 | 5.9941E−03 |
| 179 | 1474 | Entrez Gene | cystatin E/M | 33128_s_at | Day 7 Peri/Day 0 Peri | 0.549 | 1.9072E−03 |
| | | | | 33128_s_at | Day 3 Peri/Day 0 Peri | 0.605 | 2.6588E−03 |
| 180 | 9927 | Entrez Gene | mitofusin 2 | 34369_at | Day 3 Peri/Day 3 Control | 0.549 | 2.5016E−03 |
| | | | | 34369_at | Day 3 Peri/Day 3 Extra | 0.635 | 6.4361E−03 |
| 181 | 4232 | Entrez Gene | mesoderm specific transcript homolog (mouse) | 37749_at | Day 7 Intra/Day 7 Extra | 0.553 | 9.8929E−04 |
| 182 | 2145 | Entrez Gene | enhancer of zeste homolog 1 (Drosophila) | 32259_at | Day 7 Intra/Day 0 Intra | 0.554 | 8.0450E−03 |
| | | | | 32259_at | Day 3 Peri/Day 3 Control | 0.593 | 1.0957E−03 |
| 183 | 2959 | Entrez Gene | general transcription factor IIB | 1070_at | Day 3 Peri/Day 3 Control | 0.556 | 4.5246E−03 |
| 184 | U73737 | GenBank | — | 1017_at | Day 3 Intra/Day 3 Peri | 0.556 | 4.6650E−03 |
| 185 | 4736 | Entrez Gene | ribosomal protein L10a | 36786_at | Day 3 Peri/Day 3 Extra | 0.556 | 7.2273E−04 |
| | | | | 36786_at | Day 7 Peri/Day 7 Control | 0.592 | 1.4203E−03 |
| | | | | 36786_at | Day 3 Peri/Day 3 Control | 0.622 | 8.0958E−04 |
| | | | | 36786_at | Day 0 Peri/Day 0 Control | 0.668 | 3.0374E−03 |
| | | | | 36786_at | Day 7 Peri/Day 0 Peri | 0.783 | 1.0376E−03 |
| 186 | 644 | Entrez Gene | biliverdin reductase A | 32618_at | Day 3 Peri/Day 3 Control | 0.557 | 3.0605E−04 |
| | | | | 32618_at | Day 3 Peri/Day 3 Extra | 0.620 | 3.2950E−04 |
| | | | | 32618_at | Day 0 Peri/Day 0 Control | 0.677 | 7.9770E−03 |
| | | | | 32618_at | Day 3 Peri/Day 3 Intra | 0.702 | 5.1159E−03 |
| 187 | 9665 | Entrez Gene | limkain b1 | 31936_s_at | Day 3 Peri/Day 3 Control | 0.558 | 3.7112E−03 |
| 188 | 3033 | Entrez Gene | L-3-hydroxyacyl-Coenzyme A dehydrogenase, short chain | 35435_s_at | Day 3 Peri/Day 3 Extra | 0.558 | 7.5277E−03 |
| | | | | 35435_s_at | Day 7 Peri/Day 7 Extra | 0.692 | 5.5220E−03 |
| 189 | 10300 | Entrez Gene | katanin p80 (WD repeat containing) subunit B 1 | 40976_at | Day 3 Intra/Day 3 Extra | 0.559 | 9.1699E−03 |
| 190 | 55651 | Entrez Gene | nucleolar protein family A, member 2 (H/ACA small nucleolar RNPs) | 41322_s_at | Day 3 Peri/Day 3 Extra | 0.559 | 1.2634E−03 |
| | | | | 41322_s_at | Day 3 Intra/Day 3 Extra | 0.675 | 9.6475E−03 |
| 191 | 11336 | Entrez Gene | SEC6-like 1 (S. cerevisiae) | 37597_s_at | Day 3 Peri/Day 3 control | 0.560 | 4.7635E−03 |
| | | | | 37597_s_at | Day 3 Peri/Day 3 Extra | 0.566 | 9.6420E−04 |
| 192 | 7009 | Entrez Gene | testis enhanced gene transcript (BAX inhibitor 1) | 33988_at | Day 3 Peri/Day 3 Control | 0.560 | 1.0937E−03 |
| | | | | 33988_at | Day 3 Peri/Day 3 Extra | 0.582 | 8.2347E−04 |
| 193 | 23107 | Entrez Gene | mitochondrial ribosomal protein S27 | 39377_at | Day 3 Peri/Day 3 Extra | 0.561 | 3.7750E−03 |
| | | | | 39377_at | Day 3 Peri/Day 3 Control | 0.571 | 9.8955E−03 |
| | | | | 39377_at | Day 3 Peri/Day 3 Intra | 0.634 | 9.8004E−03 |
| 194 | 50 | Entrez Gene | aconitase 2, mitochondrial | 160044_g_at | Day 3 Peri/Day 3 Control | 0.562 | 5.6984E−03 |
| 195 | 2746 | Entrez Gene | glutamate dehydrogenase 1 | 31691_g_at | Day 3 Peri/Day 3 Control | 0.563 | 6.9886E−03 |
| 196 | 57209 | Entrez Gene | zinc finger protein 248 | 35139_at | Day 3 Intra/Day 0 Intra | 0.565 | 2.1978E−03 |
| 197 | 6477 | Entrez Gene | Seven in absentia homolog 1 (Drosophila) | 32161_at | Day 0 Peri/Day 0 Intra | 0.565 | 2.6625E−03 |
| 198 | 6166 | Entrez Gene | ribosomal protein L36a-like | 39856_at | Day 0 Peri/Day 0 Control | 0.568 | 7.8476E−03 |
| 199 | 7175 | Entrez Gene | translocated promoter region (to activated MET oncogene) | 421_at | Day 0 Peri/Day 0 Control | 0.569 | 3.6369E−03 |
| 200 | 8349 | Entrez Gene | histone 2, H2be | 33352_at | Day 3 Intra/Day 3 Control | 0.570 | 6.1256E−03 |
| | | | | 33352_at | Day 3 Intra/Day 3 Extra | 0.617 | 5.0561E−03 |
| 201 | 171546 | Entrez Gene | chromosome 14 open reading frame 147 | 33399_at | Day 3 Peri/Day 3 Extra | 0.571 | 4.6866E−03 |
| | | | | 33399_at | Day 3 Intra/Day 3 Extra | 0.656 | 8.8383E−03 |
| 202 | 10553 | Entrez Gene | HIV-1 Tat interactive protein 2, 30 kDa | 38824_at | Day 3 Peri/Day 3 Control | 0.571 | 7.2789E−03 |
| | | | | 38824_at | Day 3 Peri/Day 3 Extra | 0.592 | 5.7073E−03 |
| 203 | 115817 | Entrez Gene | dehydrogenase/reductase (SDR family) member 1 | 39103_s_at | Day 3 Peri/Day 3 Control | 0.571 | 2.1724E−04 |
| | | | | 39103_s_at | Day 0 Intra/Day 0 Control | 0.592 | 1.8258E−03 |
| | | | | 39103_s_at | Day 3 Peri/Day 3 Extra | 0.601 | 5.7200E−03 |
| 204 | 25813 | Entrez Gene | sorting and assembly machinery component 50 | 34845_at | Day 3 Peri/Day 3 Extra | 0.572 | 1.2889E−03 |
| | | | homolog (S. cerevisiae) | | | | |
| 205 | 25963 | Entrez Gene | DKFZP564G2022 protein | 40437_at | Day 3 Peri/Day 3 Control | 0.572 | 3.2143E−03 |
| 206 | 1212 | Entrez Gene | clathrin, light polypeptide (Lcb) | 32523_at | Day 3 Peri/Day 3 Control | 0.573 | 4.0809E−03 |
| 207 | 1652 | Entrez Gene | D-dopachrome lautomerase | 374_f_at | Day 7 Peri/Day 7 Control | 0.573 | 7.7105E−03 |
| 208 | 27335 | Entrez Gene | eukaryotic translation initiation factor 3, subunit 12 | 31492_at | Day 3 Peri/Day 3 Extra | 0.574 | 7.4544E−04 |
| 209 | 217 | Entrez Gene | aldehyde dehydrogenase 2 family (mitochondrial) | 32747_at | Day 3 Peri/Day 3 Extra | 0.574 | 5.1549E−04 |
| | | | | 32747_at | Day 7 Peri/Day 0 Peri | 0.741 | 2.4111E−03 |
| 210 | 10049 | Entrez Gene | DnaJ (Hsp40) homolog, subfamily B, member 6 | 41234_at | Day 7 Peri/Day 7 Extra | 0.577 | 3.8743E−03 |
| 211 | 8315 | Entrez Gene | BRCA1 associated protein | 41512_at | Day 3 Intra/Day 0 Intra | 0.577 | 1.1128E−03 |
| | | | | 41512_at | Day 3 Intra/Day 3 Peri | 0.677 | 7.0812E−04 |
| 212 | 8623 | Entrez Gene | acetylserotonin O-methyltransferase-like | 36553_at | Day 3 Intra/Day 0 Intra | 0.578 | 7.1773E−04 |
| | | | | 36553_at | Day 3 Peri/Day 0 Peri | 0.710 | 1.0994E−04 |
| 213 | 169611 | Entrez Gene | olfactomedin-like 2A | 38312_at | Day 3 Intra/Day 0 Intra | 0.579 | 3.6248E−03 |
| | | | | 38312_at | Day 3 Peri/Day 0 Peri | 0.719 | 4.2489E−04 |
| 214 | 11066 | Entrez Gene | U11/U12 snRNP 35K | 41029_at | Day 3 Peri/Day 3 Extra | 0.580 | 1.0146E−03 |
| | | | | 41029_at | Day 3 Intra/Day 3 Extra | 0.589 | 7.2521E−03 |
| 215 | 9936 | Entrez Gene | CD302 antigen | 34760_at | Day 0 Peri/Day 0 Intra | 0.580 | 5.4949E−04 |
| 216 | 4729 | Entrez Gene | NADH dehydrogenase (ubiquinone) flavoprotein 2, | 34893_at | Day 7 Peri/Day 7 Control | 0.581 | 9.2931E−03 |
| | | | 24 kDa | 34893_at | Day 7 Peri/Day 7 Extra | 0.600 | 1.3243E−03 |
| 217 | 987 | Entrez Gene | LPS-responsive vesicle trafficking, beach and anchor | 35371_at | Day 3 Peri/Day 3 Control | 0.582 | 1.9359E−03 |
| | | | containing | 35371_at | Day 3 Peri/Day 3 Extra | 0.618 | 2.4693E−03 |
| | | | | 35371_at | Day 3 Intra/Day 3 Control | 0.652 | 2.1271E−03 |
| 218 | 128 | Entrez Gene | alcohol dehydrogenase 5 (class III), chi polypeptide | 37708_r_at | Day 3 Intra/Day 0 Intra | 0.582 | 8.9780E−03 |
| | | | | 37708_r_at | Day 0 Peri/Day 0 Intra | 0.600 | 6.4350E−03 |
| 219 | 65055 | Entrez Gene | chromosome 2 open reading frame 23 | 36096_at | Day 3 Intra/Day 3 Peri | 0.583 | 2.9762E−03 |
| 220 | 7295 | Entrez Gene | thioredoxin | 36992_at | Day 3 Peri/Day 3 Extra | 0.583 | 3.0827E−03 |
| | | | | 36992_at | Day 3 Peri/Day 3 Intra | 0.673 | 4.8045E−03 |
| 221 | 5917 | Entrez Gene | arginyl-tRNA synthetase | 549_at | Day 7 Intra/Day 7 Extra | 0.584 | 6.5983E−03 |
| 222 | 10980 | Entrez Gene | COP9 constitutive photomorphogenic homolog subunit | 40138_at | Day 7 Peri/Day 7 Control | 0.584 | 6.5665E−03 |
| | | | 6 (Arabidopsis) | | | | |
| 223 | 2621 | Entrez Gene | growth arrest-specific 6 | 1597_at | Day 3 Intra/Day 0 Intra | 0.585 | 6.6299E−03 |
| | | | | 1597_at | Day 3 Peri/Day 0 Peri | 0.689 | 5.8701E−03 |
| 224 | 831 | Entrez Gene | calpastatin | 41257_at | Day 3 Intra/Day 0 Intra | 0.585 | 5.3045E−04 |
| | | | | 41257_at | Day 0 Peri/Day 0 Intra | 0.700 | 3.4819E−03 |
| 225 | 5927 | Entrez Gene | Jumonji, AT rich interactive domain 1A (RBBP2-like) | 1785_at | Day 3 Intra/Day 3 Peri | 0.585 | 3.1353E−03 |
| 226 | 2824 | Entrez Gene | glycoprotein M6B | 37251_s_at | Day 3 Intra/Day 3 Peri | 0.586 | 8.6602E−03 |
| 227 | 1153 | Entrez Gene | cold inducible RNA binding protein | 39864_at | Day 3 Intra/Day 0 Intra | 0.587 | 2.1268E−03 |
| | | | | 39864_at | Day 7 Intra/Day 0 Intra | 0.611 | 6.9554E−03 |
| 228 | 645 | Entrez Gene | biliverdin reductase B (flavin reductase (NADPH)) | 37002_at | Day 7 Peri/Day 7 Control | 0.588 | 8.3451E−04 |
| 229 | 8581 | Entrez Gene | lymphocyte antigen 6 complex, locus D | 36284_at | Day 7 Peri/Day 7 Extra | 0.589 | 9.2936E−03 |
| 230 | AB006780 | GenBank | lectin, galactoside-binding, soluble, 3 (galeclin 3) /// | 35367_at | Day 3 Intra/Day 0 Intra | 0.590 | 8.4864E−03 |
| | | | galectin-3 internal gene | 35367_at | Day 7 Peri/Day 0 Peri | 0.683 | 4.0159E−04 |
| | | | | 35367_at | Day 3 Peri/Day 0 Peri | 0.763 | 8.3297E−03 |
| 231 | 6929 | Entrez Gene | transcription factor 3 (E2A immunoglobulin enhancer | 1373_at | Day 3 Intra/Day 0 Intra | 0.590 | 2.7482E−03 |
| | | | binding factors E12/E47) | 1373_at | Day 3 Peri/Day 0 Peri | 0.738 | 3.1310E−04 |
| 232 | 10476 | Entrez Gene | ATP synthase, H+ transporting, mitochondrial F0 | 35760_at | Day 3 Peri/Day 3 Extra | 0.590 | 1.5912E−03 |
| | | | complex, subunit d | 35760_at | Day 0 Peri/Day 0 Control | 0.738 | 8.9277E−03 |
| 233 | 7022 | Entrez Gene | transcription factor AP-2 gamma (activating enhancer | 40303_at | Day 3 Peri/Day 3 Control | 0.591 | 7.9662E−03 |
| | | | binding protein 2 gamma) | | | | |
| 234 | 2806 | Entrez Gene | glutamic-oxaloacetic transaminase 2, mitochondrial | 40764_at | Day 3 Peri/Day 3 Extra | 0.591 | 2.0500E−03 |
| | | | (aspartate aminotransferase 2) | | | | |
| 235 | 23185 | Entrez Gene | La ribonucleoprotein domain family, member 5 | 32586_at | Day 0 Peri/Day 0 Control | 0.591 | 3.7384E−03 |
| | | | | 32586_at | Day 3 Peri/Day 3 Control | 0.685 | 8.4169E−04 |
| | | | | 32586_at | Day 3 Peri/Day 3 Extra | 0.782 | 5.1596E−03 |
| 236 | HG3254-HT343 | The Institute for | — | 1163_at | Day 3 Intra/Day 3 Control | 0.591 | 5.8755E−03 |
| | | Genomic Research | | | | | |
| 237 | 708 | Entrez Gene | complement component 1, q subcomponent binding | 37668_at | Day 3 Peri/Day 3 Extra | 0.591 | 6.3705E−03 |
| | | | protein | | | | |
| 238 | 81875 | Entrez Gene | Interferon stimulated exonuclease gene 20 kDa-like 2 | 40946_at | Day 3 Peri/Day 3 Extra | 0.593 | 3.2988E−03 |
| 239 | AI032612 | GenBank | enolase 1, (alpha) /// small nuclear ribonucleoprotein | 41403_at | Day 3 Peri/Day 3 Extra | 0.593 | 2.1695E−03 |
| | | | polypeptide F | 41403_at | Day 7 Peri/Day 7 Extra | 0.674 | 9.8788E−04 |
| 240 | 26040 | Entrez Gene | SET binding protein 1 | 34990_at | Day 3 Peri/Day 0 Peri | 0.593 | 3.4533E−04 |
| | | | | 34990_at | Day 7 Peri/Day 0 Peri | 0.711 | 9.8766E−03 |
| 241 | 8673 | Entrez Gene | vesicle-associated membrane protein 8 (endobrevin) | 32715_at | Day 3 Peri/Day 3 Extra | 0.593 | 2.2624E−03 |
| | | | | 32715_at | Day 0 Peri/Day 0 Control | 0.628 | 1.3473E−03 |
| 242 | 23353 | Entrez Gene | unc-84 homolog A (C. elegans) | 36588_at | Day 7 Intra/Day 0 Intra | 0.594 | 2.9354E−03 |
| 243 | 4255 | Entrez Gene | O-6-methylguanine-DNA methyltransferase | 2051_at | Day 7 Peri/Day 7 Control | 0.594 | 5.4697E−03 |
| 244 | 6670 | Entrez Gene | Sp3 transcription factor | 41573_at | Day 3 Intra/Day 0 Intra | 0.594 | 6.5653E−03 |
| 245 | 6249 | Entrez Gene | restin (Reed-Steinberg cell-expressed intermediate | 34350_at | Day 7 Peri/Day 7 Extra | 0.595 | 8.1420E−03 |
| | | | filament-associated protein) | | | | |
| 246 | 11188 | Entrez Gene | nischarin | 33916_at | Day 0 Peri/Day 0 Control | 0.595 | 3.7132E−03 |
| | | | | 33916_at | Day 3 Peri/Day 3 Control | 0.647 | 6.0432E−03 |
| 247 | 2355 | Entrez Gene | FOS-like antigen 2 | 40268_at | Day 0 Intra/Day 0 Peri | 0.596 | 5.8255E−03 |
| 248 | 2547 | Entrez Gene | X-ray repair complementing defective repair in Chinese | 32766_at | Day 3 Peri/Day 3 Extra | 0.597 | 8.7735E−04 |
| | | | hamster cells 6 (Ku autoantigen, 70 kDa) | | | | |
| 249 | 545 | Entrez Gene | ataxia talangiectasia and Rad3 related | 37229_at | Day 7 Intra/Day 7 Extra | 0.597 | 5.6822E−03 |
| | | | | 37229_at | Day 7 Peri/Day 7 Extra | 0.619 | 5.1546E−03 |
| 250 | 4775 | Entrez Gene | nuclear factor of activated T-cells, cytoplasmic, | 40822_at | Day 0 Peri/Day 0 Control | 0.597 | 3.4027E−03 |
| | | | calcineurin-dependent 3 | 40823_s_at | Day 3 Peri/Day 3 Extra | 0.633 | 7.3198E−03 |
| | | | | 40822_at | Day 0 Peri/Day 0 Intra | 0.746 | 7.0070E−03 |
| 251 | 8776 | Entrez Gene | myotubularin related protein 1 | 34654_at | Day 3 Intra/Day 3 Extra | 0.598 | 1.6699E−03 |
| | | | | 34654_at | Day 3 Intra/Day 3 Control | 0.631 | 5.7609E−03 |
| 252 | 4790 | Entrez Gene | nuclear factor of kappa light polypeptide gene enhancer | 1378_g_at | Day 3 Peri/Day 3 Extra | 0.598 | 6.5881E−03 |
| | | | in B-cells 1 (p105) | 1378_g_at | Day 0 Peri/Day 0 Control | 0.646 | 7.6400E−03 |
| 253 | AL080234 | GenBank | Full-length cDNA clone CS0DM001YA04 of Fetal liver | 38070_at | Day 7 Intra/Day 0 Intra | 0.598 | 4.0826E−03 |
| | | | of Homo sapiens (human) | | | | |
| 254 | 26135 | Entrez Gene | SERPINE1 mRNA binding protein 1 | 40440_at | Day 0 Peri/Day 0 Control | 0.599 | 7.7405E−03 |
| | | | | 40440_at | Day 3 Peri/Day 3 Intra | 0.727 | 9.5681E−03 |
| 255 | 23234 | Entrez Gene | DnaJ (Hsp40) homolog, subfamily C, member 9 | 41569_at | Day 7 Peri/Day 7 Extra | 0.599 | 4.9293E−03 |
| | | | | 41569_at | Day 7 Intra/Day 7 Extra | 0.615 | 8.2268E−03 |
| 256 | 94239 | Entrez Gene | H2A histone family, member V | 39046_at | Day 3 Intra/Day 0 Intra | 0.600 | 7.9321E−03 |
| | | | | 39046_at | Day 7 Intra/Day 0 Intra | 0.650 | 5.0612E−03 |
| 257 | AI557912 | GenBank | translocase of outer mitochondrial membrane 7 | 39025_at | Day 7 Intra/Day 0 Intra | 0.601 | 3.8343E−03 |
| | | | homolog (yeast) /// hypothetical protein LOC201725 | 39025_at | Day 7 Peri/Day 7 Control | 0.716 | 2.2149E−03 |
| | | | | 39025_at | Day 7 Intra/Day 7 Control | 0.727 | 5.2071E−03 |
| | | | | 39025_at | Day 7 Peri/Day 0 Peri | 0.798 | 1.0424E−03 |
| 258 | 4437 | Entrez Gene | mutS homolog 3 (E. coli) | 1719_at | Day 3 Peri/Day 3 Extra | 0.603 | 6.7926E−03 |
| 259 | 3550 | Entrez Gene | IK cytokine, down-regulator of HLA II | 218_at | Day 3 Peri/Day 3 Control | 0.606 | 4.9835E−04 |
| 260 | 5453 | Entrez Gene | POU domain class 3, transcription factor 1 | 33675_at | Day 3 Peri/Day 0 Peri | 0.606 | 4.5158E−03 |
| 261 | 6651 | Entrez Gene | SON DNA binding protein | 39096_at | Day 7 Intra/Day 0 Intra | 0.607 | 3.4000E−03 |
| | | | | 39096_at | Day 7 Intra/Day 7 Peri | 0.641 | 4.2992E−03 |
| | | | | 39096_at | Day 7 Intra/Day 3 Intra | 0.710 | 4.2168E−03 |
| 262 | 353 | Entrez Gene | adenine phosphoribosyltransferase | 34310_at | Day 3 Peri/Day 3 Control | 0.607 | 7.6231E−03 |
| | | | | 34310_at | Day 3 Peri/Day 3 Intra | 0.619 | 3.4883E−03 |
| 263 | 6427 | Entrez Gene | splicing factor, arginine/serine-rich 2 | 36111_s_at | Day 7 Peri/Day 7 Extra | 0.607 | 1.0792E−03 |
| | | | | 36111_s_at | Day 7 Intra/Day 7 Extra | 0.665 | 1.5173E−04 |
| | | | | 36111_s_at | Day 7 Intra/Day 3 Intra | 0.709 | 6.0916E−03 |
| 264 | 221749 | Entrez Gene | chromosome 6 open reading frame 145 | 34246_at | Day 3 Intra/Day 0 Intra | 0.608 | 3.6829E−03 |
| 265 | 79703 | Entrez Gene | hypothetical protein FLJ22531 | 41804_at | Day 3 Peri/Day 3 Control | 0.608 | 1.1934E−04 |
| 266 | 27292 | Entrez Gene | dimethyladenosine transferase | 39883_at | Day 7 Peri/Day 7 Extra | 0.609 | 2.6811E−03 |
| 267 | 54665 | Entrez Gene | round spermatid basic protein 1 | 37828_at | Day 3 Intra/Day 0 Intra | 0.610 | 7.8572E−04 |
| | | | | 37828_at | Day 0 Peri/Day 0 Intra | 0.626 | 4.8045E−03 |
| 268 | 513 | Entrez Gene | ATP synthase, H+ transporting, mitochondrial F1 | 37992_s_at | Day 7 Peri/Day 7 Control | 0.611 | 6.2111E−03 |
| | | | complex, delta subunit | | | | |
| 269 | AL050091 | GenBank | glutamate receptor, ionotropic, N-methyl D-aspartate- | 35736_at | Day 0 Peri/Day 0 Control | 0.611 | 4.7952E−03 |
| | | | like 1A /// GRINL1A combined protein /// similar to | | | | |
| | | | glutamate receptor, ionotropic, N-methyl D-a | | | | |
| 270 | 3301 | Entrez Gene | DnaJ (Hsp40) homolog, subfamily A, member 1 | 39118_at | Day 7 Intra/Day 7 Extra | 0.611 | 5.7425E−03 |
| 271 | 9349 | Entrez Gene | ribosomal protein L23 | 32395_r_at | Day 3 Peri/Day 3 Extra | 0.611 | 2.2508E−03 |
| 272 | 22826 | Entrez Gene | DnaJ (Hsp40) homolog, subfamily C, member 8 | 36166_at | Day 0 Intra/Day 0 Control | 0.612 | 5.7794E−03 |
| 273 | 23090 | Entrez Gene | zinc finger protein 423 | 34950_at | Day 3 Peri/Day 0 Peri | 0.613 | 2.6182E−04 |
| | | | | 34950_at | Day 7 Intra/Day 7 Peri | 0.623 | 7.6235E−03 |
| 274 | 64112 | Entrez Gene | modulator of apoptosis 1 | 34767_at | Day 3 Intra/Day 0 Intra | 0.614 | 4.4658E−03 |
| 275 | 10497 | Entrez Gene | unc-13 homolog B (C. elegans) | 40087_at | Day 3 Peri/Day 3 Control | 0.614 | 3.7491E−03 |
| | | | | 40087_at | Day 0 Intra/Day 0 Control | 0.769 | 3.3634E−03 |
| 276 | 56957 | Entrez Gene | chromosome 14 open reading frame 132 | 41837_at | Day 3 Peri/Day 0 Peri | 0.614 | 6.2326E−04 |
| | | | | 41837_at | Day 7 Peri/Day 0 Peri | 0.660 | 1.7553E−03 |
| 277 | 8560 | Entrez Gene | degenerative spermatocyte homolog 1, lipid desaturase | 33337_at | Day 3 Peri/Day 3 Control | 0.614 | 8.5255E−03 |
| | | | (Drosophila) | 33337_at | Day 7 Peri/Day 7 Control | 0.626 | 3.0395E−03 |
| | | | | 33337_at | Day 7 Peri/Day 0 Peri | 0.690 | 1.2271E−03 |
| 278 | 10975 | Entrez Gene | ubiquinol-cytochrome c reductase, 6.4 kDa subunit | 38451_at | Day 7 Peri/Day 7 Control | 0.614 | 8.2100E−05 |
| 279 | 10641 | Entrez Gene | tumor suppressor candidate 4 | 40497_at | Day 0 Peri/Day 0 Control | 0.614 | 9.7424E−03 |
| | | | | 40497_at | Day 3 Peri/Day 3 Control | 0.655 | 1.1667E−04 |
| 280 | 8663 | Entrez Gene | eukaryotic translation initiation factor 3, subunit 8, | 34841_at | Day 3 Peri/Day 3 Extra | 0.615 | 9.0352E−03 |
| | | | 110 kDa | 34841_at | Day 3 Peri/Day 0 Peri | 0.776 | 9.4126E−03 |
| 281 | 50717 | Entrez Gene | WD repeat domain 42A | 39823_at | Day 3 Peri/Day 3 Control | 0.616 | 2.9015E−03 |
| 282 | 23392 | Entrez Gene | KIAA0368 | 34414_at | Day 7 Intra/Day 0 Intra | 0.616 | 1.2883E−03 |
| | | | | 34414_at | Day 0 Peri/Day 0 Intra | 0.644 | 5.3735E−03 |
| 283 | 5934 | Entrez Gene | retinoblastoma-like 2 (p130) | 32597_at | Day 3 Intra/Day 0 Intra | 0.616 | 8.7510E−03 |
| 284 | 4781 | Entrez Gene | Nuclear factor I/B | 41229_at | Day 3 Intra/Day 0 Intra | 0.616 | 3.0477E−03 |
| 285 | 23650 | Entrez Gene | tripartite motif-containing 29 | 1898_at | Day 7 Peri/Day 0 Peri | 0.617 | 3.0138E−03 |
| 286 | 8665 | Entrez Gene | eukaryotic translation initiation factor 3, subunit 5 | 32576_at | Day 3 Peri/Day 3 Extra | 0.617 | 4.1860E−03 |
| | | | epsilon, 47 kDa | | | | |
| 287 | 7203 | Entrez Gene | chaperonin containing TCP1, subunit 3 (gamma) | 40774_at | Day 3 Peri/Day 3 Extra | 0.617 | 6.5552E−03 |
| 288 | 3418 | Entrez Gene | Isocitrate dehydrogenase 2 (NADP+), mitochondrial | 32332_at | Day 0 Intra/Day 0 Peri | 0.617 | 1.2980E−03 |
| 289 | 9 | Entrez Gene | N-acetyltransferase 1 (arylamine N-acetyltransferase) | 38187_at | Day 7 Intra/Day 7 Extra | 0.619 | 5.7524E−03 |
| 290 | 25797 | Entrez Gene | glutaminyl-peptide cyclotransferase (glutaminyl cyclase) | 35966_at | Day 3 Intra/Day 0 Intra | 0.619 | 4.9522E−03 |
| | | | | 35966_at | Day 0 Peri/Day 0 Intra | 0.663 | 6.8919E−03 |
| 291 | 5092 | Entrez Gene | 6-pyruvoyl-tetrahydropterin synthase/dimerization | 34352_at | Day 7 Peri/Day 7 Control | 0.620 | 2.4354E−03 |
| | | | cofactor of hepatocyte nuclear factor 1 alpha (TCF1) | 34352_at | Day 3 Peri/Day 3 Intra | 0.704 | 1.5982E−03 |
| 292 | 7818 | Entrez Gene | death associated protein 3 | 1356_at | Day 0 Peri/Day 0 Control | 0.621 | 1.4788E−03 |
| 293 | U05861 | GenBank | aldo-keto reductase family 1, member C1 (dihydrodiol | 32805_at | Day 3 Peri/Day 3 Extra | 0.621 | 6.7226E−03 |
| | | | dehydrogenase 1; 20-alpha (3-alpha)-hydroxysteroid | | | | |
| | | | dehydrogenase) /// aldo-keto reductase family | | | | |
| 294 | 23063 | Entrez Gene | KIAA0261 | 40086_at | Day 3 Peri/Day 3 Control | 0.623 | 5.5939E−03 |
| 295 | 9529 | Entrez Gene | BCL2-associated athanogene 5 | 36463_at | Day 3 Peri/Day 3 Extra | 0.624 | 7.6918E−03 |
| 296 | 57147 | Entrez Gene | SCY1-like 3 (S. cerevisiae) | 41329_at | Day 3 Peri/Day 3 Control | 0.624 | 2.9063E−03 |
| 297 | 8888 | Entrez Gene | MCM3 minichromosome maintenance deficient 3 (S. cerevisiae) | 40469_at | Day 7 Intra/Day 7 Extra | 0.624 | 4.8160E−03 |
| | | | associated protein | | | | |
| 298 | 10289 | Entrez Gene | translation factor sui1 homolog | 33351_at | Day 0 Peri/Day 0 Intra | 0.624 | 5.2711E−04 |
| | | | | 33351_at | Day 3 Intra/Day 0 Intra | 0.673 | 1.7268E−03 |
| 299 | 9536 | Entrez Gene | prostaglandin E synthase | 38131_at | Day 3 Peri/Day 3 Extra | 0.626 | 9.0721E−03 |
| 300 | 22883 | Entrez Gene | calsyntenin 1 | 41498_at | Day 3 Peri/Day 3 Control | 0.626 | 9.2124E−04 |
| | | | | 41498_at | Day 3 Peri/Day 0 Peri | 0.777 | 3.9252E−03 |
| 301 | 7520 | Entrez Gene | X-ray repair complementing defective repair in Chinese | 2093_s_at | Day 3 Peri/Day 3 Control | 0.627 | 1.0646E−03 |
| | | | hamster cells 5 (double-strand-break rejoining; Ku | 38733_at | Day 0 Peri/Day 0 Control | 0.644 | 9.6568E−03 |
| | | | autoantigen, 80 kDa) | | | | |
| 302 | 24139 | Entrez Gene | echinoderm microtubule associated protein like 2 | 41328_s_at | Day 0 Intra/Day 0 Peri | 0.627 | 4.4037E−03 |
| 303 | 4928 | Entrez Gene | nucleoporin 98 kDa | 38911_at | Day 3 Peri/Day 3 Extra | 0.627 | 2.3405E−03 |
| 304 | 3290 | Entrez Gene | hydroxysteroid (11-beta) dehydrogenase 1 | 35702_at | Day 7 Peri/Day 0 Peri | 0.628 | 6.5442E−03 |
| 305 | 23351 | Entrez Gene | KIAA0323 | 32592_at | Day 0 Peri/Day 0 Control | 0.628 | 8.9436E−04 |
| | | | | 32592_at | Day 0 Intra/Day 0 Control | 0.679 | 8.2136E−03 |
| 306 | 2065 | Entrez Gene | v-erb-b2 erythroblastic leukemia viral oncogene | 2089_s_at | Day 0 Peri/Day 0 Control | 0.629 | 5.0250E−03 |
| | | | homolog 3 (avian) | 32787_at | Day 7 Peri/Day 0 Peri | 0.677 | 7.5081E−03 |
| 307 | 10217 | Entrez Gene | CTD (carboxy-terminal domain, RNA polymerase II, | 40196_at | Day 3 Peri/Day 3 Control | 0.630 | 3.3471E−03 |
| | | | polypeptide A) small phosphatase-like | 40196_at | Day 7 Peri/Day 0 Peri | 0.735 | 2.0122E−03 |
| | | | | 40196_at | Day 3 Peri/Day 0 Peri | 0.770 | 5.2302E−03 |
| 308 | 23658 | Entrez Gene | LSM5 homolog, U6 small nuclear RNA associated (S. cerevisiae) | 34270_at | Day 3 Peri/Day 3 Control | 0.631 | 1.9266E−03 |
| 309 | W28948 | GenBank | Full length insert cDNA YH77E09 | 37161_at | Day 3 Peri/Day 3 Extra | 0.631 | 4.5494E−03 |
| | | | | 37161_at | Day 3 Peri/Day 3 Control | 0.640 | 6.9170E−03 |
| 310 | 22903 | Entrez Gene | BTB (POZ) domain containing 3 | 37755_at | Day 3 Intra/Day 0 Intra | 0.631 | 9.7703E−03 |
| 311 | 4681 | Entrez Gene | neuroblastoma, suppression of tumorigenicity 1 | 37005_at | Day 0 Intra/Day 0 Peri | 0.631 | 3.2813E−03 |
| | | | | 37005_at | Day 7 Peri/Day 0 Peri | 0.642 | 1.7449E−03 |
| | | | | 37005_at | Day 3 Peri/Day 0 Peri | 0.695 | 2.4042E−03 |
| 312 | 10301 | Entrez Gene | deleted in lymphocytic leukemia, 1 | 33791_at | Day 7 Intra/Day 0 Intra | 0.631 | 8.9965E−03 |
| | | | | 33791_at | Day 3 Intra/Day 0 Intra | 0.726 | 6.7457E−03 |
| 313 | AF052138 | GenBank | FLJ35348 /// Bromodomain containing 3 | 41841_at | Day 3 Intra/Day 0 Intra | 0.632 | 4.2490E−03 |
| 314 | 6209 | Entrez Gene | ribosomal protein S15 | 39916_r_at | Day 7 Peri/Day 7 Control | 0.633 | 1.4158E−03 |
| | | | | 39916_r_at | Day 3 Peri/Day 3 Control | 0.675 | 7.7101E−03 |
| 315 | 8930 | Entrez Gene | methyl-CpG binding domain protein 4 | 34386_at | Day 7 Intra/Day 7 Extra | 0.634 | 3.7388E−03 |
| 316 | 8824 | Entrez Gene | carboxylesterase 2 (intestine, liver) | 40882_at | Day 7 Peri/Day 7 Intra | 0.635 | 9.8901E−03 |
| 317 | 10961 | Entrez Gene | endoplasmic reticulum protein 29 | 36945_at | Day 3 Peri/Day 3 Control | 0.635 | 9.2309E−04 |
| 318 | 7265 | Entrez Gene | tetratricopeptide repeat domain 1 | 37321_at | Day 3 Peri/Day 3 Control | 0.635 | 7.5603E−03 |
| 319 | 1428 | Entrez Gene | crystallin, mu | 38285_at | Day 3 Intra/Day 0 Intra | 0.636 | 1.2448E−03 |
| 320 | 10614 | Entrez Gene | Hexamethylene bis-acetamide inducible 1 | 40220_at | Day 3 Intra/Day 0 Intra | 0.636 | 3.7740E−03 |
| 321 | 6128 | Entrez Gene | ribosomal protein L6 | 31952_at | Day 3 Peri/Day 3 Extra | 0.637 | 8.7667E−03 |
| 322 | 5245 | Entrez Gene | prohibitin | 36592_at | Day 3 Peri/Day 3 Extra | 0.637 | 3.4444E−03 |
| 323 | 9296 | Entrez Gene | ATPase, H+ transporting, lysosomal 14 kDa, V1 subunit F | 37395_at | Day 7 Peri/Day 7 Control | 0.638 | 2.2448E−03 |
| | | | | 37395_at | Day 3 Peri/Day 3 Control | 0.722 | 8.6817E−04 |
| | | | | 37395_at | Day 3 Peri/Day 3 Extra | 0.810 | 7.4746E−03 |
| | | | | 37395_at | Day 7 Peri/Day 0 Peri | 0.840 | 4.0330E−03 |
| 324 | 1340 | Entrez Gene | cytochrome c oxidase subunit Vib polypeptide 1 | 40872_at | Day 3 Peri/Day 3 Extra | 0.638 | 1.3105E−03 |
| | | | (ubiquitous) | 40872_at | Day 3 Intra/Day 3 Extra | 0.698 | 6.3894E−03 |
| 325 | 1891 | Entrez Gene | enoyl Coenzyme A hydratase 1, peroxisomal | 32756_at | Day 3 Peri/Day 3 Extra | 0.638 | 8.7097E−03 |
| 326 | 1632 | Entrez Gene | dodecenoyl-Coenzyme A delta isomerase (3,2 trans- | 37982_at | Day 7 Peri/Day 7 Control | 0.639 | 2.3722E−03 |
| | | | enoyl-Coenzyme A isomerase) | | | | |
| 327 | 1933 | Entrez Gene | eukaryotic translation elongation factor 1 beta 2 | 35748_at | Day 3 Peri/Day 3 Extra | 0.639 | 7.6265E−04 |
| 328 | 6141 | Entrez Gene | ribosomal protein L18 | 31546_at | Day 7 Peri/Day 7 Control | 0.639 | 6.9963E−03 |
| | | | | 31546_at | Day 3 Peri/Day 3 Extra | 0.691 | 6.7124E−03 |
| | | | | 31546_at | Day 7 Peri/Day 0 Peri | 0.760 | 9.8180E−03 |
| 329 | 4026 | Entrez Gene | LIM domain containing preferred translocation partner in | 41195_at | Day 3 Intra/Day 3 Peri | 0.639 | 3.3595E−03 |
| | | | lipoma | | | | |
| 330 | 6950 | Entrez Gene | t-complex 1 | 34791_at | Day 3 Peri/Day 3 Extra | 0.640 | 3.7040E−03 |
| 331 | 29760 | Entrez Gene | B-cell linker | 38242_at | Day 3 Peri/Day 3 Extra | 0.640 | 7.2677E−04 |
| 332 | 573 | Entrez Gene | BCL2-associated athanogene | 34798_at | Day 3 Peri/Day 3 Control | 0.642 | 4.4022E−03 |
| 333 | 2709 | Entrez Gene | gap junction protein, beta 5 (connexin 31.1) | 38903_at | Day 7 Peri/Day 0 Peri | 0.643 | 1.7967E−03 |
| 334 | 6230 | Entrez Gene | ribosomal protein S25 | 31573_at | Day 7 Intra/Day 0 Intra | 0.643 | 9.4033E−03 |
| 335 | 11083 | Entrez Gene | death associated transcription factor 1 | 32045_at | Day 0 Peri/Day 0 Control | 0.644 | 6.8658E−03 |
| 336 | 10247 | Entrez Gene | heat-responsive protein 12 | 32173_at | Day 7 Peri/Day 7 Extra | 0.645 | 6.3967E−03 |
| | | | | 32173_at | Day 3 Peri/Day 3 Control | 0.659 | 6.9500E−03 |
| 337 | 27067 | Entrez Gene | staufen, RNA binding protein, homolog 2 (Drosophila) | 38341_at | Day 3 Intra/Day 3 Extra | 0.645 | 8.2036E−03 |
| 338 | 899 | Entrez Gene | cyclin F | 35907_at | Day 3 Peri/Day 3 Extra | 0.646 | 8.5538E−03 |
| 339 | HG162-HT3165 | The Institute for | — | 1278_at | Day 3 Peri/Day 0 Peri | 0.647 | 6.0118E−03 |
| | | Genomic Research | | | | | |
| 340 | 6139 | Entrez Gene | ribosomal protein L17 | 32440_at | Day 3 Peri/Day 3 Extra | 0.647 | 6.1617E−03 |
| 341 | 9521 | Entrez Gene | eukaryotic translation elongation factor 1 epsilon 1 | 40587_s_at | Day 7 Intra/Day 3 Intra | 0.647 | 1.9928E−03 |
| | | | | 40587_s_at | Day 3 Peri/Day 3 Intra | 0.697 | 4.8656E−04 |
| | | | | 40587_s_at | Day 3 Peri/Day 3 Extra | 0.718 | 6.3530E−03 |
| 342 | 1054 | Entrez Gene | CCAAT/enhancer binding protein (C/EBP), gamma | 39219_at | Day 7 Peri/Day 7 Extra | 0.647 | 8.7653E−03 |
| | | | | 39219_at | Day 7 Intra/Day 7 Extra | 0.676 | 1.0916E−03 |
| 343 | 7739 | Entrez Gene | zinc finger protein 185 (LIM domain) | 32139_at | Day 0 Peri/Day 0 Control | 0.648 | 5.7560E−03 |
| 344 | 8664 | Entrez Gene | eukaryotic translation initiation factor 3, subunit 7 zeta, | 35298_at | Day 3 Peri/Day 3 Extra | 0.649 | 2.0180E−03 |
| | | | 66/67 kDa | | | | |
| 345 | 57418 | Entrez Gene | WD repeat domain 18 | 35983_at | Day 3 Peri/Day 3 Extra | 0.651 | 3.7380E−03 |
| 346 | 6187 | Entrez Gene | ribosomal protein S2 | 31527_at | Day 3 Peri/Day 3 Extra | 0.651 | 6.9801E−03 |
| 347 | 11079 | Entrez Gene | RER1 retention in endoplasmic reticulum 1 homolog (S. cerevisiae) | 41551_at | Day 7 Peri/Day 7 Extra | 0.651 | 8.1132E−03 |
| 348 | 26211 | Entrez Gene | olfactory receptor, family 2, subfamily F, member 1 | 31921_at | Day 3 Peri/Day 3 Control | 0.652 | 2.1176E−03 |
| 349 | 6206 | Entrez Gene | ribosomal protein S12 | 33117_r_at | Day 3 Peri/Day 3 Extra | 0.652 | 2.4740E−03 |
| 350 | 9798 | Entrez Gene | KIAA0174 | 36942_at | Day 0 Peri/Day 0 Control | 0.653 | 5.6656E−03 |
| 351 | 132556 | Entrez Gene | similar to Transcription factor BTF3 homolog 3 | 31519_f_at | Day 0 Peri/Day 0 Intra | 0.653 | 9.2891E−03 |
| 352 | 8732 | Entrez Gene | RNA guanylyltransferase and 5′-phosphatase | 35202_at | Day 7 Intra/Day 7 Extra | 0.654 | 1.2688E−03 |
| 353 | 4070 | Entrez Gene | tumor-associated calcium signal transducer 2 | 291_s_at | Day 0 Peri/Day 0 Control | 0.654 | 9.5683E−03 |
| | | | | 291_s_at | Day 3 Peri/Day 3 Extra | 0.655 | 8.7414E−03 |
| 354 | 7152 | Entrez Gene | topoisomerase (DNA) I pseudogene 2 | 31680_at | Day 7 Peri/Day 7 Extra | 0.654 | 8.0454E−03 |
| 355 | 4615 | Entrez Gene | myeloid differentiation primary response gene (88) | 38369_at | Day 7 Peri/Day 7 Extra | 0.654 | 5.5186E−03 |
| 356 | 51567 | Entrez Gene | TRAF and TNF receptor associated protein | 34825_at | Day 3 Intra/Day 3 Extra | 0.655 | 3.0500E−05 |
| 357 | 9377 | Entrez Gene | cytochrome c oxidase subunit Va | 41223_at | Day 3 Peri/Day 3 Extra | 0.656 | 7.0969E−03 |
| 358 | 7386 | Entrez Gene | ubiquinol-cytochrome c reductase, Rieske iron-sulfur | 34401_at | Day 3 Peri/Day 3 Extra | 0.656 | 5.8159E−03 |
| | | | polypeptide 1 | | | | |
| 359 | 4835 | Entrez Gene | NAD(P)H dehydrogenase, quinone 2 | 36880_at | Day 7 Peri/Day 7 Control | 0.657 | 7.2935E−03 |
| 360 | 3298 | Entrez Gene | heat shock transcription factor 2 | 202_at | Day 7 Intra/Day 0 Intra | 0.658 | 7.1609E−03 |
| 361 | 4677 | Entrez Gene | asparaginyl-tRNA synthetase | 41241_at | Day 0 Intra/Day 0 Control | 0.658 | 3.0519E−03 |
| 362 | HG1980-HT202 | The institute for | — | 956_at | Day 7 Peri/Day 7 Extra | 0.658 | 8.0238E−03 |
| | | Genomic Research | | | | | |
| 363 | 23384 | Entrez Gene | KIAA0376 protein | 34837_at | Day 3 Intra/Day 0 Intra | 0.659 | 2.3071E−03 |
| 364 | 54107 | Entrez Gene | polymerase (DNA directed), epsilon 3 (p17 subunit) | 38702_at | Day 3 Peri/Day 3 Extra | 0.660 | 3.6355E−03 |
| 365 | 6635 | Entrez Gene | small nuclear ribonucleoprotein polypeptide E | 38679_g_at | Day 7 Intra/Day 7 Extra | 0.661 | 3.7759E−04 |
| | | | | 38679_g_at | Day 7 Intra/Day 3 Intra | 0.697 | 1.8800E−05 |
| 366 | 8192 | Entrez Gene | CIpP caseinolytic peptidase, ATP-dependent, | 32528_at | Day 7 Peri/Day 7 Intra | 0.661 | 7.3475E−03 |
| | | | proteolytic subunit homolog (E. coli) | | | | |
| 367 | 51690 | Entrez Gene | LSM7 homolog, U6 small nuclear RNA associated (S. cerevisiae) | 36846_s_at | Day 7 Peri/Day 7 Control | 0.662 | 8.8105E−04 |
| 368 | 5936 | Entrez Gene | RNA binding motif protein 4 | 35351_at | Day 3 Peri/Day 3 Extra | 0.662 | 1.3875E−03 |
| 369 | 7004 | Entrez Gene | TEA domain family member 4 | 41037_at | Day 7 Peri/Day 7 Extra | 0.662 | 6.4186E−04 |
| | | | | 41037_at | Day 7 Intra/Day 7 Extra | 0.710 | 7.7243E−03 |
| | | | | 41037_at | Day 0 Intra/Day 0 Peri | 0.755 | 4.0427E−03 |
| 370 | 3094 | Entrez Gene | histidine triad nucleotide binding protein 1 | 1009_at | Day 3 Peri/Day 3 Extra | 0.662 | 6.3134E−03 |
| | | | | 1009_at | Day 3 Peri/Day 3 Intra | 0.704 | 8.1604E−03 |
| 371 | 4832 | Entrez Gene | non-metastatic cells 3, protein expressed in | 197_at | Day 3 Intra/Day 3 Control | 0.663 | 6.9663E−03 |
| 372 | 10557 | Entrez Gene | ribonuclease P/MRP 38 kDa subunit | 41040_at | Day 7 Peri/Day 7 Extra | 0.663 | 2.3436E−03 |
| | | | | 41040_at | Day 7 Intra/Day 7 Extra | 0.667 | 4.5174E−03 |
| 373 | 26156 | Entrez Gene | ribosomal L1 domain containing 1 | 39418_at | Day 7 Peri/Day 7 Extra | 0.664 | 2.4392E−03 |
| | | | | 39418_at | Day 7 Intra/Day 3 Intra | 0.691 | 5.6288E−03 |
| | | | | 39418_at | Day 3 Peri/Day 3 Extra | 0.730 | 4.3297E−03 |
| 374 | 6122 | Entrez Gene | ribosomal protein L3 | 31722_at | Day 3 Peri/Day 3 Extra | 0.665 | 8.4932E−03 |
| | | | | 31722_at | Day 7 Peri/Day 0 Peri | 0.792 | 9.5624E−03 |
| 375 | 6176 | Entrez Gene | ribosomal protein, large, P1 | 31957_r_at | Day 7 Peri/Day 0 Peri | 0.666 | 3.7416E−03 |
| 376 | 26053 | Entrez Gene | autism susceptibility candidate 2 | 35648_at | Day 3 Intra/Day 0 Intra | 0.667 | 3.9622E−03 |
| 377 | 56339 | Entrez Gene | Methyltransferase like 3 | 32244_at | Day 3 Intra/Day 0 Intra | 0.667 | 8.9345E−04 |
| | | | | 32244_at | Day 3 Intra/Day 3 Extra | 0.726 | 7.6780E−03 |
| 378 | 10014 | Entrez Gene | histone deacetylase 5 | 38810_at | Day 3 Peri/Day 3 Control | 0.667 | 1.7702E−04 |
| | | | | 38810_at | Day 7 Peri/Day 0 Peri | 0.778 | 7.8739E−03 |
| | | | | 38810_at | Day 3 Peri/Day 0 Peri | 0.784 | 9.3572E−04 |
| 379 | 10610 | Entrez Gene | ST6 (alpha-N-acetyl-neuraminyl-2,3-beta-galactosyl- | 34693_at | Day 7 Peri/Day 0 Peri | 0.668 | 5.8340E−04 |
| | | | 1,3)-N-acetylgalactosaminide alpha-2,6- | | | | |
| | | | sialyltransferase 2 | | | | |
| 380 | HG662-HT662 | The Institute for | — | 1151_at | Day 3 Peri/Day 3 Control | 0.669 | 1.5550E−03 |
| | | Genomic Research | | | | | |
| 381 | 10694 | Entrez Gene | chaperonin containing TCP1, subunit 8 (theta) | 39767_at | Day 3 Peri/Day 3 Extra | 0.669 | 4.6792E−03 |
| 382 | 10038 | Entrez Gene | poly (ADP-ribose) polymerase family, member 2 | 34756_g_at | Day 7 Intra/Day 7 Extra | 0.669 | 7.1429E−03 |
| 383 | 4856 | Entrez Gene | nephroblastoma overexpressed gene | 39250_at | Day 3 Peri/Day 0 Peri | 0.670 | 5.1134E−03 |
| 384 | 9314 | Entrez Gene | Kruppel-like factor 4 (gut) | 36214_at | Day 7 Peri/Day 0 Peri | 0.670 | 7.5246E−03 |
| 385 | 4694 | Entrez Gene | NADH dehydrogenase (ubiquinone) 1 alpha | 36169_at | Day 3 Peri/Day 3 Control | 0.671 | 5.9310E−04 |
| | | | subcomplex, 1, 7.5 kDa | 36169_at | Day 3 Peri/Day 3 Extra | 0.706 | 1.3204E−03 |
| | | | | 36169_at | Day 7 Peri/Day 7 Control | 0.710 | 9.3036E−03 |
| | | | | 36169_at | Day 3 Peri/Day 3 Intra | 0.754 | 3.5482E−03 |
| 386 | 10904 | Entrez Gene | bladder cancer associated protein | 35267_g_at | Day 7 Intra/Day 0 Intra | 0.672 | 2.1451E−03 |
| | | | | 35266_at | Day 3 Peri/Day 0 Peri | 0.815 | 4.1112E−03 |
| 387 | 7411 | Entrez Gene | von Hippel-Lindau binding protein 1 | 171_at | Day 7 Intra/Day 7 Extra | 0.672 | 7.7370E−03 |
| 388 | 10682 | Entrez Gene | emopamil binding protein (sterol isomerase) | 32536_at | Day 3 Peri/Day 3 Extra | 0.672 | 3.7237E−03 |
| 389 | 8667 | Entrez Gene | eukaryotic translation initiation factor 3, subunit 3 | 35327_at | Day 3 Peri/Day 3 Control | 0.672 | 5.0660E−03 |
| | | | gamma, 40 kDa | | | | |
| 390 | 1349 | Entrez Gene | cytochrome c oxidase subunit VIIb | 36687_at | Day 7 Intra/Day 3 Intra | 0.673 | 1.8885E−03 |
| 391 | 54462 | Entrez Gene | KIAA1128 | 37617_at | Day 3 Intra/Day 0 Intra | 0.674 | 5.5416E−03 |
| | | | | 37617_at | Day 3 Intra/Day 3 Control | 0.696 | 9.3133E−03 |
| 392 | 80003 | Entrez Gene | pecanex-like 2 (Drosophila) | 39650_s_at | Day 3 Intra/Day 0 Intra | 0.674 | 6.1109E−03 |
| 393 | 25972 | Entrez Gene | unc-50 homolog (C. elegans) | 39442_at | Day 7 Intra/Day 7 Extra | 0.675 | 9.6835E−03 |
| 394 | 64795 | Entrez Gene | hypothetical protein FLJ13910 | 36580_at | Day 3 Intra/Day 0 Intra | 0.676 | 3.2610E−03 |
| 395 | 5441 | Entrez Gene | polymerase (RNA) II (DNA directed) polypeptide L, | 35841_at | Day 7 Peri/Day 7 Control | 0.676 | 2.4659E−03 |
| | | | 7.6 kDa | | | | |
| 396 | 2146 | Entrez Gene | enhancer of zeste homolog 2 (Drosophila) | 37305_at | Day 7 Peri/Day 7 Extra | 0.677 | 5.6094E−03 |
| | | | | 37305_at | Day 3 Peri/Day 3 Extra | 0.701 | 9.0063E−03 |
| 397 | 7743 | Entrez Gene | zinc finger protein 189 | 36072_at | Day 3 Intra/Day 0 Intra | 0.678 | 3.7246E−03 |
| 398 | 2950 | Entrez Gene | glutathione S-transferase pI | 33396_at | Day 3 Peri/Day 3 Extra | 0.679 | 6.0265E−03 |
| | | | | 33396_at | Day 7 Peri/Day 7 Extra | 0.695 | 5.1049E−03 |
| 399 | 50813 | Entrez Gene | COP9 constitutive photomorphogenic homolog subunit | 34404_at | Day 0 Intra/Day 0 Peri | 0.681 | 4.7805E−03 |
| | | | 7A (Arabidopsis) | | | | |
| 400 | 3156 | Entrez Gene | 3-hydroxy-3-methylglutaryl-Coenzyme A reductase | 39328_at | Day 3 Intra/Day 3 Extra | 0.681 | 6.1525E−03 |
| 401 | 6156 | Entrez Gene | ribosomal protein L30 | 31708_at | Day 3 Peri/Day 3 Extra | 0.682 | 4.7648E−03 |
| 402 | 516 | Entrez Gene | ATP synthase, H+ transporting, mitochondrial F0 | 38076_at | Day 3 Peri/Day 3 Extra | 0.682 | 3.2512E−04 |
| | | | complex, subunit c (subunit 9), isoform 1 | | | | |
| 403 | 9128 | Entrez Gene | PRP4 pre-mRNA processing factor 4 homolog (yeast) | 37936_at | Day 3 Peri/Day 3 Extra | 0.682 | 1.2100E−03 |
| | | | | 37936_at | Day 3 Peri/Day 3 Intra | 0.809 | 4.3656E−03 |
| 404 | 3028 | Entrez Gene | hydroxyacyl-Coenzyme A dehydrogenase, type II | 40778_at | Day 3 Peri/Day 3 Control | 0.683 | 9.5528E−03 |
| | | | | 40778_at | Day 3 Peri/Day 3 Extra | 0.687 | 5.4834E−03 |
| | | | | 40778_at | Day 0 Peri/Day 0 Control | 0.700 | 6.7401E−03 |
| | | | | 40778_at | Day 7 Peri/Day 0 Peri | 0.785 | 3.6406E−03 |
| 405 | 2961 | Entrez Gene | general transcription factor IIE, polypeptide 2, beta | 37295_at | Day 7 Peri/Day 7 Extra | 0.683 | 5.1041E−03 |
| | | | 34 kDa | 37295_at | Day 7 Intra/Day 7 Extra | 0.796 | 1.4862E−03 |
| 406 | 10969 | Entrez Gene | EBNA1 binding protein 2 | 36135_at | Day 3 Peri/Day 3 Extra | 0.683 | 7.3375E−03 |
| 407 | 6748 | Entrez Gene | signal sequence receptor, delta (translocon-associated | 38635_at | Day 7 Peri/Day 7 Control | 0.684 | 3.2000E−03 |
| | | | protein delta) | | | | |
| 408 | 4610 | Entrez Gene | v-myc myelocytomatosis viral oncogene homolog 1, | 1490_at | Day 3 Peri/Day 0 Peri | 0.684 | 2.9494E−04 |
| | | | lung carcinoma derived (avian) | | | | |
| 409 | 4170 | Entrez Gene | myeloid cell leukemia sequence 1 (BCL2-related) | 277_at | Day 3 Intra/Day 3 Extra | 0.684 | 9.6905E−03 |
| 410 | 23075 | Entrez Gene | SWAP-70 protein | 31869_at | Day 0 Peri/Day 0 Intra | 0.684 | 5.9906E−03 |
| 411 | 1537 | Entrez Gene | cytochrome c-1 | 1160_at | Day 3 Peri/Day 3 Control | 0.685 | 9.9616E−03 |
| 412 | 6768 | Entrez Gene | suppression of tumorigenicity 14 (colon carcinoma, | 35309_at | Day 3 Peri/Day 3 Extra | 0.685 | 2.8284E−03 |
| | | | matriptase, epithin) | | | | |
| 413 | 6138 | Entrez Gene | ribosomal protein L15 | 32433_at | Day 0 Peri/Day 0 Intra | 0.686 | 4.8021E−03 |
| 414 | 23112 | Entrez Gene | trinucleotide repeat containing 6B | 37487_at | Day 3 Intra/Day 3 Peri | 0.688 | 3.9849E−03 |
| 415 | 64976 | Entrez Gene | mitochondrial ribosomal protein L40 | 32745_at | Day 3 Peri/Day 3 Control | 0.688 | 7.4413E−03 |
| 416 | 10420 | Entrez Gene | testis-specific kinase 2 | 33164_at | Day 7 Peri/Day 0 Peri | 0.688 | 8.0384E−03 |
| 417 | 9759 | Entrez Gene | histone deacetylase 4 | 38271_at | Day 3 Intra/Day 0 Intra | 0.689 | 4.9833E−03 |
| | | | | 38271_at | Day 0 Peri/Day 0 Intra | 0.776 | 6.2327E−03 |
| 418 | 4676 | Entrez Gene | nucleosome assembly protein 1-like 4 | 32575_at | Day 3 Peri/Day 3 Control | 0.689 | 7.9600E−05 |
| | | | | 32575_at | Day 7 Intra/Day 7 Extra | 0.790 | 4.9890E−03 |
| | | | | 32575_at | Day 3 Peri/Day 3 Intra | 0.820 | 9.3028E−03 |
| 419 | 5425 | Entrez Gene | polymerase (DNA directed), delta 2, regulatory subunit | 1470_at | Day 3 Peri/Day 3 Intra | 0.689 | 5.7659E−03 |
| | | | 50 kDa | | | | |
| 420 | 6390 | Entrez Gene | succinate dehydrogenase complex, subunit B, iron | 35751_at | Day 3 Peri/Day 3 Extra | 0.689 | 9.9046E−03 |
| | | | sulfur (Ip) | | | | |
| 421 | 162 | Entrez Gene | adaptor-related protein complex 1, beta 1 subunit | 40745_at | Day 3 Peri/Day 3 Extra | 0.689 | 1.6623E−03 |
| 422 | 1749 | Entrez Gene | distal-less homeo box 5 | 41087_at | Day 3 Intra/Day 0 Intra | 0.689 | 3.6526E−03 |
| 423 | 6749 | Entrez Gene | structure specific recognition protein 1 | 37739_at | Day 3 Peri/Day 3 Extra | 0.690 | 4.8988E−03 |
| 424 | 4134 | Entrez Gene | microtubule-associated protein 4 | 33850_at | Day 3 Peri/Day 0 Peri | 0.690 | 9.2919E−04 |
| | | | | 33850_at | Day 7 Peri/Day 0 Peri | 0.747 | 8.7450E−03 |
| 425 | 6832 | Entrez Gene | suppressor of var1, 3-like 1 (S. cerevisiae) | 41408_at | Day 3 Peri/Day 3 Extra | 0.691 | 6.4202E−03 |
| 426 | 10939 | Entrez Gene | AFG3 ATPase family gene 3-like 2 (yeast) | 34315_at | Day 3 Peri/Day 3 Extra | 0.692 | 3.6562E−04 |
| 427 | 22794 | Entrez Gene | cancer susceptibility candidate 3 | 38437_at | Day 7 Intra/Day 0 Intra | 0.692 | 2.7057E−03 |
| | | | | 38437_at | Day 3 Intra/Day 0 Intra | 0.742 | 4.1247E−03 |
| 428 | 322 | Entrez Gene | amyloid beta (A4) precursor protein-binding, family B, | 1101_at | Day 7 Intra/Day 0 Intra | 0.692 | 1.6594E−03 |
| | | | member 1 (Fe65) | | | | |
| 429 | 10570 | Entrez Gene | dihydropyrimidinase-like 4 | 39503_s_at | Day 7 Peri/Day 0 Peri | 0.693 | 9.8616E−03 |
| 430 | 6637 | Entrez Gene | small nuclear ribonucleoprotein polypeptide G | 37337_at | Day 7 Intra/Day 3 Intra | 0.693 | 3.9690E−03 |
| 431 | 8337 | Entrez Gene | histone 2, H2aa | 286_at | Day 3 Peri/Day 3 Control | 0.693 | 7.8013E−03 |
| 432 | 6193 | Entrez Gene | ribosomal protein S5 | 32437_at | Day 3 Peri/Day 3 Extra | 0.693 | 1.4564E−03 |
| | | | | 32437_at | Day 7 Peri/Day 0 Peri | 0.740 | 3.6964E−03 |
| | | | | 32437_at | Day 3 Peri/Day 0 Peri | 0.808 | 6.6422E−03 |
| 433 | 23492 | Entrez Gene | chromobox homolog 7 | 36894_at | Day 3 Peri/Day 3 Control | 0.693 | 8.8784E−03 |
| | | | | 36894_at | Day 3 Peri/Day 0 Peri | 0.742 | 1.6692E−03 |
| | | | | 36894_at | Day 7 Peri/Day 0 Peri | 0.742 | 3.4050E−03 |
| 434 | 4809 | Entrez Gene | NHP2 non-histone chromosome protein 2-like 1 (S. cerevisiae) | 41746_at | Day 3 Peri/Day 3 Control | 0.693 | 5.4886E−03 |
| | | | | 41746_at | Day 3 Peri/Day 0 Peri | 0.830 | 6.1703E−03 |
| 435 | 889 | Entrez Gene | KRIT1, ankyrin repeat containing | 34031_i_at | Day 3 Intra/Day 3 Peri | 0.694 | 4.9207E−03 |
| 436 | 79090 | Entrez Gene | trafficking protein particle complex 6A | 36529_at | Day 3 Peri/Day 0 Peri | 0.694 | 6.8032E−03 |
| 437 | 328 | Entrez Gene | APEX nuclease (multifunctional DNA repair enzyme) 1 | 2025_s_at | Day 3 Peri/Day 3 Control | 0.695 | 4.9002E−03 |
| | | | | 2025_s_at | Day 3 Peri/Day 3 Extra | 0.746 | 9.3963E−03 |
| 438 | 84525 | Entrez Gene | homeodomain-only protein | 39698_at | Day 7 Peri/Day 0 Peri | 0.695 | 8.7289E−03 |
| 439 | 2549 | Entrez Gene | GRB2-associated binding protein 1 | 33997_at | Day 7 Intra/Day 0 Intra | 0.695 | 2.4091E−04 |
| | | | | 33997_at | Day 3 Intra/Day 0 Intra | 0.710 | 2.0825E−03 |
| | | | | 33997_at | Day 0 Peri/Day 0 Intra | 0.731 | 4.0375E−03 |
| 440 | 533 | Entrez Gene | ATPase, H+ transporting, lysosomal 21 kDa, V0 subunit | 36167_at | Day 7 Peri/Day 7 Control | 0.696 | 9.2513E−03 |
| | | | c″ | | | | |
| 441 | 4717 | Entrez Gene | NADH dehydrogenase (ubiquinone) 1, subcomplex | 38485_at | Day 3 Peri/Day 3 Extra | 0.696 | 2.0589E−03 |
| | | | unknown, 1, 6 kDa | | | | |
| 442 | 23385 | Entrez Gene | nicastrin | 34835_at | Day 3 Peri/Day 3 Extra | 0.696 | 1.6857E−03 |
| | | | | 34835_at | Day 3 Peri/Day 3 Intra | 0.790 | 4.8084E−03 |
| 443 | 23125 | Entrez Gene | calmodulin binding transcription activator 2 | 41421_at | Day 7 Intra/Day 0 Intra | 0.696 | 8.6760E−03 |
| 444 | 2926 | Entrez Gene | G-rich RNA sequence binding factor 1 | 32595_at | Day 7 Intra/Day 7 Extra | 0.696 | 6.0979E−03 |
| 445 | 4678 | Entrez Gene | nuclear autoantigenic sperm protein (histone-binding) | 33255_at | Day 3 Peri/Day 3 Control | 0.696 | 9.0975E−03 |
| 446 | 1847 | Entrez Gene | dual specificity phosphatase 5 | 529_at | Day 7 Peri/Day 7 Extra | 0.697 | 3.7478E−03 |
| 447 | 22849 | Entrez Gene | cytoplasmic polyadenylation element binding protein 3 | 34931_at | Day 3 Intra/Day 0 Intra | 0.697 | 6.8691E−03 |
| 448 | 10106 | Entrez Gene | CTD (carboxy-terminal domain, RNA polymerase II, | 41202_s_at | Day 3 Intra/Day 0 Intra | 0.698 | 3.5449E−03 |
| | | | polypeptide A) small phosphatase 2 | | | | |
| 449 | 57017 | Entrez Gene | chromosome 16 open reading frame 49 | 34810_at | Day 7 Peri/Day 7 Control | 0.700 | 1.2592E−03 |
| | | | | 34810_at | Day 7 Peri/Day 0 Peri | 0.810 | 4.2261E−03 |
| 450 | 10329 | Entrez Gene | transmembrane protein 5 | 37445_at | Day 3 Intra/Day 3 Extra | 0.700 | 2.4850E−03 |
| 451 | 26959 | Entrez Gene | HMG-box transcription factor 1 | 39809_at | Day 3 Intra/Day 0 Intra | 0.701 | 9.4669E−03 |
| 452 | 539 | Entrez Gene | ATP synthase, H+ transporting, mitochondrial F1 | 37029_at | Day 3 Peri/Day 3 Intra | 0.701 | 5.7342E−03 |
| | | | complex, O subunit (oligomycin sensitivity conferring | 37029_at | Day 7 Intra/Day 7 Control | 0.784 | 7.2352E−03 |
| | | | protein) | | | | |
| 453 | 10542 | Entrez Gene | hepatitis B virus x interacting protein | 38054_at | Day 3 Peri/Day 3 Control | 0.704 | 9.1709E−03 |
| | | | | 38054_at | Day 7 Intra/Day 7 Extra | 0.782 | 3.3831E−03 |
| 454 | 8624 | Entrez Gene | Down syndrome critical region gene 2 | 36088_at | Day 7 Intra/Day 3 Intra | 0.704 | 8.0554E−03 |
| 455 | 694 | Entrez Gene | B-cell translocation gene 1, anti-proliferative | 37294_at | Day 3 Peri/Day 3 Extra | 0.704 | 5.5679E−03 |
| 456 | 5431 | Entrez Gene | polymerase (RNA) II (DNA directed) polypeptide B, | 39746_at | Day 7 Intra/Day 7 Extra | 0.705 | 2.3347E−03 |
| | | | 140 kDa | | | | |
| 457 | 4174 | Entrez Gene | MCM5 minichromosome maintenance deficient 5, cell | 982_at | Day 7 Peri/Day 3 Peri | 0.706 | 4.9235E−04 |
| | | | division cycle 46 (S. cerevisiae) | | | | |
| 458 | 10278 | Entrez Gene | embryonal Fyn-associated substrate | 33883_at | Day 7 Peri/Day 0 Peri | 0.706 | 2.0000E−05 |
| | | | | 33883_at | Day 3 Peri/Day 0 Peri | 0.718 | 6.1275E−04 |
| 459 | 593 | Entrez Gene | branched chain keto acid dehydrogenase E1, alpha | 37704_at | Day 3 Peri/Day 3 Control | 0.707 | 1.4726E−03 |
| | | | polypeptide (maple syrup urine disease) | 37704_at | Day 3 Peri/Day 3 Extra | 0.720 | 1.7216E−03 |
| 460 | 4357 | Entrez Gene | mercaptopyruvate sulfurtransferase | 36124_at | Day 7 Peri/Day 0 Peri | 0.707 | 7.1338E−04 |
| 461 | 3727 | Entrez Gene | jun D proto-oncogene | 41483_s_at | Day 7 Peri/Day 0 Peri | 0.707 | 9.7391E−03 |
| 462 | 51635 | Entrez Gene | dehydrogenase/reductase (SDR family) member 7 | 39814_s_at | Day 3 Peri/Day 3 Extra | 0.708 | 6.9810E−03 |
| | | | | 39814_s_at | Day 3 Peri/Day 3 Intra | 0.740 | 6.8403E−03 |
| 463 | 22870 | Entrez Gene | SAPS domain family, member 1 | 36862_at | Day 3 Peri/Day 3 Control | 0.709 | 6.5040E−03 |
| 464 | 22882 | Entrez Gene | zinc fingers and homeoboxes 2 | 41503_at | Day 3 Peri/Day 0 Peri | 0.709 | 9.7700E−05 |
| | | | | 41503_at | Day 7 Intra/Day 7 Peri | 0.750 | 6.4066E−03 |
| | | | | 41503_at | Day 7 Peri/Day 0 Peri | 0.830 | 5.9812E−03 |
| 465 | 613 | Entrez Gene | breakpoint cluster region | 34679_at | Day 3 Peri/Day 3 Extra | 0.709 | 4.4827E−03 |
| 466 | 7150 | Entrez Gene | topoisomerase (DNA) I | 1030_s_at | Day 7 Peri/Day 7 Extra | 0.711 | 1.5853E−03 |
| 467 | 1329 | Entrez Gene | cytochrome c oxidase subunit Vb | 39921_at | Day 3 Peri/Day 3 Extra | 0.711 | 8.1965E−03 |
| | | | | 39921_at | Day 7 Intra/Day 7 Control | 0.718 | 5.7364E−03 |
| 468 | 51020 | Entrez Gene | HD domain containing 2 | 34359_at | Day 7 Intra/Day 0 Intra | 0.712 | 2.4876E−03 |
| | | | | 34359_at | Day 7 Intra/Day 7 Extra | 0.777 | 2.8509E−03 |
| 469 | 6888 | Entrez Gene | transaldolase 1 | 37311_at | Day 3 Peri/Day 3 Extra | 0.713 | 2.9353E−04 |
| 470 | 3184 | Entrez Gene | heterogeneous nuclear ribonucleoprotein D (AU-rich | 38016_at | Day 0 Peri/Day 0 Intra | 0.714 | 4.2703E−03 |
| | | | element RNA binding protein 1, 37 kDa) | | | | |
| 471 | 6050 | Entrez Gene | ribonuclease/angiogenin inhibitor 1 | 36187_at | Day 3 Peri/Day 3 Extra | 0.715 | 1.3879E−03 |
| | | | | 36187_at | Day 3 Peri/Day 3 Control | 0.730 | 5.3879E−03 |
| 472 | 2064 | Entrez Gene | v-erb-b2 erythroblastic leukemia viral oncogene | 33218_at | Day 3 Peri/Day 0 Peri | 0.718 | 4.9044E−04 |
| | | | homolog 2, neuro/glioblastoma derived oncogene | 33218_at | Day 7 Peri/Day 0 Peri | 0.740 | 6.2999E−04 |
| | | | homolog (avian) | | | | |
| 473 | 3054 | Entrez Gene | host cell factor C1 (VP16-accessory protein) | 37910_at | Day 0 Intra/Day 0 Peri | 0.718 | 6.3341E−03 |
| 474 | 79026 | Entrez Gene | AHNAK nucleoprotein (desmoyokin) | 37027_at | Day 7 Intra/Day 0 Intra | 0.718 | 8.1471E−03 |
| | | | | 37027_at | Day 7 Peri/Day 0 Peri | 0.748 | 8.8700E−05 |
| | | | | 37027_at | Day 3 Peri/Day 0 Peri | 0.780 | 3.1374E−04 |
| 475 | U77948 | GenBank | general transcription factor II, i /// general transcription | 35450_s_at | Day 0 Peri/Day 0 Control | 0.718 | 8.5556E−03 |
| | | | factor II, i, pseudogene 1 | | | | |
| 476 | 949 | Entrez Gene | scavenger receptor class B, member 1 | 41200_at | Day 7 Peri/Day 0 Peri | 0.720 | 1.3768E−03 |
| | | | | 41200_at | Day 3 Peri/Day 0 Peri | 0.721 | 1.6250E−03 |
| 477 | 1917 | Entrez Gene | eukaryotic translation elongation factor 1 alpha 2 | 35174_i_at | Day 7 Peri/Day 0 Peri | 0.720 | 6.7372E−04 |
| 478 | 6205 | Entrez Gene | ribosomal protein S11 | 32330_at | Day 7 Peri/Day 0 Peri | 0.721 | 3.3215E−03 |
| 479 | 10611 | Entrez Gene | PDZ and LIM domain 5 | 37366_at | Day 3 Intra/Day 3 Extra | 0.722 | 2.3752E−03 |
| 480 | HG1614-HT161 | The Institute for | — | 954_s_at | Day 7 Peri/Day 7 Control | 0.722 | 8.4646E−03 |
| | | Genomic Research | | 954_s_at | Day 7 Peri/Day 7 Intra | 0.747 | 1.0730E−03 |
| 481 | 79095 | Entrez Gene | chromosome 9 open reading frame 16 | 41047_at | Day 3 Peri/Day 0 Peri | 0.725 | 5.9374E−03 |
| 482 | 9813 | Entrez Gene | KIAA0494 | 41830_at | Day 3 Intra/Day 0 Intra | 0.725 | 3.4757E−03 |
| 483 | 22924 | Entrez Gene | microtubule-associated protein, RP/EB family, member 3 | 40825_at | Day 7 Peri/Day 0 Peri | 0.725 | 1.1882E−03 |
| | | | | 40825_at | Day 3 Peri/Day 0 Peri | 0.765 | 2.4008E−03 |
| 484 | 26578 | Entrez Gene | osteoclast stimulating factor 1 | 467_at | Day 7 Peri/Day 7 Extra | 0.725 | 7.2710E−03 |
| 485 | 1347 | Entrez Gene | cytochrome c oxidase subunit VIIa polypeptide 2 (liver) | 41760_at | Day 3 Peri/Day 3 Extra | 0.726 | 1.4279E−03 |
| 486 | 3615 | Entrez Gene | IMP (inosine monophosphate) dehydrogenase 2 | 36624_at | Day 3 Peri/Day 3 Extra | 0.726 | 5.8955E−03 |
| | | | | 36624_at | Day 7 Peri/Day 0 Peri | 0.740 | 8.1540E−04 |
| 487 | 1981 | Entrez Gene | eukaryotic translation initiation factor 4 gamma, 1 | 32844_at | Day 7 Peri/Day 0 Peri | 0.727 | 9.4700E−03 |
| 488 | 22934 | Entrez Gene | ribose 5-phosphate isomerase A (ribose 5-phosphate | 38036_at | Day 0 Peri/Day 0 Intra | 0.729 | 4.9732E−03 |
| | | | epimerase) | | | | |
| 489 | 26100 | Entrez Gene | WIPI49-like protein 2 | 33392_at | Day 3 Intra/Day 3 Extra | 0.731 | 6.9435E−03 |
| 490 | 11311 | Entrez Gene | vacuolar protein sorting 45A (yeast) | 35779_at | Day 0 Peri/Day 0 Intra | 0.731 | 7.1397E−03 |
| 491 | 3916 | Entrez Gene | lysosomal-associated membrane protein 1 | 39758_f_at | Day 3 Peri/Day 3 Extra | 0.732 | 1.6743E−03 |
| 492 | 23020 | Entrez Gene | activating signal cointegrator 1 complex subunit 3-like 1 | 41224_at | Day 3 Peri/Day 3 Extra | 0.732 | 7.2017E−03 |
| 493 | 10473 | Entrez Gene | high mobility group nucleosomal binding domain 4 | 35738_at | Day 3 Peri/Day 3 Control | 0.732 | 4.8156E−03 |
| 494 | 10075 | Entrez Gene | HECT, UBA and WWE domain containing 1 | 34374_g_at | Day 0 Intra/Day 0 Peri | 0.733 | 2.9867E−03 |
| | | | | 34372_at | Day 3 Peri/Day 0 Peri | 0.802 | 9.1907E−03 |
| 495 | 6812 | Entrez Gene | syntaxin binding protein 1 | 33942_s_at | Day 3 Peri/Day 3 Extra | 0.734 | 2.7323E−03 |
| 496 | 23511 | Entrez Gene | nucleoporin 188 kDa | 32644_at | Day 3 Peri/Day 3 Extra | 0.735 | 1.7224E−03 |
| 497 | HG2238HT232- | The Institute for | — | 329_s_at | Day 3 Peri/Day 0 Peri | 0.736 | 9.9413E−03 |
| | | Genomic Research | | | | | |
| 498 | 283638 | Entrez Gene | KIAA0284 | 38592_s_at | Day 3 Peri/Day 3 Extra | 0.736 | 1.6693E−03 |
| | | | | 38592_s_at | Day 3 Peri/Day 3 Control | 0.788 | 5.7373E−03 |
| 499 | 30968 | Entrez Gene | stomatin (EPB72)-like 2 | 34380_at | Day 3 Peri/Day 3 Extra | 0.736 | 6.0626E−03 |
| 500 | 23259 | Entrez Gene | DDHD domain containing 2 | 35177_at | Day 7 Intra/Day 0 Intra | 0.736 | 6.5771E−03 |
| 501 | 10634 | Entrez Gene | growth arrest-specific 2 like 1 | 31874_at | Day 7 Peri/Day 0 Peri | 0.737 | 7.5700E−05 |
| | | | | 31874_at | Day 3 Peri/Day 0 Peri | 0.791 | 1.9327E−03 |
| 502 | 3420 | Entrez Gene | isocitrate dehydrogenase 3 (NAD+) beta | 40111_g_at | Day 3 Peri/Day 3 Extra | 0.737 | 9.0628E−03 |
| 503 | 10424 | Entrez Gene | progesterone receptor membrane component 2 | 38821_at | Day 7 Peri/Day 0 Peri | 0.736 | 3.9496E−03 |
| | | | | 38821_at | Day 3 Peri/Day 0 Peri | 0.746 | 7.1002E−03 |
| 504 | 8531 | Entrez Gene | cold shock domain protein A | 39839_at | Day 7 Peri/Day 0 Peri | 0.738 | 8.5534E−03 |
| 505 | 5439 | Entrez Gene | polymerase (RNA) II (DNA directed) polypeptide J, | 1486_at | Day 7 Peri/Day 0 Peri | 0.740 | 2.7928E−03 |
| | | | 13.3 kDa | 1486_at | Day 7 Peri/Day 3 Peri | 0.772 | 1.7983E−03 |
| 506 | 10929 | Entrez Gene | Splicing factor, arginine/serine-rich, 46 kD | 32038_s_at | Day 3 Peri/Day 3 Control | 0.740 | 6.3052E−03 |
| 507 | 6227 | Entrez Gene | ribosomal protein S21 | 32744_at | Day 7 Peri/Day 0 Peri | 0.740 | 7.2902E−03 |
| 508 | 6169 | Entrez Gene | ribosomal protein L38 | 34085_at | Day 7 Peri/Day 0 Peri | 0.743 | 6.3557E−03 |
| 509 | 51304 | Entrez Gene | zinc finger, DHHC-type containing 3 | 39751_at | Day 3 Intra/Day 3 Extra | 0.744 | 7.7468E−03 |
| 510 | 6218 | Entrez Gene | ribosomal protein S17 | 34592_at | Day 7 Peri/Day 0 Peri | 0.745 | 2.8146E−03 |
| 511 | 9540 | Entrez Gene | tumor protein p53 inducible protein 3 | 36079_at | Day 3 Peri/Day 3 Intra | 0.746 | 5.1335E−03 |
| 512 | 1327 | Entrez Gene | cytochrome c oxidase subunit IV isoform 1 | 39027_at | Day 3 Peri/Day 3 Extra | 0.746 | 6.3689E−03 |
| 513 | 10541 | Entrez Gene | acidic (leucine-rich) nuclear phosphoprotein 32 family, | 38479_at | Day 7 Intra/Day 7 Extra | 0.747 | 1.6161E−03 |
| | | | member B | | | | |
| 514 | 3030 | Entrez Gene | hydroxyacyl-Coenzyme A dehydrogenase/3-ketoacyl- | 36952_at | Day 3 Peri/Day 3 Extra | 0.750 | 6.1291E−03 |
| | | | Coenzyme A thiolase/enoyl-Coenzyme A hydratase | 36952_at | Day 0 Peri/Day 0 Control | 0.835 | 2.4582E−03 |
| | | | (trifunctional protein), alpha subunit | | | | |
| 515 | 23379 | Entrez Gene | KIAA0947 protein | 41595_at | Day 7 Intra/Day 7 Extra | 0.751 | 2.7370E−03 |
| 516 | 3032 | Entrez Gene | hydroxyacyl-Coenzyme A dehydrogenase/3-ketoacyl- | 39741_at | Day 3 Peri/Day 3 Extra | 0.752 | 2.0159E−03 |
| | | | Coenzyme A thiolase/enoyl-Coenzyme A hydratase | | | | |
| | | | (trifunctional protein), beta subunit | | | | |
| 517 | 5223 | Entrez Gene | phosphoglycerate mutase 1 (brain) | 41221_at | Day 3 Peri/Day 3 Extra | 0.752 | 1.6814E−03 |
| 518 | 10638 | Entrez Gene | S-phase response (cyclin-related) | 1685_at | Day 3 Peri/Day 0 Peri | 0.754 | 1.2093E−03 |
| 519 | 9215 | Entrez Gene | like-glycosyltransferase | 41346_at | Day 3 Intra/Day 0 Intra | 0.754 | 4.1376E−03 |
| 520 | 10572 | Entrez Gene | CD27-binding (Siva) protein | 39020_at | Day 3 Peri/Day 3 Control | 0.754 | 9.5129E−03 |
| 521 | 10241 | Entrez Gene | nuclear domain 10 protein | 40063_at | Day 3 Intra/Day 0 Intra | 0.755 | 7.6513E−03 |
| 522 | 162427 | Entrez Gene | hypothetical protein LOC162427 | 38423_at | Day 0 Peri/Day 0 Control | 0.755 | 7.1257E−03 |
| | | | | 38423_at | Day 3 Peri/Day 0 Peri | 0.845 | 2.2722E−03 |
| | | | | 38423_at | Day 7 Peri/Day 0 Peri | 0.848 | 1.7317E−03 |
| 523 | 10036 | Entrez Gene | chromatin assembly factor 1, subunit A (p150) | 32589_at | Day 7 Peri/Day 7 Extra | 0.756 | 2.8970E−03 |
| 524 | 6202 | Entrez Gene | ribosomal protein S8 | 31583_at | Day 7 Peri/Day 0 Peri | 0.756 | 8.6210E−03 |
| 525 | 55218 | Entrez Gene | chromosome 14 open reading frame 114 | 35283_at | Day 0 Peri/Day 0 Control | 0.759 | 7.7904E−04 |
| 526 | 1955 | Entrez Gene | EGF-like-domain, multiple 5 | 36488_at | Day 3 Intra/Day 0 Intra | 0.759 | 7.4685E−03 |
| 527 | 9724 | Entrez Gene | UTP14, U3 small nucleolar ribonucleoprotein, homolog | 39405_at | Day 0 Peri/Day 0 Intra | 0.760 | 3.9078E−03 |
| | | | C (yeast) | | | | |
| 528 | 26003 | Entrez Gene | golgi reassembly stacking protein 2, 55 kDa | 35805_at | Day 3 Peri/Day 0 Peri | 0.761 | 2.3291E−04 |
| | | | | 35805_at | Day 7 Peri/Day 0 Peri | 0.770 | 2.6315E−03 |
| 529 | 56829 | Entrez Gene | zinc finger CCCH-type, antiviral 1 | 35682_at | Day 3 Peri/Day 3 Control | 0.761 | 2.0789E−04 |
| 530 | 4601 | Entrez Gene | MAX interactor 1 | 39072_at | Day 3 Intra/Day 0 Intra | 0.762 | 4.0454E−03 |
| | | | | 654_at | Day 7 Peri/Day 0 Peri | 0.783 | 5.2959E−03 |
| | | | | 39072_at | Day 3 Peri/Day 0 Peri | 0.839 | 6.8595E−03 |
| 531 | 6293 | Entrez Gene | vacuolar protein sorting 52 (yeast) | 32658_at | Day 0 Peri/Day 0 Control | 0.762 | 7.4217E−03 |
| | | | | 32658_at | Day 7 Peri/Day 0 Peri | 0.858 | 6.8088E−03 |
| 532 | 1998 | Entrez Gene | E74-like factor 2 (ets domain transcription factor) | 507_s_at | Day 7 Intra/Day 0 Intra | 0.763 | 4.9954E−03 |
| 533 | 10067 | Entrez Gene | secretory carrier membrane protein 3 | 32799_at | Day 7 Peri/Day 0 Peri | 0.763 | 8.9346E−03 |
| 534 | 5437 | Entrez Gene | polymerase (RNA) II (DNA directed) polypeptide H | 35631_at | Day 3 Peri/Day 3 Extra | 0.764 | 7.2269E−03 |
| | | | | 35631_at | Day 3 Peri/Day 3 Intra | 0.772 | 5.4217E−03 |
| 535 | 6203 | Entrez Gene | ribosomal protein S9 | 31511_at | Day 7 Peri/Day 0 Peri | 0.764 | 7.8708E−03 |
| 536 | 55744 | Entnez Gene | hypothetical protein FLJ10803 | 37610_at | Day 3 Peri/Day 3 Extra | 0.764 | 2.5200E−03 |
| 537 | 7693 | Entrez Gene | zinc finger protein 134 (clone pHZ-15) | 36295_at | Day 3 Intra/Day 0 Intra | 0.764 | 9.8397E−03 |
| 538 | 5036 | Entrez Gene | proliferation-associated 2G4, 38 kDa | 41600_at | Day 7 Peri/Day 0 Peri | 0.765 | 6.9426E−03 |
| 539 | 9249 | Entrez Gene | dehydrogenase/reductase (SDR family) member 3 | 40782_at | Day 3 Peri/Day 0 Peri | 0.765 | 6.4510E−03 |
| 540 | 1337 | Entrez Gene | cytochrome c oxidase subunit Via polypeptide 1 | 41206_r_at | Day 7 Peri/Day 0 Peri | 0.766 | 1.7769E−03 |
| 541 | 23623 | Entrez Gene | RUN and SH3 domain containing 1 | 34264_at | Day 3 Peri/Day 3 Intra | 0.768 | 9.1280E−03 |
| 542 | 8890 | Entrez Gene | eukaryotic translation initiation factor 2B, subunit 4 | 32659_at | Day 3 Peri/Day 3 Control | 0.768 | 8.3725E−03 |
| | | | delta, 67 kDa | | | | |
| 543 | 203069 | Entrez Gene | R3H domain and coiled-coil containing 1 | 35156_at | Day 7 Peri/Day 0 Peri | 0.769 | 8.4083E−03 |
| 544 | 7529 | Entrez Gene | tyrosine 3-monooxygenase/tryptophan 5- | 32324_at | Day 3 Peri/ Day 3 Extra | 0.769 | 5.4500E−03 |
| | | | monooxygenase activation protein, beta polypeptide | | | | |
| 545 | 23294 | Entrez Gene | ankyrin repeat and sterile alpha motif domain containing 1 | 40971_at | Day 3 Intra/Day 0 Intra | 0.769 | 7.4093E−03 |
| | | | | 40971_at | Day 3 Peri/Day 0 Peri | 0.796 | 3.4853E−03 |
| 546 | 29 | Entrez Gene | active BCR-related gene | 39058_at | Day 3 Peri/Day 3 Control | 0.771 | 7.8298E−03 |
| | | | | 39058_at | Day 3 Peri/Day 3 Extra | 0.796 | 8.8684E−03 |
| 547 | 1937 | Entrez Gene | eukaryotic translation elongation factor 1 gamma | 1676_s_at | Day 7 Peri/Day 0 Peri | 0.771 | 8.5875E−03 |
| 548 | 5636 | Entrez Gene | phosphoribosyl pyrophosphate synthetase-associated | 41853_at | Day 3 Intra/Day 3 Extra | 0.772 | 9.6928E−03 |
| | | | protein 2 | | | | |
| 549 | 147179 | Entrez Gene | WIRE protein | 40787_at | Day 3 Peri/Day 0 Peri | 0.772 | 8.6910E−04 |
| 550 | 9703 | Entrez Gene | KIAA0100 gene product | 39783_at | Day 0 Intra/Day 0 Peri | 0.773 | 4.2046E−03 |
| 551 | 10294 | Entrez Gene | DnaJ (Hsp40) homolog, subfamily A, member 2 | 34201_at | Day 3 Intra/Day 0 Intra | 0.775 | 2.1191E−03 |
| 552 | 9741 | Entrez Gene | lysosomal-associated protein transmembrane 4 alpha | 39019_at | Day 3 Intra/Day 0 Intra | 0.776 | 6.0768E−03 |
| | | | | 39019_at | Day 7 Intra/Day 0 Intra | 0.815 | 9.6992E−03 |
| 553 | 8480 | Entrez Gene | RAE1 RNA export 1 homolog (S. pombe) | 32757_at | Day 3 Peri/Day 3 Extra | 0.778 | 1.2850E−03 |
| | | | | 32758_g_at | Day 7 Peri/Day 7 Intra | 0.786 | 2.2519E−03 |
| 554 | 10556 | Entrez Gene | ribonuclease P/MRP 30 kDa subunit | 39702_at | Day 3 Intra/Day 3 Extra | 0.778 | 8.7433E−03 |
| 555 | 9562 | Entrez Gene | multiple inositol polyphosphate histidine phosphatase, 1 | 38325_at | Day 3 Intra/Day 3 Extra | 0.779 | 8.9541E−03 |
| 556 | 9528 | Entrez Gene | transmembrane protein 59 | 39033_at | Day 3 Intra/Day 3 Control | 0.779 | 6.9035E−03 |
| | | | | 39033_at | Day 3 Intra/Day 0 Intra | 0.787 | 2.6172E−04 |
| 557 | 5326 | Entrez Gene | pleiomorphic adenoma gene-like 2 | 40061_at | Day 3 Intra/Day 3 Peri | 0.779 | 7.8774E−03 |
| 558 | 3163 | Entrez Gene | heme oxygenase (decycling) 2 | 37916_at | Day 7 Peri/Day 3 Peri | 0.781 | 9.4119E−03 |
| 559 | 22864 | Entrez Gene | KIAA1002 protein | 41366_at | Day 3 Peri/Day 0 Peri | 0.781 | 8.7647E−03 |
| 560 | 23307 | Entrez Gene | KIAA0674 | 31826_at | Day 0 Peri/Day 0 Control | 0.783 | 2.4933E−03 |
| 561 | 670 | Entrez Gene | biphenyl hydrolase-like (serine hydrolase; breast | 40912_s_at | Day 0 Intra/Day 0 Control | 0.784 | 7.7779E−03 |
| | | | epithelial mucin-associated antigen) | | | | |
| 562 | 9903 | Entrez Gene | kelch-like 21 (Drosophila) | 37230_at | Day 3 Peri/Day 0 Peri | 0.786 | 5.6275E−03 |
| | | | | 37230_at | Day 7 Peri/Day 0 Peri | 0.812 | 4.4808E−03 |
| 563 | 5236 | Entrez Gene | phosphoglucomutase 1 | 32210_at | Day 3 Peri/Day 0 Peri | 0.789 | 7.7233E−03 |
| 564 | 51780 | Entrez Gene | jumoriji domain containing 1B | 33870_at | Day 3 Intra/Day 0 Intra | 0.789 | 6.0617E−03 |
| 565 | 2975 | Entrez Gene | general transcription factor IIIC, polypeptide 1, alpha | 35671_at | Day 7 Peri/Day 0 Peri | 0.791 | 3.1660E−03 |
| | | | 220 kDa | | | | |
| 566 | 10956 | Entrez Gene | amplified in osteosarcoma | 36996_at | Day 7 Peri/Day 0 Peri | 0.791 | 9.0785E−03 |
| | | | | 36996_at | Day 3 Peri/Day 0 Peri | 0.818 | 6.2854E−03 |
| 567 | 1201 | Entrez Gene | ceroid-lipofuscinosis, neuronal 3, juvenile (Batten, | 497_at | Day 3 Peri/Day 3 Control | 0.792 | 5.8473E−03 |
| | | | Spielmeyer-Vogt disease) | | | | |
| 568 | 56270 | Entrez Gene | WDR45-like | 39185_at | Day 3 Intra/Day 3 Control | 0.792 | 6.7923E−03 |
| 569 | 527 | Entrez Gene | ATPase, H+ transporting, lysosomal 16 kDa, V0 subunit c | 36994_at | Day 7 Peri/Day 0 Peri | 0.794 | 8.6116E−03 |
| 570 | 8720 | Entrez Gene | membrane-bound transcription factor peptidase, site 1 | 36964_at | Day 3 Peri/Day 0 Peri | 0.795 | 9.8019E−03 |
| 571 | 3151 | Entrez Gene | high-mobility group nucleosomal binding domain 2 | 41231_f_at | Day 3 Peri/Day 3 Extra | 0.796 | 7.6383E−04 |
| | | | | 41231_f_at | Day 3 Peri/Day 3 Intra | 0.827 | 4.3201E−03 |
| 572 | 286440 | Entrez Gene | hypothetical protein LOC286440 | 41655_at | Day 3 Peri/Day 0 Peri | 0.796 | 8.4134E−03 |
| 573 | 6136 | Entrez Gene | ribosomal protein L12 | 33668_at | Day 7 Peri/Day 0 Peri | 0.797 | 2.3409E−03 |
| 574 | 79073 | Entrez Gene | hypothetical protein MGC5508 | 39693_at | Day 3 Intra/Day 3 Extra | 0.797 | 7.6719E−03 |
| 575 | 445 | Entrez Gene | argininosuccinate synthetase | 40541_at | Day 7 Peri/Day 3 Peri | 0.799 | 7.5623E−03 |
| 576 | 11224 | Entrez Gene | ribosomal protein L35 | 41765_at | Day 7 Peri/Day 0 Peri | 0.800 | 6.9298E−03 |
| 577 | 11258 | Entrez Gene | dynactin 3 (p22) | 40410_at | Day 3 Peri/Day 3 Control | 0.808 | 6.4549E−03 |
| 578 | 4779 | Entrez Gene | nuclear factor (erythroid-derived 2)-like 1 | 38439_at | Day 7 Peri/Day 0 Peri | 0.811 | 5.5209E−03 |
| 579 | 149603 | Entrez Gene | ring finger protein 187 | 39722_at | Day 3 Peri/Day 0 Peri | 0.812 | 7.5346E−03 |
| 580 | 8818 | Entrez Gene | dolichyl-phosphate mannosyltransferase polypeptide 2, | 38726_at | Day 7 Peri/Day 3 Peri | 0.814 | 3.7920E−03 |
| | | | regulatory subunit | | | | |
| 581 | 823 | Entrez Gene | calpain 1, (mu/l) large subunit | 33908_at | Day 7 Peri/Day 3 Peri | 0.815 | 9.2186E−03 |
| 582 | 5438 | Entrez Gene | polymerase (RNA) II (DNA directed) polypeptide I, | 34828_at | Day 0 Peri/Day 0 Control | 0.816 | 8.6878E−03 |
| | | | 14.5 kDa | | | | |
| 583 | 6720 | Entrez Gene | sterol regulatory element binding transcription factor 1 | 32135_at | Day 3 Peri/Day 0 Peri | 0.819 | 6.4366E−03 |
| 584 | 23633 | Entrez Gene | Karyopherin alpha 6 (importin alpha 7) | 40275_at | Day 3 Peri/Day 0 Peri | 0.824 | 1.6981E−03 |
| | | | | 40275_at | Day 7 Peri/Day 0 Peri | 0.847 | 5.5244E−03 |
| 585 | 7534 | Entrez Gene | tyrosine 3-monooxygenase/tryptophan 5- | 1235_at | Day 3 Peri/Day 3 Extra | 0.824 | 9.9973E−03 |
| | | | monooxygenase activation protein, zeta polypeptide | 1235_at | Day 3 Peri/Day 3 Intra | 0.853 | 7.0175E−03 |
| 586 | 23481 | Entrez Gene | pescadillo homolog 1, containing BRCT domain | 41869_at | Day 7 Peri/Day 0 Peri | 0.831 | 9.3885E−03 |
| | | | (zebrafish) | | | | |
| 587 | 369 | Entrez Gene | v-raf murine sarcoma 3611 viral oncogene homolog | 1706_at | Day 7 Intra/Day 3 Intra | 0.832 | 4.3502E−04 |
| 588 | 23 | Entrez Gene | ATP-binding cassette, sub-family F (GCN20), member 1 | 39141_at | Day 7 Intra/Day 3 Intra | 0.837 | 5.3611E−03 |
| 589 | 25966 | Entrez Gene | chromosome 21 open reading frame 25 | 32107_at | Day 7 Intra/Day 7 Extra | 0.845 | 5.8404E−03 |
| 590 | 23644 | Entrez Gene | autoantigen | 36670_at | Day 3 Intra/Day 0 Intra | 0.866 | 7.3351E−03 |

TABLE 2
Diagnostic Down

| | Public | | |
|---|---|---|---|
| Gene ID | Identifier | Data Source | Gene Name |
| 1 | 4250 | Entrez Gene | secretoglobin, family 2A, member 2 |
| 3 | 10647 | Entrez Gene | secretoglobin, family 1D, member 2 |
| 5 | 2167 | Entrez Gene | fatty acid binding protein 4, adipocyte |
| 8 | 9415 | Entrez Gene | fatty acid desaturase 2 |
| 11 | 553168 | Entrez Gene | chromosome 1 open reading frame 68 |
| 13 | 125 | Entrez Gene | alcohol dehydrogenase IB (class I), beta polypeptide |
| 14 | 3205 | Entrez Gene | homeo box A9 |
| 15 | 7136 | Entrez Gene | troponin I type 2 (skeletal, fast) |
| 17 | 6210 | Entrez Gene | ribosomal protein S15a |
| 18 | 7123 | Entrez Gene | C-type lectin domain family 3, member B |
| 20 | AB011538 | GenBank | CDNA clone IMAGE: 5922621 |
| 21 | 3131 | Entrez Gene | Hepatic leukemia factor |
| 22 | M57951 | GenBank | UDP glucuronosyltransferase 1 family, polypeptide A10 |
| | | | /// UDP glucuronosyltransferase 1 family, polypeptide |
| | | | A8 /// UDP glucuronosyltransferase 1 fami |
| 24 | 2053 | Entrez Gene | epoxide hydrolase 2, cytoplasmic |
| 26 | 5271 | Entrez Gene | serpin peptidase inhibitor, clade B (ovalbumin), member 8 |
| 28 | 10781 | Entrez Gene | zinc finger protein 266 |
| 29 | 4128 | Entrez Gene | monoamine oxidase A |
| 31 | 8483 | Entrez Gene | cartilage intermediate layer protein, nucleotide |
| | | | pyrophosphohydrolase |
| 34 | 10458 | Entrez Gene | BAI1-associated protein 2 |
| 36 | 2273 | Entrez Gene | four and a half LIM domains 1 |
| 37 | 23016 | Entrez Gene | exosome component 7 |
| 38 | 23352 | Entrez Gene | retinoblastoma-associated factor 600 |
| 39 | 7138 | Entrez Gene | troponin T type 1 (skeletal, slow) |
| 42 | 10521 | Entrez Gene | DEAD (Asp-Glu-Ala-Asp) box polypeptide 17 |
| 43 | 8710 | Entrez Gene | serpin peptidase inhibitor, clade B (ovalbumin), member 7 |
| 44 | 2194 | Entrez Gene | fatty acid synthase |
| 45 | 3202 | Entrez Gene | homeo box A5 |
| 46 | 54 | Entrez Gene | acid phosphatase 5, tartrate resistant |
| 49 | 63928 | Entrez Gene | hepatocellular carcinoma antigen gene 520 |
| 50 | 5055 | Entrez Gene | serpin peptidase inhibitor, clade B (ovalbumin), member 2 |
| 51 | 5354 | Entrez Gene | proteolipid protein 1 (Pelizaeus-Merzbacher disease, |
| | | | spastic paraplegia 2, uncomplicated) |
| 52 | 642 | Entrez Gene | bleomycin hydrolase |
| 53 | 10443 | Entrez Gene | Hypothetical gene CG012 |
| 54 | 224 | Entrez Gene | aldehyde dehydrogenase 3 family, member A2 |
| 55 | 90634 | Entrez Gene | hypothetical gene CG018 |
| 56 | 1675 | Entrez Gene | D component of complement (adipsin) |
| 57 | 23242 | Entrez Gene | cordon-bleu homolog (mouse) |
| 58 | 10158 | Entrez Gene | PDZK1 interacting protein 1 |
| 59 | 10001 | Entrez Gene | mediator of RNA polymerase II transcription, subunit 6 |
| | | | homolog (yeast) |
| 60 | 97 | Entrez Gene | acylphosphatase 1, erythrocyte (common) type |
| 63 | 51097 | Entrez Gene | saccharopine dehydrogenase (putative) |
| 64 | 2217 | Entrez Gene | Fc fragment of IgG, receptor, transporter, alpha |
| 65 | 1983 | Entrez Gene | eukaryotic translation initiation factor 5 |
| 66 | 51601 | Entrez Gene | lipoyltransferase 1 |
| 70 | 2618 | Entrez Gene | phosphoribosylglycinamide formyltransferase, |
| | | | phosphoribosylglycinamide synthetase, |
| | | | phosphoribosylaminoimidazole synthetase |
| 71 | HG3570- | The Institute for | — |
| | HT377 | Genomic Research | |
| 72 | 53335 | Entrez Gene | B-cell CLL/lymphoma 11A (zinc finger protein) |
| 74 | 8906 | Entrez Gene | adaptor-related protein complex 1, gamma 2 subunit |
| 77 | 9524 | Entrez Gene | glycoprotein, synaptic 2 |
| 78 | 91137 | Entrez Gene | hypothetical protein BC017169 |
| 79 | 56288 | Entrez Gene | par-3 partitioning defective 3 homolog (C. elegans) |
| 80 | 26154 | Entrez Gene | ATP-binding cassette, sub-family A (ABC1), member 12 |
| 81 | 260294 | Entrez Gene | Williams Beuren syndrome chromosome region 20C |
| 82 | 1410 | Entrez Gene | crystallin, alpha B |
| 83 | 131544 | Entrez Gene | hypothetical protein DKFZp667G2110 |
| 84 | 10733 | Entrez Gene | polo-like kinase 4 (Drosophila) |
| 86 | 10181 | Entrez Gene | RNA binding motif protein 5 |
| 90 | 6924 | Entrez Gene | Transcription elongation factor B (SIII), polypeptide 3 |
| | | | (110 kDa, elongin A) |
| 93 | 1152 | Entrez Gene | creatine kinase, brain |
| 94 | 10450 | Entrez Gene | peptidylprolyl isomerase E (cyclophilin E) |
| 95 | HG1112- | The Institute for | — |
| | HT111 | Genomic Research | |
| 97 | 4129 | Entrez Gene | monoamine oxidase B |
| 99 | 10314 | Entrez Gene | LanC lantibiotic synthetase component C-like 1 |
| | | | (bacterial) |
| 101 | 8532 | Entrez Gene | carboxypeptidase Z |
| 102 | 6038 | Entrez Gene | ribonuclease, RNase A family, 4 |
| 103 | 10924 | Entrez Gene | sphingomyelin phosphodiesterase, acid-like 3A |
| 106 | 8634 | Entrez Gene | RNA terminal phosphate cyclase domain 1 |
| 107 | 4163 | Entrez Gene | mutated in colorectal cancers |
| 108 | 7262 | Entrez Gene | pleckstrin homology-like domain, family A, member 2 |
| 109 | 26039 | Entrez Gene | synovial sarcoma translocation gene on chromosome |
| | | | 18-like 1 |
| 110 | 1525 | Entrez Gene | coxsackie virus and adenovirus receptor |
| 112 | 11072 | Entrez Gene | dual specificity phosphatase 14 |
| 113 | 6490 | Entrez Gene | sliver homolog (mouse) |
| 115 | 4176 | Entrez Gene | MCM7 minichromosome maintenance deficient 7 (S. cerevisiae) |
| 116 | 2197 | Entrez Gene | Finkel-Biskis-Reilly murine sarcoma virus (FBR-MuSV) |
| | | | ubiquitously expressed (fox derived); ribosomal protein |
| | | | S30 |
| 118 | 4898 | Entrez Gene | nardilysin (N-arginine dibasic convertase) |
| 119 | 6662 | Entrez Gene | SRY (sex determining region Y)-box 9 (campornelic |
| | | | dysplasia, autosomal sex-reversal) |
| 120 | 2114 | Entrez Gene | v-ets erythroblastosis virus E26 oncogene homolog 2 |
| | | | (avian) |
| 121 | 1158 | Entrez Gene | creatine kinase, muscle |
| 123 | 1573 | Entrez Gene | cytochrome P450, family 2, subfamily J, polypeptide 2 |
| 129 | 9833 | Entrez Gene | maternal embryonic leucine zipper kinase |
| 130 | U18300 | GenBank | damage-specific DNA binding protein 2, 48 kDa /// LIM |
| | | | homeobox 3 |
| 131 | 23051 | Entrez Gene | zinc fingers and homeoboxes 3 |
| 132 | 51706 | Entrez Gene | cytochrome b5 reductase 1 |
| 136 | 2013 | Entrez Gene | epithelial membrane protein 2 |
| 137 | 81563 | Entrez Gene | chromosome 1 open reading frame 21 |
| 138 | 1638 | Entrez Gene | dopachrome tautomerase (dopachrome delta- |
| | | | isomerase, tyrosine-related protein 2) |
| 141 | 57613 | Entrez Gene | KIAA1467 protein |
| 142 | 2647 | Entrez Gene | biogenesis of lysosome-related organelies complex-1, |
| | | | subunit 1 |
| 144 | AF096870 | GenBank | tripartite motif-containing 16 /// similar to tripartite motif- |
| | | | containing 16; estrogen-responsive B box protein |
| 146 | 4968 | Entrez Gene | 8-oxoguanine DNA glycosylase |
| 148 | 4713 | Entrez Gene | NADH dehydrogenase (ubiquinone) 1 beta subcomplex, |
| | | | 7, 18 kDa |
| 149 | 1396 | Entrez Gene | cysteine-rich protein 1 (intestinal) |
| 150 | 771 | Entrez Gene | carbonic anhydrase XII |
| 151 | 55187 | Entrez Gene | vacuolar protein sorting 13D (yeast) |
| 152 | 498 | Entrez Gene | ATP synthase, H+ transporting, mitochondrial F1 |
| | | | complex, alpha subunit, isoform 1, cardiac muscle |
| 154 | 242 | Entrez Gene | arachidonate 12-lipoxygenase, 12R type |
| 156 | 5985 | Entrez Gene | replication factor C (activator 1) 5, 36.5 kDa |
| 157 | 26610 | Entrez Gene | elongation protein 4 homolog (S. cerevisiae) |
| 159 | 5110 | Entrez Gene | Protein-L-isoaspartate (D-aspartate) O- |
| | | | methyltransferase |
| 160 | 113146 | Entrez Gene | chromosome 14 open reading frame 78 |
| 165 | 10124 | Entrez Gene | ADP-ribosylation factor-like 4 |
| 166 | 10653 | Entrez Gene | serine peptidase inhibitor, Kunitz type, 2 |
| 167 | 10434 | Entrez Gene | lysophospholipase I |
| 169 | 123 | Entrez Gene | adipose differentiation-related protein |
| 170 | 80308 | Entrez Gene | Fad1, flavin adenine dinucleotide synthetase, homolog |
| | | | (yeast) |
| 173 | 23345 | Entrez Gene | spectrin repeat containing, nuclear envelope 1 |
| 175 | 80208 | Entrez Gene | hypothetical protein FLJ21439 |
| 176 | 202 | Entrez Gene | absent in melanoma 1 |
| 178 | 5268 | Entrez Gene | serpin peptidase inhibitor, clade B (ovalbumin), member 5 |
| 178 | 5268 | Entrez Gene | serpin peptidese inhibitor, clade B (ovalbumin), member 5 |
| 179 | 1474 | Entrez Gene | cystatin E/M |
| 180 | 9927 | Entrez Gene | mitofusin 2 |
| 182 | 2145 | Entrez Gene | enhancer of zeste homolog 1 (Drosophila) |
| 183 | 2959 | Entrez Gene | general transcription factor IIB |
| 185 | 4736 | Entrez Gene | ribosomal protein L10a |
| 186 | 644 | Entrez Gene | biliverdin reductase A |
| 187 | 9665 | Entrez Gene | limkain b1 |
| 188 | 3033 | Entrez Gene | L-3-hydroxyacyl-Coenzyme A dehydrogenase, short |
| | | | chain |
| 190 | 55651 | Entrez Gene | nucleolar protein family A, member 2 (H/ACA small |
| | | | nucleolar RNPs) |
| 191 | 11336 | Entrez Gene | SEC6-like 1 (S. cerevisiae) |
| 192 | 7009 | Entrez Gene | testis enhanced gene transcript (BAX inhibitor 1) |
| 193 | 23107 | Entrez Gene | mitochondrial ribosomal protein S27 |
| 194 | 50 | Entrez Gene | aconitase 2, mitochondrial |
| 195 | 2746 | Entrez Gene | glutamate dehydrogenase 1 |
| 197 | 6477 | Entrez Gene | Seven in absentia homolog 1 (Drosophila) |
| 198 | 6166 | Entrez Gene | ribosomal protein L36a-like |
| 199 | 7175 | Entrez Gene | translocated promoter region (to activated MET |
| | | | oncogene) |
| 201 | 171546 | Entrez Gene | chromosome 14 open reading frame 147 |
| 202 | 10553 | Entrez Gene | HIV-1 Tat interactive protein 2, 30 kDa |
| 203 | 115817 | Entrez Gene | dehydrogenase/reductase (SDR family) member 1 |
| 204 | 25813 | Entrez Gene | sorting and assembly machinery component 50 |
| | | | homolog (S. cerevisiae) |
| 205 | 25953 | Entrez Gene | DKFZP564G2022 protein |
| 206 | 1212 | Entrez Gene | clathrin, light polypeptide (Lcb) |
| 207 | 1652 | Entrez Gene | D-dopachrome tautomerase |
| 208 | 27335 | Entrez Gene | eukaryotic translation initiation factor 3, subunit 12 |
| 209 | 217 | Entrez Gene | aldehyde dehydrogenase 2 family (mitochondrial) |
| 210 | 10049 | Entrez Gene | DnaJ (Hsp40) homolog, subfamily B, member 6 |
| 212 | 8623 | Entrez Gene | acetylserotonin O-methyltransferase-like |
| 213 | 169611 | Entrez Gene | olfactomedin-like 2A |
| 214 | 11066 | Entrez Gene | U11/U12 snRNP 35K |
| 215 | 9936 | Entrez Gene | CD302 antigen |
| 216 | 4729 | Entrez Gene | NADH dehydrogenase (ubiquinone) flavoprotein 2, |
| | | | 24 kDa |
| 217 | 987 | Entrez Gene | LPS-responsive vesicle trafficking, beach and anchor |
| | | | containing |
| 218 | 128 | Entrez Gene | alcohol dehydrogenase 5 (class III), chi polypeptide |
| 220 | 7295 | Entrez Gene | thioredoxin |
| 222 | 10980 | Entrez Gene | COP9 constitutive photomorphogenic homolog subunit |
| | | | 6 (Arabidopsis) |
| 223 | 2621 | Entrez Gene | growth arrest-specific 6 |
| 224 | 831 | Entrez Gene | calpastatin |
| 228 | 645 | Entrez Gene | biliverdin reductase B (flavin reductase (NADPH)) |
| 229 | 8581 | Entrez Gene | lymphocyte antigen 6 complex, locus D |
| 230 | AB006780 | GenBank | lectin, galectoside-binding, soluble, 3 (galectin 3) /// |
| | | | galectin-3 internal gene |
| 231 | 6929 | Entrez Gene | transcription factor 3 (E2A immunoglobulin enhancer |
| | | | binding factors E12/E47) |
| 232 | 10476 | Entrez Gene | ATP synthase, H+ transporting, mitochondrial F0 |
| | | | complex, subunit d |
| 233 | 7022 | Entrez Gene | transcription factor AP-2 gamma (activating enhancer |
| | | | binding protein 2 gamma) |
| 234 | 2806 | Entrez Gene | glutamic-oxaloacetic transaminase 2, mitochondrial |
| | | | (aspartate aminotransferase 2) |
| 235 | 23185 | Entrez Gene | La ribonucleoprotein domain family, member 5 |
| 237 | 708 | Entrez Gene | complement component 1, q subcomponent binding |
| | | | protein |
| 238 | 81875 | Entrez Gene | Interferon stimulated exonuclease gene 20 kDa-like 2 |
| 239 | AI032612 | GenBank | enolase 1, (alpha) /// small nuclear ribonucleoprotein |
| | | | polypeptide F |
| 240 | 26040 | Entrez Gene | SET binding protein 1 |
| 241 | 8673 | Entrez Gene | vesicle-associated membrane protein 8 (endobrevin) |
| 243 | 4255 | Entrez Gene | O-6-methylguanine-DNA methyltransferase |
| 245 | 6249 | Entrez Gene | restin (Reed-Steinberg cell-expressed intermediate |
| | | | filament-associated protein) |
| 246 | 11188 | Entrez Gene | nischarin |
| 248 | 2547 | Entrez Gene | X-ray repair complementing defective repair in Chinese |
| | | | hamster cells 6 (Ku autoantigen, 70 kDa) |
| 249 | 545 | Entrez Gene | ataxia telangiectasia and Rad3 related |
| 250 | 4775 | Entrez Gene | nuclear factor of activated T-cells, cytoplasmic, |
| | | | calcineurin-dependent 3 |
| 252 | 4790 | Entrez Gene | nuclear factor of kappa light polypeptide gene enhancer |
| | | | in B-cells 1 (p105) |
| 254 | 26135 | Entrez Gene | SERPINE1 mRNA binding protein 1 |
| 255 | 23234 | Entrez Gene | DnaJ (Hsp40) homolog, subfamily C, member 9 |
| 257 | AI557912 | GenBank | translocase of outer mitochondrial membrane 7 |
| | | | homolog (yeast) /// hypothetical protein LOC201725 |
| 258 | 4437 | Entrez Gene | mutS homolog 3 (E. coli) |
| 259 | 3550 | Entrez Gene | IK cytokine, down-regulator of HLA II |
| 260 | 5453 | Entrez Gene | POU domain, class 3, transcription factor 1 |
| 262 | 353 | Entrez Gene | adenine phosphoribosyltransferase |
| 263 | 6427 | Entrez Gene | splicing factor, arginine/serine-rich 2 |
| 265 | 79703 | Entrez Gene | hypothetical protein FLJ22531 |
| 266 | 27292 | Entrez Gene | dimethyladenosine transferase |
| 267 | 54665 | Entrez Gene | round spermatid basic protein 1 |
| 268 | 513 | Entrez Gene | ATP synthase, H+ transporting, mitochondrial F1 |
| | | | complex, delta subunit |
| 269 | AL050091 | GenBank | glutamate receptor, ionotropic, N-methyl D-aspartate- |
| | | | like 1A /// GRINL1A combined protein /// similar to |
| | | | glutamate receptor, ionotropic, N-methyl D-a |
| 271 | 9349 | Entrez Gene | ribosomal protein L23 |
| 273 | 23090 | Entrez Gene | zinc finger protein 423 |
| 275 | 10497 | Entrez Gene | unc-13 homolog B (C. elegans) |
| 276 | 56967 | Entrez Gene | chromosome 14 open reading frame 132 |
| 276 | 56967 | Entrez Gene | chromosome 14 open reading frame 132 |
| 277 | 8560 | Entrez Gene | degenerative spermatocyte homolog 1, lipid desaturase |
| | | | (Drosophila) |
| 278 | 10975 | Entrez Gene | ubiquinol-cytochrome c reductase, 6.4 kDa subunit |
| 279 | 10641 | Entrez Gene | tumor suppressor candidate 4 |
| 280 | 8663 | Entrez Gene | eukaryotic translation initiation factor 3, subunit 8, |
| | | | 110 kDa |
| 281 | 50717 | Entrez Gene | WD repeat domain 42A |
| 282 | 23392 | Entrez Gene | KIAA0368 |
| 285 | 23650 | Entrez Gene | tripartite motif-containing 29 |
| 286 | 8665 | Entrez Gene | eukaryotic translation initiation factor 3, subunit 5 |
| | | | epsilon, 47 kDa |
| 287 | 7203 | Entrez Gene | chaperonin containing TCP1, subunit 3 (gamma) |
| 290 | 25797 | Entrez Gene | glutaminyl-peptide cyclotransferase (glutaminyl cyclase) |
| 291 | 5092 | Entrez Gene | 6-pyruvoyl-tetrahydropterin synthase/dimerization |
| | | | cofactor of hepatocyte nuclear factor 1 alpha (TCF1) |
| 292 | 7818 | Entrez Gene | death associated protein 3 |
| 293 | U05861 | GenBank | aldo-keto reductase family 1, member C1 (dihydrodiol |
| | | | dehydrogenase 1; 20-alpha (3-alpha)-hydroxysteroid |
| | | | dehydrogenase) /// aldo-keto reductase family |
| 294 | 23063 | Entrez Gene | KIAA0261 |
| 295 | 9529 | Entrez Gene | BCL2-associated athanogene 5 |
| 296 | 57147 | Entrez Gene | SCY1-like 3 (S. cerevisiae) |
| 298 | 10289 | Entrez Gene | translation factor sui1 homolog |
| 299 | 9536 | Entrez Gene | prostaglandin E synthase |
| 300 | 22883 | Entrez Gene | calsyntenin 1 |
| 301 | 7520 | Entrez Gene | X-ray repair complementing defective repair in Chinese |
| | | | hamster cells 5 (double-strand-break rejoining; Ku |
| | | | autoantigen, 60 kDa) |
| 303 | 4928 | Entrez Gene | nucleoporin 98 kDa |
| 304 | 3290 | Entrez Gene | hydroxysteroid (11-beta) dehydrogenase 1 |
| 305 | 23351 | Entrez Gene | KIAA0323 |
| 306 | 2065 | Entrez Gene | v-erb-b2 erythroblastic leukemia viral oncogene |
| | | | homolog 3 (avian) |
| 307 | 10217 | Entrez Gene | CTD (carboxy-terminal domain, RNA polymerase II, |
| | | | polypeptide A) small phosphatase-like |
| 308 | 23658 | Entrez Gene | LSM5 homolog, U6 small nuclear RNA associated (S. cerevisiae) |
| 309 | W28948 | GenBank | Full length insert cDNA YH77E09 |
| 311 | 4681 | Entrez Gene | neuroblastoma, suppression of tumorigenicity 1 |
| 314 | 6209 | Entrez Gene | ribosomal protein S15 |
| 316 | 8824 | Entrez Gene | carboxylesterase 2 (intestine, liver) |
| 317 | 10961 | Entrez Gene | endoplasmic reticulum protein 29 |
| 318 | 7265 | Entrez Gene | tetratricopeptide repeat domain 1 |
| 321 | 6128 | Entrez Gene | ribosomal protein L6 |
| 322 | 5245 | Entrez Gene | prohibitin |
| 323 | 9296 | Entrez Gene | ATPase, H+ transporting, lysosomal 14 kDa, V1 subunit F |
| 324 | 1340 | Entrez Gene | cytochrome c oxidase subunit Vib polypeptide 1 |
| | | | (ubiquitous) |
| 325 | 1891 | Entrez Gene | enoyl Coenzyme A hydratase 1, peroxisomal |
| 326 | 1632 | Entrez Gene | dodecenoyl-Coenzyme A delta isomerase (3,2 trans- |
| | | | enoyl-Coenzyme A isomerase) |
| 327 | 1933 | Entrez Gene | eukaryotic translation elongation factor 1 beta 2 |
| 328 | 6141 | Entrez Gene | ribosomal protein L18 |
| 330 | 6950 | Entrez Gene | t-complex 1 |
| 331 | 29760 | Entrez Gene | B-cell linker |
| 332 | 573 | Entrez Gene | BCL2-associated athanogene |
| 333 | 2709 | Entrez Gene | gap junction protein, beta 5 (connexin 31.1) |
| 335 | 11083 | Entrez Gene | death associated transcription factor 1 |
| 336 | 10247 | Entrez Gene | heat-responsive protein 12 |
| 338 | 899 | Entrez Gene | cyclin F |
| 339 | HG162- | The Institute for | — |
| | HT3165 | Genomic Research | |
| 340 | 6139 | Entrez Gene | ribosomal protein L17 |
| 341 | 9521 | Entrez Gene | eukaryotic translation elongation factor 1 epsilon 1 |
| 342 | 1054 | Entrez Gene | CCAAT/enhancer binding protein (C/EBP), gamma |
| 343 | 7739 | Entrez Gene | zinc finger protein 185 (LIM domain) |
| 344 | 8664 | Entrez Gene | eukaryotic translation initiation factor 3, subunit 7 zeta, |
| | | | 66/67 kDa |
| 345 | 57418 | Entrez Gene | WD repeat domain 18 |
| 346 | 6187 | Entrez Gene | ribosomal protein S2 |
| 347 | 11079 | Entrez Gene | RER1 retention in endoplasmic reticulum 1 homolog (S. cerevisiae) |
| 348 | 26211 | Entrez Gene | olfactory receptor, family 2, subfamily F, member 1 |
| 349 | 6206 | Entrez Gene | ribosomal protein S12 |
| 350 | 9798 | Entrez Gene | KIAA0174 |
| 351 | 132556 | Entrez Gene | similar to Transcription factor BTF3 homolog 3 |
| 353 | 4070 | Entrez Gene | tumor-associated calcium signal transducer 2 |
| 354 | 7152 | Entrez Gene | topoisomerase (DNA) I pseudogene 2 |
| 355 | 4615 | Entrez Gene | myeloid differentiation primary response gene (88) |
| 357 | 9377 | Entrez Gene | cytochrome c oxidase subunit Va |
| 358 | 7386 | Entrez Gene | ubiquinol-cytochrome c reductase, Rieske iron-sulfur |
| | | | polypeptide 1 |
| 359 | 4835 | Entrez Gene | NAD(P)H dehydrogenase, quinone 2 |
| 362 | HG1980- | The Institute for | — |
| | HT202 | Genomic Research | |
| 364 | 54107 | Entrez Gene | polymerase (DNA directed), epsilon 3 (p17 subunit) |
| 366 | 8192 | Entrez Gene | ClpP caseinolytic peptidase, ATP-dependent, proteolytic |
| | | | subunit homolog (E. coli) |
| 367 | 51690 | Entrez Gene | LSM7 homolog, U6 small nuclear RNA associated (S. cerevisiae) |
| 368 | 5936 | Entrez Gene | RNA binding motif protein 4 |
| 369 | 7004 | Entrez Gene | TEA domain family member 4 |
| 370 | 3094 | Entrez Gene | histidine triad nucleotide binding protein 1 |
| 372 | 10557 | Entrez Gene | ribonuclease P/MRP 38 kDa subunit |
| 373 | 26156 | Entrez Gene | ribosomal L1 domain containing 1 |
| 374 | 6122 | Entrez Gene | ribosomal protein L3 |
| 375 | 6176 | Entrez Gene | ribosomal protein, large, P1 |
| 378 | 10014 | Entrez Gene | histone deacetylase 5 |
| 379 | 10610 | Entrez Gene | ST6 (alpha-N-acetyl-neuraminyl-2,3-beta-galactosyl-1,3) |
| | | | N-acetylgalactosaminide alpha-2,6-sialyltransferase 2 |
| 380 | HG662-HT662 | The Institute for | — |
| | | Genomic Research | |
| 381 | 10694 | Entrez Gene | chaperonin containing TCP1, subunit 8 (theta) |
| 383 | 4856 | Entrez Gene | nephroblastoma overexpressed gene |
| 384 | 9314 | Entrez Gene | Kruppel-like factor 4 (gut) |
| 385 | 4694 | Entrez Gene | NADH dehydrogenase (ubiquinone) 1 alpha |
| | | | subcomplex, 1, 7.5 kDa |
| 386 | 10904 | Entrez Gene | bladder cancer associated protein |
| 388 | 10682 | Entrez Gene | emopamil binding protein (sterol isomerase) |
| 389 | 8667 | Entrez Gene | eukaryotic translation initiation factor 3, subunit 3 |
| | | | gamma, 40 kDa |
| 395 | 5441 | Entrez Gene | polymerase (RNA) II (DNA directed) polypeptide L, |
| | | | 7.6 kDa |
| 396 | 2146 | Entrez Gene | enhancer of zeste homolog 2 (Drosophila) |
| 398 | 2950 | Entrez Gene | glutathione S-transferase pi |
| 401 | 6156 | Entrez Gene | ribosomal protein L30 |
| 402 | 516 | Entrez Gene | ATP synthase, H+ transporting, mitochondrial F0 |
| | | | complex, subunit c (subunit 9), isoform 1 |
| 403 | 9128 | Entrez Gene | PRP4 pre-mRNA processing factor 4 homolog (yeast) |
| 404 | 3028 | Entrez Gene | hydroxyacyl-Coenzyme A dehydrogenase, type II |
| 405 | 2961 | Entrez Gene | general transcription factor IIE, polypeptide 2, beta |
| | | | 34 kDa |
| 406 | 10969 | Entrez Gene | EBNA1 binding protein 2 |
| 407 | 6748 | Entrez Gene | signal sequence receptor, delta (translocon-associated |
| | | | protein delta) |
| 408 | 4610 | Entrez Gene | v-myc myelocytomatosis viral oncogene homolog 1, |
| | | | lung carcinoma derived (avian) |
| 410 | 23075 | Entrez Gene | SWAP-70 protein |
| 411 | 1537 | Entrez Gene | cytochrome c-1 |
| 412 | 6768 | Entrez Gene | suppression of tumorigenicity 14 (colon carcinoma, |
| | | | matriptase, epithin) |
| 413 | 6138 | Entrez Gene | ribosomal protein L15 |
| 415 | 64976 | Entrez Gene | mitochondrial ribosomal protein L40 |
| 416 | 10420 | Entrez Gene | testis-specific kinase 2 |
| 417 | 9759 | Entrez Gene | histone deacetylase 4 |
| 418 | 4676 | Entrez Gene | nucleosome assembly protein 1-like 4 |
| 419 | 5425 | Entrez Gene | polymerase (DNA directed), delta 2, regulatory subunit |
| | | | 50 kDa |
| 420 | 6390 | Entrez Gene | succinate dehydrogenase complex, subunit B, iron |
| | | | sulfur (Ip) |
| 421 | 162 | Entrez Gene | adaptor-related protein complex 1, beta 1 subunit |
| 423 | 6749 | Entrez Gene | structure specific recognition protein 1 |
| 424 | 4134 | Entrez Gene | microtubule-associated protein 4 |
| 425 | 6832 | Entrez Gene | suppressor of var1, 3-like 1 (S. cerevisiae) |
| 426 | 10939 | Entrez Gene | AFG3 ATPase family gene 3-like 2 (yeast) |
| 429 | 10570 | Entrez Gene | dihydropyrimidinase-like 4 |
| 431 | 8337 | Entrez Gene | histone 2, H2aa |
| 432 | 6193 | Entrez Gene | ribosomal protein S5 |
| 433 | 23492 | Entrez Gene | chromobox homolog 7 |
| 434 | 4809 | Entrez Gene | NHP2 non-histone chromosome protein 2-like 1 (S. cerevisiae) |
| 434 | 4809 | Entrez Gene | NHP2 non-histone chromosome protein 2-like 1 (S. cerevisiae) |
| 436 | 79090 | Entrez Gene | trafficking protein particle complex 6A |
| 437 | 328 | Entrez Gene | APEX nuclease (multifunctional DNA repair enzyme) 1 |
| 438 | 84525 | Entrez Gene | homeodomain-only protein |
| 439 | 2549 | Entrez Gene | GRB2-associated binding protein 1 |
| 440 | 533 | Entrez Gene | ATPase, H+ transporting, lysosomal 21 kDa, V0 subunit |
| | | | c″ |
| 441 | 4717 | Entrez Gene | NADH dehydrogenase (ubiquinone) 1, subcomplex |
| | | | unknown, 1, 6 kDa |
| 442 | 23385 | Entrez Gene | nicastrin |
| 445 | 4678 | Entrez Gene | nuclear autoantigenic sperm protein (histone-binding) |
| 446 | 1847 | Entrez Gene | dual specificity phosphatase 5 |
| 449 | 57017 | Entrez Gene | chromosome 16 open reading frame 49 |
| 452 | 539 | Entrez Gene | ATP synthase, H+ transporting, mitochondrial F1 |
| | | | complex, O subunit (oligomycin sensitivity conferring |
| | | | protein) |
| 453 | 10542 | Entrez Gene | hepatitis B virus x interacting protein |
| 455 | 694 | Entrez Gene | B cell translocation gene 1, anti-proliferative |
| 457 | 4174 | Entrez Gene | MCM5 minichromosome maintenance deficient 5, cell |
| | | | division cycle 46 (S. cerevisiae) |
| 458 | 10278 | Entrez Gene | embryonal Fyn-associated substrate |
| 459 | 593 | Entrez Gene | branched chain keto acid dehydrogenase E1, alpha |
| | | | polypeptide (maple syrup urine disease) |
| 460 | 4357 | Entrez Gene | mercaptopyruvate sulfurtransferase |
| 461 | 3727 | Entrez Gene | jun D proto-oncogene |
| 462 | 51635 | Entrez Gene | dehydrogenase/reductase (SDR family) member 7 |
| 463 | 22870 | Entrez Gene | SAPS domain family, member 1 |
| 464 | 22882 | Entrez Gene | zinc fingers and homeoboxes 2 |
| 465 | 613 | Entrez Gene | breakpoint cluster region |
| 466 | 7150 | Entrez Gene | topoisomerase (DNA) I |
| 467 | 1329 | Entrez Gene | cytochrome c oxidase subunit Vb |
| 469 | 6888 | Entrez Gene | transaldolase 1 |
| 470 | 3184 | Entrez Gene | heterogeneous nuclear ribonucleoprotein D (AU-rich |
| | | | element RNA binding protein 1, 37 kDa) |
| 471 | 6050 | Entrez Gene | ribonuclease/angiogenin inhibitor 1 |
| 472 | 2064 | Entrez Gene | v-erb-b2 erythroblastic leukemia viral oncogene |
| | | | homolog 2, neuro/glioblastoma derived oncogene |
| | | | homolog (avian) |
| 474 | 79026 | Entrez Gene | AHNAK nucleoprotein (desmoyokin) |
| 475 | U77948 | GenBank | general transcription factor II, i /// general transcription |
| | | | factor II, i, pseudogene 1 |
| 476 | 949 | Entrez Gene | scavenger receptor class B, member 1 |
| 477 | 1917 | Entrez Gene | eukaryotic translation elongation factor 1 alpha 2 |
| 478 | 6205 | Entrez Gene | ribosomal protein S11 |
| 480 | HG1614- | The Institute for | — |
| | HT161 | Genomic Research | |
| 481 | 79095 | Entrez Gene | chromosome 9 open reading frame 16 |
| 483 | 22924 | Entrez Gene | microtubule-associated protein, RP/EB family, member 3 |
| 484 | 26578 | Entrez Gene | osteoclast stimulating factor 1 |
| 485 | 1347 | Entrez Gene | cytochrome c oxidase subunit VIIa polypeptide 2 (liver) |
| 486 | 3615 | Entrez Gene | IMP (inosine monophosphate) dehydrogenase 2 |
| 487 | 1981 | Entrez Gene | eukaryotic translation initiation factor 4 gamma, 1 |
| 488 | 22934 | Entrez Gene | ribose 5-phosphate isomerase A (ribose 5-phosphate |
| | | | epimerase) |
| 490 | 11311 | Entrez Gene | vacuolar protein sorting 45A (yeast) |
| 491 | 3916 | Entrez Gene | lysosomal-associated membrane protein 1 |
| 492 | 23020 | Entrez Gene | activating signal cointegrator 1 complex subunit 3-like 1 |
| 493 | 10473 | Entrez Gene | high mobility group nucleosomal binding domain 4 |
| 494 | 10075 | Entrez Gene | HECT, UBA and WWE domain containing 1 |
| 495 | 6812 | Entrez Gene | syntaxin binding protein 1 |
| 496 | 23511 | Entrez Gene | nucleoporin 188 kDa |
| 497 | HG2238- | The Institute for | — |
| | HT232 | Genomic Research | |
| 498 | 283638 | Entrez Gene | KIAA0284 |
| 499 | 30968 | Entrez Gene | stomatin (EPB72)-like 2 |
| 501 | 10634 | Entrez Gene | growth arrest-specific 2 like 1 |
| 502 | 3420 | Entrez Gene | isocitrate dehydrogenase 3 (NAD+) beta |
| 503 | 10424 | Entrez Gene | progesterone receptor membrane component 2 |
| 504 | 8531 | Entrez Gene | cold shock domain protein A |
| 505 | 5439 | Entrez Gene | polymerase (RNA) II (DNA directed) polypeptide J, |
| | | | 13.3 kDa |
| 506 | 10929 | Entrez Gene | Splicing factor, arginine/serine-rich, 46 kD |
| 507 | 6227 | Entrez Gene | ribosomal protein S21 |
| 508 | 6169 | Entrez Gene | ribosomal protein L38 |
| 510 | 6218 | Entrez Gene | ribosomal protein S17 |
| 511 | 9540 | Entrez Gene | tumor protein p53 inducible protein 3 |
| 512 | 1327 | Entrez Gene | cytochrome c oxidase subunit IV isoform 1 |
| 514 | 3030 | Entrez Gene | hydroxyacyl-Coenzyme A dehydrogenase/3-ketoacyl- |
| | | | Coenzyme A thiolase/enoyl-Coenzyme A hydratase |
| | | | (trifunctional protein), alpha subunit |
| 516 | 3032 | Entrez Gene | hydroxyacyl-Coenzyme A dehydrogenase/3-ketoacyl- |
| | | | Coenzyme A thiolase/enoyl-Coenzyme A hydratase |
| | | | (trifunctional protein), beta subunit |
| 517 | 5223 | Entrez Gene | phosphoglycerate mutase 1 (brain) |
| 518 | 10638 | Entrez Gene | S-phase response (cyclin-related) |
| 520 | 10572 | Entrez Gene | CD27-binding (Siva) protein |
| 522 | 162427 | Entrez Gene | hypothetical protein LOC162427 |
| 523 | 10036 | Entrez Gene | chromatin assembly factor 1, subunit A (p150) |
| 524 | 6202 | Entrez Gene | ribosomal protein S8 |
| 525 | 55218 | Entrez Gene | chromosome 14 open reading frame 114 |
| 527 | 9724 | Entrez Gene | UTP14, U3 small nucleolar ribonucleoprotein, homolog |
| | | | C (yeast) |
| 528 | 26003 | Entrez Gene | golgi reassembly stacking protein 2, 55 kDa |
| 529 | 56829 | Entrez Gene | zinc finger CCCH-type, antiviral 1 |
| 530 | 4601 | Entrez Gene | MAX interactor 1 |
| 531 | 6293 | Entrez Gene | vacuolar protein sorting 52 (yeast) |
| 533 | 10067 | Entrez Gene | secretory carrier membrane protein 3 |
| 534 | 5437 | Entrez Gene | polymerase (RNA) II (DNA directed) polypeptide H |
| 535 | 6203 | Entrez Gene | ribosomal protein S9 |
| 536 | 55744 | Entrez Gene | hypothetical protein FLJ10803 |
| 538 | 5036 | Entrez Gene | proliferation-associated 2G4, 38 kDa |
| 539 | 9249 | Entrez Gene | dehydrogenase/reductase (SDR family) member 3 |
| 540 | 1337 | Entrez Gene | cytochrome c oxidase subunit VIa polypeptide 1 |
| 541 | 23623 | Entrez Gene | RUN and SH3 domain containing 1 |
| 542 | 8890 | Entrez Gene | eukaryotic translation initiation factor 2B, subunit 4 |
| | | | delta, 67 kDa |
| 543 | 203069 | Entrez Gene | R3H domain and coiled-coil containing 1 |
| 544 | 7529 | Entrez Gene | tyrosine 3-monooxygenase/tryptophan 5- |
| | | | monooxygenase activation protein, beta polypeptide |
| 545 | 23294 | Entrez Gene | ankyrin repeat and sterile alpha motif domain containing 1 |
| 546 | 29 | Entrez Gene | active BCR-related gene |
| 547 | 1937 | Entrez Gene | eukaryotic translation elongation factor 1 gamma |
| 549 | 147179 | Entrez Gene | WIRE protein |
| 553 | 8480 | Entrez Gene | RAE1 RNA export 1 homolog (S. pombe) |
| 558 | 3163 | Entrez Gene | heme oxygenase (decycling) 2 |
| 559 | 22864 | Entrez Gene | KIAA1002 protein |
| 560 | 23307 | Entrez Gene | KIAA0674 |
| 562 | 9903 | Entrez Gene | kelch-like 21 (Drosophila) |
| 563 | 5236 | Entrez Gene | phosphoglucomutase 1 |
| 565 | 2975 | Entrez Gene | general transcription factor IIIC, polypeptide 1, alpha |
| | | | 220 kDa |
| 566 | 10956 | Entrez Gene | amplified in osteosarcoma |
| 567 | 1201 | Entrez Gene | ceroid-lipofuscinosis, neuronal 3, juvenile (Batten, |
| | | | Spielmeyer-Vogt disease) |
| 569 | 527 | Entrez Gene | ATPase, H+ transporting, lysosomal 16 kDa, V0 subunit c |
| 570 | 8720 | Entrez Gene | membrane-bound transcription factor peptidase, site 1 |
| 571 | 3151 | Entrez Gene | high-mobility group nucleosomal binding domain 2 |
| 572 | 286440 | Entrez Gene | hypothetical protein LOC286440 |
| 573 | 6136 | Entrez Gene | ribosomal protein L12 |
| 575 | 445 | Entrez Gene | argininosuccinate synthetase |
| 576 | 11224 | Entrez Gene | ribosomal protein L35 |
| 577 | 11258 | Entrez Gene | dynactin 3 (p22) |
| 578 | 4779 | Entrez Gene | nuclear factor (erythroid-derived 2)-like 1 |
| 579 | 149603 | Entrez Gene | ring finger protein 187 |
| 580 | 8818 | Entrez Gene | dolichyl-phosphate mannosyltransferase polypeptide 2, |
| | | | regulatory subunit |
| 581 | 823 | Entrez Gene | calpain 1, (mu/l) large subunit |
| 582 | 5438 | Entrez Gene | polymerase (RNA) II (DNA directed) polypeptide I, |
| | | | 14.5 kDa |
| 583 | 6720 | Entrez Gene | sterol regulatory element binding transcription factor 1 |
| 584 | 23633 | Entrez Gene | Karyopherin alpha 6 (importin alpha 7) |
| 585 | 7534 | Entrez Gene | tyrosine 3-monooxygenase/tryptophan 5- |
| | | | monooxygenase activation protein, zeta polypeptide |
| 586 | 23481 | Entrez Gene | pescadillo homolog 1, containing BRCT domain |
| | | | (zebrafish) |

| Gene ID | Probe_ID | Comparison | Fold Change | P value |
|---|---|---|---|---|
| 1 | 36329_at | Day 3 Peri/Day 3 Control | 0.131 | 2.0793E−03 |
| 3 | 32880_at | Day 3 Peri/Day 3 Control | 0.211 | 2.5582E−03 |
| 5 | 38430_at | Day 0 Peri/Day 0 Control | 0.219 | 8.7014E−03 |
| 8 | 32190_at | Day 3 Peri/Day 0 Peri | 0.365 | 8.5757E−03 |
| 11 | 31326_at | Day 7 Peri/Day 7 Control | 0.138 | 7.6909E−03 |
| | 31326_at | Day 3 Peri/Day 3 Control | 0.214 | 6.7718E−03 |
| | 31326_at | Day 7 Peri/Day 0 Peri | 0.229 | 1.6500E−05 |
| | 31326_at | Day 7 Peri/Day 7 Extra | 0.288 | 7.6740E−03 |
| | 31326_at | Day 3 Peri/Day 0 Peri | 0.487 | 3.0725E−03 |
| 13 | 35730_at | Day 0 Peri/Day 0 Intra | 0.252 | 5.3255E−03 |
| 14 | 37809_at | Day 3 Peri/Day 3 Control | 0.180 | 5.0202E−03 |
| | 37809_at | Day 7 Peri/Day 7 Extra | 0.237 | 8.7426E−03 |
| | 37809_at | Day 3 Peri/Day 3 Extra | 0.314 | 9.1991E−03 |
| 15 | 38457_at | Day 0 Peri/Day 0 Control | 0.185 | 5.1425E−04 |
| 17 | 34316_at | Day 3 Peri/Day 3 Control | 0.241 | 3.8100E−05 |
| | 34316_at | Day 7 Peri/Day 7 Control | 0.432 | 2.3230E−04 |
| | 34316_at | Day 3 Peri/Day 3 Extra | 0.476 | 1.6785E−04 |
| 18 | 36569_at | Day 3 Peri/Day 0 Peri | 0.356 | 1.8400E−05 |
| | 36569_at | Day 7 Peri/Day 0 Peri | 0.428 | 5.8746E−04 |
| 20 | 35324_at | Day 3 Peri/Day 0 Peri | 0.447 | 1.3446E−04 |
| | 35324_at | Day 7 Peri/Day 0 Peri | 0.623 | 4.2479E−03 |
| 21 | 38627_at | Day 0 Peri/Day 0 Intra | 0.556 | 5.7849E−03 |
| 22 | 32392_s_at | Day 3 Peri/Day 3 Extra | 0.320 | 3.1014E−03 |
| 24 | 41473_at | Day 3 Peri/Day 0 Peri | 0.521 | 6.6949E−03 |
| 26 | 36312_at | Day 7 Peri/Day 7 Extra | 0.331 | 4.7432E−04 |
| 28 | 41621_i_at | Day 0 Peri/Day 0 Control | 0.350 | 5.4116E−04 |
| 29 | 41772_at | Day 3 Peri/Day 3 Extra | 0.351 | 3.2109E−03 |
| | 41771_g_at | Day 3 Peri/Day 3 Control | 0.464 | 2.4030E−03 |
| | 41772_at | Day 7 Peri/Day 7 Extra | 0.614 | 6.2007E−03 |
| 31 | 34985_at | Day 3 Peri/Day 0 Peri | 0.354 | 4.8214E−04 |
| | 34985_at | Day 7 Peri/Day 0 Peri | 0.381 | 2.9109E−03 |
| 34 | 37761_at | Day 7 Peri/Day 0 Peri | 0.358 | 3.2292E−04 |
| 36 | 32542_at | Day 3 Peri/Day 0 Peri | 0.598 | 1.0604E−03 |
| | 32542_at | Day 7 Peri/Day 0 Peri | 0.643 | 1.4304E−03 |
| 37 | 33294_at | Day 0 Peri/Day 0 Control | 0.369 | 2.9368E−04 |
| | 33294_at | Day 3 Peri/Day 3 Control | 0.453 | 3.7416E−04 |
| 38 | 33860_at | Day 0 Peri/Day 0 Control | 0.369 | 5.2550E−04 |
| | 33860_at | Day 3 Peri/Day 3 Control | 0.374 | 1.4026E−03 |
| | 33860_at | Day 7 Peri/Day 7 Control | 0.452 | 4.0374E−03 |
| | 33860_at | Day 3 Peri/Day 3 Extra | 0.549 | 6.8632E−03 |
| | 33860_at | Day 7 Peri/Day 7 Extra | 0.579 | 3.6353E−03 |
| 39 | 36113_s_at | Day 7 Peri/Day 7 Control | 0.371 | 6.4628E−03 |
| | 36113_s_at | Day 3 Peri/Day 3 Control | 0.452 | 4.3070E−03 |
| 42 | 35363_at | Day 0 Peri/Day 0 Control | 0.383 | 2.2472E−03 |
| | 35363_at | Day 7 Peri/Day 7 Extra | 0.577 | 6.8649E−03 |
| 43 | 35577_at | Day 0 Peri/Day 0 Control | 0.385 | 4.8105E−04 |
| 44 | 38429_at | Day 3 Peri/Day 0 Peri | 0.512 | 8.4627E−03 |
| 45 | 873_at | Day 3 Peri/Day 3 Extra | 0.387 | 6.1920E−03 |
| 46 | 677_s_at | Day 7 Peri/Day 7 Control | 0.389 | 2.8442E−04 |
| | 677_s_at | Day 7 Peri/Day 7 Extra | 0.672 | 8.0184E−03 |
| 49 | 33007_at | Day 3 Peri/Day 3 Control | 0.394 | 7.4079E−03 |
| 50 | 37185_at | Day 0 Peri/Day 0 Control | 0.399 | 5.5226E−03 |
| 51 | 41158_at | Day 0 Peri/Day 0 Control | 0.544 | 4.8737E−03 |
| | 41158_at | Day 3 Peri/Day 0 Peri | 0.715 | 7.5707E−03 |
| 52 | 394_at | Day 3 Peri/Day 3 Control | 0.404 | 6.8138E−03 |
| 53 | 1532_g_at | Day 0 Peri/Day 0 Control | 0.406 | 9.9594E−04 |
| | 1532_g_at | Day 3 Peri/Day 3 Control | 0.567 | 4.1852E−03 |
| | 1532_g_at | Day 0 Peri/Day 0 Intra | 0.660 | 5.2680E−03 |
| 54 | 40409_at | Day 3 Peri/Day 3 Control | 0.445 | 7.4233E−03 |
| 55 | 1527_s_at | Day 0 Peri/Day 0 Control | 0.410 | 2.0450E−03 |
| | 1527_s_at | Day 3 Peri/Day 3 Control | 0.515 | 2.5140E−03 |
| 56 | 40282_s_at | Day 7 Peri/Day 0 Peri | 0.410 | 3.2238E−03 |
| 57 | 35669_at | Day 7 Peri/Day 0 Peri | 0.543 | 1.1342E−03 |
| | 35669_at | Day 3 Peri/Day 0 Peri | 0.585 | 1.2820E−03 |
| 58 | 31610_at | Day 3 Peri/Day 3 Extra | 0.421 | 4.1131E−03 |
| 59 | 35430_at | Day 3 Peri/Day 3 Control | 0.427 | 4.0549E−03 |
| 60 | 33334_at | Day 0 Peri/Day 0 Control | 0.427 | 3.3326E−03 |
| 63 | 34863_s_at | Day 3 Peri/Day 3 Control | 0.430 | 9.0435E−03 |
| 64 | 31431_at | Day 7 Peri/Day 7 Control | 0.430 | 7.9132E−04 |
| 65 | 167_at | Day 3 Peri/Day 3 Control | 0.431 | 3.2376E−04 |
| | 167_at | Day 7 Peri/Day 7 Extra | 0.519 | 7.0199E−03 |
| 66 | 37441_at | Day 0 Peri/Day 0 Intra | 0.432 | 3.4529E−03 |
| 70 | 38384_at | Day 3 Peri/Day 3 Extra | 0.436 | 8.9104E−03 |
| 71 | 936_s_at | Day 3 Peri/Day 3 Control | 0.437 | 1.3929E−04 |
| | 936_s_at | Day 7 Peri/Day 7 Control | 0.554 | 6.0851E−03 |
| | 936_s_at | Day 7 Peri/Day 0 Peri | 0.679 | 6.6192E−03 |
| 72 | 41356_at | Day 3 Peri/Day 3 Extra | 0.438 | 5.3163E−03 |
| 74 | 38798_s_at | Day 0 Peri/Day 0 Control | 0.441 | 6.9369E−03 |
| | 38798_s_at | Day 3 Peri/Day 3 Control | 0.480 | 6.4090E−03 |
| 77 | 38966_at | Day 7 Peri/Day 7 Control | 0.444 | 3.9851E−03 |
| | 38966_at | Day 3 Peri/Day 3 Control | 0.530 | 1.4148E−03 |
| | 38966_at | Day 7 Peri/Day 0 Peri | 0.614 | 2.1060E−03 |
| 78 | 37178_at | Day 3 Peri/Day 3 Control | 0.449 | 3.9314E−03 |
| 79 | 40973_at | Day 0 Peri/Day 0 Control | 0.451 | 2.5568E−03 |
| 80 | 31754_at | Day 3 Peri/Day 3 Extra | 0.453 | 4.6700E−03 |
| 81 | 41258_at | Day 0 Peri/Day 0 Control | 0.453 | 3.7268E−03 |
| 82 | 32242_at | Day 3 Peri/Day 3 Extra | 0.454 | 8.8596E−03 |
| 83 | 41274_at | Day 0 Peri/Day 0 Control | 0.454 | 4.4963E−03 |
| | 41274_at | Day 3 Peri/Day 3 Control | 0.589 | 7.6530E−03 |
| 84 | 975_at | Day 7 Peri/Day 7 Extra | 0.459 | 4.3469E−03 |
| 86 | 1556_at | Day 0 Peri/Day 0 Control | 0.460 | 1.4047E−03 |
| 90 | 32049_f_at | Day 0 Peri/Day 0 Intra | 0.469 | 3.1906E−03 |
| 93 | 40863_r_at | Day 7 Peri/Day 0 Peri | 0.507 | 3.2271E−04 |
| 94 | 34365_at | Day 3 Peri/Day 3 Control | 0.473 | 5.7231E−03 |
| | 34365_at | Day 7 Peri/Day 0 Peri | 0.638 | 2.8273E−03 |
| 95 | 1839_at | Day 3 Peri/Day 3 Extra | 0.473 | 1.6274E−03 |
| | 1839_at | Day 3 Peri/Day 3 Control | 0.555 | 9.8116E−03 |
| 97 | 37628_at | Day 3 Peri/Day 3 Extra | 0.475 | 8.4099E−04 |
| 99 | 39441_at | Day 0 Peri/Day 0 Control | 0.482 | 2.7594E−03 |
| 101 | 37248_at | Day 7 Peri/Day 0 Peri | 0.483 | 2.2125E−04 |
| | 37248_at | Day 3 Peri/Day 0 Peri | 0.522 | 7.1260E−04 |
| 102 | 32664_at | Day 3 Peri/Day 0 Peri | 0.636 | 5.8524E−04 |
| | 32664_at | Day 7 Peri/Day 0 Peri | 0.691 | 3.6796E−03 |
| 103 | 39950_at | Day 3 Peri/Day 3 Control | 0.483 | 8.6125E−03 |
| 106 | 35195_at | Day 7 Peri/Day 7 Extra | 0.489 | 1.6326E−03 |
| 107 | 35561_at | Day 7 Peri/Day 0 Peri | 0.606 | 7.2171E−03 |
| 108 | 31888_s_at | Day 3 Peri/Day 3 Extra | 0.490 | 4.0424E−03 |
| 109 | 31903_at | Day 0 Peri/Day 0 Control | 0.493 | 1.3274E−03 |
| 110 | 37534_at | Day 3 Peri/Day 3 Extra | 0.493 | 7.3178E−03 |
| 112 | 38272_at | Day 7 Peri/Day 7 Extra | 0.494 | 1.4839E−03 |
| 113 | 38327_at | Day 3 Peri/Day 3 Extra | 0.497 | 4.4983E−04 |
| | 38327_at | Day 3 Peri/Day 3 Control | 0.512 | 5.3933E−04 |
| | 38327_at | Day 3 Peri/Day 0 Peri | 0.710 | 9.2062E−03 |
| 115 | 947_at | Day 0 Peri/Day 0 Control | 0.498 | 7.8436E−04 |
| 116 | 31955_at | Day 7 Peri/Day 7 Control | 0.498 | 8.3003E−03 |
| 118 | 34787_at | Day 3 Peri/Day 3 Control | 0.499 | 2.6275E−03 |
| 119 | 33436_at | Day 3 Peri/Day 3 Extra | 0.500 | 5.3219E−03 |
| 120 | 38739_at | Day 3 Peri/Day 3 Extra | 0.501 | 4.4662E−03 |
| 121 | 32486_at | Day 0 Peri/Day 0 Intra | 0.501 | 6.1425E−03 |
| 123 | 501_g_at | Day 3 Peri/Day 3 Control | 0.505 | 2.0051E−03 |
| 129 | 38847_at | Day 7 Peri/Day 7 Extra | 0.510 | 9.6404E−03 |
| 130 | 1243_at | Day 3 Peri/Day 3 Control | 0.511 | 4.6848E−04 |
| | 1243_at | Day 0 Peri/Day 0 Control | 0.556 | 6.7443E−03 |
| | 1243_at | Day 7 Peri/Day 0 Peri | 0.749 | 8.6022E−03 |
| 131 | 40461_at | Day 3 Peri/Day 0 Peri | 0.738 | 3.2244E−03 |
| 132 | 35329_at | Day 3 Peri/Day 3 Control | 0.513 | 1.8894E−03 |
| 136 | 39631_at | Day 3 Peri/Day 3 Extra | 0.517 | 3.1103E−03 |
| | 39631_at | Day 7 Peri/Day 0 Peri | 0.629 | 5.1967E−04 |
| 137 | 41679_at | Day 7 Peri/Day 0 Peri | 0.517 | 1.2344E−03 |
| | 41679_at | Day 3 Peri/Day 0 Peri | 0.607 | 8.6951E−03 |
| 138 | 41182_at | Day 7 Peri/Day 0 Peri | 0.518 | 2.7300E−05 |
| | 41182_at | Day 3 Peri/Day 0 Peri | 0.596 | 1.9071E−04 |
| 141 | 41826_at | Day 0 Peri/Day 0 Control | 0.520 | 5.9389E−03 |
| 142 | 39133_at | Day 7 Peri/Day 7 Control | 0.520 | 1.5729E−03 |
| 144 | 38881_i_at | Day 3 Peri/Day 3 Extra | 0.521 | 1.0549E−04 |
| 146 | 38335_at | Day 0 Peri/Day 0 Intra | 0.759 | 8.2179E−03 |
| 148 | 35773_i_at | Day 7 Peri/Day 7 Control | 0.525 | 1.8840E−03 |
| | 35773_i_at | Day 7 Peri/Day 0 Peri | 0.748 | 5.4735E−03 |
| | 35773_i_at | Day 7 Peri/Day 3 Peri | 0.791 | 1.2691E−03 |
| 149 | 33232_at | Day 7 Peri/Day 0 Peri | 0.526 | 3.2907E−04 |
| | 33232_at | Day 3 Peri/Day 0 Peri | 0.596 | 1.5417E−03 |
| 150 | 36454_at | Day 0 Peri/Day 0 Control | 0.526 | 3.3461E−03 |
| | 35275_at | Day 3 Peri/Day 3 Extra | 0.650 | 2.8445E−03 |
| 151 | 32743_at | Day 0 Peri/Day 0 Control | 0.619 | 1.5268E−03 |
| | 32743_at | Day 3 Peri/Day 0 Peri | 0.772 | 6.0079E−03 |
| 152 | 40096_at | Day 3 Peri/Day 3 Control | 0.527 | 6.5302E−03 |
| | 40096_at | Day 0 Peri/Day 0 Control | 0.563 | 2.0338E−04 |
| 154 | 33029_at | Day 7 Peri/Day 7 Extra | 0.530 | 9.3336E−03 |
| 156 | 653_at | Day 0 Peri/Day 0 Control | 0.533 | 6.1850E−03 |
| 157 | 38347_at | Day 0 Peri/Day 0 Intra | 0.533 | 6.3391E−03 |
| 159 | 37738_g_at | Day 3 Peri/Day 3 Control | 0.678 | 3.6789E−03 |
| 160 | 36497_at | Day 3 Peri/Day 3 Control | 0.535 | 3.4244E−03 |
| | 36497_at | Day 7 Peri/Day 0 Peri | 0.545 | 2.9700E−05 |
| | 36497_at | Day 3 Peri/Day 0 Peri | 0.577 | 3.7100E−05 |
| 165 | 33796_at | Day 3 Peri/Day 3 Control | 0.537 | 6.8833E−03 |
| 166 | 34348_at | Day 3 Peri/Day 3 Control | 0.537 | 9.4449E−03 |
| | 34348_at | Day 0 Peri/Day 0 Control | 0.573 | 7.9326E−03 |
| 167 | 39396_at | Day 3 Peri/Day 3 Control | 0.541 | 7.9687E−03 |
| 169 | 34378_at | Day 7 Peri/Day 7 Extra | 0.542 | 1.2411E−03 |
| 170 | 39074_at | Day 3 Peri/Day 3 Control | 0.544 | 3.5087E−04 |
| 173 | 38113_at | Day 0 Peri/Day 0 Control | 0.546 | 1.8607E−03 |
| 175 | 40615_at | Day 0 Peri/Day 0 Control | 0.546 | 5.1650E−03 |
| 176 | 32112_s_at | Day 3 Peri/Day 3 Extra | 0.547 | 1.9891E−03 |
| 178 | 862_at | Day 3 Peri/Day 3 Extra | 0.548 | 8.5245E−03 |
| 178 | 862_at | Day 7 Peri/Day 7 Extra | 0.576 | 5.9941E−03 |
| 179 | 33128_s_at | Day 7 Peri/Day 0 Peri | 0.549 | 1.9072E−03 |
| | 33128_s_at | Day 3 Peri/Day 0 Peri | 0.605 | 2.6588E−03 |
| 180 | 34369_at | Day 3 Peri/Day 3 Control | 0.549 | 2.5016E−03 |
| | 34369_at | Day 3 Peri/Day 3 Extra | 0.635 | 6.4361E−03 |
| 182 | 32259_at | Day 3 Peri/Day 3 Control | 0.593 | 1.0957E−03 |
| 183 | 1070_at | Day 3 Peri/Day 3 Control | 0.556 | 4.5246E−03 |
| 185 | 36786_at | Day 3 Peri/Day 3 Extra | 0.556 | 7.2273E−04 |
| | 36786_at | Day 7 Peri/Day 7 Control | 0.592 | 1.4203E−03 |
| | 36786_at | Day 3 Peri/Day 3 Control | 0.622 | 8.0958E−04 |
| | 36786_at | Day 0 Peri/Day 0 Control | 0.668 | 3.0374E−03 |
| | 36786_at | Day 7 Peri/Day 0 Peri | 0.783 | 1.0376E−03 |
| 186 | 32618_at | Day 3 Peri/Day 3 Control | 0.557 | 3.0605E−04 |
| | 32618_at | Day 3 Peri/Day 3 Extra | 0.620 | 3.2950E−04 |
| | 32618_at | Day 0 Peri/Day 0 Control | 0.677 | 7.9770E−03 |
| | 32618_at | Day 3 Peri/Day 3 Intra | 0.702 | 5.1159E−03 |
| 187 | 31936_s_at | Day 3 Peri/Day 3 Control | 0.558 | 3.7112E−03 |
| 188 | 35435_s_at | Day 3 Peri/Day 3 Extra | 0.558 | 7.5277E−03 |
| | 354345_s_at | Day 7 Peri/Day 7 Extra | 0.692 | 5.5220E−03 |
| 190 | 41322_s_at | Day 3 Peri/Day 3 Extra | 0.559 | 1.2634E−03 |
| 191 | 37597_s_at | Day 3 Peri/Day 3 Control | 0.560 | 4.7635E−03 |
| | 37597_s_at | Day 3 Peri/Day 3 Extra | 0.566 | 9.6420E−04 |
| 192 | 33988_at | Day 3 Peri/Day 3 Control | 0.560 | 1.0937E−03 |
| | 33988_at | Day 3 Peri/Day 3 Extra | 0.582 | 8.2347E−04 |
| 193 | 39377_at | Day 3 Peri/Day 3 Extra | 0.561 | 3.7750E−03 |
| | 39377_at | Day 3 Peri/Day 3 Control | 0.571 | 9.8955E−03 |
| | 39377_at | Day 3 Peri/Day 3 Intra | 0.634 | 9.8004E−03 |
| 194 | 160044_g_at | Day 3 Peri/Day 3 Control | 0.562 | 5.6984E−03 |
| 195 | 31691_g_at | Day 3 Peri/Day 3 Control | 0.563 | 6.9886E−03 |
| 197 | 32161_at | Day 0 Peri/Day 0 Intra | 0.565 | 2.6625E−03 |
| 198 | 39856_at | Day 0 Peri/Day 0 Control | 0.568 | 7.8476E−03 |
| 199 | 421_at | Day 0 Peri/Day 0 Control | 0.569 | 3.6369E−03 |
| 201 | 33399_at | Day 3 Peri/Day 3 Extra | 0.571 | 4.6866E−03 |
| 202 | 38824_at | Day 3 Peri/Day 3 Control | 0.571 | 7.2789E−03 |
| | 38824_at | Day 3 Peri/Day 3 Extra | 0.592 | 5.7073E−03 |
| 203 | 39103_s_at | Day 3 Peri/Day 3 Control | 0.571 | 2.1724E−04 |
| | 39103_s_at | Day 3 Peri/Day 3 Extra | 0.601 | 5.7200E−03 |
| 204 | 34845_at | Day 3 Peri/Day 3 Extra | 0.572 | 1.2889E−03 |
| 205 | 40437_at | Day 3 Peri/Day 3 Control | 0.572 | 3.2143E−03 |
| 206 | 32523_at | Day 3 Peri/Day 3 Control | 0.573 | 4.0809E−03 |
| 207 | 374_f_at | Day 7 Peri/Day 7 Control | 0.573 | 7.7105E−03 |
| 208 | 31492_at | Day 3 Peri/Day 3 Extra | 0.574 | 7.4544E−04 |
| 209 | 32747_at | Day 3 Peri/Day 3 Extra | 0.574 | 5.1549E−04 |
| | 32747_at | Day 7 Peri/Day 0 Peri | 0.741 | 2.4111E−03 |
| 210 | 41234_at | Day 7 Peri/Day 7 Extra | 0.577 | 3.8743E−03 |
| 212 | 36553_at | Day 3 Peri/Day 0 Peri | 0.710 | 1.0994E−04 |
| 213 | 38312_at | Day 3 Peri/Day 0 Peri | 0.719 | 4.2489E−04 |
| 214 | 41029_at | Day 3 Peri/Day 3 Extra | 0.580 | 1.0146E−03 |
| 215 | 34760_at | Day 0 Peri/Day 0 Intra | 0.580 | 5.4949E−04 |
| 216 | 34893_at | Day 7 Peri/Day 7 Control | 0.581 | 9.2931E−03 |
| | 34893_at | Day 7 Peri/Day 7 Extra | 0.600 | 1.3243E−03 |
| 217 | 35371_at | Day 3 Peri/Day 3 Control | 0.582 | 1.9359E−03 |
| | 35371_at | Day 3 Peri/Day 3 Extra | 0.618 | 2.4693E−03 |
| 218 | 37708_r_at | Day 0 Peri/Day 0 Intra | 0.600 | 6.4350E−03 |
| 220 | 36992_at | Day 3 Peri/Day 3 Extra | 0.583 | 3.0827E−03 |
| | 36992_at | Day 3 Peri/Day 3 Intra | 0.673 | 4.8045E−03 |
| 222 | 40138_at | Day 7 Peri/Day 7 Control | 0.584 | 6.5665E−03 |
| 223 | 1597_at | Day 3 Peri/Day 0 Peri | 0.689 | 5.8701E−03 |
| 224 | 41257_at | Day 0 Peri/Day 0 Intra | 0.700 | 3.4819E−03 |
| 228 | 37002_at | Day 7 Peri/Day 7 Control | 0.588 | 8.3451E−04 |
| 229 | 36284_at | Day 7 Peri/Day 7 Extra | 0.589 | 9.2936E−03 |
| 230 | 35367_at | Day 7 Peri/Day 0 Peri | 0.683 | 4.0159E−04 |
| | 35367_at | Day 3 Peri/Day 0 Peri | 0.763 | 8.3297E−03 |
| 231 | 1373_at | Day 3 Peri/Day 0 Peri | 0.738 | 3.1310E−04 |
| 232 | 35760_at | Day 3 Peri/Day 3 Extra | 0.590 | 1.5912E−03 |
| | 35760_at | Day 0 Peri/Day 0 Control | 0.738 | 8.9277E−03 |
| 233 | 40303_at | Day 3 Peri/Day 3 Control | 0.591 | 7.9662E−03 |
| 234 | 40764_at | Day 3 Peri/Day 3 Extra | 0.591 | 2.0500E−03 |
| 235 | 32586_at | Day 0 Peri/Day 0 Control | 0.591 | 3.7384E−03 |
| | 32586_at | Day 3 Peri/Day 3 Control | 0.685 | 8.4169E−04 |
| | 32586_at | Day 3 Peri/Day 3 Extra | 0.782 | 5.1596E−03 |
| 237 | 37668_at | Day 3 Peri/Day 3 Extra | 0.591 | 6.3705E−03 |
| 238 | 40946_at | Day 3 Peri/Day 3 Extra | 0.593 | 3.2988E−03 |
| 239 | 41403_at | Day 3 Peri/Day 3 Extra | 0.593 | 2.1695E−03 |
| | 41403_at | Day 7 Peri/Day 7 Extra | 0.674 | 9.8788E−04 |
| 240 | 34990_at | Day 3 Peri/Day 0 Peri | 0.593 | 3.4533E−04 |
| | 34990_at | Day 7 Peri/Day 0 Peri | 0.711 | 9.8766E−03 |
| 241 | 32715_at | Day 3 Peri/Day 3 Extra | 0.593 | 2.2624E−03 |
| | 32715_at | Day 0 Peri/Day 0 Control | 0.628 | 1.3473E−03 |
| 243 | 2051_at | Day 7 Peri/Day 7 Control | 0.594 | 5.4697E−03 |
| 245 | 34350_at | Day 7 Peri/Day 7 Extra | 0.595 | 8.1420E−03 |
| 246 | 33916_at | Day 0 Peri/Day 0 Control | 0.595 | 3.7132E−03 |
| | 33916_at | Day 3 Peri/Day 3 Control | 0.647 | 6.0432E−03 |
| 248 | 32766_at | Day 3 Peri/Day 3 Extra | 0.597 | 8.7735E−04 |
| 249 | 37229_at | Day 7 Peri/Day 7 Extra | 0.619 | 5.1546E−03 |
| 250 | 40822_at | Day 0 Peri/Day 0 Control | 0.597 | 3.4027E−03 |
| | 40823_s_at | Day 3 Peri/Day 3 Extra | 0.633 | 7.3198E−03 |
| | 40822_at | Day 0 Peri/Day 0 Intra | 0.746 | 7.0070E−03 |
| 252 | 1378_g_at | Day 3 Peri/Day 3 Extra | 0.598 | 6.5881E−03 |
| | 1378_g_at | Day 0 Peri/Day 0 Control | 0.646 | 7.6400E−03 |
| 254 | 40440_at | Day 0 Peri/Day 0 Control | 0.599 | 7.7405E−03 |
| | 40440_at | Day 3 Peri/Day 3 Intra | 0.727 | 9.5681E−03 |
| 255 | 41569_at | Day 7 Peri/Day 7 Extra | 0.599 | 4.9293E−03 |
| 257 | 39025_at | Day 7 Peri/Day 7 Control | 0.716 | 2.2149E−03 |
| | 39025_at | Day 7 Peri/Day 0 Peri | 0.798 | 1.0424E−03 |
| 258 | 1719_at | Day 3 Peri/Day 3 Extra | 0.603 | 6.7926E−03 |
| 259 | 218_at | Day 3 Peri/Day 3 Control | 0.606 | 4.9835E−04 |
| 260 | 33675_at | Day 3 Peri/Day 0 Peri | 0.606 | 4.5158E−03 |
| 262 | 34310_at | Day 3 Peri/Day 3 Control | 0.607 | 7.6231E−03 |
| | 34310_at | Day 3 Peri/Day 3 Intra | 0.619 | 3.4883E−03 |
| 263 | 36111_s_at | Day 7 Peri/Day 7 Extra | 0.607 | 1.0792E−03 |
| 265 | 41804_at | Day 3 Peri/Day 3 Control | 0.608 | 1.1934E−04 |
| 266 | 39883_at | Day 7 Peri/Day 7 Extra | 0.609 | 2.6811E−03 |
| 267 | 37828_at | Day 0 Peri/Day 0 Intra | 0.626 | 4.8045E−03 |
| 268 | 37992_s_at | Day 7 Peri/Day 7 Control | 0.611 | 6.2111E−03 |
| 269 | 35736_at | Day 0 Peri/Day 0 Control | 0.611 | 4.7952E−03 |
| 271 | 32395_r_at | Day 3 Peri/Day 3 Extra | 0.611 | 2.2508E−03 |
| 273 | 34950_at | Day 3 Peri/Day 0 Peri | 0.613 | 2.6182E−04 |
| 275 | 40087_at | Day 3 Peri/Day 3 Control | 0.614 | 3.7491E−03 |
| 276 | 41837_at | Day 3 Peri/Day 0 Peri | 0.614 | 6.2326E−04 |
| 276 | 41837_at | Day 7 Peri/Day 0 Peri | 0.660 | 1.7553E−03 |
| 277 | 33337_at | Day 3 Peri/Day 3 Control | 0.614 | 8.5255E−03 |
| | 33337_at | Day 7 Peri/Day 7 Control | 0.626 | 3.0395E−03 |
| | 33337_at | Day 7 Peri/Day 0 Peri | 0.690 | 1.2271E−03 |
| 278 | 38451_at | Day 7 Peri/Day 7 Control | 0.614 | 8.2100E−05 |
| 279 | 40497_at | Day 0 Peri/Day 0 Control | 0.614 | 9.7424E−03 |
| | 40497_at | Day 3 Peri/Day 3 Control | 0.655 | 1.1667E−04 |
| 280 | 34841_at | Day 3 Peri/Day 3 Extra | 0.615 | 9.0352E−03 |
| | 34841_at | Day 3 Peri/Day 0 Peri | 0.776 | 9.4126E−03 |
| 281 | 39823_at | Day 3 Peri/Day 3 Control | 0.616 | 2.9015E−03 |
| 282 | 34414_at | Day 0 Peri/Day 0 Intra | 0.644 | 5.3735E−03 |
| 285 | 1898_at | Day 7 Peri/Day 0 Peri | 0.617 | 3.0138E−03 |
| 286 | 32576_at | Day 3 Peri/Day 3 Extra | 0.617 | 4.1860E−03 |
| 287 | 40774_at | Day 3 Peri/Day 3 Extra | 0.617 | 6.5552E−3 |
| 290 | 35966_at | Day 0 Peri/Day 0 Intra | 0.663 | 6.8919E−03 |
| 291 | 34352_at | Day 7 Peri/Day 7 Control | 0.620 | 2.4354E−03 |
| | 34352_at | Day 3 Peri/Day 3 Intra | 0.704 | 1.5982E−03 |
| 292 | 1356_at | Day 0 Peri/Day 0 Control | 0.621 | 1.4788E−03 |
| 293 | 32805_at | Day 3 Peri/Day 3 Extra | 0.621 | 6.7226E−03 |
| 294 | 40086_at | Day 3 Peri/Day 3 Control | 0.623 | 5.5939E−03 |
| 295 | 36463_at | Day 3 Peri/Day 3 Extra | 0.624 | 7.6918E−03 |
| 296 | 41329_at | Day 3 Peri/Day 3 Control | 0.624 | 2.9063E−03 |
| 298 | 33351_at | Day 0 Peri/Day 0 Intra | 0.624 | 5.2711E−04 |
| 299 | 38131_at | Day 3 Peri/Day 3 Extra | 0.626 | 9.0721E−03 |
| 300 | 41498_at | Day 3 Peri/Day 3 Control | 0.626 | 9.2124E−04 |
| | 41498_at | Day 3 Peri/Day 0 Peri | 0.777 | 3.9252E−03 |
| 301 | 2093_s_at | Day 3 Peri/Day 3 Control | 0.627 | 1.0646E−03 |
| | 38733_at | Day 0 Peri/Day 0 Control | 0.644 | 9.6568E−03 |
| 303 | 38911_at | Day 3 Peri/Day 3 Extra | 0.627 | 2.3405E−03 |
| 304 | 35702_at | Day 7 Peri/Day 0 Peri | 0.628 | 6.5442E−03 |
| 305 | 32592_at | Day 0 Peri/Day 0 Control | 0.628 | 8.9436E−04 |
| 306 | 2089_s_at | Day 0 Peri/Day 0 Control | 0.629 | 5.0250E−03 |
| | 32787_at | Day 7 Peri/Day 0 Peri | 0.677 | 7.5081E−03 |
| 307 | 40196_at | Day 3 Peri/Day 3 Control | 0.630 | 3.3471E−03 |
| | 40196_at | Day 7 Peri/Day 0 Peri | 0.735 | 2.0122E−03 |
| | 40196_at | Day 3 Peri/Day 0 Peri | 0.770 | 5.2302E−03 |
| 308 | 34270_at | Day 3 Peri/Day 3 Control | 0.631 | 1.9266E−03 |
| 309 | 37161_at | Day 3 Peri/Day 3 Extra | 0.631 | 4.5494E−03 |
| | 37161_at | Day 3 Peri/Day 3 Control | 0.640 | 6.9170E−03 |
| 311 | 37005_at | Day 7 Peri/Day 0 Peri | 0.642 | 1.7449E−03 |
| | 37005_at | Day 3 Peri/Day 0 Peri | 0.695 | 2.4042E−03 |
| 314 | 39916_r_at | Day 7 Peri/Day 7 Control | 0.633 | 1.4158E−03 |
| | 39916_r_at | Day 3 Peri/Day 3 Control | 0.675 | 7.7101E−03 |
| 316 | 40882_at | Day 7 Peri/Day 7 Intra | 0.635 | 9.8901E−03 |
| 317 | 36945_at | Day 3 Peri/Day 3 Control | 0.635 | 9.2309E−04 |
| 318 | 37321_at | Day 3 Peri/Day 3 Control | 0.635 | 7.5603E−03 |
| 321 | 31952_at | Day 3 Peri/Day 3 Extra | 0.637 | 8.7667E−03 |
| 322 | 36592_at | Day 3 Peri/Day 3 Extra | 0.637 | 3.4444E−03 |
| 323 | 37395_at | Day 7 Peri/Day 7 Control | 0.638 | 2.2448E−03 |
| | 37395_at | Day 3 Peri/Day 3 Control | 0.722 | 8.6817E−04 |
| | 37395_at | Day 3 Peri/Day 3 Extra | 0.810 | 7.4746E−03 |
| | 37395_at | Day 7 Peri/Day 0 Peri | 0.840 | 4.0330E−03 |
| 324 | 40872_at | Day 3 Peri/Day 3 Extra | 0.638 | 1.3105E−03 |
| 325 | 32756_at | Day 3 Peri/Day 3 Extra | 0.638 | 8.7097E−03 |
| 326 | 37982_at | Day 7 Peri/Day 7 Control | 0.639 | 2.3722E−03 |
| 327 | 35748_at | Day 3 Peri/Day 3 Extra | 0.639 | 7.6265E−04 |
| 328 | 31546_at | Day 7 Peri/Day 7 Control | 0.639 | 6.9963E−03 |
| | 31546_at | Day 3 Peri/Day 3 Extra | 0.691 | 6.7124E−03 |
| | 31546_at | Day 7 Peri/Day 0 Peri | 0.760 | 9.8180E−03 |
| 330 | 34791_at | Day 3 Peri/Day 3 Extra | 0.640 | 3.7040E−03 |
| 331 | 38242_at | Day 3 Peri/Day 3 Extra | 0.640 | 7.2677E−04 |
| 332 | 34798_at | Day 3 Peri/Day 3 Control | 0.642 | 4.4022E−03 |
| 333 | 38903_at | Day 7 Peri/Day 0 Peri | 0.643 | 1.7967E−03 |
| 335 | 32045_at | Day 0 Peri/Day 0 Control | 0.644 | 6.8658E−03 |
| 336 | 32173_at | Day 7 Peri/Day 7 Extra | 0.645 | 6.3967E−03 |
| | 32173_at | Day 3 Peri/Day 3 Control | 0.659 | 6.9500E−03 |
| 338 | 35907_at | Day 3 Peri/Day 3 Extra | 0.646 | 8.5538E−03 |
| 339 | 1278_at | Day 3 Peri/Day 0 Peri | 0.647 | 6.0118E−03 |
| 340 | 32440_at | Day 3 Peri/Day 3 Extra | 0.647 | 6.1617E−03 |
| 341 | 40587_s_at | Day 3 Peri/Day 3 Intra | 0.697 | 4.8656E−04 |
| | 40587_s_at | Day 3 Peri/Day 3 Extra | 0.718 | 6.3530E−03 |
| 342 | 39219_at | Day 7 Peri/Day 7 Extra | 0.647 | 8.7653E−03 |
| 343 | 32139_at | Day 0 Peri/Day 0 Control | 0.648 | 5.7560E−03 |
| 344 | 35298_at | Day 3 Peri/Day 3 Extra | 0.649 | 2.0180E−03 |
| 345 | 35983_at | Day 3 Peri/Day 3 Extra | 0.651 | 3.7380E−03 |
| 346 | 31527_at | Day 3 Peri/Day 3 Extra | 0.651 | 6.9801E−03 |
| 347 | 41551_at | Day 7 Peri/Day 7 Extra | 0.651 | 8.1132E−03 |
| 348 | 31921_at | Day 3 Peri/Day 3 Control | 0.652 | 2.1176E−03 |
| 349 | 33117_r_at | Day 3 Peri/Day 3 Extra | 0.652 | 2.4740E−03 |
| 350 | 36942_at | Day 0 Peri/Day 0 Control | 0.653 | 5.6656E−03 |
| 351 | 31519_f_at | Day 0 Peri/Day 0 Intra | 0.653 | 9.2891E−03 |
| 353 | 291_s_at | Day 0 Peri/Day 0 Control | 0.654 | 9.5683E−03 |
| | 291_s_at | Day 3 Peri/Day 3 Extra | 0.655 | 8.7414E−03 |
| 354 | 31680_at | Day 7 Peri/Day 7 Extra | 0.654 | 8.0454E−03 |
| 355 | 38369_at | Day 7 Peri/Day 7 Extra | 0.654 | 5.5186E−03 |
| 357 | 41223_at | Day 3 Peri/Day 3 Extra | 0.656 | 7.0969E−03 |
| 358 | 34401_at | Day 3 Peri/Day 3 Extra | 0.656 | 5.8159E−03 |
| 359 | 36880_at | Day 7 Peri/Day 7 Control | 0.657 | 7.2935E−03 |
| 362 | 956_at | Day 7 Peri/Day 7 Extra | 0.658 | 8.0238E−03 |
| 364 | 38702_at | Day 3 Peri/Day 3 Extra | 0.660 | 3.6355E−03 |
| 366 | 32528_at | Day 7 Peri/Day 7 Intra | 0.661 | 7.3475E−03 |
| 367 | 36846_s_at | Day 7 Peri/Day 7 Control | 0.662 | 8.8105E−04 |
| 368 | 35351_at | Day 3 Peri/Day 3 Extra | 0.662 | 1.3875E−03 |
| 369 | 41037_at | Day 7 Peri/Day 7 Extra | 0.662 | 6.4186E−04 |
| 370 | 1009_at | Day 3 Peri/Day 3 Extra | 0.662 | 6.3134E−03 |
| | 1009_at | Day 3 Peri/Day 3 Intra | 0.704 | 8.1604E−03 |
| 372 | 41040_at | Day 7 Peri/Day 7 Extra | 0.663 | 2.3436E−03 |
| 373 | 39418_at | Day 7 Peri/Day 7 Extra | 0.664 | 2.4392E−03 |
| | 39418_at | Day 3 Peri/Day 3 Extra | 0.730 | 4.3297E−03 |
| 374 | 31722_at | Day 3 Peri/Day 3 Extra | 0.665 | 8.4932E−03 |
| | 31722_at | Day 7 Peri/Day 0 Peri | 0.792 | 9.5624E−03 |
| 375 | 31957_r_at | Day 7 Peri/Day 0 Peri | 0.666 | 3.7416E−03 |
| 378 | 38810_at | Day 3 Peri/Day 3 Control | 0.667 | 1.7702E−04 |
| | 38810_at | Day 7 Peri/Day 0 Peri | 0.778 | 7.8739E−03 |
| | 38810_at | Day 3 Peri/Day 0 Peri | 0.784 | 9.3572E−04 |
| 379 | 34693_at | Day 7 Peri/Day 0 Peri | 0.668 | 5.8340E−04 |
| 380 | 1151_at | Day 3 Peri/Day 3 Control | 0.669 | 1.5550E−03 |
| 381 | 39767_at | Day 3 Peri/Day 3 Extra | 0.669 | 4.6792E−03 |
| 383 | 39250_at | Day 3 Peri/Day 0 Peri | 0.670 | 5.1134E−03 |
| 384 | 36214_at | Day 7 Peri/Day 0 Peri | 0.670 | 7.5246E−03 |
| 385 | 36169_at | Day 3 Peri/Day 3 Control | 0.671 | 5.9310E−04 |
| | 36169_at | Day 3 Peri/Day 3 Extra | 0.706 | 1.3204E−03 |
| | 36169_at | Day 7 Peri/Day 7 Control | 0.710 | 9.3036E−03 |
| | 36169_at | Day 3 Peri/Day 3 Intra | 0.754 | 3.5482E−03 |
| 386 | 35266_at | Day 3 Peri/Day 0 Peri | 0.815 | 4.1112E−03 |
| 388 | 32536_at | Day 3 Peri/Day 3 Extra | 0.672 | 3.7237E−03 |
| 389 | 35327_at | Day 3 Peri/Day 3 Control | 0.672 | 5.0660E−03 |
| 395 | 35841_at | Day 7 Peri/Day 7 Control | 0.676 | 2.4659E−03 |
| 396 | 37305_at | Day 7 Peri/Day 7 Extra | 0.677 | 5.6094E−03 |
| | 37305_at | Day 3 Peri/Day 3 Extra | 0.701 | 9.0063E−03 |
| 398 | 33396_at | Day 3 Peri/Day 3 Extra | 0.679 | 6.0265E−03 |
| | 33396_at | Day 7 Peri/Day 7 Extra | 0.695 | 5.1049E−03 |
| 401 | 31708_at | Day 3 Peri/Day 3 Extra | 0.682 | 4.7648E−03 |
| 402 | 38076_at | Day 3 Peri/Day 3 Extra | 0.682 | 3.2512E−04 |
| 403 | 37936_at | Day 3 Peri/Day 3 Extra | 0.682 | 1.2100E−03 |
| | 37936_at | Day 3 Peri/Day 3 Intra | 0.809 | 4.3656E−03 |
| 404 | 40778_at | Day 3 Peri/Day 3 Control | 0.683 | 9.5528E−03 |
| | 40778_at | Day 3 Peri/Day 3 Extra | 0.687 | 5.4834E−03 |
| | 40778_at | Day 0 Peri/Day 0 Control | 0.700 | 6.7401E−03 |
| | 40778_at | Day 7 Peri/Day 0 Peri | 0.785 | 3.6406E−03 |
| 405 | 37295_at | Day 7 Peri/Day 7 Extra | 0.683 | 5.1041E−03 |
| 406 | 36135_at | Day 3 Peri/Day 3 Extra | 0.683 | 7.3375E−03 |
| 407 | 38635_at | Day 7 Peri/Day 7 Control | 0.684 | 3.2000E−03 |
| 408 | 1490_at | Day 3 Peri/Day 0 Peri | 0.684 | 2.9494E−04 |
| 410 | 31869_at | Day 0 Peri/Day 0 Intra | 0.684 | 5.9906E−03 |
| 411 | 1160_at | Day 3 Peri/Day 3 Control | 0.685 | 9.9616E−03 |
| 412 | 35309_at | Day 3 Peri/Day 3 Extra | 0.685 | 2.8284E−03 |
| 413 | 32433_at | Day 0 Peri/Day 0 Intra | 0.686 | 4.8021E−03 |
| 415 | 32745_at | Day 3 Peri/Day 3 Control | 0.688 | 7.4413E−03 |
| 416 | 33164_at | Day 7 Peri/Day 0 Peri | 0.688 | 8.0384E−03 |
| 417 | 38271_at | Day 0 Peri/Day 0 Intra | 0.776 | 6.2327E−03 |
| 418 | 32575_at | Day 3 Peri/Day 3 Control | 0.689 | 7.9600E−05 |
| | 32575_at | Day 3 Peri/Day 3 Intra | 0.820 | 9.3028E−03 |
| 419 | 1470_at | Day 3 Peri/Day 3 Intra | 0.689 | 5.7659E−03 |
| 420 | 35751_at | Day 3 Peri/Day 3 Extra | 0.689 | 9.9046E−03 |
| 421 | 40745_at | Day 3 Peri/Day 3 Extra | 0.689 | 1.6623E−03 |
| 423 | 37739_at | Day 3 Peri/Day 3 Extra | 0.690 | 4.8988E−03 |
| 424 | 33850_at | Day 3 Peri/Day 0 Peri | 0.690 | 9.2919E−04 |
| | 33850_at | Day 7 Peri/Day 0 Peri | 0.747 | 8.7450E−03 |
| 425 | 41408_at | Day 3 Peri/Day 3 Extra | 0.691 | 6.4202E−03 |
| 426 | 34315_at | Day 3 Peri/Day 3 Extra | 0.692 | 3.6562E−04 |
| 429 | 39503_s_at | Day 7 Peri/Day 0 Peri | 0.693 | 9.8616E−03 |
| 431 | 286_at | Day 3 Peri/Day 3 Control | 0.693 | 7.8013E−03 |
| 432 | 32437_at | Day 3 Peri/Day 3 Extra | 0.693 | 1.4564E−03 |
| | 32437_at | Day 7 Peri/Day 0 Peri | 0.740 | 3.6964E−03 |
| | 32437_at | Day 3 Peri/Day 0 Peri | 0.808 | 6.6422E−03 |
| 433 | 36894_at | Day 3 Peri/Day 3 Control | 0.693 | 8.8784E−03 |
| | 36894_at | Day 3 Peri/Day 0 Peri | 0.742 | 1.6692E−03 |
| | 36894_at | Day 7 Peri/Day 0 Peri | 0.742 | 3.4050E−03 |
| 434 | 41746_at | Day 3 Peri/Day 3 Control | 0.693 | 5.4886E−03 |
| 434 | 41746_at | Day 3 Peri/Day 0 Peri | 0.830 | 6.1703E−03 |
| 436 | 36529_at | Day 3 Peri/Day 0 Peri | 0.694 | 6.8032E−03 |
| 437 | 2025_s_at | Day 3 Peri/Day 3 Control | 0.695 | 4.9002E−03 |
| | 2025_s_at | Day 3 Peri/Day 3 Extra | 0.746 | 9.3963E−03 |
| 438 | 39698_at | Day 7 Peri/Day 0 Peri | 0.695 | 8.7289E−03 |
| 439 | 33997_at | Day 0 Peri/Day 0 Intra | 0.731 | 4.0375E−03 |
| 440 | 36167_at | Day 7 Peri/Day 7 Control | 0.696 | 9.2513E−03 |
| 441 | 38485_at | Day 3 Peri/Day 3 Extra | 0.696 | 2.0589E−03 |
| 442 | 34835_at | Day 3 Peri/Day 3 Extra | 0.696 | 1.6857E−03 |
| | 34835_at | Day 3 Peri/Day 3 Intra | 0.790 | 4.8084E−03 |
| 445 | 33255_at | Day 3 Peri/Day 3 Control | 0.696 | 9.0975E−03 |
| 446 | 529_at | Day 7 Peri/Day 7 Extra | 0.697 | 3.7478E−03 |
| 449 | 34810_at | Day 7 Peri/Day 7 Control | 0.700 | 1.2592E−03 |
| | 34810_at | Day 7 Peri/Day 0 Peri | 0.810 | 4.2261E−03 |
| 452 | 37029_at | Day 3 Peri/Day 3 Intra | 0.701 | 5.7342E−03 |
| 453 | 38054_at | Day 3 Peri/Day 3 Control | 0.704 | 9.1709E−03 |
| 455 | 37294_at | Day 3 Peri/Day 3 Extra | 0.704 | 5.5679E−03 |
| 457 | 982_at | Day 7 Peri/Day 3 Peri | 0.706 | 4.9235E−04 |
| 458 | 33883_at | Day 7 Peri/Day 0 Peri | 0.706 | 2.0000E−05 |
| | 33883_at | Day 3 Peri/Day 0 Peri | 0.718 | 6.1275E−04 |
| 459 | 37704_at | Day 3 Peri/Day 3 Control | 0.707 | 1.4726E−03 |
| | 37704_at | Day 3 Peri/Day 3 Extra | 0.720 | 1.7216E−03 |
| 460 | 36124_at | Day 7 Peri/Day 0 Peri | 0.707 | 7.1338E−04 |
| 461 | 41483_s_at | Day 7 Peri/Day 0 Peri | 0.707 | 9.7391E−03 |
| 462 | 39814_s_at | Day 3 Peri/Day 3 Extra | 0.708 | 6.9810E−03 |
| | 39814_s_at | Day 3 Peri/Day 3 Intra | 0.740 | 6.8403E−03 |
| 463 | 36862_at | Day 3 Peri/Day 3 Control | 0.709 | 6.5040E−03 |
| 464 | 41503_at | Day 3 Peri/Day 0 Peri | 0.709 | 9.7700E−05 |
| | 41503_at | Day 7 Peri/Day 0 Peri | 0.830 | 5.9812E−03 |
| 465 | 34679_at | Day 3 Peri/Day 3 Extra | 0.709 | 4.4827E−03 |
| 466 | 1030_s_at | Day 7 Peri/Day 7 Extra | 0.711 | 1.5853E−03 |
| 467 | 39921_at | Day 3 Peri/Day 3 Extra | 0.711 | 8.1965E−03 |
| 469 | 37311_at | Day 3 Peri/Day 3 Extra | 0.713 | 2.9353E−04 |
| 470 | 38016_at | Day 0 Peri/Day 0 Intra | 0.714 | 4.2703E−03 |
| 471 | 36187_at | Day 3 Peri/Day 3 Extra | 0.715 | 1.3879E−03 |
| | 36187_at | Day 3 Peri/Day 3 Control | 0.730 | 5.3879E−03 |
| 472 | 33218_at | Day 3 Peri/Day 0 Peri | 0.718 | 4.9044E−04 |
| | 33218_at | Day 7 Peri/Day 0 Peri | 0.740 | 6.2999E−04 |
| 474 | 37027_at | Day 7 Peri/Day 0 Peri | 0.748 | 8.8700E−05 |
| | 37027_at | Day 3 Peri/Day 0 Peri | 0.780 | 3.1374E−04 |
| 475 | 35450_s_at | Day 0 Peri/Day 0 Control | 0.718 | 8.5556E−03 |
| 476 | 41200_at | Day 7 Peri/Day 0 Peri | 0.720 | 1.3768E−03 |
| | 41200_at | Day 3 Peri/Day 0 Peri | 0.721 | 1.6250E−03 |
| 477 | 35174_i_at | Day 7 Peri/Day 0 Peri | 0.720 | 6.7372E−04 |
| 478 | 32330_at | Day 7 Peri/Day 0 Peri | 0.721 | 3.3215E−03 |
| 480 | 954_s_at | Day 7 Peri/Day 7 Control | 0.722 | 8.4646E−03 |
| | 954_s_at | Day 7 Peri/Day 7 Intra | 0.747 | 1.0730E−03 |
| 481 | 41047_at | Day 3 Peri/Day 0 Peri | 0.725 | 5.9374E−03 |
| 483 | 40825_at | Day 7 Peri/Day 0 Peri | 0.725 | 1.1882E−03 |
| | 40825_at | Day 3 Peri/Day 0 Peri | 0.765 | 2.4008E−03 |
| 484 | 467_at | Day 7 Peri/Day 7 Extra | 0.725 | 7.2710E−03 |
| 485 | 41760_at | Day 3 Peri/Day 3 Extra | 0.726 | 1.4279E−03 |
| 486 | 36624_at | Day 3 Peri/Day 3 Extra | 0.726 | 5.8955E−03 |
| | 36624_at | Day 7 Peri/Day 0 Peri | 0.740 | 8.1540E−04 |
| 487 | 32844_at | Day 7 Peri/Day 0 Peri | 0.727 | 9.4700E−03 |
| 488 | 38036_at | Day 0 Peri/Day 0 Intra | 0.729 | 4.9732E−03 |
| 490 | 35779_at | Day 0 Peri/Day 0 Intra | 0.731 | 7.1397E−03 |
| 491 | 39758_f_at | Day 3 Peri/Day 3 Extra | 0.732 | 1.6743E−03 |
| 492 | 41224_at | Day 3 Peri/Day 3 Extra | 0.732 | 7.2017E−03 |
| 493 | 35738_at | Day 3 Peri/Day 3 Control | 0.732 | 4.8156E−03 |
| 494 | 34372_at | Day 3 Peri/Day 0 Peri | 0.802 | 9.1907E−03 |
| 495 | 33942_s_at | Day 3 Peri/Day 3 Extra | 0.734 | 2.7323E−03 |
| 496 | 32644_at | Day 3 Peri/Day 3 Extra | 0.735 | 1.7224E−03 |
| 497 | 329_s_at | Day 3 Peri/Day 0 Peri | 0.736 | 9.9413E−03 |
| 498 | 38592_s_at | Day 3 Peri/Day 3 Extra | 0.736 | 1.6693E−03 |
| | 38592_s_at | Day 3 Peri/Day 3 Control | 0.788 | 5.7373E−03 |
| 499 | 34380_at | Day 3 Peri/Day 3 Extra | 0.736 | 6.0626E−03 |
| 501 | 31874_at | Day 7 Peri/Day 0 Peri | 0.737 | 7.5700E−05 |
| | 31874_at | Day 3 Peri/Day 0 Peri | 0.791 | 1.9327E−03 |
| 502 | 40111_g_at | Day 3 Peri/Day 3 Extra | 0.737 | 9.0628E−03 |
| 503 | 38821_at | Day 7 Peri/Day 0 Peri | 0.738 | 3.9496E−03 |
| | 38821_at | Day 3 Peri/Day 0 Peri | 0.746 | 7.1002E−03 |
| 504 | 39839_at | Day 7 Peri/Day 0 Peri | 0.738 | 8.5534E−03 |
| 505 | 1486_at | Day 7 Peri/Day 0 Peri | 0.740 | 2.7928E−03 |
| | 1486_at | Day 7 Peri/Day 3 Peri | 0.772 | 1.7983E−03 |
| 506 | 32038_s_at | Day 3 Peri/Day 3 Control | 0.740 | 6.3052E−03 |
| 507 | 32744_at | Day 7 Peri/Day 0 Peri | 0.740 | 7.2902E−03 |
| 508 | 34085_at | Day 7 Peri/Day 0 Peri | 0.743 | 6.3557E−03 |
| 510 | 34592_at | Day 7 Peri/Day 0 Peri | 0.745 | 2.8146E−03 |
| 511 | 36079_at | Day 3 Peri/Day 3 Intra | 0.746 | 5.1335E−03 |
| 512 | 39027_at | Day 3 Peri/Day 3 Extra | 0.746 | 6.3689E−03 |
| 514 | 36952_at | Day 3 Peri/Day 3 Extra | 0.750 | 6.1291E−03 |
| | 36952_at | Day 0 Peri/Day 0 Control | 0.835 | 2.4582E−03 |
| 516 | 39741_at | Day 3 Peri/Day 3 Extra | 0.752 | 2.0159E−03 |
| 517 | 41221_at | Day 3 Peri/Day 3 Extra | 0.752 | 1.6814E−03 |
| 518 | 1685_at | Day 3 Peri/Day 0 Peri | 0.754 | 1.2093E−03 |
| 520 | 39020_at | Day 3 Peri/Day 3 Control | 0.754 | 9.5129E−03 |
| 522 | 38423_at | Day 0 Peri/Day 0 Control | 0.755 | 7.1257E−03 |
| | 38423_at | Day 3 Peri/Day 0 Peri | 0.845 | 2.2722E−03 |
| | 38423_at | Day 7 Peri/Day 0 Peri | 0.848 | 1.7317E−03 |
| 523 | 32589_at | Day 7 Peri/Day 7 Extra | 0.756 | 2.8970E−03 |
| 524 | 31583_at | Day 7 Peri/Day 0 Peri | 0.756 | 8.6210E−03 |
| 525 | 35283_at | Day 0 Peri/Day 0 Control | 0.759 | 7.7904E−04 |
| 527 | 39405_at | Day 0 Peri/Day 0 Intra | 0.760 | 3.9078E−03 |
| 528 | 35805_at | Day 3 Peri/Day 0 Peri | 0.761 | 2.3291E−04 |
| | 35805_at | Day 7 Peri/Day 0 Peri | 0.770 | 2.6315E−03 |
| 529 | 35682_at | Day 3 Peri/Day 3 Control | 0.761 | 2.0789E−04 |
| 530 | 654_at | Day 7 Peri/Day 0 Peri | 0.783 | 5.2959E−03 |
| | 39072_at | Day 3 Peri/Day 0 Peri | 0.839 | 6.8595E−03 |
| 531 | 32658_at | Day 0 Peri/Day 0 Control | 0.762 | 7.4217E−03 |
| | 32658_at | Day 7 Peri/Day 0 Peri | 0.858 | 6.8088E−03 |
| 533 | 32799_at | Day 7 Peri/Day 0 Peri | 0.763 | 8.9346E−03 |
| 534 | 35631_at | Day 3 Peri/Day 3 Extra | 0.764 | 7.2269E−03 |
| | 35631_at | Day 3 Peri/Day 3 Intra | 0.772 | 5.4217E−03 |
| 535 | 31511_at | Day 7 Peri/Day 0 Peri | 0.764 | 7.8708E−03 |
| 536 | 37610_at | Day 3 Peri/Day 3 Extra | 0.764 | 2.5200E−03 |
| 538 | 41600_at | Day 7 Peri/Day 0 Peri | 0.765 | 6.9426E−03 |
| 539 | 40782_at | Day 3 Peri/Day 0 Peri | 0.765 | 6.4510E−03 |
| 540 | 41206_r_at | Day 7 Peri/Day 0 Peri | 0.766 | 1.7769E−03 |
| 541 | 34264_at | Day 3 Peri/Day 3 Intra | 0.768 | 9.1280E−03 |
| 542 | 32659_at | Day 3 Peri/Day 3 Control | 0.768 | 8.3725E−03 |
| 543 | 35156_at | Day 7 Peri/Day 0 Peri | 0.769 | 8.4083E−03 |
| 544 | 32324_at | Day 3 Peri/Day 3 Extra | 0.769 | 5.4500E−03 |
| 545 | 40971_at | Day 3 Peri/Day 0 Peri | 0.796 | 3.4853E−03 |
| 546 | 39058_at | Day 3 Peri/Day 3 Control | 0.771 | 7.8298E−03 |
| | 39058_at | Day 3 Peri/Day 3 Extra | 0.796 | 8.8684E−03 |
| 547 | 1676_s_at | Day 7 Peri/Day 0 Peri | 0.771 | 8.5875E−03 |
| 549 | 40787_at | Day 3 Peri/Day 0 Peri | 0.772 | 8.6910E−04 |
| 553 | 32757_at | Day 3 Peri/Day 3 Extra | 0.778 | 1.2850E−03 |
| | 32758_g_at | Day 7 Peri/Day 7 Intra | 0.786 | 2.2519E−03 |
| 558 | 37916_at | Day 7 Peri/Day 3 Peri | 0.781 | 9.4119E−03 |
| 559 | 41366_at | Day 3 Peri/Day 0 Peri | 0.781 | 8.7647E−03 |
| 560 | 31826_at | Day 0 Peri/Day 0 Control | 0.783 | 2.4933E−03 |
| 562 | 37230_at | Day 3 Peri/Day 0 Peri | 0.786 | 5.6275E−03 |
| | 37230_at | Day 7 Peri/Day 0 Peri | 0.812 | 4.4808E−03 |
| 563 | 32210_at | Day 3 Peri/Day 0 Peri | 0.789 | 7.7233E−03 |
| 565 | 35671_at | Day 7 Peri/Day 0 Peri | 0.791 | 3.1660E−03 |
| 566 | 36996_at | Day 7 Peri/Day 0 Peri | 0.791 | 9.0785E−03 |
| | 36996_at | Day 3 Peri/Day 0 Peri | 0.818 | 6.2854E−03 |
| 567 | 497_at | Day 3 Peri/Day 3 Control | 0.792 | 5.8473E−03 |
| 569 | 36994_at | Day 7 Peri/Day 0 Peri | 0.794 | 8.6116E−03 |
| 570 | 36964_at | Day 3 Peri/Day 0 Peri | 0.795 | 9.8019E−03 |
| 571 | 41231_f_at | Day 3 Peri/Day 3 Extra | 0.796 | 7.6383E−04 |
| | 41231_f_at | Day 3 Peri/Day 3 Intra | 0.827 | 4.3201E−03 |
| 572 | 41655_at | Day 3 Peri/Day 0 Peri | 0.796 | 8.4134E−03 |
| 573 | 33668_at | Day 7 Peri/Day 0 Peri | 0.797 | 2.3409E−03 |
| 575 | 40541_at | Day 7 Peri/Day 3 Peri | 0.799 | 7.5623E−03 |
| 576 | 41765_at | Day 7 Peri/Day 0 Peri | 0.800 | 6.9298E−03 |
| 577 | 40410_at | Day 3 Peri/Day 3 Control | 0.808 | 6.4549E−03 |
| 578 | 38439_at | Day 7 Peri/Day 0 Peri | 0.811 | 5.5209E−03 |
| 579 | 39722_at | Day 3 Peri/Day 0 Peri | 0.812 | 7.5346E−03 |
| 580 | 38726_at | Day 7 Peri/Day 3 Peri | 0.814 | 3.7920E−03 |
| 581 | 33908_at | Day 7 Peri/Day 3 Peri | 0.815 | 9.2186E−03 |
| 582 | 34828_at | Day 0 Peri/Day 0 Control | 0.816 | 8.6878E−03 |
| 583 | 32135_at | Day 3 Peri/Day 0 Peri | 0.819 | 6.4366E−03 |
| 584 | 40275_at | Day 3 Peri/Day 0 Peri | 0.824 | 1.6981E−03 |
| | 40275_at | Day 7 Peri/Day 0 Peri | 0.847 | 5.5244E−03 |
| 585 | 1235_at | Day 3 Peri/Day 3 Extra | 0.824 | 9.9973E−03 |
| | 1235_at | Day 3 Peri/Day 3 Intra | 0.853 | 7.0175E−03 |
| 586 | 41869_at | Day 7 Peri/Day 0 Peri | 0.831 | 9.3885E−03 |

TABLE 3
Diagnostic Down

| Gene | Public | | |
|---|---|---|---|
| ID | Identifier | Data Source | Gene Name |
| 1 | 4250 | Entrez Gene | secretoglobin, family 2A, member 2 |
| 3 | 10647 | Entrez Gene | secretoglobin, family 1D, member 2 |
| 5 | 2167 | Entrez Gene | fatty acid binding protein 4, adipocyte |
| 11 | 553168 | Entrez Gene | chromosome 1 open reading frame 68 |
| 14 | 3205 | Entrez Gene | homeo box A9 |
| 15 | 7136 | Entrez Gene | troponin I type 2 (skeletal, fast) |
| 17 | 6210 | Entrez Gene | ribosomal protein S15a |
| 28 | 10781 | Entrez Gene | zinc finger protein 266 |
| 29 | 4128 | Entrez Gene | monoamine oxidase A |
| 37 | 23016 | Entrez Gene | exosome component 7 |
| 38 | 23352 | Entrez Gene | retinoblastoma-associated factor 600 |
| 39 | 7138 | Entrez Gene | troponin T type 1 (skeletal, slow) |
| 42 | 10521 | Entrez Gene | DEAD (Asp-Glu-Ala-Asp) box polypeptide 17 |
| 43 | 8710 | Entrez Gene | serpin peptidase inhibitor, clade B (ovalbumin), |
| | | | member 7 |
| 46 | 54 | Entrez Gene | acid phosphatase 5, tartrate resistant |
| 49 | 63928 | Entrez Gene | hepatocellular carcinoma antigen gene 520 |
| 50 | 5055 | Entrez Gene | serpin peptidase inhibitor, clade B (ovalbumin), |
| | | | member 2 |
| 51 | 5354 | Entrez Gene | proteolipld protein 1 (Pelizaeus-Merzbacher disease, |
| | | | spastic paraplegia 2, uncomplicated) |
| 52 | 642 | Entrez Gene | bleomycin hydrolase |
| 53 | 10443 | Entrez Gene | Hypothetical gene CG012 |
| 54 | 224 | Entrez Gene | aldehyde dehydrogenase 3 family, member A2 |
| 55 | 90634 | Entrez Gene | hypothetical gene CG018 |
| 59 | 10001 | Entrez Gene | mediator of RNA polymerase II transcription, |
| | | | subunit 6 homolog (yeast) |
| 60 | 97 | Entrez Gene | acylphosphatase 1, erythrocyte (common) type |
| 63 | 51097 | Entrez Gene | saccharopine dehydrogenase (putative) |
| 64 | 2217 | Entrez Gene | Fc fragment of IgG, receptor, transporter, alpha |
| 65 | 1983 | Entrez Gene | eukaryotic translation initiation factor 5 |
| 71 | HG3570- | The Institute for | — |
| | HT377 | Genomic Research | |
| 74 | 8906 | Entrez Gene | adaptor-related protein complex 1, gamma 2 subunit |
| 77 | 9524 | Entrez Gene | glycoprotein, synaptic 2 |
| 78 | 91137 | Entrez Gene | hypothetical protein BC017169 |
| 79 | 56288 | Entrez Gene | par-3 partitioning defective 3 homolog (C. elegans) |
| 81 | 260294 | Entrez Gene | Williams Beuren syndrome chromosome region 20C |
| 83 | 131544 | Entrez Gene | hypothetical protein DKFZp667G2110 |
| 86 | 10181 | Entrez Gene | RNA binding motif protein 5 |
| 94 | 10450 | Entrez Gene | peptidylprolyl isomerase E (cyclophilin E) |
| 95 | HG1112- | The Institute | — |
| | HT111 | for Genomic | |
| | | Research | |
| 99 | 10314 | Entrez Gene | LanC lantibiotic synthetase component C-like 1 |
| | | | (bacterial) |
| 103 | 10924 | Entrez Gene | sphingomyelin phosphodiesterase, acid-like 3A |
| 109 | 26039 | Entrez Gene | synovial sarcoma translocation gene on chromosome |
| | | | 18-like 1 |
| 113 | 6490 | Entrez Gene | silver homolog (mouse) |
| 115 | 4176 | Entrez Gene | MCM7 minichromosome maintenance |
| | | | deficient 7 (S. cerevisiae) |
| 116 | 2197 | Entrez Gene | Finkel-Biskis-Reilly murine sarcoma virus (FBR-MuSV) |
| | | | ubiquitously expressed (fox derived): ribosomal protein |
| | | | S30 |
| 118 | 4898 | Entrez Gene | nardilysin (N-arginine dibasic convertase) |
| 123 | 1573 | Entrez Gene | cytochrome P450, family 2, subfamily J, polypeptide 2 |
| 130 | U18300 | GenBank | damage-specific DNA binding protein 2, 48 kDa /// LIM |
| | | | homeobox 3 |
| 132 | 51706 | Entrez Gene | cytochrome b5 reductase 1 |
| 141 | 57613 | Entrez Gene | KIAA1467 protein |
| 142 | 2647 | Entrez Gene | biogenesis of lysosome-related organelles complex-1, |
| | | | subunit 1 |
| 148 | 4713 | Entrez Gene | NADH dehydrogenase (ubiquinone) 1 beta |
| | | | subcomplex, 7, 18 kDa |
| 150 | 771 | Entrez Gene | carbonic anhydrase XII |
| 151 | 55187 | Entrez Gene | vacuolar protein sorting 13D (yeast) |
| 152 | 498 | Entrez Gene | ATP synthase, H+ transporting, mitochondrial F1 |
| | | | complex, alpha subunit, isoform 1, cardiac muscle |
| 156 | 5985 | Entrez Gene | replication factor C (activator 1) 5, 36.5 kDa |
| 159 | 5110 | Entrez Gene | Protein-L-isoaspartate (D-aspartate) O- |
| | | | methyltransferase |
| 160 | 113146 | Entrez Gene | chromosome 14 open reading frame 78 |
| 165 | 10124 | Entrez Gene | ADP-ribosylation factor-like 4 |
| 166 | 10653 | Entrez Gene | serine peptidase inhibitor, Kunitz type, 2 |
| 167 | 10434 | Entrez Gene | lysophospholipase I |
| 170 | 80308 | Entrez Gene | Fad1, flavin adenine dinucleotide synthetase, homolog |
| | | | (yeast) |
| 173 | 23345 | Entrez Gene | spectrin repeat containing, nuclear envelope 1 |
| 175 | 80208 | Entrez Gene | hypothetical protein FLJ21439 |
| 180 | 9927 | Entrez Gene | mitofusin 2 |
| 182 | 2145 | Entrez Gene | enhancer of zeste homolog 1 (Drosophila) |
| 183 | 2959 | Entrez Gene | general transcription factor IIB |
| 185 | 4736 | Entrez Gene | ribosomal protein L10a |
| 186 | 644 | Entrez Gene | biliverdin reductase A |
| 187 | 9665 | Entrez Gene | limkain b1 |
| 191 | 11336 | Entrez Gene | SEC6-like 1 (S. cerevisiae) |
| 192 | 7009 | Entrez Gene | testis enhanced gene transcript (BAX inhibitor 1) |
| 193 | 23107 | Entrez Gene | mitochondrial ribosomal protein S27 |
| 194 | 50 | Entrez Gene | aconitase 2, mitochondrial |
| 195 | 2746 | Entrez Gene | glutamate dehydrogenase 1 |
| 198 | 6166 | Entrez Gene | ribosomal protein L36a-like |
| 199 | 7175 | Entrez Gene | translocated promoter region (to activated MET |
| | | | oncogene) |
| 202 | 10553 | Entrez Gene | HIV-1 Tat interactive protein 2, 30 kDa |
| 203 | 115817 | Entrez Gene | dehydrogenase/reductase (SDR family) member 1 |
| 205 | 25963 | Entrez Gene | DKFZP564G2022 protein |
| 206 | 1212 | Entrez Gene | clathrin, light polypeptide (Lcb) |
| 207 | 1652 | Entrez Gene | D-dopachrome tautomerase |
| 216 | 4729 | Entrez Gene | NADH dehydrogenase (ubiquinone) flavoprotein 2, |
| | | | 24 kDa |
| 217 | 987 | Entrez Gene | LPS-responsive vesicle trafficking, beach and anchor |
| | | | containing |
| 222 | 10980 | Entrez Gene | COP9 constitutive photomorphogenic homolog subunit |
| | | | 6 (Arabidopsis) |
| 228 | 645 | Entrez Gene | biliverdin reductase B (flavin reductase (NADPH)) |
| 232 | 10476 | Entrez Gene | ATP synthase, H+ transporting, mitochondrial F0 |
| | | | complex, subunit d |
| 233 | 7022 | Entrez Gene | transcription factor AP-2 gamma (activating enhancer |
| | | | binding protein 2 gamma) |
| 235 | 23185 | Entrez Gene | La ribonucleoprotein domain family, member 5 |
| 241 | 8673 | Entrez Gene | vesicle-associated membrane protein 8 (endobrevin) |
| 243 | 4255 | Entrez Gene | O-6-methylguanine-DNA methyltransferase |
| 246 | 11188 | Entrez Gene | nischarin |
| 250 | 4775 | Entrez Gene | nuclear factor of activated T-cells, cytoplasmic, |
| | | | calcineurin-dependent 3 |
| 252 | 4790 | Entrez Gene | nuclear factor of kappa light polypeptide gene |
| | | | enhancer in B-cells 1 (p105) |
| 254 | 26135 | Entrez Gene | SERPINE1 mRNA binding protein 1 |
| 257 | AI557912 | GenBank | translocase of outer mitochondrial membrane 7 |
| | | | homolog (yeast) /// hypothetical protein LOC201725 |
| 259 | 3550 | Entrez Gene | IK cytokine, down-regulator of HLA II |
| 262 | 353 | Entrez Gene | adenine phosphoribosyltransferase |
| 265 | 79703 | Entrez Gene | hypothetical protein FLJ22531 |
| 268 | 513 | Entrez Gene | ATP synthase, H+ transporting, mitochondrial F1 |
| | | | complex, delta subunit |
| 269 | AL050091 | GenBank | glutamate receptor, ionotropic, N-methyl D-aspartate- |
| | | | like 1A /// GRINL1A combined protein /// similar to |
| | | | glutamate receptor, ionotropic, N-methyl D-a |
| 275 | 10497 | Entrez Gene | unc-13 homolog B (C. elegans) |
| 277 | 8560 | Entrez Gene | degenerative spermatocyte homolog 1, lipid |
| | | | desaturase (Drosophila) |
| 278 | 10975 | Entrez Gene | ubiquinol-cytochrome c reductase, 6.4 kDa subunit |
| 279 | 10641 | Entrez Gene | tumor suppressor candidate 4 |
| 281 | 50717 | Entrez Gene | WD repeat domain 42A |
| 291 | 5092 | Entrez Gene | 6-pyruvoyl-tetrahydropterin synthase/dimerization |
| | | | cofactor of hepatocyte nuclear factor 1 alpha (TCF1) |
| 292 | 7818 | Entrez Gene | death associated protein 3 |
| 294 | 23063 | Entrez Gene | KIAA0261 |
| 296 | 57147 | Entrez Gene | SCY1-like 3 (S. cerevisiae) |
| 300 | 22883 | Entrez Gene | calsyntenin 1 |
| 301 | 7520 | Entrez Gene | X-ray repair complementing defective repair in |
| | | | Chinese hamster cells 5 (double-strand-break |
| | | | rejoining; Ku autoantigen, 80 kDa) |
| 305 | 23351 | Entrez Gene | KIAA0323 |
| 306 | 2065 | Entrez Gene | v-erb-b2 erythroblastic leukemia viral oncogene |
| | | | homolog 3 (avian) |
| 307 | 10217 | Entrez Gene | CTD (carboxy-terminal domain, RNA polymerase II, |
| | | | polypeptide A) small phosphatase-like |
| 308 | 23658 | Entrez Gene | LSM5 homolog, U6 small nuclear RNA |
| | | | associated (S. cerevisiae) |
| 309 | W28948 | GenBank | Full length insert cDNA YH77E09 |
| 314 | 6209 | Entrez Gene | ribosomal protein S15 |
| 317 | 10961 | Entrez Gene | endoplasmic reticulum protein 29 |
| 318 | 7265 | Entrez Gene | tetratricopeptide repeat domain 1 |
| 323 | 9296 | Entrez Gene | ATPase, H+ transporting, lysosomal 14 kDa, V1 |
| | | | subunit F |
| 326 | 1632 | Entrez Gene | dodecenoyl-Coenzyme A delta isomerase (3,2 transenoyl- |
| | | | Coenzyme A isomerase) |
| 328 | 6141 | Entrez Gene | ribosomal protein L18 |
| 332 | 573 | Entrez Gene | BCL2-associated athanogene |
| 335 | 11083 | Entrez Gene | death associated transcription factor 1 |
| 336 | 10247 | Entrez Gene | heat-responsive protein 12 |
| 343 | 7739 | Entrez Gene | zinc finger protein 185 (LIM domain) |
| 348 | 26211 | Entrez Gene | olfactory receptor, family 2, subfamily F, member 1 |
| 350 | 9798 | Entrez Gene | KIAA0174 |
| 353 | 4070 | Entrez Gene | tumor-associated calcium signal transducer 2 |
| 359 | 4835 | Entrez Gene | NAD(P)H dehydrogenase, quinone 2 |
| 367 | 51690 | Entrez Gene | LSM7 homolog, U6 small nuclear RNA |
| | | | associated (S. cerevisiae) |
| 378 | 10014 | Entrez Gene | histone deacetylase 5 |
| 380 | HG662-HT662 | The Institute | — |
| | | for Genomic | |
| | | Research | |
| 385 | 4694 | Entrez Gene | NADH dehydrogenase (ubiquinone) 1 alpha |
| | | | subcomplex, 1, 7.5 kDa |
| 389 | 8667 | Entrez Gene | eukaryotic translation initiation factor 3, subunit 3 |
| | | | gamma, 40 kDa |
| 395 | 5441 | Entrez Gene | polymerase (RNA) II (DNA directed) polypeptide L, |
| | | | 7.6 kDa |
| 404 | 3028 | Entrez Gene | hydroxyacyl-Coenzyme A dehydrogenase, type II |
| 407 | 6748 | Entrez Gene | signal sequence receptor, delta (translocon-associated |
| | | | protein delta) |
| 411 | 1537 | Entrez Gene | cytochrome c-1 |
| 415 | 64976 | Entrez Gene | mitochondrial ribosomal protein L40 |
| 418 | 4676 | Entrez Gene | nucleosome assembly protein 1-like 4 |
| 431 | 8337 | Entrez Gene | histone 2, H2aa |
| 433 | 23492 | Entrez Gene | chromobox homolog 7 |
| 434 | 4809 | Entrez Gene | NHP2 non-histone chromosome protein |
| | | | 2-like 1 (S. cerevisiae) |
| 437 | 328 | Entrez Gene | APEX nuclease (multifunctional DNA repair enzyme) 1 |
| 440 | 533 | Entrez Gene | ATPase, H+ transporting, lysosomal 21 kDa, V0 |
| | | | subunit c″ |
| 445 | 4678 | Entrez Gene | nuclear autoantigenic sperm protein (histone-binding) |
| 449 | 57017 | Entrez Gene | chromosome 16 open reading frame 49 |
| 453 | 10542 | Entrez Gene | hepatitis B virus x interacting protein |
| 459 | 593 | Entrez Gene | branched chain keto acid dehydrogenase E1, alpha |
| | | | polypeptide (maple syrup urine disease) |
| 463 | 22870 | Entrez Gene | SAPS domain family, member 1 |
| 471 | 6050 | Entrez Gene | ribonuclease/angiogenin inhibitor 1 |
| 475 | U77948 | GenBank | general transcription factor II, I /// general transcription |
| | | | factor II, i, pseudogene 1 |
| 480 | HG1614- | The Institute | — |
| | HT161 | for Genomic | |
| | | Research | |
| 493 | 10473 | Entrez Gene | high mobility group nucleosomal binding domain 4 |
| 498 | 283638 | Entrez Gene | KIAA0284 |
| 506 | 10929 | Entrez Gene | Splicing factor, arginine/serine-rich, 46 kD |
| 514 | 3030 | Entrez Gene | hydroxyacyl-Coenzyme A dehydrogenase/3-ketoacyl- |
| | | | Coenzyme A thiolase/enoyl-Coenzyme A hydratase |
| | | | (trifunctional protein), alpha subunit |
| 520 | 10572 | Entrez Gene | CD27-binding (Siva) protein |
| 522 | 162427 | Entrez Gene | hypothetical protein LOC162427 |
| 525 | 55218 | Entrez Gene | chromosome 14 open reading frame 114 |
| 529 | 56829 | Entrez Gene | zinc finger CCCH-type, antiviral 1 |
| 531 | 6293 | Entrez Gene | vacuolar protein sorting 52 (yeast) |
| 542 | 8890 | Entrez Gene | eukaryotic translation initiation factor 2B, subunit 4 |
| | | | delta, 67 kDa |
| 546 | 29 | Entrez Gene | active BCR-related gene |
| 560 | 23307 | Entrez Gene | KIAA0674 |
| 567 | 1201 | Entrez Gene | ceroid-lipofuscinosis, neuronal 3, juvenile (Batten, |
| | | | Spielmeyer-Vogt disease) |
| 577 | 11258 | Entrez Gene | dynactin 3 (p22) |
| 582 | 5438 | Entrez Gene | polymerase (RNA) II (DNA directed) polypeptide I, |
| | | | 14.5 kDa |

| Gene | | | Fold | |
|---|---|---|---|---|
| ID | Probe_ID | Comparison | Change | P value |
| 1 | 36329_at | Day 3 Peri/Day 3 Control | 0.131 | 2.0793E−03 |
| 3 | 32880_at | Day 3 Peri/Day 3 Control | 0.211 | 2.5582E−03 |
| 5 | 38430_at | Day 0 Peri/Day 0 Control | 0.219 | 8.7014E−03 |
| 11 | 31326_at | Day 7 Peri/Day 7 Control | 0.138 | 7.6909E−03 |
| | 31326_at | Day 3 Peri/Day 3 Control | 0.214 | 6.7718E−03 |
| 14 | 37809_at | Day 3 Peri/Day 3 Control | 0.180 | 5.0202E−03 |
| 15 | 38457_at | Day 0 Peri/Day 0 Control | 0.185 | 5.1425E−04 |
| 17 | 34316_at | Day 3 Peri/Day 3 Control | 0.241 | 3.8100E−05 |
| | 34316_at | Day 7 Peri/Day 7 Control | 0.432 | 2.3230E−04 |
| 28 | 41621_i_at | Day 0 Peri/Day 0 Control | 0.350 | 5.4116E−04 |
| 29 | 41771_g_at | Day 3 Peri/Day 3 Control | 0.464 | 2.4030E−03 |
| 37 | 33294_at | Day 0 Peri/Day 0 Control | 0.369 | 2.9368E−04 |
| | 33294_at | Day 3 Peri/Day 3 Control | 0.453 | 3.7416E−04 |
| 38 | 33860_at | Day 0 Peri/Day 0 Control | 0.369 | 5.2550E−04 |
| | 33860_at | Day 3 Peri/Day 3 Control | 0.374 | 1.4026E−03 |
| | 33860_at | Day 7 Peri/Day 7 Control | 0.452 | 4.0374E−03 |
| 39 | 36113_s_at | Day 7 Peri/Day 7 Control | 0.371 | 6.4628E−03 |
| | 36113_s_at | Day 3 Peri/Day 3 Control | 0.452 | 4.3070E−03 |
| 42 | 35363_at | Day 0 Peri/Day 0 Control | 0.383 | 2.2472E−03 |
| 43 | 35577_at | Day 0 Peri/Day 0 Control | 0.385 | 4.8105E−04 |
| 46 | 677_s_at | Day 7 Peri/Day 7 Control | 0.389 | 2.8442E−04 |
| 49 | 33007_at | Day 3 Peri/Day 3 Control | 0.394 | 7.4079E−03 |
| 50 | 37185_at | Day 0 Peri/Day 0 Control | 0.399 | 5.5226E−03 |
| 51 | 41158_at | Day 0 Peri/Day 0 Control | 0.544 | 4.8737E−03 |
| 52 | 394_at | Day 3 Peri/Day 3 Control | 0.404 | 6.8138E−03 |
| 53 | 1532_g_at | Day 0 Peri/Day 0 Control | 0.406 | 9.9594E−04 |
| | 1532_g_at | Day 3 Peri/Day 3 Control | 0.567 | 4.1852E−03 |
| 54 | 40409_at | Day 3 Peri/Day 3 Control | 0.445 | 7.4233E−03 |
| 55 | 1527_s_at | Day 0 Peri/Day 0 Control | 0.410 | 2.0450E−03 |
| | 1527_s_at | Day 3 Peri/Day 3 Control | 0.515 | 2.5140E−03 |
| 59 | 35430_at | Day 3 Peri/Day 3 Control | 0.427 | 4.0549E−03 |
| 60 | 33334_at | Day 0 Peri/Day 0 Control | 0.427 | 3.3326E−03 |
| 63 | 34863_s_at | Day 3 Peri/Day 3 Control | 0.430 | 9.0435E−03 |
| 64 | 31431_at | Day 7 Peri/Day 7 Control | 0.430 | 7.9132E−04 |
| 65 | 167_at | Day 3 Peri/Day 3 Control | 0.431 | 3.2376E−04 |
| 71 | 936_s_at | Day 3 Peri/Day 3 Control | 0.437 | 1.3929E−04 |
| | 936_s_at | Day 7 Peri/Day 7 Control | 0.554 | 6.0851E−03 |
| 74 | 38798_s_at | Day 0 Peri/Day 0 Control | 0.441 | 6.9369E−03 |
| | 38798_s_at | Day 3 Peri/Day 3 Control | 0.480 | 6.4090E−03 |
| 77 | 38966_at | Day 7 Peri/Day 7 Control | 0.444 | 3.9851E−03 |
| | 38966_at | Day 3 Peri/Day 3 Control | 0.530 | 1.4148E−03 |
| 78 | 37178_at | Day 3 Peri/Day 3 Control | 0.449 | 3.9314E−03 |
| 79 | 40973_at | Day 0 Peri/Day 0 Control | 0.451 | 2.5568E−03 |
| 81 | 41258_at | Day 0 Peri/Day 0 Control | 0.453 | 3.7268E−03 |
| 83 | 41274_at | Day 0 Peri/Day 0 Control | 0.454 | 4.4963E−03 |
| | 41274_at | Day 3 Peri/Day 3 Control | 0.589 | 7.6530E−03 |
| 86 | 1556_at | Day 0 Peri/Day 0 Control | 0.460 | 1.4047E−03 |
| 94 | 34365_at | Day 3 Peri/Day 3 Control | 0.473 | 5.7231E−03 |
| 95 | 1839_at | Day 3 Peri/Day 3 Control | 0.555 | 9.8116E−03 |
| 99 | 39441_at | Day 0 Peri/Day 0 Control | 0.482 | 2.7594E−03 |
| 103 | 39950_at | Day 3 Peri/Day 3 Control | 0.483 | 8.6125E−03 |
| 109 | 31903_at | Day 0 Peri/Day 0 Control | 0.493 | 1.3274E−03 |
| 113 | 38327_at | Day 3 Peri/Day 3 Control | 0.512 | 5.3933E−04 |
| 115 | 947_at | Day 0 Peri/Day 0 Control | 0.498 | 7.8436E−04 |
| 116 | 31955_at | Day 7 Peri/Day 7 Control | 0.498 | 8.3003E−03 |
| 118 | 34787_at | Day 3 Peri/Day 3 Control | 0.499 | 2.6275E−03 |
| 123 | 501_g_at | Day 3 Peri/Day 3 Control | 0.505 | 2.0051E−03 |
| 130 | 1243_at | Day 3 Peri/Day 3 Control | 0.511 | 4.6848E−04 |
| | 1243_at | Day 0 Peri/Day 0 Control | 0.556 | 6.7443E−03 |
| 132 | 35329_at | Day 3 Peri/Day 3 Control | 0.513 | 1.8894E−03 |
| 141 | 41826_at | Day 0 Peri/Day 0 Control | 0.520 | 5.9389E−03 |
| 142 | 39133_at | Day 7 Peri/Day 7 Control | 0.520 | 1.5729E−03 |
| 148 | 35773_i_at | Day 7 Peri/Day 7 Control | 0.525 | 1.8840E−03 |
| 150 | 36454_at | Day 0 Peri/Day 0 Control | 0.526 | 3.3461E−03 |
| 151 | 32743_at | Day 0 Peri/Day 0 Control | 0.619 | 1.5268E−03 |
| 152 | 40096_at | Day 3 Peri/Day 3 Control | 0.527 | 6.5302E−03 |
| | 40096_at | Day 0 Peri/Day 0 Control | 0.563 | 2.0338E−04 |
| 156 | 653_at | Day 0 Peri/Day 0 Control | 0.533 | 6.1850E−03 |
| 159 | 37738_g_at | Day 3 Peri/Day 3 Control | 0.678 | 3.6789E−03 |
| 160 | 36497_at | Day 3 Peri/Day 3 Control | 0.535 | 3.4244E−03 |
| 165 | 33796_at | Day 3 Peri/Day 3 Control | 0.537 | 6.8833E−03 |
| 166 | 34348_at | Day 3 Peri/Day 3 Control | 0.537 | 9.4449E−03 |
| | 34348_at | Day 0 Peri/Day 0 Control | 0.573 | 7.9326E−03 |
| 167 | 39396_at | Day 3 Peri/Day 3 Control | 0.541 | 7.9687E−03 |
| 170 | 39074_at | Day 3 Peri/Day 3 Control | 0.544 | 3.5087E−04 |
| 173 | 38113_at | Day 0 Peri/Day 0 Control | 0.546 | 1.8607E−03 |
| 175 | 40615_at | Day 0 Peri/Day 0 Control | 0.546 | 5.1650E−03 |
| 180 | 34369_at | Day 3 Peri/Day 3 Control | 0.549 | 2.5016E−03 |
| 182 | 32259_at | Day 3 Peri/Day 3 Control | 0.593 | 1.0957E−03 |
| 183 | 1070_at | Day 3 Peri/Day 3 Control | 0.556 | 4.5246E−03 |
| 185 | 36786_at | Day 7 Peri/Day 7 Control | 0.592 | 1.4203E−03 |
| | 36786_at | Day 3 Peri/Day 3 Control | 0.622 | 8.0958E−04 |
| | 36786_at | Day 0 Peri/Day 0 Control | 0.668 | 3.0374E−03 |
| 186 | 32618_at | Day 3 Peri/Day 3 Control | 0.557 | 3.0605E−04 |
| | 32618_at | Day 0 Peri/Day 0 Control | 0.677 | 7.9770E−03 |
| 187 | 31936_s_at | Day 3 Peri/Day 3 Control | 0.558 | 3.7112E−03 |
| 191 | 37597_s_at | Day 3 Peri/Day 3 Control | 0.560 | 4.7635E−03 |
| 192 | 33988_at | Day 3 Peri/Day 3 Control | 0.560 | 1.0937E−03 |
| 193 | 39377_at | Day 3 Peri/Day 3 Control | 0.571 | 9.8955E−03 |
| 194 | 160044_g_at | Day 3 Peri/Day 3 Control | 0.562 | 5.6984E−03 |
| 195 | 31691_g_at | Day 3 Peri/Day 3 Control | 0.563 | 6.9886E−03 |
| 198 | 39856_at | Day 0 Peri/Day 0 Control | 0.568 | 7.8476E−03 |
| 199 | 421_at | Day 0 Peri/Day 0 Control | 0.569 | 3.6369E−03 |
| 202 | 38824_at | Day 3 Peri/Day 3 Control | 0.571 | 7.2789E−03 |
| 203 | 39103_s_at | Day 3 Peri/Day 3 Control | 0.571 | 2.1724E−04 |
| 205 | 40437_at | Day 3 Peri/Day 3 Control | 0.572 | 3.2143E−03 |
| 206 | 32523_at | Day 3 Peri/Day 3 Control | 0.573 | 4.0809E−03 |
| 207 | 374_f_at | Day 7 Peri/Day 7 Control | 0.573 | 7.7105E−03 |
| 216 | 34893_at | Day 7 Peri/Day 7 Control | 0.581 | 9.2931E−03 |
| 217 | 35371_at | Day 3 Peri/Day 3 Control | 0.582 | 1.9359E−03 |
| 222 | 40138_at | Day 7 Peri/Day 7 Control | 0.584 | 6.5665E−03 |
| 228 | 37002_at | Day 7 Peri/Day 7 Control | 0.588 | 8.3451E−04 |
| 232 | 35760_at | Day 0 Peri/Day 0 Control | 0.738 | 8.9277E−03 |
| 233 | 40303_at | Day 3 Peri/Day 3 Control | 0.591 | 7.9662E−03 |
| 235 | 32586_at | Day 0 Peri/Day 0 Control | 0.591 | 3.7384E−03 |
| | 32586_at | Day 3 Peri/Day 3 Control | 0.685 | 8.4169E−04 |
| 241 | 32715_at | Day 0 Peri/Day 0 Control | 0.628 | 1.3473E−03 |
| 243 | 2051_at | Day 7 Peri/Day 7 Control | 0.594 | 5.4697E−03 |
| 246 | 33916_at | Day 0 Peri/Day 0 Control | 0.595 | 3.7132E−03 |
| | 33916_at | Day 3 Peri/Day 3 Control | 0.647 | 6.0432E−03 |
| 250 | 40822_at | Day 0 Peri/Day 0 Control | 0.597 | 3.4027E−03 |
| 252 | 1378_g_at | Day 0 Peri/Day 0 Control | 0.646 | 7.6400E−03 |
| 254 | 40440_at | Day 0 Peri/Day 0 Control | 0.599 | 7.7405E−03 |
| 257 | 39025_at | Day 7 Peri/Day 7 Control | 0.716 | 2.2149E−03 |
| 259 | 218_at | Day 3 Peri/Day 3 Control | 0.606 | 4.9835E−04 |
| 262 | 34310_at | Day 3 Peri/Day 3 Control | 0.607 | 7.6231E−03 |
| 265 | 41804_at | Day 3 Peri/Day 3 Control | 0.608 | 1.1934E−04 |
| 268 | 37992_s_at | Day 7 Peri/Day 7 Control | 0.611 | 6.2111E−03 |
| 269 | 35736_at | Day 0 Peri/Day 0 Control | 0.611 | 4.7952E−03 |
| 275 | 40087_at | Day 3 Peri/Day 3 Control | 0.614 | 3.7491E−03 |
| 277 | 33337_at | Day 3 Peri/Day 3 Control | 0.614 | 8.5255E−03 |
| | 33337_at | Day 7 Peri/Day 7 Control | 0.626 | 3.0395E−03 |
| 278 | 38451_at | Day 7 Peri/Day 7 Control | 0.614 | 8.2100E−05 |
| 279 | 40497_at | Day 0 Peri/Day 0 Control | 0.614 | 9.7424E−03 |
| | 40497_at | Day 3 Peri/Day 3 Control | 0.655 | 1.1667E−04 |
| 281 | 39823_at | Day 3 Peri/Day 3 Control | 0.616 | 2.9015E−03 |
| 291 | 34352_at | Day 7 Peri/Day 7 Control | 0.620 | 2.4354E−03 |
| 292 | 1356_at | Day 0 Peri/Day 0 Control | 0.621 | 1.4788E−03 |
| 294 | 40086_at | Day 3 Peri/Day 3 Control | 0.623 | 5.5939E−03 |
| 296 | 41329_at | Day 3 Peri/Day 3 Control | 0.624 | 2.9063E−03 |
| 300 | 41498_at | Day 3 Peri/Day 3 Control | 0.626 | 9.2124E−04 |
| 301 | 2093_s_at | Day 3 Peri/Day 3 Control | 0.627 | 1.0646E−03 |
| | 38733_at | Day 0 Peri/Day 0 Control | 0.644 | 9.6568E−03 |
| 305 | 32592_at | Day 0 Peri/Day 0 Control | 0.628 | 8.9436E−04 |
| 306 | 2089_s_at | Day 0 Peri/Day 0 Control | 0.629 | 5.0250E−03 |
| 307 | 40196_at | Day 3 Peri/Day 3 Control | 0.630 | 3.3471E−03 |
| 308 | 34270_at | Day 3 Peri/Day 3 Control | 0.631 | 1.9266E−03 |
| 309 | 37161_at | Day 3 Peri/Day 3 Control | 0.640 | 6.9170E−03 |
| 314 | 39916_r_at | Day 7 Peri/Day 7 Control | 0.633 | 1.4158E−03 |
| | 39916_r_at | Day 3 Peri/Day 3 Control | 0.675 | 7.7101E−03 |
| 317 | 36945_at | Day 3 Peri/Day 3 Control | 0.635 | 9.2309E−04 |
| 318 | 37321_at | Day 3 Peri/Day 3 Control | 0.635 | 7.5603E−03 |
| 323 | 37395_at | Day 7 Peri/Day 7 Control | 0.638 | 2.2448E−03 |
| | 37395_at | Day 3 Peri/Day 3 Control | 0.722 | 8.6817E−04 |
| 326 | 37982_at | Day 7 Peri/Day 7 Control | 0.639 | 2.3722E−03 |
| 328 | 31546_at | Day 7 Peri/Day 7 Control | 0.639 | 6.9963E−03 |
| 332 | 34798_at | Day 3 Peri/Day 3 Control | 0.642 | 4.4022E−03 |
| 335 | 32045_at | Day 0 Peri/Day 0 Control | 0.644 | 6.8658E−03 |
| 336 | 32173_at | Day 3 Peri/Day 3 Control | 0.659 | 6.9500E−03 |
| 343 | 32139_at | Day 0 Peri/Day 0 Control | 0.648 | 5.7560E−03 |
| 348 | 31921_at | Day 3 Peri/Day 3 Control | 0.652 | 2.1176E−03 |
| 350 | 36942_at | Day 0 Peri/Day 0 Control | 0.653 | 5.6656E−03 |
| 353 | 291_s_at | Day 0 Peri/Day 0 Control | 0.654 | 9.5683E−03 |
| 359 | 36880_at | Day 7 Peri/Day 7 Control | 0.657 | 7.2935E−03 |
| 367 | 36846_s_at | Day 7 Peri/Day 7 Control | 0.662 | 8.8105E−04 |
| 378 | 38810_at | Day 3 Peri/Day 3 Control | 0.667 | 1.7702E−04 |
| 380 | 1151_at | Day 3 Peri/Day 3 Control | 0.669 | 1.5550E−03 |
| 385 | 36169_at | Day 3 Peri/Day 3 Control | 0.671 | 5.9310E−04 |
| | 36169_at | Day 7 Peri/Day 7 Control | 0.710 | 9.3036E−03 |
| 389 | 35327_at | Day 3 Peri/Day 3 Control | 0.672 | 5.0660E−03 |
| 395 | 35841_at | Day 7 Peri/Day 7 Control | 0.676 | 2.4659E−03 |
| 404 | 40778_at | Day 3 Peri/Day 3 Control | 0.683 | 9.5528E−03 |
| | 40778_at | Day 0 Peri/Day 0 Control | 0.700 | 6.7401E−03 |
| 407 | 38635_at | Day 7 Peri/Day 7 Control | 0.684 | 3.2000E−03 |
| 411 | 1160_at | Day 3 Peri/Day 3 Control | 0.685 | 9.9616E−03 |
| 415 | 32745_at | Day 3 Peri/Day 3 Control | 0.688 | 7.4413E−03 |
| 418 | 32575_at | Day 3 Peri/Day 3 Control | 0.689 | 7.9600E−05 |
| 431 | 286_at | Day 3 Peri/Day 3 Control | 0.693 | 7.8013E−03 |
| 433 | 36894_at | Day 3 Peri/Day 3 Control | 0.693 | 8.8784E−03 |
| 434 | 41746_at | Day 3 Peri/Day 3 Control | 0.693 | 5.4886E−03 |
| 437 | 2025_s_at | Day 3 Peri/Day 3 Control | 0.695 | 4.9002E−03 |
| 440 | 36167_at | Day 7 Peri/Day 7 Control | 0.696 | 9.2513E−03 |
| 445 | 33255_at | Day 3 Peri/Day 3 Control | 0.696 | 9.0975E−03 |
| 449 | 34810_at | Day 7 Peri/Day 7 Control | 0.700 | 1.2592E−03 |
| 453 | 38054_at | Day 3 Peri/Day 3 Control | 0.704 | 9.1709E−03 |
| 459 | 37704_at | Day 3 Peri/Day 3 Control | 0.707 | 1.4726E−03 |
| 463 | 36862_at | Day 3 Peri/Day 3 Control | 0.709 | 6.5040E−03 |
| 471 | 36187_at | Day 3 Peri/Day 3 Control | 0.730 | 5.3879E−03 |
| 475 | 35450_s_at | Day 0 Peri/Day 0 Control | 0.718 | 8.5556E−03 |
| 480 | 954_s_at | Day 7 Peri/Day 7 Control | 0.722 | 8.4646E−03 |
| 493 | 35738_at | Day 3 Peri/Day 3 Control | 0.732 | 4.8156E−03 |
| 498 | 38592_s_at | Day 3 Peri/Day 3 Control | 0.788 | 5.7373E−03 |
| 506 | 32038_s_at | Day 3 Peri/Day 3 Control | 0.740 | 6.3052E−03 |
| 514 | 36952_at | Day 0 Peri/Day 0 Control | 0.835 | 2.4582E−03 |
| 520 | 39020_at | Day 3 Peri/Day 3 Control | 0.754 | 9.5129E−03 |
| 522 | 38423_at | Day 0 Peri/Day 0 Control | 0.755 | 7.1257E−03 |
| 525 | 35283_at | Day 0 Peri/Day 0 Control | 0.759 | 7.7904E−04 |
| 529 | 35682_at | Day 3 Peri/Day 3 Control | 0.761 | 2.0789E−04 |
| 531 | 32658_at | Day 0 Peri/Day 0 Control | 0.762 | 7.4217E−03 |
| 542 | 32659_at | Day 3 Peri/Day 3 Control | 0.768 | 8.3725E−03 |
| 546 | 39058_at | Day 3 Peri/Day 3 Control | 0.771 | 7.8298E−03 |
| 560 | 31826_at | Day 0 Peri/Day 0 Control | 0.783 | 2.4933E−03 |
| 567 | 497_at | Day 3 Peri/Day 3 Control | 0.792 | 5.8473E−03 |
| 577 | 40410_at | Day 3 Peri/Day 3 Control | 0.808 | 6.4549E−03 |
| 582 | 34828_at | Day 0 Peri/Day 0 Control | 0.816 | 8.6878E−03 |

TABLE 4
Diagnostic Down

| Gene | Public | | | | | Fold | |
|---|---|---|---|---|---|---|---|
| ID | Identifier | Data Source | Gene Name | Probe_ID | Comparison | Change | P value |
| 166 | 10653 | Entrez Gene | serine peptidase inhibitor, Kunitz type, 2 | 34348_at | Day 3 Peri/Day 3 Control | 0.537 | 9.4449E−03 |
| | | | | 34348_at | Day 0 Peri/Day 0 Control | 0.573 | 7.9326E−03 |

TABLE 5
Diagnostic Down

| | Public | | | | | | |
|---|---|---|---|---|---|---|---|
| Gene ID | Identifier | Data Source | Gene Name | Probe_ID | Comparison | Fold Change | P value |
| 141 | 57613 | Entrez Gene | KIAA1467 protein | 41826_at | Day 0 Peri/Day 0 Control | 0.520 | 5.9389E−03 |
| 300 | 22883 | Entrez Gene | calsyntenin 1 | 41498_at | Day 3 Peri/Day 3 Control | 0.626 | 9.2124E−04 |

TABLE 6
Diagnostic Down

| | Public | | | | | | |
|---|---|---|---|---|---|---|---|
| Gene ID | Identifier | Data Source | Gene Name | Probe_ID | Comparison | Fold Change | P value |
| 250 | 4775 | Entrez Gene | nuclear factor of activated T-cells, | 40822_at | Day 0 Peri/Day 0 Control | 0.597 | 3.4027E−03 |
| | | | cytoplasmic, calcineurin-dependent 3 | | | | |
| 252 | 4790 | Entrez Gene | nuclear factor of kappa light | 1378_g_at | Day 0 Peri/ Day 0 Control | 0.646 | 7.6400E−03 |
| | | | polypeptide gene enhancer in | | | | |
| | | | 8-cells 1 (p105) | | | | |
| 378 | 10014 | Entrez Gene | histone deacetylase 5 | 38810_at | Day 3 Peri/Day 3 Control | 0.667 | 1.7702E−04 |

TABLE 7
Diagnostic Down

| | Public | | | | | | |
|---|---|---|---|---|---|---|---|
| Gene ID | Identifier | Data Source | Gene Name | Probe_ID | Comparison | Fold Change | P value |
| 202 | 10553 | Entrez Gene | HIV-1 Tat interactive protein 2, | 38824_at | Day 3 Peri/Day 3 Control | 0.571 | 7.2789E−03 |
| | | | 30 kDa | | | | |

TABLE 8
Diagnostic Down

| | Public | | |
|---|---|---|---|
| Gene ID | Identifier | Data Source | Gene Name |
| 11 | 553168 | Entrez Gene | chromosome 1 open reading frame 68 |
| 14 | 3205 | Entrez Gene | homeo box A9 |
| 17 | 6210 | Entrez Gene | ribosomal protein S15a |
| 22 | M57951 | GenBank | UDP glucuronosyltransferase 1 family, polypeptide |
| | | | A10 /// UDP glucuronosyltransferase 1 family, |
| | | | polypeptide A8 /// UDP glucuronosyltransferase 1 |
| | | | fami |
| 26 | 5271 | Entrez Gene | serpin peptidase inhibitor, clade B (ovalbumin), |
| | | | member 8 |
| 29 | 4128 | Entrez Gene | monoamine oxidase A |
| 38 | 23352 | Entrez Gene | retinoblastoma-associated factor 600 |
| 42 | 10521 | Entrez Gene | DEAD (Asp-Glu-Ala-Asp) box polypeptide 17 |
| 45 | 3202 | Entrez Gene | homeo box A5 |
| 46 | 54 | Entrez Gene | acid phosphatase 5, tartrate resistant |
| 58 | 10158 | Entrez Gene | PDZK1 interacting protein 1 |
| 65 | 1983 | Entrez Gene | eukaryotic translation initiation factor 5 |
| 70 | 2618 | Entrez Gene | phosphoribosylglycinamide formyltransferase, |
| | | | phosphoribosylglycinamide synthetase, |
| | | | phosphoribosylaminoimidazole synthetase |
| 72 | 53335 | Entrez Gene | B-cell CLL/lymphoma 11A (zinc finger protein) |
| 80 | 26154 | Entrez Gene | ATP-binding cassette, sub-family A (ABC1), member |
| | | | 12 |
| 82 | 1410 | Entrez Gene | crystallin, alpha B |
| 84 | 10733 | Entrez Gene | polo-like kinase 4 (Drosophila) |
| 95 | HG1112- | The Institute for | — |
| | HT111 | Genomic Research | |
| 97 | 4129 | Entrez Gene | monoamine oxidase B |
| 106 | 8634 | Entrez Gene | RNA terminal phosphate cyclase domain 1 |
| 108 | 7262 | Entrez Gene | pleckstrin homology-like domain, family A, member 2 |
| 110 | 1525 | Entrez Gene | coxsackie virus and adenovirus receptor |
| 112 | 11072 | Entrez Gene | dual specificity phosphatase 14 |
| 113 | 6490 | Entrez Gene | silver homolog (mouse) |
| 119 | 6662 | Entrez Gene | SRY (sex determining region Y)-box 9 (campomelic |
| | | | dysplasia, autosomal sex-reversal) |
| 120 | 2114 | Entrez Gene | v-ets erythroblastosis virus E26 oncogene homolog 2 |
| | | | (avian) |
| 129 | 9833 | Entrez Gene | maternal embryonic leucina zipper kinase |
| 136 | 2013 | Entrez Gene | epithelial membrane protein 2 |
| 144 | AF096870 | GenBank | tripartite motif-containing 16 /// similar to tripartite |
| | | | motif-containing 16; estrogen-responsive B box |
| | | | protein |
| 150 | 771 | Entrez Gene | carbonic anhydrase XII |
| 154 | 242 | Entrez Gene | arachidonate 12-lipoxygenase, 12R type |
| 169 | 123 | Entrez Gene | adipose differentiation-related protein |
| 176 | 202 | Entrez Gene | absent in melanoma 1 |
| 178 | 5268 | Entrez Gene | serpin peptidase inhibitor, clade B (ovalbumin), |
| | | | members 5 |
| 180 | 9927 | Entrez Gene | mitofusin 2 |
| 185 | 4736 | Entrez Gene | ribosomal protein L10a |
| 186 | 644 | Entrez Gene | biliverdin reductase A |
| 188 | 3033 | Entrez Gene | L-3-hydroxyacyl-Coenzyme A dehydrogenase, short |
| | | | chain |
| 190 | 55651 | Entrez Gene | nucleolar protein family A, member 2 (H/ACA small |
| | | | nucleolar RNPs) |
| 191 | 11336 | Entrez Gene | SEC6-llke 1 (S. cerevisiae) |
| 192 | 7009 | Entrez Gene | testis enhanced gene transcript (BAX inhibitor 1) |
| 193 | 23107 | Entrez Gene | mitochondrial ribosomal protein S27 |
| 201 | 171546 | Entrez Gene | chromosome 14 open reading frame 147 |
| 202 | 10553 | Entrez Gene | HIV-1 Tat interactive protein 2, 30 kDa |
| 203 | 115817 | Entrez Gene | dehydrogenase/reductase (SDR family) member 1 |
| 204 | 25813 | Entrez Gene | sorting and assembly machinery component 50 |
| | | | homolog (s. cerevisiae) |
| 208 | 27335 | Entrez Gene | eukaryotic translation Initiation factor 3, subunit 12 |
| 209 | 217 | Entrez Gene | aldehyde dehydrogenase 2 family (mitochondrial) |
| 210 | 10049 | Entrez Gene | DnaJ (Hsp40) homolog, subfamily 8, member 6 |
| 214 | 11066 | Entrez Gene | U11/U12 snRNP 35K |
| 216 | 4729 | Entrez Gene | NADH dehydrogenase (ubiquinone) flavoprotein 2, |
| | | | 24 kDa |
| 217 | 987 | Entrez Gene | LPS-responsive vesicle trafficking, beach and anchor |
| | | | containing |
| 220 | 7295 | Entrez Gene | thioredoxin |
| 229 | 8581 | Entrez Gene | lymphocyte antigen 6 complex, locus D |
| 232 | 10476 | Entrez Gene | ATP synthase, H+ transporting, mitochondrial F0 |
| | | | complex, subunit d |
| 234 | 2806 | Entrez Gene | glutamic-oxaloacetic transaminase 2, mitochondrial |
| | | | (aspartate aminotransferase 2) |
| 235 | 23185 | Entrez Gene | La ribonucleoprotein domain family, member 5 |
| 237 | 708 | Entrez Gene | complement component 1, q subcomponent binding |
| | | | protein |
| 238 | 81875 | Entrez Gene | interferon stimulated exonuclease gene 20 kDa-like 2 |
| 239 | AI032612 | GenBank | enolase 1, (alpha) /// small nuclear ribonucleoprotein |
| | | | polypeptide F |
| 241 | 8673 | Entrez Gene | vesicle-associated membrane protein 8 (endobrevin) |
| 245 | 6249 | Entrez Gene | restin (Reed-Steinberg cell-expressed intermediate |
| | | | filament-associated protein) |
| 248 | 2547 | Entrez Gene | X-ray repair complementing defective repair in |
| | | | Chinese hamster cells 6 (Ku autoantigen, 70 kDa) |
| 249 | 545 | Entrez Gene | ataxia telangiectasia and Rad3 related |
| 250 | 4775 | Entrez Gene | nuclear factor of activated T-cells, cytoplasmic, |
| | | | calcineurin-dependent 3 |
| 252 | 4790 | Entrez Gene | nuclear factor of kappa light polypeptide gene |
| | | | enhancer in B-cells 1 (p105) |
| 255 | 23234 | Entrez Gene | DnaJ (Hsp40) homolog, subfamily C, member 9 |
| 258 | 4437 | Entrez Gene | mutS homolog 3 (E. coli) |
| 263 | 6427 | Entrez Gene | splicing factor, arginine/serine-rich 2 |
| 266 | 27292 | Entrez Gene | dimethyladenosine transferase |
| 271 | 9349 | Entrez Gene | ribosomal protein L23 |
| 280 | 8663 | Entrez Gene | eukaryotic translation initiation factor 3, subunit 8, |
| | | | 110 kDa |
| 286 | 8665 | Entrez Gene | eukaryotic translation initiation factor 3, subunit 5 |
| | | | epsilon, 47 kDa |
| 287 | 7203 | Entrez Gene | chaperonin containing TCP1, subunit 3 (gamma) |
| 293 | U05861 | GenBank | aldo-keto reductase family 1, member C1 |
| | | | (dihydrodiol dehydrogenase 1; 20-alpha (3-alpha)- |
| | | | hydroxysteroid dehydrogenase) /// aldo-keto |
| | | | reductase family |
| 295 | 9529 | Entrez Gene | BCL2-associated athanogene 5 |
| 299 | 9536 | Entrez Gene | prostaglandin E synthase |
| 303 | 4928 | Entrez Gene | nucleoporin 98 kDa |
| 309 | W28948 | GenBank | Full length insert cDNA YH77E09 |
| 321 | 6128 | Entrez Gene | ribosomal protein L6 |
| 322 | 5245 | Entrez Gene | prohibitin |
| 323 | 9296 | Entrez Gene | ATPase, H+ transporting, lysosomal 14 kDa, V1 |
| | | | subunit F |
| 324 | 1340 | Entrez Gene | cytochrome c oxidase subunit Vib polypeptide 1 |
| | | | (ubiquitous) |
| 325 | 1891 | Entrez Gene | enoyl Coenzyme A hydratase 1, peroxisomal |
| 327 | 1933 | Entrez Gene | eukaryotic translation elongation factor 1 beta 2 |
| 328 | 6141 | Entrez Gene | ribosomal protein L18 |
| 330 | 6950 | Entrez Gene | t-complex 1 |
| 331 | 29760 | Entrez Gene | B-cell linker |
| 336 | 10247 | Entrez Gene | heat-responsive protein 12 |
| 338 | 899 | Entrez Gene | cyclin F |
| 340 | 6139 | Entrez Gene | ribosomal protein L17 |
| 341 | 9521 | Entrez Gene | eukaryotic translation elongation factor 1 epsilon 1 |
| 342 | 1054 | Entrez Gene | CCAAT/enhancer binding protein (C/EBP), gamma |
| 344 | 8664 | Entrez Gene | eukaryotic translation initiation factor 3, subunit 7 |
| | | | zeta, 66/67 kDa |
| 345 | 57418 | Entrez Gene | WD repeat domain 18 |
| 346 | 6187 | Entrez Gene | ribosomal protein S2 |
| 347 | 11079 | Entrez Gene | RER1 retention in endoplasmic reticulum 1 homolog |
| | | | (S. cerevisiae) |
| 349 | 6206 | Entrez Gene | ribosomal protein S12 |
| 353 | 4070 | Entrez Gene | tumor-associated calcium signal transducer 2 |
| 354 | 7152 | Entrez Gene | topoisomerase (DNA) I pseudogene 2 |
| 355 | 4615 | Entrez Gene | myeloid differentiation primary response gene (88) |
| 357 | 9377 | Entrez Gene | cytochrome c oxidase subunit Va |
| 358 | 7386 | Entrez Gene | ubiquinol-cytochrome c reductase, Rieske iron-sulfur |
| | | | polypeptide 1 |
| 362 | HG1980- | The Institute for | — |
| | HT202 | Genomic Research | |
| 364 | 54107 | Entrez Gene | polymerase (DNA directed), epsilon 3 (p17 subunit) |
| 368 | 5936 | Entrez Gene | RNA binding motif protein 4 |
| 369 | 7004 | Entrez Gene | TEA domain family member 4 |
| 370 | 3094 | Entrez Gene | histidine triad nucleotide binding protein 1 |
| 372 | 10557 | Entrez Gene | ribonuclease P/MRP 38 kDa subunit |
| 373 | 26156 | Entrez Gene | ribosomal L1 domain containing 1 |
| 374 | 6122 | Entrez Gene | ribosomal protein L3 |
| 381 | 10694 | Entrez Gene | chaperonin containing TCP1, subunit 8 (theta) |
| 385 | 4694 | Entrez Gene | NADH dehydrogenase (ubiquinone) 1 alpha |
| | | | subcomplex, 1, 7.5 kDa |
| 388 | 10682 | Entrez Gene | emopamil binding protein (sterol isomerase) |
| 396 | 2146 | Entrez Gene | enhancer of zeste homolog 2 (Drosophila) |
| 398 | 2950 | Entrez Gene | glutathione S-transferase pl |
| 401 | 6156 | Entrez Gene | ribosomal protein L30 |
| 402 | 516 | Entrez Gene | ATP synthase, H+ transporting, mitochondrial F0 |
| | | | complex, subunit c (subunit 9), isoform 1 |
| 403 | 9128 | Entrez Gene | PRP4 pre-mRNA processing factor 4 homolog |
| | | | (yeast) |
| 404 | 3028 | Entrez Gene | hydroxyacyl-Coenzyme A dehydrogenase, type II |
| 405 | 2961 | Entrez Gene | general transcription factor IIE, polypeptide 2, beta |
| | | | 34 kDa |
| 406 | 10969 | Entrez Gene | EBNA1 binding protein 2 |
| 412 | 6768 | Entrez Gene | suppression of tumorigenicity 14 (colon carcinoma, |
| | | | matriptase, epithin) |
| 420 | 6390 | Entrez Gene | succinate dehydrogenase complex, subunit B, iron |
| | | | sulfur (Ip) |
| 421 | 162 | Entrez Gene | adaptor-related protein complex 1, beta 1 subunit |
| 423 | 6749 | Entrez Gene | structure specific recognition protein 1 |
| 425 | 6832 | Entrez Gene | suppressor of var1, 3-like 1 (S. cerevisiae) |
| 426 | 10939 | Entrez Gene | AFG3 ATPase family gene 3-like 2 (yeast) |
| 432 | 6193 | Entrez Gene | ribosomal protein S5 |
| 437 | 328 | Entrez Gene | APEX nuclease (multifunctional DNA repair enzyme) 1 |
| 441 | 4717 | Entrez Gene | NADH dehydrogenase (ubiquinone) 1, subcomplex |
| | | | unknown, 1, 6 kDa |
| 442 | 23385 | Entrez Gene | nicastrin |
| 446 | 1847 | Entrez Gene | dual specificity phosphatase 5 |
| 455 | 694 | Entrez Gene | B-cell translocation gene 1, anti-proliferative |
| 459 | 593 | Entrez Gene | branched chain keto acid dehydrogenase E1, alpha |
| | | | polypeptide (maple syrup urine disease) |
| 462 | 51635 | Entrez Gene | dehydrogenase/reductase (SDR family) member 7 |
| 465 | 613 | Entrez Gene | breakpoint cluster region |
| 466 | 7150 | Entrez Gene | topoisomerase (DNA) I |
| 467 | 1329 | Entrez Gene | cytochrome c oxidase subunit Vb |
| 469 | 6888 | Entrez Gene | transaldolase 1 |
| 471 | 6050 | Entrez Gene | ribonuclease/angiogenin inhibitor 1 |
| 484 | 26578 | Entrez Gene | osteoclast stimulating factor 1 |
| 485 | 1347 | Entrez Gene | cytochrome c oxidase subunit VIIa polypeptide 2 |
| | | | (liver) |
| 486 | 3615 | Entrez Gene | IMP (inosine monophosphate) dehydrogenase 2 |
| 491 | 3916 | Entrez Gene | lysosomal-associated membrane protein 1 |
| 492 | 23020 | Entrez Gene | activating signal cointegrator 1 complex subunit 3-like 1 |
| 495 | 6812 | Entrez Gene | syntaxin binding protein 1 |
| 496 | 23511 | Entrez Gene | nucleoporin 188 kDa |
| 498 | 283638 | Entrez Gene | KIAA0284 |
| 499 | 30968 | Entrez Gene | stomatin (EPB72)-like 2 |
| 502 | 3420 | Entrez Gene | isocitrate dehydrogenase 3 (NAD+) beta |
| 512 | 1327 | Entrez Gene | cytochrome c oxidase subunit IV isoform 1 |
| 514 | 3030 | Entrez Gene | hydroxyacyl-Coenzyme A dehydrogenase/3-ketoacyl- |
| | | | Coenzyme A thiolase/enoyl-Coenzyme A hydratase |
| | | | (trifunctional protein), alpha subunit |
| 516 | 3032 | Entrez Gene | hydroxyacyl-Coenzyme A dehydrogenase/3-ketoacyl- |
| | | | Coenzyme A thiolase/enoyl-Coenzyme A hydratase |
| | | | (trifunctional protein), beta subunit |
| 517 | 5223 | Entrez Gene | phosphoglycerate mutase 1 (brain) |
| 523 | 10036 | Entrez Gene | chromatin assembly factor 1, subunit A (p150) |
| 534 | 5437 | Entrez Gene | polymerase (RNA) II (DNA directed) polypeptide H |
| 536 | 55744 | Entrez Gene | hypothetical protein FLJ10803 |
| 544 | 7529 | Entrez Gene | tyrosine 3-monooxygenase/tryptophan 5- |
| | | | monooxygenase activation protein, beta polypeptide |
| 546 | 29 | Entrez Gene | active BCR-related gene |
| 553 | 8480 | Entrez Gene | RAE1 RNA export 1 homolog (S. pombe) |
| 571 | 3151 | Entrez Gene | high-mobility group nucleosomal binding domain 2 |
| 585 | 7534 | Entrez Gene | tyrosine 3-monooxygenase/tryptophan 5- |
| | | | monooxygenase activation protein, zeta polypeptide |

| Gene ID | Probe_ID | Comparison | Fold Change | P value |
|---|---|---|---|---|
| 11 | 31326_at | Day 7 Peri/Day 7 Extra | 0.288 | 7.6740E−03 |
| 14 | 37809_at | Day 7 Peri/Day 7 Extra | 0.237 | 8.7426E−03 |
| | 37809_at | Day 3 Peri/Day 3 Extra | 0.314 | 9.1991E−03 |
| 17 | 34316_at | Day 3 Peri/Day 3 Extra | 0.476 | 1.6785E−04 |
| 22 | 32392_s_at | Day 3 Peri/Day 3 Extra | 0.320 | 3.1014E−03 |
| 26 | 36312_at | Day 7 Peri/Day 7 Extra | 0.331 | 4.7432E−04 |
| 29 | 41772_at | Day 3 Peri/Day 3 Extra | 0.351 | 3.2109E−03 |
| | 41772_at | Day 7 Peri/Day 7 Extra | 0.614 | 6.2007E−03 |
| 38 | 33860_at | Day 3 Peri/Day 3 Extra | 0.549 | 6.8632E−03 |
| | 33860_at | Day 7 Peri/Day 7 Extra | 0.579 | 3.6353E−03 |
| 42 | 35363_at | Day 7 Peri/Day 7 Extra | 0.577 | 6.8649E−03 |
| 45 | 873_at | Day 3 Peri/Day 3 Extra | 0.387 | 6.1920E−03 |
| 46 | 677_s_at | Day 7 Peri/Day 7 Extra | 0.672 | 8.0184E−03 |
| 58 | 31610_at | Day 3 Peri/Day 3 Extra | 0.421 | 4.1131E−03 |
| 65 | 167_at | Day 7 Peri/Day 7 Extra | 0.519 | 7.0199E−03 |
| 70 | 38384_at | Day 3 Peri/Day 3 Extra | 0.436 | 8.9104E−03 |
| 72 | 41356_at | Day 3 Peri/Day 3 Extra | 0.438 | 5.3163E−03 |
| 80 | 31754_at | Day 3 Peri/Day 3 Extra | 0.453 | 4.6700E−03 |
| 82 | 32242_at | Day 3 Peri/Day 3 Extra | 0.454 | 8.8596E−03 |
| 84 | 975_at | Day 7 Peri/Day 7 Extra | 0.459 | 4.3469E−03 |
| 95 | 1839_at | Day 3 Peri/Day 3 Extra | 0.473 | 1.6274E−03 |
| 97 | 37628_at | Day 3 Peri/Day 3 Extra | 0.475 | 8.4099E−04 |
| 106 | 35195_at | Day 7 Peri/Day 7 Extra | 0.489 | 1.6326E−03 |
| 108 | 31888_s_at | Day 3 Peri/Day 3 Extra | 0.490 | 4.0424E−03 |
| 110 | 37534_at | Day 3 Peri/Day 3 Extra | 0.493 | 7.3178E−03 |
| 112 | 38272_at | Day 7 Peri/Day 7 Extra | 0.494 | 1.4839E−03 |
| 113 | 38327_at | Day 3 Peri/Day 3 Extra | 0.497 | 4.4983E−04 |
| 119 | 33436_at | Day 3 Peri/Day 3 Extra | 0.500 | 5.3219E−03 |
| 120 | 38739_at | Day 3 Peri/Day 3 Extra | 0.501 | 4.4662E−03 |
| 129 | 38847_at | Day 7 Peri/Day 7 Extra | 0.510 | 9.6404E−03 |
| 136 | 39631_at | Day 3 Peri/Day 3 Extra | 0.517 | 3.1103E−03 |
| 144 | 38881_i_at | Day 3 Peri/Day 3 Extra | 0.521 | 1.0549E−04 |
| 150 | 35275_at | Day 3 Peri/Day 3 Extra | 0.650 | 2.8445E−03 |
| 154 | 33029_at | Day 7 Peri/Day 7 Extra | 0.530 | 9.3336E−03 |
| 169 | 34378_at | Day 7 Peri/Day 7 Extra | 0.542 | 1.2411E−03 |
| 176 | 32112_s_at | Day 3 Peri/Day 3 Extra | 0.547 | 1.9891E−03 |
| 178 | 862_at | Day 3 Peri/Day 3 Extra | 0.548 | 8.5245E−03 |
| | 862_at | Day 7 Peri/Day 7 Extra | 0.576 | 5.9941E−03 |
| 180 | 34369_at | Day 3 Peri/Day 3 Extra | 0.635 | 6.4361E−03 |
| 185 | 36786_at | Day 3 Peri/Day 3 Extra | 0.556 | 7.2273E−04 |
| 186 | 32618_at | Day 3 Peri/Day 3 Extra | 0.620 | 3.2960E−04 |
| 188 | 35435_s_at | Day 3 Peri/Day 3 Extra | 0.558 | 7.5277E−03 |
| | 35435_at | Day 7 Peri/Day 7 Extra | 0.692 | 5.5220E−03 |
| 190 | 41322_s_at | Day 3 Peri/Day 3 Extra | 0.559 | 1.2634E−03 |
| 191 | 37597_s_at | Day 3 Peri/Day 3 Extra | 0.566 | 9.6420E−04 |
| 192 | 33988_at | Day 3 Peri/Day 3 Extra | 0.582 | 8.2347E−04 |
| 193 | 39377_at | Day 3 Peri/Day 3 Extra | 0.561 | 3.7750E−03 |
| 201 | 33399_at | Day 3 Peri/Day 3 Extra | 0.571 | 4.6866E−03 |
| 202 | 38824_at | Day 3 Peri/Day 3 Extra | 0.592 | 5.7073E−03 |
| 203 | 39103_s_at | Day 3 Peri/Day 3 Extra | 0.601 | 5.7200E−03 |
| 204 | 34845_at | Day 3 Peri/Day 3 Extra | 0.572 | 1.2889E−03 |
| 208 | 31492_at | Day 3 Peri/Day 3 Extra | 0.574 | 7.4544E−04 |
| 209 | 32747_at | Day 3 Peri/Day 3 Extra | 0.574 | 5.1549E−04 |
| 210 | 41234_at | Day 7 Peri/Day 7 Extra | 0.577 | 3.8743E−03 |
| 214 | 41029_at | Day 3 Peri/Day 3 Extra | 0.580 | 1.0146E−03 |
| 216 | 34893_at | Day 7 Peri/Day 7 Extra | 0.600 | 1.3243E−03 |
| 217 | 35371_at | Day 3 Peri/Day 3 Extra | 0.618 | 2.4693E−03 |
| 220 | 36992_at | Day 3 Peri/Day 3 Extra | 0.583 | 3.0827E−03 |
| 229 | 36284_at | Day 7 Peri/Day 7 Extra | 0.589 | 9.2936E−03 |
| 232 | 35760_at | Day 3 Peri/Day 3 Extra | 0.590 | 1.5912E−03 |
| 234 | 40764_at | Day 3 Peri/Day 3 Extra | 0.591 | 2.0500E−03 |
| 235 | 32585_at | Day 3 Peri/Day 3 Extra | 0.782 | 5.1596E−03 |
| 237 | 37668_at | Day 3 Peri/Day 3 Extra | 0.591 | 6.3705E−03 |
| 238 | 40946_at | Day 3 Peri/Day 3 Extra | 0.593 | 3.2988E−03 |
| 239 | 41403_at | Day 3 Peri/Day 3 Extra | 0.593 | 2.1695E−03 |
| | 41403_at | Day 7 Peri/Day 7 Extra | 0.674 | 9.8788E−04 |
| 241 | 32715_at | Day 3 Peri/Day 3 Extra | 0.593 | 2.2624E−03 |
| 245 | 34350_at | Day 7 Peri/Day 7 Extra | 0.595 | 8.1420E−03 |
| 248 | 32766_at | Day 3 Peri/Day 3 Extra | 0.597 | 8.7735E−04 |
| 249 | 37229_at | Day 7 Peri/Day 7 Extra | 0.619 | 5.1546E−03 |
| 250 | 40823_s_at | Day 3 Peri/Day 3 Extra | 0.633 | 7.3198E−03 |
| 252 | 1378_g_at | Day 3 Peri/Day 3 Extra | 0.598 | 6.5881E−03 |
| 255 | 41569_at | Day 7 Peri/Day 7 Extra | 0.599 | 4.9293E−03 |
| 258 | 1719_at | Day 3 Peri/Day 3 Extra | 0.603 | 6.7926E−03 |
| 263 | 36111_s_at | Day 7 Peri/Day 7 Extra | 0.607 | 1.0792E−03 |
| 266 | 39883_at | Day 7 Peri/Day 7 Extra | 0.609 | 2.6811E−03 |
| 271 | 32395_r_at | Day 3 Peri/Day 3 Extra | 0.611 | 2.2508E−03 |
| 280 | 34841_at | Day 3 Peri/ Day 3 Extra | 0.615 | 9.0352E−03 |
| 286 | 32576_at | Day 3 Peri/Day 3 Extra | 0.617 | 4.1860E−03 |
| 287 | 40774_at | Day 3 Peri/Day 3 Extra | 0.617 | 6.5552E−03 |
| 293 | 32805_at | Day 3 Peri/Day 3 Extra | 0.621 | 6.7226E−03 |
| 295 | 36463_at | Day 3 Peri/Day 3 Extra | 0.624 | 7.6918E−03 |
| 299 | 38131_at | Day 3 Peri/Day 3 Extra | 0.626 | 9.0721E−03 |
| 303 | 38911_at | Day 3 Peri/Day 3 Extra | 0.627 | 2.3405E−03 |
| 309 | 37161_at | Day 3 Peri/Day 3 Extra | 0.631 | 4.5494E−03 |
| 321 | 31952_at | Day 3 Peri/Day 3 Extra | 0.637 | 8.7667E−03 |
| 322 | 36592_at | Day 3 Peri/Day 3 Extra | 0.637 | 3.4444E−03 |
| 323 | 37395_at | Day 3 Peri/Day 3 Extra | 0.810 | 7.4746E−03 |
| 324 | 40872_at | Day 3 Peri/Day 3 Extra | 0.638 | 1.3105E−03 |
| 325 | 32756_at | Day 3 Peri/Day 3 Extra | 0.638 | 8.7097E−03 |
| 327 | 35748_at | Day 3 Peri/Day 3 Extra | 0.639 | 7.6265E−04 |
| 328 | 31546_at | Day 3 Peri/Day 3 Extra | 0.691 | 6.7124E−03 |
| 330 | 34791_at | Day 3 Peri/Day 3 Extra | 0.640 | 3.7040E−03 |
| 331 | 38242_at | Day 3 Peri/Day 3 Extra | 0.640 | 7.2677E−04 |
| 336 | 32173_at | Day 7 Peri/Day 7 Extra | 0.645 | 6.3967E−03 |
| 338 | 35907_at | Day 3 Peri/Day 3 Extra | 0.646 | 8.5538E−03 |
| 340 | 32440_at | Day 3 Peri/Day 3 Extra | 0.647 | 6.1617E−03 |
| 341 | 40587_s_at | Day 3 Peri/Day 3 Extra | 0.718 | 6.3530E−03 |
| 342 | 39219_at | Day 7 Peri/Day 7 Extra | 0.647 | 8.7653E−03 |
| 344 | 35298_at | Day 3 Peri/Day 3 Extra | 0.649 | 2.0180E−03 |
| 345 | 35983_at | Day 3 Peri/Day 3 Extra | 0.651 | 3.7380E−03 |
| 346 | 31527_at | Day 3 Peri/Day 3 Extra | 0.651 | 6.9801E−03 |
| 347 | 41551_at | Day 7 Peri/Day 7 Extra | 0.651 | 8.1132E−03 |
| 349 | 33117_r_at | Day 3 Peri/Day 3 Extra | 0.652 | 2.4740E−03 |
| 353 | 291_s_at | Day 3 Peri/Day 3 Extra | 0.655 | 8.7414E−03 |
| 354 | 31680_at | Day 7 Peri/Day 7 Extra | 0.654 | 8.0454E−03 |
| 355 | 38369_at | Day 7 Peri/Day 7 Extra | 0.654 | 5.5186E−03 |
| 357 | 41223_at | Day 3 Peri/Day 3 Extra | 0.656 | 7.0969E−03 |
| 358 | 34401_at | Day 3 Peri/Day 3 Extra | 0.656 | 5.8159E−03 |
| 362 | 956_at | Day 7 Peri/Day 7 Extra | 0.658 | 8.0238E−03 |
| 364 | 38702_at | Day 3 Peri/Day 3 Extra | 0.660 | 3.6355E−03 |
| 368 | 35351_at | Day 3 Peri/Day 3 Extra | 0.662 | 1.3875E−03 |
| 369 | 41037_at | Day 7 Peri/Day 7 Extra | 0.662 | 6.4186E−04 |
| 370 | 1009_at | Day 3 Peri/Day 3 Extra | 0.662 | 6.3134E−03 |
| 372 | 41040_at | Day 7 Peri/Day 7 Extra | 0.663 | 2.3436E−03 |
| 373 | 39418_at | Day 7 Peri/Day 7 Extra | 0.664 | 2.4392E−03 |
| | 39418_at | Day 3 Peri/Day 3 Extra | 0.730 | 4.3297E−03 |
| 374 | 31722_at | Day 3 Peri/Day 3 Extra | 0.665 | 8.4932E−03 |
| 381 | 39767_at | Day 3 Peri/Day 3 Extra | 0.669 | 4.6792E−03 |
| 385 | 36169_at | Day 3 Peri/Day 3 Extra | 0.706 | 1.3204E−03 |
| 388 | 32536_at | Day 3 Peri/Day 3 Extra | 0.672 | 3.7237E−03 |
| 396 | 37305_at | Day 7 Peri/ Day 7 Extra | 0.677 | 5.6094E−03 |
| | 37305_at | Day 3 Peri/Day 3 Extra | 0.701 | 9.0063E−03 |
| 398 | 33396_at | Day 3 Peri/Day 3 Extra | 0.679 | 6.0265E−03 |
| | 33396_at | Day 7 Peri/Day 7 Extra | 0.695 | 5.1049E−03 |
| 401 | 31708_at | Day 3 Peri/Day 3 Extra | 0.682 | 4.7648E−03 |
| 402 | 38076_at | Day 3 Peri/Day 3 Extra | 0.682 | 3.2512E−04 |
| 403 | 37936_at | Day 3 Peri/Day 3 Extra | 0.682 | 1.2100E−03 |
| 404 | 40778_at | Day 3 Peri/Day 3 Extra | 0.687 | 5.4834E−03 |
| 405 | 37295_at | Day 7 Peri/Day 7 Extra | 0.683 | 5.1041E−03 |
| 406 | 36135_at | Day 3 Peri/Day 3 Extra | 0.683 | 7.3375E−03 |
| 412 | 35309_at | Day 3 Peri/Day 3 Extra | 0.685 | 2.8284E−03 |
| 420 | 35751_at | Day 3 Peri/Day 3 Extra | 0.689 | 9.9046E−03 |
| 421 | 40745_at | Day 3 Peri/Day 3 Extra | 0.689 | 1.6623E−03 |
| 423 | 37739_at | Day 3 Peri/Day 3 Extra | 0.690 | 4.8988E−03 |
| 425 | 41408_at | Day 3 Peri/Day 3 Extra | 0.691 | 6.4202E−03 |
| 426 | 34315_at | Day 3 Peri/Day 3 Extra | 0.692 | 3.6562E−04 |
| 432 | 32437_at | Day 3 Peri/Day 3 Extra | 0.693 | 1.4564E−03 |
| 437 | 2025_s_at | Day 3 Peri/Day 3 Extra | 0.746 | 9.3963E−03 |
| 441 | 38485_at | Day 3 Peri/Day 3 Extra | 0.696 | 2.0589E−03 |
| 442 | 34835_at | Day 3 Peri/Day 3 Extra | 0.696 | 1.6857E−03 |
| 446 | 529_at | Day 7 Peri/Day 7 Extra | 0.697 | 3.7478E−03 |
| 455 | 37294_at | Day 3 Peri/Day 3 Extra | 0.704 | 5.5679E−03 |
| 459 | 37704_at | Day 3 Peri/Day 3 Extra | 0.720 | 1.7216E−03 |
| 462 | 39814_s_at | Day 3 Peri/Day 3 Extra | 0.708 | 6.9810E−03 |
| 465 | 34679_at | Day 3 Peri/Day 3 Extra | 0.709 | 4.4827E−03 |
| 466 | 1030_s_at | Day 7 Peri/Day 7 Extra | 0.711 | 1.5853E−03 |
| 467 | 39921_at | Day 3 Peri/Day 3 Extra | 0.711 | 8.1965E−03 |
| 469 | 37311_at | Day 3 Peri/Day 3 Extra | 0.713 | 2.9353E−04 |
| 471 | 36187_at | Day 3 Peri/Day 3 Extra | 0.715 | 1.3879E−03 |
| 484 | 467_at | Day 7 Peri/Day 7 Extra | 0.725 | 7.2710E−03 |
| 485 | 41760_at | Day 3 Peri/Day 3 Extra | 0.726 | 1.4279E−03 |
| 486 | 36624_at | Day 3 Peri/Day 3 Extra | 0.726 | 5.8955E−03 |
| 491 | 39758_f_at | Day 3 Peri/Day 3 Extra | 0.732 | 1.6743E−03 |
| 492 | 41224_at | Day 3 Peri/Day 3 Extra | 0.732 | 7.2017E−03 |
| 495 | 33942_s_at | Day 3 Peri/ Day 3 Extra | 0.734 | 2.7323E−03 |
| 496 | 32644_at | Day 3 Peri/Day 3 Extra | 0.735 | 1.7224E−03 |
| 498 | 38592_s_at | Day 3 Peri/Day 3 Extra | 0.736 | 1.6693E−03 |
| 499 | 34380_at | Day 3 Peri/Day 3 Extra | 0.736 | 6.0626E−03 |
| 502 | 40111_g_at | Day 3 Peri/Day 3 Extra | 0.737 | 9.0628E−03 |
| 512 | 39027_at | Day 3 Peri/Day 3 Extra | 0.746 | 6.3689E−03 |
| 514 | 36952_at | Day 3 Peri/Day 3 Extra | 0.750 | 6.1291E−03 |
| 516 | 39741_at | Day 3 Peri/Day 3 Extra | 0.752 | 2.0159E−03 |
| 517 | 41221_at | Day 3 Peri/Day 3 Extra | 0.752 | 1.6814E−03 |
| 523 | 32589_at | Day 7 Peri/Day 7 Extra | 0.756 | 2.8970E−03 |
| 534 | 35631_at | Day 3 Peri/Day 3 Extra | 0.764 | 7.2269E−03 |
| 536 | 37610_at | Day 3 Peri/Day 3 Extra | 0.764 | 2.5200E−03 |
| 544 | 32324_at | Day 3 Peri/Day 3 Extra | 0.769 | 5.4500E−03 |
| 546 | 39058_at | Day 3 Peri/Day 3 Extra | 0.796 | 8.8684E−03 |
| 553 | 32757_at | Day 3 Peri/Day 3 Extra | 0.778 | 1.2850E−03 |
| 571 | 41231_f_at | Day 3 Peri/Day 3 Extra | 0.796 | 7.6383E−04 |
| 585 | 1235_at | Day 3 Peri/Day 3 Extra | 0.824 | 9.9973E−03 |

TABLE 9
Diagnostic Down

| | Public | | | | | | |
|---|---|---|---|---|---|---|---|
| Gene ID | Identifier | Data Source | Gene Name | Probe_ID | Comparison | Fold Change | P value |
| 178 | 5268 | Entrez Gene | serpin peptidase inhibitor, | 862_at | Day 3 Peri/Day 3 Extra | 0.548 | 8.5245E−03 |
| | | | clade B (ovalbumin), member 5 | 862_at | Day 7 Peri/Day 7 Extra | 0.576 | 5.9941E−03 |
| 220 | 7295 | Entrez Gene | thioredoxin | 36992_at | Day 3 Peri/Day 3 Extra | 0.583 | 3.0827E−03 |

TABLE 10
Diagnostic Down

| | Public | | | | | | |
|---|---|---|---|---|---|---|---|
| Gene ID | Identifier | Data Source | Gene Name | Probe_ID | Comparison | Fold Change | P value |
| 110 | 1525 | Entrez Gene | coxsackie virus and | 37534_at | Day 3 Peri/Day 3 Extra | 0.493 | 7.3178E−03 |
| | | | adenovirus receptor | | | | |
| 229 | 8581 | Entrez Gene | lymphocyte antigen 6 | 36284_at | Day 7 Peri/Day 7 Extra | 0.589 | 9.2936E−03 |
| | | | complex, locus D | | | | |

TABLE 11
Diagnostic Down

| | Public | | | | | | |
|---|---|---|---|---|---|---|---|
| Gene ID | Identifier | Data Source | Gene Name | Probe_ID | Comparison | Fold Change | P value |
| 250 | 4775 | Entrez Gene | nuclear factor of activated T-cells, | 40823_s_at | Day 3 Peri/Day 3 Extra | 0.633 | 7.3198E−03 |
| | | | cytoplasmic, calcineurin-dependent 3 | | | | |
| 252 | 4790 | Entrez Gene | nuclear factor of kappa light polypeptide | 1378_g_at | Day 3 Peri/Day 3 Extra | 0.598 | 6.5881E−03 |
| | | | gene enhancer in B-cells 1 (p105) | | | | |
| 355 | 4615 | Entrez Gene | myeloid differentiation primary | 38369_at | Day 7 Peri/Day 7 Extra | 0.654 | 5.5186E−03 |
| | | | response gene (88) | | | | |

TABLE 12
Diagnostic Down

| | Public | | | | | | |
|---|---|---|---|---|---|---|---|
| Gene ID | Identifier | Data Source | Gene Name | Probe_ID | Comparison | Fold Change | P value |
| 202 | 10553 | Entrez Gene | HIV-1 Tat interactive protein 2, | 38824_at | Day 3 Peri/Day 3 Extra | 0.592 | 5.7073E−03 |
| | | | 30 kDa | | | | |

TABLE 13
Diagnostic Down

| Gene | Public | | | | | Fold | |
|---|---|---|---|---|---|---|---|
| ID | Identifier | Data Source | Gene Name | Probe_ID | Comparison | Change | P value |
| 8 | 9415 | Entrez Gene | fatty acid desaturase 2 | 32190_at | Day 3 Peri/Day 0 Peri | 0.365 | 8.5757E−03 |
| 11 | 553168 | Entrez Gene | chromosome 1 open reading frame 68 | 31326_at | Day 7 Peri/Day 0 Peri | 0.229 | 1.6500E−05 |
| | | | | 31326_at | Day 3 Peri/Day 0 Peri | 0.487 | 3.0725E−03 |
| 18 | 7123 | Entrez Gene | C-type lectin domain family 3, member B | 36569_at | Day 3 Peri/Day 0 Peri | 0.356 | 1.8400E−05 |
| | | | | 36569_at | Day 7 Peri/Day 0 Peri | 0.428 | 5.8746E−04 |
| 20 | AB011538 | GenBank | CDNA clone IMAGE: 5922621 | 35324_at | Day 3 Peri/Day 0 Peri | 0.447 | 1.3446E−04 |
| | | | | 35324_at | Day 7 Peri/Day 0 Peri | 0.623 | 4.2479E−03 |
| 24 | 2053 | Entrez Gene | epoxide hydrolase 2, cytoplasmic | 41473_at | Day 3 Peri/Day 0 Peri | 0.521 | 6.6949E−03 |
| 31 | 8483 | Entrez Gene | cartilage intermediate layer protein, nucleotide | 34985_at | Day 3 Peri/Day 0 Peri | 0.354 | 4.8214E−04 |
| | | | pyrophosphohydrolase | 34985_at | Day 7 Peri/Day 0 Peri | 0.381 | 2.9109E−03 |
| 34 | 10458 | Entrez Gene | BAI1-associated protein 2 | 37761_at | Day 7 Peri/Day 0 Peri | 0.358 | 3.2292E−04 |
| 36 | 2273 | Entrez Gene | four and a half LIM domains 1 | 32542_at | Day 3 Peri/Day 0 Peri | 0.598 | 1.0604E−03 |
| | | | | 32542_at | Day 7 Peri/Day 0 Peri | 0.643 | 1.4304E−03 |
| 44 | 2194 | Entrez Gene | fatty acid synthase | 38429_at | Day 3 Peri/Day 0 Peri | 0.512 | 8.4627E−03 |
| 51 | 5354 | Entrez Gene | proteolipid protein 1 (Pelizaeus-Merzbacher | 41158_at | Day 3 Peri/Day 0 Peri | 0.715 | 7.5707E−03 |
| | | | disease, spastic paraplegia 2, uncomplicated) | | | | |
| 56 | 1675 | Entrez Gene | D component of complement (adipsin) | 40282_s_at | Day 7 Peri/Day 0 Peri | 0.410 | 3.2238E−03 |
| 57 | 23242 | Entrez Gene | cordon-bleu homolog (mouse) | 35669_at | Day 7 Peri/Day 0 Peri | 0.543 | 1.1342E−03 |
| | | | | 35669_at | Day 3 Peri/Day 0 Peri | 0.585 | 1.2820E−03 |
| 71 | HG3570- | The Institute for | — | 936_s_at | Day 7 Peri/Day 0 Peri | 0.679 | 6.6192E−03 |
| | HT377 | Genomic Research | | | | | |
| 77 | 9524 | Entrez Gene | glycoprotein, synaptic 2 | 38966_at | Day 7 Peri/Day 0 Peri | 0.614 | 2.1060E−03 |
| 93 | 1152 | Entrez Gene | creatine kinase, brain | 40863_r_at | Day 7 Peri/Day 0 Peri | 0.507 | 3.2271E−04 |
| 94 | 10450 | Entrez Gene | peptidylprolyl isomerase E (cyclophilin E) | 34365_at | Day 7 Peri/Day 0 Peri | 0.638 | 2.8273E−03 |
| 101 | 8532 | Entrez Gene | carboxypeptidase Z | 37248_at | Day 7 Peri/Day 0 Peri | 0.483 | 2.2125E−04 |
| | | | | 37248_at | Day 3 Peri/Day 0 Peri | 0.522 | 7.1260E−04 |
| 102 | 6038 | Entrez Gene | ribonuclease, RNase A family, 4 | 32664_at | Day 3 Peri/Day 0 Peri | 0.636 | 5.8524E−04 |
| | | | | 32664_at | Day 7 Peri/Day 0 Peri | 0.691 | 3.6796E−03 |
| 107 | 4163 | Entrez Gene | mutated in colorectal cancers | 35561_at | Day 7 Peri/Day 0 Peri | 0.606 | 7.2171E−03 |
| 113 | 6490 | Entrez Gene | silver homolog (mouse) | 38327_at | Day 3 Peri/Day 0 Peri | 0.710 | 9.2062E−03 |
| 130 | U18300 | GenBank | damage-specific DNA binding protein 2, | 1243_at | Day 7 Peri/Day 0 Peri | 0.749 | 8.6022E−03 |
| | | | 48 kDa /// LIM homeobox 3 | | | | |
| 131 | 23051 | Entrez Gene | zinc fingers and homeoboxes 3 | 40461_at | Day 3 Peri/Day 0 Peri | 0.738 | 3.2244E−03 |
| 136 | 2013 | Entrez Gene | epithelial membrane protein 2 | 39631_at | Day 7 Peri/Day 0 Peri | 0.629 | 5.1967E−04 |
| 137 | 81563 | Entrez Gene | chromosome 1 open reading frame 21 | 41679_at | Day 7 Peri/Day 0 Peri | 0.517 | 1.2344E−03 |
| | | | | 41679_at | Day 3 Peri/Day 0 Peri | 0.607 | 8.6951E−03 |
| 138 | 1638 | Entrez Gene | dopachrome tautornerase (dopachrome delta- | 41182_at | Day 7 Peri/Day 0 Peri | 0.518 | 2.7300E−05 |
| | | | isomerase, tyrosine-related protein 2) | 41182_at | Day 3 Peri/Day 0 Peri | 0.596 | 1.9071E−04 |
| 148 | 4713 | Entrez Gene | NADH dehydrogenase (ubiquinone) 1 beta | 35773_i_at | Day 7 Peri/Day 0 Peri | 0.748 | 5.4735E−03 |
| | | | subcomplex, 7, 18 kDa | 35774_r_at | Day 7 Peri/Day 3 Peri | 0.791 | 1.2691E−03 |
| 149 | 1396 | Entrez Gene | cysteine-rich protein 1 (intestinal) | 33232_at | Day 7 Peri/Day 0 Peri | 0.526 | 3.2907E−04 |
| | | | | 33232_at | Day 3 Peri/Day 0 Peri | 0.596 | 1.5417E−03 |
| 151 | 55187 | Entrez Gene | vacuolar protein sorting 13D (yeast) | 32743_at | Day 3 Peri/Day 0 Peri | 0.772 | 6.0079E−03 |
| 160 | 113146 | Entrez Gene | chromosome 14 open reading frame 78 | 36497_at | Day 7 Peri/Day 0 Peri | 0.545 | 2.9700E−05 |
| | | | | 36497_at | Day 3 Peri/Day 0 Peri | 0.577 | 3.7100E−05 |
| 179 | 1474 | Entrez Gene | cystatin E/M | 33128_s_at | Day 7 Peri/Day 0 Peri | 0.549 | 1.9072E−03 |
| | | | | 33128_s_at | Day 3 Peri/Day 0 Peri | 0.605 | 2.6588E−03 |
| 185 | 4736 | Entrez Gene | ribosomal protein L10a | 36786_at | Day 7 Peri/Day 0 Peri | 0.783 | 1.0376E−03 |
| 209 | 217 | Entrez Gene | aldehyde dehydrogenase 2 family | 32747_at | Day 7 Peri/Day 0 Peri | 0.741 | 2.4111E−03 |
| | | | (mitochondrial) | | | | |
| 212 | 8623 | Entrez Gene | acetylserotonin O-methyltransferase-like | 36553_at | Day 3 Peri/Day 0 Peri | 0.710 | 1.0994E−04 |
| 213 | 169611 | Entrez Gene | olfactomedin-like 2A | 38312_at | Day 3 Peri/Day 0 Peri | 0.719 | 4.2489E−04 |
| 223 | 2621 | Entrez Gene | growth arrest-specific 6 | 1597_at | Day 3 Peri/Day 0 Peri | 0.689 | 5.8701E−03 |
| 230 | AB006780 | GenBank | lectin, galactoside-binding, soluble, 3 | 35367_at | Day 7 Peri/Day 0 Peri | 0.683 | 4.0159E−04 |
| | | | (galectin 3) /// galectin-3 internal gene | 35367_at | Day 3 Peri/Day 0 Peri | 0.763 | 8.3297E−03 |
| 231 | 6929 | Entrez Gene | transcription factor 3 (E2A immunoglobulin | 1373_at | Day 3 Peri/Day 0 Peri | 0.738 | 3.1310E−04 |
| | | | enhancer binding factors E12/E47) | | | | |
| 240 | 26040 | Entrez Gene | SET binding protein 1 | 34990_at | Day 3 Peri/Day 0 Peri | 0.593 | 3.4533E−04 |
| | | | | 34990_at | Day 7 Peri/Day 0 Peri | 0.711 | 9.8766E−03 |
| 257 | AI557912 | GenBank | translocase of outer mitochondrial membrane 7 | 39025_at | Day 7 Peri/Day 0 Peri | 0.798 | 1.0424E−03 |
| | | | homolog (yeast) /// hypothetical protein | | | | |
| | | | LOC201725 | | | | |
| 260 | 5453 | Entrez Gene | POU domain, class 3, transcription factor 1 | 33675_at | Day 3 Peri/Day 0 Peri | 0.606 | 4.5158E−03 |
| 273 | 23090 | Entrez Gene | zinc finger protein 423 | 34950_at | Day 3 Peri/Day 0 Peri | 0.613 | 2.6182E−04 |
| 276 | 56967 | Entrez Gene | chromosome 14 open reading frame 132 | 41837_at | Day 3 Peri/Day 0 Peri | 0.614 | 6.2326E−04 |
| | | | | 41837_at | Day 7 Peri/Day 0 Peri | 0.660 | 1.7553E−03 |
| 277 | 8560 | Entrez Gene | degenerative spermatocyte homolog 1, lipid | 33337_at | Day 7 Peri/Day 0 Peri | 0.690 | 1.2271E−03 |
| | | | desaturase (Drosophila) | | | | |
| 280 | 8663 | Entrez Gene | eukaryotic translation initiation factor 3, | 34841_at | Day 3 Peri/Day 0 Peri | 0.776 | 9.4126E−03 |
| | | | subunit 8, 110 kDa | | | | |
| 285 | 23650 | Entrez Gene | tripartite motif-containing 29 | 1898_at | Day 7 Peri/Day 0 Peri | 0.617 | 3.0138E−03 |
| 300 | 22883 | Entrez Gene | calsyntenin 1 | 41498_at | Day 3 Peri/Day 0 Peri | 0.777 | 3.9252E−03 |
| 304 | 3290 | Entrez Gene | hydroxysteroid (11-beta) dehydrogenase 1 | 35702_at | Day 7 Peri/Day 0 Peri | 0.628 | 6.5442E−03 |
| 306 | 2065 | Entrez Gene | v-erb-b2 erythroblastic leukemia viral oncogene | 32787_at | Day 7 Peri/Day 0 Peri | 0.677 | 7.5081E−03 |
| | | | homolog 3 (avian) | | | | |
| 307 | 10217 | Entrez Gene | CTD (carboxy-terminal domain, RNA | 40196_at | Day 7 Peri/Day 0 Peri | 0.735 | 2.0122E−03 |
| | | | polymerase II, polypeptide A) small | 40196_at | Day 3 Peri/Day 0 Peri | 0.770 | 5.2302E−03 |
| | | | phosphatase-like | | | | |
| 311 | 4681 | Entrez Gene | neuroblastoma, suppression of tumorigenicity 1 | 37005_at | Day 7 Peri/Day 0 Peri | 0.642 | 1.7449E−03 |
| | | | | 37005_at | Day 3 Peri/Day 0 Peri | 0.695 | 2.4042E−03 |
| 323 | 9296 | Entrez Gene | ATPase, H+ transporting, lysosomal 14 kDa, | 37395_at | Day 7 Peri/Day 0 Peri | 0.840 | 4.0330E−03 |
| | | | V1 subunit F | | | | |
| 328 | 6141 | Entrez Gene | ribosomal protein L18 | 31546_at | Day 7 Peri/Day 0 Peri | 0.760 | 9.8180E−03 |
| 333 | 2709 | Entrez Gene | gap junction protein, beta 5 (connexin 31.1) | 38903_at | Day 7 Peri/Day 0 Peri | 0.643 | 1.7967E−03 |
| 339 | HG162- | The Institute for | — | 1278_at | Day 3 Peri/Day 0 Peri | 0.647 | 6.0118E−03 |
| | HT3165 | Genomic Research | | | | | |
| 374 | 6122 | Entrez Gene | ribosomal protein L3 | 31722_at | Day 7 Peri/Day 0 Peri | 0.792 | 9.5624E−03 |
| 375 | 6176 | Entrez Gene | ribosomal protein, large, P1 | 31957_r_at | Day 7 Peri/Day 0 Peri | 0.666 | 3.7416E−03 |
| 378 | 10014 | Entrez Gene | histone deacetylase 5 | 38810_at | Day 7 Peri/Day 0 Peri | 0.778 | 7.8739E−03 |
| | | | | 38810_at | Day 3 Peri/Day 0 Peri | 0.784 | 9.3572E−04 |
| 379 | 10610 | Entrez Gene | ST6 (alpha-N-acetyl-neuraminyl-2,3-beta- | 34693_at | Day 7 Peri/Day 0 Peri | 0.668 | 5.8340E−04 |
| | | | galactosyl-1,3)-N-acetylgalactosaminide | | | | |
| | | | alpha-2,6-sialyltransferase 2 | | | | |
| 383 | 4856 | Entrez Gene | nephroblastoma overexpressed gene | 39250_at | Day 3 Peri/Day 0 Peri | 0.670 | 5.1134E−03 |
| 384 | 9314 | Entrez Gene | Kruppel-like factor 4 (gut) | 36214_at | Day 7 Peri/Day 0 Peri | 0.670 | 7.5246E−03 |
| 386 | 10904 | Entrez Gene | bladder cancer associated protein | 35266_at | Day 3 Peri/Day 0 Peri | 0.815 | 4.1112E−03 |
| 404 | 3028 | Entrez Gene | hydroxyacyl-Coenzyme A dehydrogenase, | 40778_at | Day 7 Peri/Day 0 Peri | 0.785 | 3.6406E−03 |
| | | | type II | | | | |
| 408 | 4610 | Entrez Gene | v-myc myelocytomatosis viral oncogene | 1490_at | Day 3 Peri/Day 0 Peri | 0.684 | 2.9494E−04 |
| | | | homolog 1, lung carcinoma derived (avian) | | | | |
| 416 | 10420 | Entrez Gene | testis-specific kinase 2 | 33164_at | Day 7 Peri/Day 0 Peri | 0.688 | 8.0384E−03 |
| 424 | 4134 | Entrez Gene | microtubule-associated protein 4 | 33850_at | Day 3 Peri/Day 0 Peri | 0.690 | 9.2919E−04 |
| | | | | 33850_at | Day 7 Peri/Day 0 Peri | 0.747 | 8.7450E−03 |
| 429 | 10570 | Entrez Gene | dihydropyrimidinase-like 4 | 39503_s_at | Day 7 Peri/Day 0 Peri | 0.693 | 9.8616E−03 |
| 432 | 6193 | Entrez Gene | ribosomal protein S5 | 32437_at | Day 7 Peri/Day 0 Peri | 0.740 | 3.6964E−03 |
| | | | | 32437_at | Day 3 Peri/Day 0 Peri | 0.808 | 6.6422E−03 |
| 433 | 23492 | Entrez Gene | chromobox homolog 7 | 36894_at | Day 3 Peri/Day 0 Peri | 0.742 | 1.6692E−03 |
| | | | | 36894_at | Day 7 Peri/Day 0 Peri | 0.742 | 3.4050E−03 |
| 434 | 4809 | Entrez Gene | NHP2 non-histone chromosome protein | 41746_at | Day 3 Peri/Day 0 Peri | 0.830 | 6.1703E−03 |
| | | | 2-like 1 (S. cerevisiae) | | | | |
| 436 | 79090 | Entrez Gene | trafficking protein particle complex 6A | 36529_at | Day 3 Peri/Day 0 Peri | 0.694 | 6.8032E−03 |
| 438 | 84525 | Entrez Gene | homeodomain-only protein | 39698_at | Day 7 Peri/Day 0 Peri | 0.695 | 8.7289E−03 |
| 449 | 57017 | Entrez Gene | chromosome 16 open reading frame 49 | 34810_at | Day 7 Peri/Day 0 Peri | 0.810 | 4.2261E−03 |
| 457 | 4174 | Entrez Gene | MCM5 minichromosome maintenance deficient | 982_at | Day 7 Peri/Day 3 Peri | 0.706 | 4.9235E−04 |
| | | | 5, cell division cycle 46 (S. cerevisiae) | | | | |
| 458 | 10278 | Entrez Gene | embryonal Fyn-associated substrate | 33883_at | Day 7 Peri/Day 0 Peri | 0.706 | 2.0000E−05 |
| | | | | 33883_at | Day 3 Peri/Day 0 Peri | 0.718 | 6.1275E−04 |
| 460 | 4357 | Entrez Gene | mercaptopyruvate sulfurtransferase | 36124_at | Day 7 Peri/Day 0 Peri | 0.707 | 7.1338E−04 |
| 461 | 3727 | Entrez Gene | jun D proto-oncogene | 41483_s_at | Day 7 Day 0 Peri | 0.707 | 9.7391E−03 |
| 464 | 22882 | Entrez Gene | zinc fingers and homeoboxes 2 | 41503_at | Day 3 Peri/Day 0 Peri | 0.709 | 9.7700E−05 |
| | | | | 41503_at | Day 7 Peri/Day 0 Peri | 0.830 | 5.9812E−03 |
| 472 | 2064 | Entrez Gene | v-erb-b2 erythroblastic leukemia viral oncogene | 33218_at | Day 3 Peri/Day 0 Peri | 0.718 | 4.9044E−04 |
| | | | homolog 2, neuro/glioblastoma derived | 33218_at | Day 7 Peri/Day 0 Peri | 0.740 | 6.2999E−04 |
| | | | oncogene homolog (avian) | | | | |
| 474 | 79026 | Entrez Gene | AHNAK nucleoprotein (desmoyokin) | 37027_at | Day 7 Peri/Day 0 Peri | 0.748 | 8.8700E−05 |
| | | | | 37027_at | Day 3 Peri/Day 0 Peri | 0.780 | 3.1374E−04 |
| 476 | 949 | Entrez Gene | scavenger receptor class B, member 1 | 41200_at | Day 7 Peri/Day 0 Peri | 0.720 | 1.3768E−03 |
| | | | | 41200_at | Day 3 Peri/Day 0 Peri | 0.721 | 1.6250E−03 |
| 477 | 1917 | Entrez Gene | eukaryotic translation elongation factor 1 | 35174_i_at | Day 7 Peri/Day 0 Peri | 0.720 | 6.7372E−04 |
| | | | alpha 2 | | | | |
| 478 | 6205 | Entrez Gene | ribosomal protein S11 | 32330_at | Day 7 Peri/Day 0 Peri | 0.721 | 3.3215E−03 |
| 481 | 79095 | Entrez Gene | chromosome 9 open reading frame 16 | 41047_at | Day 3 Peri/Day 0 Peri | 0.725 | 5.9374E−03 |
| 483 | 22924 | Entrez Gene | microtubule-associated protein, RP/EB family, | 40825_at | Day 7 Peri/Day 0 Peri | 0.725 | 1.1882E−03 |
| | | | member 3 | 40825_at | Day 3 Peri/Day 0 Peri | 0.765 | 2.4008E−03 |
| 486 | 3615 | Entrez Gene | IMP (inosine monophosphate) dehydrogenase 2 | 36624_at | Day 7 Peri/Day 0 Peri | 0.740 | 8.1540E−04 |
| 487 | 1981 | Entrez Gene | eukaryotic translation initiation factor 4 | 32844_at | Day 7 Peri/Day 0 Peri | 0.727 | 9.4700E−03 |
| | | | gamma, 1 | | | | |
| 494 | 10075 | Entrez Gene | HECT, UBA and WWE domain containing 1 | 34372_at | Day 3 Peri/Day 0 Peri | 0.802 | 9.1907E−03 |
| 497 | HG2238- | The Institute for | — | 329_s_at | Day 3 Peri/Day 0 Peri | 0.736 | 9.9413E−03 |
| | HT232 | Genomic Research | | | | | |
| 501 | 10634 | Entrez Gene | growth arrest-specific 2 like 1 | 31874_at | Day 7 Peri/Day 0 Peri | 0.737 | 7.5700E−05 |
| | | | | 31874_at | Day 3 Peri/Day 0 Peri | 0.791 | 1.9327E−03 |
| 503 | 10424 | Entrez Gene | progesterone receptor membrane component 2 | 38821_at | Day 7 Peri/Day 0 Peri | 0.738 | 3.9496E−03 |
| | | | | 38821_at | Day 3 Peri/Day 0 Peri | 0.746 | 7.1002E−03 |
| 504 | 8531 | Entrez Gene | cold shock domain protein A | 39839_at | Day 7 Peri/Day 0 Peri | 0.738 | 8.5534E−03 |
| 505 | 5439 | Entrez Gene | polymerase (RNA) II (DNA directed) | 1486_at | Day 7 Peri/Day 0 Peri | 0.740 | 2.7928E−03 |
| | | | polypeptide J, 13.3 kDa | 1486_at | Day 7 Peri/Day 3 Peri | 0.772 | 1.7983E−03 |
| 507 | 6227 | Entrez Gene | ribosomal protein S21 | 32744_at | Day 7 Peri/Day 0 Peri | 0.740 | 7.2902E−03 |
| 508 | 6169 | Entrez Gene | ribosomal protein L38 | 34085_at | Day 7 Peri/Day 0 Peri | 0.743 | 6.3557E−03 |
| 510 | 6218 | Entrez Gene | ribosomal protein S17 | 34592_at | Day 7 Peri/Day 0 Peri | 0.745 | 2.8146E−03 |
| 518 | 10638 | Entrez Gene | S-phase response (cyclin-related) | 1685_at | Day 3 Peri/Day 0 Peri | 0.754 | 1.2093E−03 |
| 522 | 162427 | Entrez Gene | hypothetical protein LOC162427 | 38423_at | Day 3 Peri/Day 0 Peri | 0.845 | 2.2722E−03 |
| | | | | 38423_at | Day 7 Peri/Day 0 Peri | 0.848 | 1.7317E−03 |
| 524 | 6202 | Entrez Gene | ribosomal protein S8 | 31583_at | Day 7 Peri/Day 0 Peri | 0.756 | 8.6210E−03 |
| 528 | 26003 | Entrez Gene | golgi reassembly stacking protein 2, 55 kDa | 35805_at | Day 3 Peri/Day 0 Peri | 0.761 | 2.3291E−04 |
| | | | | 35805_at | Day 7 Peri/Day 0 Peri | 0.770 | 2.6315E−03 |
| 530 | 4601 | Entrez Gene | MAX interactor 1 | 654_at | Day 7 Peri/Day 0 Peri | 0.783 | 5.2959E−03 |
| | | | | 39072_at | Day 3 Peri/Day 0 Peri | 0.839 | 6.8595E−03 |
| 531 | 6293 | Entrez Gene | vacuolar protein sorting 52 (yeast) | 32658_at | Day 7 Peri/Day 0 Peri | 0.858 | 6.8088E−03 |
| 533 | 10067 | Entrez Gene | secretory carrier membrane protein 3 | 32799_at | Day 7 Peri/Day 0 Peri | 0.763 | 8.9346E−03 |
| 535 | 6203 | Entrez Gene | ribosomal protein S9 | 31511_at | Day 7 Peri/Day 0 Peri | 0.764 | 7.8708E−03 |
| 538 | 5036 | Entrez Gene | proliferation-associated 2G4, 38 kDa | 41600_at | Day 7 Peri/Day 0 Peri | 0.765 | 6.9426E−03 |
| 539 | 9249 | Entrez Gene | dehydrogenase/reductase (SDR family) | 40782_at | Day 3 Peri/Day 0 Peri | 0.765 | 6.4510E−03 |
| | | | member 3 | | | | |
| 540 | 1337 | Entrez Gene | cytochrome c oxidase subunit VIa | 41206_r_at | Day 7 Peri/Day 0 Peri | 0.765 | 1.7769E−03 |
| | | | polypeptide 1 | | | | |
| 543 | 203069 | Entrez Gene | R3H domain and coiled-coil containing 1 | 35156_at | Day 7 Peri/Day 0 Peri | 0.769 | 8.4083E−03 |
| 545 | 23294 | Entrez Gene | ankyrin repeat and sterile alpha motif domain | 40971_at | Day 3 Peri/Day 0 Peri | 0.796 | 3.4853E−03 |
| | | | containing 1 | | | | |
| 547 | 1937 | Entrez Gene | eukaryotic translation elongation factor 1 | 1676_s_at | Day 7 Peri/Day 0 Peri | 0.771 | 8.5875E−03 |
| | | | gamma | | | | |
| 549 | 147179 | Entrez Gene | WIRE protein | 40787_at | Day 3 Peri/Day 0 Peri | 0.772 | 8.6910E−04 |
| 558 | 3163 | Entrez Gene | heme oxygenase (decycling) 2 | 37916_at | Day 7 Peri/Day 3 Peri | 0.781 | 9.4119E−03 |
| 559 | 22864 | Entrez Gene | KIAA1002 protein | 41366_at | Day 3 Peri/Day 0 Peri | 0 781 | 8.7647E−03 |
| 562 | 9903 | Entrez Gene | kelch-like 21 (Drosophila) | 37230_at | Day 3 Peri/Day 0 Peri | 0.786 | 5.6275E−03 |
| | | | | 37230_at | Day 7 Peri/Day 0 Peri | 0.812 | 4.4808E−03 |
| 563 | 5236 | Entrez Gene | phosphoglucomutase 1 | 32210_at | Day 3 Peri/Day 0 Peri | 0.789 | 7.7233E−03 |
| 565 | 2975 | Entrez Gene | general transcription factor IIIC, polypeptide 1, | 35671_at | Day 7 Peri/Day 0 Peri | 0.791 | 3.1660E−03 |
| | | | alpha 220 kDa | | | | |
| 566 | 10956 | Entrez Gene | amplified in osteosarcoma | 36996_at | Day 7 Peri/Day 0 Peri | 0.791 | 9.0785E−03 |
| | | | | 36996_at | Day 3 Peri/Day 0 Peri | 0.818 | 6.2854E−03 |
| 569 | 527 | Entrez Gene | ATPase, H+ transporting, lysosomal 16 kDa, | 36994_at | Day 7 Peri/Day 0 Peri | 0.794 | 8.6116E−03 |
| | | | V0 subunit c | | | | |
| 570 | 8720 | Entrez Gene | membrane-bound transcription factor peptidase, | 36964_at | Day 3 Peri/Day 0 Peri | 0.795 | 9.8019E−03 |
| | | | site 1 | | | | |
| 572 | 286440 | Entrez Gene | hypothetical protein LOC286440 | 41655_at | Day 3 Peri/Day 0 Peri | 0.796 | 8.4134E−03 |
| 573 | 6136 | Entrez Gene | ribosomal protein L12 | 33668_at | Day 7 Peri/Day 0 Peri | 0.797 | 2.3409E−03 |
| 575 | 445 | Entrez Gene | argininosuccinate synthetase | 40541_at | Day 7 Peri/Day 3 Peri | 0.799 | 7.5623E−03 |
| 576 | 11224 | Entrez Gene | ribosomal protein L35 | 41765_at | Day 7 Peri/Day 0 Peri | 0.800 | 6.9298E−03 |
| 578 | 4779 | Entrez Gene | nuclear factor (erythroid-derived 2)-like 1 | 38439_at | Day 7 Peri/Day 0 Peri | 0.811 | 5.5209E−03 |
| 579 | 149603 | Entrez Gene | ring finger protein 187 | 39722_at | Day 3 Peri/Day 0 Peri | 0.812 | 7.5346E−03 |
| 580 | 8818 | Entrez Gene | dolichyl-phosphate mannosyltransferase | 38726_at | Day 7 Peri/Day 3 Peri | 0.814 | 3.7920E−03 |
| | | | polypeptide 2, regulatory subunit | | | | |
| 581 | 823 | Entrez Gene | calpain 1, (mu/l) large subunit | 33908_at | Day 7 Peri/Day 3 Peri | 0.815 | 9.2186E−03 |
| 583 | 6720 | Entrez Gene | sterol regulatory element binding transcription | 32135_at | Day 3 Peri/Day 0 Peri | 0.819 | 6.4366E−03 |
| | | | factor 1 | | | | |
| 584 | 23633 | Entrez Gene | Karyopherin alpha 6 (importin alpha 7) | 40275_at | Day 3 Peri/Day 0 Peri | 0.824 | 1.6981E−03 |
| | | | | 40275_at | Day 7 Peri/Day 0 Peri | 0.847 | 5.5244E−03 |
| 586 | 23481 | Entrez Gene | pescadillo homolog 1, containing BRCT | 41869_at | Day 7 Peri/Day 0 Peri | 0.831 | 9.3885E−03 |
| | | | domain (zebrafish) | | | | |

TABLE 14
Diagnostic Down

| | Public | | | | | | |
|---|---|---|---|---|---|---|---|
| Gene_ID | Identifier | Data Source | Gene Name | Probe_ID | Comparison | Fold Change | P value |
| 300 | 22883 | Entrez Gene | calsyntenin 1 | 41498_at | Day 3 Peri/Day 0 Peri | 0.777 | 3.9252E−03 |
| 458 | 10278 | Entrez Gene | embryonal Fyn-associated substrate | 33883_at | Day 7 Peri/Day 0 Peri | 0.706 | 2.0000E−05 |
| | | | | 33883_at | Day 3 Peri/Day 0 Peri | 0.718 | 6.1275E−04 |
| 476 | 949 | Entrez Gene | scavenger receptor class B, | 41200_at | Day 7 Peri/Day 0 Peri | 0.720 | 1.3768E−03 |
| | | | member 1 | 41200_at | Day 3 Peri/Day 0 Peri | 0.721 | 1.6250E−03 |

TABLE 15
Diagnostic Down

| | Public | | | | | Fold | |
|---|---|---|---|---|---|---|---|
| Gene ID | Identifier | Data Source | Gene Name | Probe_ID | Comparison | Change | P value |
| 24 | 2053 | Entrez Gene | epoxide hydrolase 2, cytoplasmic | 41473_at | Day 3 Peri/Day 0 Peri | 0.521 | 6.6949E−03 |
| 378 | 10014 | Entrez Gene | histone deacetylase 5 | 38810_at | Day 7 Peri/Day 0 Peri | 0.778 | 7.8739E−03 |
| | | | | 38810_at | Day 3 Peri/Day 0 Peri | 0.784 | 9.3572E−04 |
| 578 | 4779 | Entrez Gene | nuclear factor (erythroid- | 38439_at | Day 7 Peri/Day 0 Peri | 0.811 | 5.5209E−03 |
| | | | derived 2)-like 1 | | | | |

TABLE 16
Diagnostic Down

| | Public | | | | | | |
|---|---|---|---|---|---|---|---|
| Gene ID | Identifier | Data Source | Gene Name | Probe_ID | Comparison | Fold Change | P value |
| 102 | 6038 | Entrez Gene | ribonuclease, RNase A family, 4 | 32664_at | Day 3 Peri/Day 0 Peri | 0.636 | 5.8524E−04 |
| | | | | 32664_at | Day 7 Peri/Day 0 Peri | 0.691 | 3.6796E−03 |

TABLE 17
Diagnostic Down

| Gene | Public | | | | | Fold | |
|---|---|---|---|---|---|---|---|
| ID | Identifier | Data Source | Gene Name | Probe_ID | Comparison | Change | P value |
| 13 | 125 | Entrez Gene | alcohol dehydrogenase IB (class I), beta polypeptide | 35730_at | Day 0 Peri/Day 0 Intra | 0.252 | 5.3255E−03 |
| 21 | 3131 | Entrez Gene | Hepatic leukemia factor | 38627_at | Day 0 Peri/Day 0 Intra | 0.556 | 5.7849E−03 |
| 53 | 10443 | Entrez Gene | Hypothetical gene CG012 | 1532_g_at | Day 0 Peri/Day 0 Intra | 0.660 | 5.2680E−03 |
| 66 | 51601 | Entrez Gene | lipoyltransferase 1 | 37441_at | Day 0 Peri/Day 0 Intra | 0.432 | 3.4529E−03 |
| 90 | 6924 | Entrez Gene | Transcription elongation factor B (SIII), polypeptide 3 | 32049_f_at | Day 0 Peri/Day 0 Intra | 0.469 | 3.1906E−03 |
| | | | (110 kDa, elongin A) | | | | |
| 121 | 1158 | Entrez Gene | creatine kinase, muscle | 32486_at | Day 0 Peri/Day 0 Intra | 0.501 | 6.1425E−03 |
| 146 | 4968 | Entrez Gene | 8-oxoguanine DNA glycosylase | 38335_at | Day 0 Peri/Day 0 Intra | 0.759 | 8.2179E−03 |
| 157 | 26610 | Entrez Gene | elongation protein 4 homolog (S. cerevisiae) | 38347_at | Day 0 Peri/Day 0 Intra | 0.533 | 6.3391E−03 |
| 186 | 644 | Entrez Gene | biliverdin reductase A | 32618_at | Day 3 Peri/Day 3 Intra | 0.702 | 5.1159E−03 |
| 193 | 23107 | Entrez Gene | mitochondrial ribosomal protein S27 | 39377_at | Day 3 Peri/Day 3 Intra | 0.634 | 9.8004E−03 |
| 197 | 6477 | Entrez Gene | Seven in absentia homolog 1 (Drosophila) | 32161_at | Day 0 Peri/Day 0 Intra | 0.565 | 2.6625E−03 |
| 215 | 9936 | Entrez Gene | CD302 antigen | 34760_at | Day 0 Peri/Day 0 Intra | 0.580 | 5.4949E−04 |
| 218 | 128 | Entrez Gene | alcohol dehydrogenase 5 (class III), chl polypeptide | 37708_r_at | Day 0 Peri/Day 0 Intra | 0.600 | 6.4350E−03 |
| 220 | 7295 | Entrez Gene | thioredoxin | 36992_at | Day 3 Peri/Day 3 Intra | 0.673 | 4.8045E−03 |
| 224 | 831 | Entrez Gene | calpastatin | 41257_at | Day 0 Peri/Day 0 Intra | 0.700 | 3.4819E−03 |
| 250 | 4775 | Entrez Gene | nuclear factor of activated T-cells, cytoplasmic, | 40822_at | Day 0 Peri/Day 0 Intra | 0.746 | 7.0070E−03 |
| | | | calcineurin-dependent 3 | | | | |
| 254 | 26135 | Entrez Gene | SERPINE1 mRNA binding protein 1 | 40440_at | Day 3 Peri/Day 3 Intra | 0.727 | 9.5681E−03 |
| 262 | 353 | Entrez Gene | adenine phosphoribosyltransferase | 34310_at | Day 3 Peri/Day 3 Intra | 0.619 | 3.4883E−03 |
| 267 | 54665 | Entrez Gene | round spermatid basic protein 1 | 37828_at | Day 0 Peri/Day 0 Intra | 0.626 | 4.8045E−03 |
| 282 | 23392 | Entrez Gene | KIAA0368 | 34414_at | Day 0 Peri/Day 0 Intra | 0.644 | 5.3735E−03 |
| 290 | 25797 | Entrez Gene | glutaminyl-peptide cyclotransferase (glutaminyl | 35966_at | Day 0 Peri/Day 0 Intra | 0.663 | 6.8919E−03 |
| | | | cyclase) | | | | |
| 291 | 5092 | Entrez Gene | 6-pyruvoyl-tetrahydropterin synthase/dimerization | 34352_at | Day 3 Peri/Day 3 Intra | 0.704 | 1.5982E−03 |
| | | | cofactor of hepatocyte nuclear factor 1 alpha (TCF1) | | | | |
| 298 | 10289 | Entrez Gene | translation factor sui1 homolog | 33351_at | Day 0 Peri/Day 0 Intra | 0.624 | 5.2711E−04 |
| 316 | 8824 | Entrez Gene | carboxylesterase 2 (intestine, liver) | 40882_at | Day 7 Peri/Day 7 Intra | 0.635 | 9.8901E−03 |
| 341 | 9521 | Entrez Gene | eukaryotic translation elongation factor 1 epsilon 1 | 40587_s_at | Day 3 Peri/Day 3 Intra | 0.697 | 4.8656E−04 |
| 351 | 132556 | Entrez Gene | similar to Transcription factor BTF3 homolog 3 | 31519_f_at | Day 0 Peri/Day 0 Intra | 0.653 | 9.2891E−03 |
| 366 | 8192 | Entrez Gene | CipP caseinolytic peptidase, ATP-dependent, | 32528_at | Day 7 Peri/Day 7 Intra | 0.661 | 7.3475E−03 |
| | | | proteolytic subunit homolog (E. coli) | | | | |
| 370 | 3094 | Entrez Gene | histidine triad nucleotide binding protein 1 | 1009_at | Day 3 Peri/Day 3 Intra | 0.704 | 8.1604E−03 |
| 385 | 4694 | Entrez Gene | NADH dehydrogenase (ubiquinone) 1 alpha | 36169_at | Day 3 Peri/Day 3 Intra | 0.754 | 3.5482E−03 |
| | | | subcomplex, 1, 7.5 kDa | | | | |
| 403 | 9128 | Entrez Gene | PRP4 pre-mRNA processing factor 4 homolog (yeast) | 37936_at | Day 3 Peri/Day 3 Intra | 0.809 | 4.3656E−03 |
| 410 | 23075 | Entrez Gene | SWAP-70 protein | 31869_at | Day 0 Peri/Day 0 Intra | 0.684 | 5.9906E−03 |
| 413 | 6138 | Entrez Gene | ribosomal protein L15 | 32433_at | Day 0 Peri/Day 0 Intra | 0.686 | 4.8021E−03 |
| 417 | 9759 | Entrez Gene | histone deacetylase 4 | 38271_at | Day 0 Peri/Day 0 Intra | 0.776 | 6.2327E−03 |
| 418 | 4676 | Entrez Gene | nucleosome assembly protein 1-like 4 | 32575_at | Day 3 Peri/Day 3 Intra | 0.820 | 9.3028E−03 |
| 419 | 5425 | Entrez Gene | polymerase (DNA directed), delta 2, regulatory subunit | 1470_at | Day 3 Peri/Day 3 Intra | 0.689 | 5.7659E−03 |
| | | | 50 kDa | | | | |
| 439 | 2549 | Entrez Gene | GRB2-associated binding protein 1 | 33997_at | Day 0 Peri/Day 0 Intra | 0.731 | 4.0375E−03 |
| 442 | 23385 | Entrez Gene | nicastrin | 34835_at | Day 3 Peri/Day 3 Intra | 0.790 | 4.8084E−03 |
| 452 | 539 | Entrez Gene | ATP synthase, H+ transporting, mitochondrial F1 | 37029_at | Day 3 Peri/Day 3 Intra | 0.701 | 5.7342E−03 |
| | | | complex, O subunit (oligomycin sensitivity | | | | |
| | | | conferring protein) | | | | |
| 462 | 51635 | Entrez Gene | dehydrogenase/reductase (SDR family) member 7 | 39814_s_at | Day 3 Peri/Day 3 Intra | 0.740 | 6.8403E−03 |
| 470 | 3184 | Entrez Gene | heterogeneous nuclear ribonucleoprotein D (AU-rich | 38016_at | Day 0 Peri/Day 0 Intra | 0.714 | 4.2703E−03 |
| | | | element RNA binding protein 1, 37 kDa) | | | | |
| 480 | HG1614- | The Institute | — | 954_s_at | Day 7 Peri/Day 7 Intra | 0.747 | 1.0730E−03 |
| | HT161 | for | | | | | |
| | | Genomic | | | | | |
| | | Research | | | | | |
| 488 | 22934 | Entrez Gene | ribose 5-phosphate isomerase A (ribose 5-phosphate | 38036_at | Day 0 Peri/Day 0 Intra | 0.729 | 4.9732E−03 |
| | | | epimerase) | | | | |
| 490 | 11311 | Entrez Gene | vacuolar protein sorting 45A (yeast) | 35779_at | Day 0 Peri/Day 0 Intra | 0.731 | 7.1397E−03 |
| 511 | 9540 | Entrez Gene | tumor protein p53 inducible protein 3 | 36079_at | Day 3 Peri/Day 3 Intra | 0.746 | 5.1335E−03 |
| 527 | 9724 | Entrez Gene | UTP14, U3 small nucleolar ribonucleoprotein, | 39405_at | Day 0 Peri/Day 0 Intra | 0.760 | 3.9078E−03 |
| | | | homolog C (yeast) | | | | |
| 534 | 5437 | Entrez Gene | polymerase (RNA) II (DNA directed) polypeptide H | 35631_at | Day 3 Peri/Day 3 Intra | 0.772 | 5.4217E−03 |
| 541 | 23623 | Entrez Gene | RUN and SH3 domain containing 1 | 34264_at | Day 3 Peri/Day 3 Intra | 0.768 | 9.1280E−03 |
| 553 | 8480 | Entrez Gene | RAE1 RNA export 1 homolog (S. pombe) | 32758_g_at | Day 7 Peri/Day 7 Intra | 0.786 | 2.2519E−03 |
| 571 | 3151 | Entrez Gene | high-mobility group nucleosomal binding domain 2 | 41231_f_at | Day 3 Peri/Day 3 Intra | 0.827 | 4.3201E−03 |
| 585 | 7534 | Entrez Gene | tyrosine 3-monooxygenase/tryptophan | 1235_at | Day 3 Peri/Day 3 Intra | 0.853 | 7.0175E−03 |
| | | | 5-monooxygenase activation protein, | | | | |
| | | | zeta polypeptide | | | | |

TABLE 18
Diagnostic Down

| | Public | | | | | | |
|---|---|---|---|---|---|---|---|
| Gene ID | Identifier | Data Source | Gene Name | Probe_ID | Comparison | Fold Change | P value |
| 220 | 7295 | Entrez Gene | thioredoxin | 36992_at | Day 3 Peri/Day 3 Intra | 0.673 | 4.8045E−03 |

TABLE 19
Diagnostic Down

| | Public | | | | | | |
|---|---|---|---|---|---|---|---|
| Seq_ID | Identifier | Data Source | Gene Name | Probe_ID | Comparison | Fold Change | P value |
| 250 | 4775 | Entrez Gene | nuclear factor of activated T-cells, cytoplasmic, | 40822_at | Day 0 Peri/Day 0 Intra | 0.746 | 7.0070E−03 |
| | | | calcineurin-dependent 3 | | | | |
| 417 | 9759 | Entrez Gene | histone deacetylase 4 | 38271_at | Day 0 Peri/Day 0 Intra | 0.776 | 6.2327E−03 |
| 490 | 11311 | Entrez Gene | vacuolar protein sorting 45A (yeast) | 35779_at | Day 0 Peri/Day 0 Intra | 0.731 | 7.1397E−03 |

TABLE 20
Diagnostic Down

| Gene | Public | | | | | Fold | |
|---|---|---|---|---|---|---|---|
| ID | Identifier | Data Source | Gene Name | Probe_ID | Comparison | Change | P value |
| 1 | 4250 | Entrez Gene | secretoglobin, family 2A, member 2 | 36329_at | Day 0 Intra/Day 0 Control | 0.009 | 7.1701E−04 |
| | | | | 36329_at | Day 3 Intra/Day 3 Control | 0.017 | 3.3179E−04 |
| | | | | 36329_at | Day 7 Intra/Day 7 Control | 0.021 | 2.8985E−03 |
| | | | | 36329_at | Day 3 Intra/Day 3 Extra | 0.023 | 2.7212E−03 |
| 2 | 5304 | Entrez Gene | prolactin-induced protein | 41094_at | Day 0 Intra/Day 0 Control | 0.032 | 5.1676E−04 |
| | | | | 41094_at | Day 7 Intra/Day 7 Control | 0.076 | 9.5561E−03 |
| | | | | 41094_at | Day 3 Intra/Day 3 Control | 0.094 | 1.9791E−04 |
| 3 | 10647 | Entrez Gene | secretoglobin, family 1D, member 2 | 32880_at | Day 0 Intra/Day 0 Control | 0.033 | 4.4000E−05 |
| | | | | 32880_at | Day 7 Intra/Day 7 Control | 0.065 | 7.5456E−03 |
| | | | | 32880_at | Day 3 Intra/Day 3 Control | 0.075 | 8.2511E−04 |
| | | | | 32880_at | Day 3 Intra/Day 3 Extra | 0.102 | 5.4085E−03 |
| 4 | HG1763- | The Institute | — | 325_s_at | Day 3 Intra/Day 3 Extra | 0.038 | 6.4653E−03 |
| | HT178 | for | | 325_s_at | Day 7 Intra/Day 7 Control | 0.060 | 1.7598E−03 |
| | | Genomic | | 325_s_at | Day 3 Intra/Day 3 Control | 0.073 | 2.6304E−04 |
| | | Research | | 325_s_at | Day 0 Intra/Day 0 Control | 0.091 | 6.8631E−04 |
| 5 | 2167 | Entrez Gene | fatty acid binding protein 4, adipocyte | 38430_at | Day 7 Intra/Day 7 Extra | 0.053 | 1.3371E−03 |
| | | | | 38430_at | Day 7 Intra/Day 7 Control | 0.075 | 4.2884E−03 |
| 6 | 3283 | Entrez Gene | hydroxy-delta-5-steroid dehydrogenase, 3 | 35721_at | Day 0 Intra/Day 0 Peri | 0.062 | 4.4497E−03 |
| | | | beta- and steroid delta-isomerase 1 | | | | |
| 7 | 6288 | Entrez Gene | serum amyloid A1 | 33272_at | Day 3 Intra/Day 3 Extra | 0.069 | 6.8147E−03 |
| | | | | 33272_at | Day 7 Intra/Day 7 Extra | 0.117 | 4.3324E−03 |
| 8 | 9415 | Entrez Gene | fatty acid desaturase 2 | 32190_at | Day 0 Intra/Day 0 Peri | 0.081 | 5.6381E−04 |
| | | | | 32190_at | Day 7 Intra/Day 7 Peri | 0.201 | 7.6424E−03 |
| 9 | 247 | Entrez Gene | arachidonate 15-lipoxygenase, second type | 37430_at | Day 0 Intra/Day 0 Peri | 0.096 | 4.5492E−03 |
| 10 | 1690 | Entrez Gene | coagulation factor C homolog, cochlin | 34190_at | Day 3 Intra/Day 3 Extra | 0.129 | 1.6089E−03 |
| | | | (Limulus polyphemus) | | | | |
| 12 | HG371- | The Institute | — | 700_s_at | Day 0 Intra/Day 0 Peri | 0.143 | 4.4849E−03 |
| | HT2638 | for | | | | | |
| | | Genomic | | | | | |
| | | Research | | | | | |
| 13 | 125 | Entrez Gene | alcohol dehydrogenase IB (class I), beta | 35730_at | Day 3 Intra/Day 0 Intra | 0.162 | 9.2302E−03 |
| | | | polypeptide | | | | |
| 16 | L09190 | GenBank | Full-length cDNA clone CS0DI027YJ05 of | 37635_at | Day 3 Intra/Day 3 Peri | 0.206 | 2.8404E−03 |
| | | | Placenta Cot 25-normalized of Homo sapiens | | | | |
| | | | (human) | | | | |
| 18 | 7123 | Entrez Gene | C-type lectin domain family 3, member B | 36569_at | Day 3 Intra/Day 0 Intra | 0.243 | 7.7786E−03 |
| 19 | 10351 | Entrez Gene | ATP-binding cassette, sub-family A (ABC1), | 35717_at | Day 3 Intra/Day 0 Intra | 0.279 | 3.1166E−03 |
| | | | member 8 | 35717_at | Day 3 Intra/Day 3 Peri | 0.305 | 5.8783E−04 |
| 20 | AB011538 | GenBank | CDNA clone IMAGE: 5922621 | 35324_at | Day 3 Intra/Day 0 Intra | 0.280 | 3.8006E−03 |
| 21 | 3131 | Entrez Gene | Hepatic leukemia factor | 38627_at | Day 3 Intra/Day 0 Intra | 0.289 | 1.1838E−03 |
| | | | | 38627_at | Day 7 Intra/Day 0 Intra | 0.325 | 6.7101E−03 |
| 23 | 4211 | Entrez Gene | Meis1, myeloid ecotropic viral integration site | 40763_at | Day 3 Intra/Day 0 Intra | 0.324 | 4.5206E−03 |
| | | | 1 homolog (mouse) | | | | |
| 24 | 2053 | Entrez Gene | epoxide hydrolase 2, cytoplasmic | 41473_at | Day 3 Intra/Day 0 Intra | 0.327 | 3.5922E−03 |
| 25 | 4253 | Entrez Gene | CTAGE family, member 5 | 41615_at | Day 3 Intra/Day 0 Intra | 0.330 | 3.8948E−03 |
| 26 | 5271 | Entrez Gene | serpin peptidase inhibitor, clade B | 36312_at | Day 7 Intra/Day 7 Extra | 0.482 | 4.6083E−03 |
| | | | (ovalbumin), member 8 | | | | |
| 27 | 3119 | Entrez Gene | major histocompatibility complex, class II, | 36878_f_at | Day 0 Intra/Day 0 Control | 0.350 | 9.9683E−03 |
| | | | DQ bela 1 | | | | |
| 30 | 3885 | Entrez Gene | keratin, hair, acidic, 4 | 34012_at | Day 0 Intra/Day 0 Peri | 0.352 | 4.5590E−03 |
| 32 | 11075 | Entrez Gene | stathmin-like 2 | 38800_at | Day 3 Intra/Day 0 Intra | 0.356 | 5.6882E−03 |
| 33 | 3075 | Entrez Gene | complement factor H | 32250_at | Day 7 Intra/Day 0 Intra | 0.356 | 2.4648E−03 |
| 34 | 10458 | Entrez Gene | BAI1-associated protein 2 | 37760_at | Day 7 Intra/Day 3 Intra | 0.799 | 8.1747E−03 |
| 35 | 9086 | Entrez Gene | eukaryotic translation initiation factor 1A, Y- | 40097_at | Day 3 Intra/Day 0 Intra | 0.358 | 8.6154E−03 |
| | | | linked | | | | |
| 36 | 2273 | Entrez Gene | four and a half LIM domains 1 | 32542_at | Day 3 Intra/Day 0 Intra | 0.367 | 5.7500E−03 |
| 40 | 4223 | Entrez Gene | mesenchyme homeo box 2 (growth arrest- | 40398_s_at | Day 3 Intra/Day 0 Intra | 0.376 | 7.2046E−03 |
| | | | specific homeo box) | | | | |
| 41 | 3400 | Entrez Gene | inhibitor of DNA binding 4, dominant negative | 41536_at | Day 3 Intra/Day 0 Intra | 0.381 | 8.0900E−05 |
| | | | helix-loop-helix protein | 41536_at | Day 7 Intra/Day 0 Intra | 0.410 | 2.1354E−03 |
| | | | | 41536_at | Day 3 Intra/Day 3 Control | 0.488 | 2.0560E−03 |
| 43 | 8710 | Entrez Gene | serpin peptidase inhibitor, clade B | 35577_at | Day 0 Intra/Day 0 Control | 0.489 | 6.7433E−03 |
| | | | (ovalbumin), member 7 | 35577_at | Day 3 Intra/Day 3 Control | 0.566 | 9.3574E−03 |
| 44 | 2194 | Entrez Gene | fatty acid synthase | 38429_at | Day 0 Intra/Day 0 Peri | 0.386 | 9.3714E−03 |
| 47 | 406 | Entrez Gene | aryl hydrocarbon receptor nuclear | 36896_s_at | Day 3 Intra/Day 3 Control | 0.391 | 9.1974E−03 |
| | | | translocator-like | | | | |
| 48 | 9452 | Entrez Gene | integral membrane protein 2A | 40775_at | Day 3 Intra/Day 0 Intra | 0.392 | 3.3491E−03 |
| | | | | 40775_at | Day 7 Intra/Day 0 Intra | 0.400 | 2.0273E−03 |
| 49 | 63928 | Entrez Gene | hepatocellular carcinoma antigen gene 520 | 33007_at | Day 3 Intra/Day 3 Control | 0.466 | 7.7206E−03 |
| 51 | 5354 | Entrez Gene | proteolipid protein 1 (Pelizaeus-Merzbacher | 41158_at | Day 3 Intra/Day 0 Intra | 0.403 | 1.1357E−04 |
| | | | disease, spastic paraplegia 2, | 41158_at | Day 7 Intra/Day 0 Intra | 0.503 | 1.7500E−03 |
| | | | uncomplicated) | | | | |
| 54 | 224 | Entrez Gene | aldehyde dehydrogenase 3 family, member | 40409_at | Day 3 Intra/Day 3 Control | 0.410 | 5.0007E−03 |
| | | | A2 | 40409_at | Day 3 Intra/Day 0 Intra | 0.466 | 8.5061E−04 |
| 55 | 90634 | Entrez Gene | hypothetical gene CG018 | 34239_at | Day 3 Intra/Day 3 Control | 0.472 | 5.9625E−03 |
| 57 | 23242 | Entrez Gene | cordon-bleu homolog (mouse) | 35669_at | Day 3 Intra/Day 0 Intra | 0.414 | 5.3389E−03 |
| | | | | 35669_at | Day 3 Intra/Day 3 Control | 0.417 | 3.3977E−03 |
| | | | | 35669_at | Day 3 Intra/Day 3 Extra | 0.522 | 4.0595E−04 |
| 61 | M11119 | GenBank | — | 38850_at | Day 3 Intra/Day 0 Intra | 0.428 | 8.8745E−03 |
| 62 | 9687 | Entrez Gene | GREB1 protein | 38875_r_at | Day 7 Intra/Day 0 Intra | 0.428 | 1.0961E−04 |
| 63 | 51097 | Entrez Gene | saccharopine dehydrogenase (putative) | 34862_at | Day 7 Intra/Day 0 Intra | 0.680 | 6.5823E−03 |
| 67 | 9145 | Entrez Gene | synaptogyrin 1 | 35354_at | Day 7 Intra/Day 0 Intra | 0.432 | 1.9735E−03 |
| | | | | 35354_at | Day 3 Intra/Day 0 Intra | 0.469 | 5.7597E−03 |
| 68 | 1117 | Entrez Gene | chitinase 3-like 2 | 31891_at | Day 0 Intra/Day 0 Peri | 0.432 | 5.3219E−04 |
| 69 | 13 | Entrez Gene | arylacetamide deacetylase (esterase) | 36512_at | Day 3 Intra/Day 3 Extra | 0.434 | 1.5905E−04 |
| 73 | 57798 | Entrez Gene | GATA zinc finger domain containing 1 | 34195_at | Day 3 Intra/Day 3 Peri | 0.441 | 6.2766E−04 |
| 75 | 26 | Entrez Gene | amiloride binding protein 1 (amine oxidase | 37186_s_at | Day 3 Intra/Day 3 Extra | 0.443 | 5.1668E−03 |
| | | | (copper-containing)) | | | | |
| 76 | AL080082 | GenBank | MRNA; cDNA DKFZp564G1162 (from clone | 35581_at | Day 3 Intra/Day 0 Intra | 0.444 | 9.2254E−03 |
| | | | DKFZp564G1162) | | | | |
| 79 | 56288 | Entrez Gene | par-3 partitioning defective 3 homolog | 40973_at | Day 3 Intra/Day 3 Control | 0.533 | 5.0405E−03 |
| | | | (C. elegans) | | | | |
| 80 | 26154 | Entrez Gene | ATP-binding cassette, sub-family A (ABC1), | 31754_at | Day 3 Intra/Day 3 Extra | 0.557 | 5.7474E−03 |
| | | | member 12 | | | | |
| 82 | 1410 | Entrez Gene | crystallin, alpha B | 32243_g_at | Day 3 Intra/Day 3 Extra | 0.468 | 5.9480E−03 |
| 84 | 10733 | Entrez Gene | polo-like kinase 4 (Drosophila) | 975_at | Day 7 Intra/Day 7 Extra | 0.455 | 5.4588E−03 |
| 85 | 8848 | Entrez Gene | TSC22 domain family, member 1 | 39032_at | Day 3 Intra/Day 0 Intra | 0.455 | 1.8273E−03 |
| 87 | 5264 | Entrez Gene | phytanoyl-CoA hydroxylase (Refsum | 32724_at | Day 3 Intra/Day 0 Intra | 0.465 | 4.1244E−03 |
| | | | disease) | | | | |
| 88 | 10529 | Entrez Gene | nebulette | 34873_at | Day 3 Intra/Day 3 Control | 0.466 | 5.5136E−03 |
| 89 | 60481 | Entrez Gene | ELOVL family member 5, elongation of long | 33821_at | Day 0 Intra/Day 0 Peri | 0.468 | 8.7794E−03 |
| | | | chain fatty acids (FEN1/Elo2, SUR4/Elo3- | | | | |
| | | | like, yeast) | | | | |
| 91 | 10248 | Entrez Gene | processing of precursor 7, ribonuclease P | 32213_at | Day 7 Intra/Day 3 Intra | 0.470 | 9.0083E−03 |
| | | | subunit (S. cerevisiae) | | | | |
| 92 | 1545 | Entrez Gene | cytochrome P450, family 1, subfamily B, | 40071_at | Day 7 Intra/Day 7 Peri | 0.471 | 9.8480E−03 |
| | | | polypeptide 1 | | | | |
| 93 | 1152 | Entrez Gene | creatine kinase, brain | 40863_r_at | Day 7 Intra/Day 7 Control | 0.472 | 4.0142E−03 |
| 94 | 10450 | Entrez Gene | peptidylprolyl isomerase E (cyclophilin E) | 34365_at | Day 3 Intra/Day 3 Control | 0.518 | 4.1270E−03 |
| 96 | 10090 | Entrez Gene | uronyl-2-sulfotransferase | 41859_at | Day 3 Intra/Day 0 Intra | 0.474 | 1.8927E−03 |
| | | | | 41859_at | Day 7 Intra/Day 0 Intra | 0.521 | 1.4198E−04 |
| 98 | 352961 | Entrez Gene | MHC class I mRNA fragment 3.8-1 | 34934_at | Day 3 Intra/Day 0 Intra | 0.479 | 6.5297E−03 |
| 99 | 10314 | Entrez Gene | LanC lantibiotic synthetase component C-like | 39441_at | Day 0 Intra/Day 0 Control | 0.488 | 6.0119E−04 |
| | | | 1 (bacterial) | | | | |
| 100 | 10628 | Entrez Gene | thioredoxin interacting protein | 31508_at | Day 3 Intra/Day 0 Intra | 0.482 | 9.0159E−03 |
| 102 | 6038 | Entrez Gene | ribonuclease, RNase A family, 4 | 32664_at | Day 3 Intra/Day 0 Intra | 0.483 | 2.7472E−03 |
| | | | | 32664_at | Day 7 Intra/Day 0 Intra | 0.569 | 4.9291E−04 |
| 104 | 6414 | Entrez Gene | selenoprotein P, plasma, 1 | 34363_at | Day 3 Intra/Day 0 Intra | 0.488 | 6.0330E−03 |
| 105 | 9987 | Entrez Gene | Heterogeneous nuclear ribonucleoprotein D- | 32393_s_at | Day 3 Intra/Day 3 Peri | 0.489 | 1.9407E−03 |
| | | | like | | | | |
| 107 | 4163 | Entrez Gene | mutated in colorectal cancers | 35561_at | Day 0 Intra/Day 0 Peri | 0.489 | 6.2800E−05 |
| 111 | 2947 | Entrez Gene | glutathione S-transferase M3 (brain) | 1120_at | Day 7 Intra/Day 0 Intra | 0.494 | 7.1503E−03 |
| 114 | 8864 | Entrez Gene | period homolog 2 (Drosophila) | 35008_at | Day 3 Intra/Day 0 Intra | 0.497 | 4.3990E−03 |
| 117 | 104 | Entrez Gene | adenosine deaminase, RNA-specific, B1 | 38748_at | Day 3 Intra/Day 0 Intra | 0.499 | 8.5077E−04 |
| | | | (RED1 homolog rat) | 38748_at | Day 7 Intra/Day 0 Intra | 0.541 | 1.9004E−03 |
| 119 | 6662 | Entrez Gene | SRY (sex determining region Y)-box 9 | 33436_at | Day 0 Intra/Day 0 Peri | 0.521 | 8.5777E−03 |
| | | | (campomelic dysplasia, autosomal sex- | | | | |
| | | | reversal) | | | | |
| 122 | 9831 | Entrez Gene | zinc finger protein 623 | 39954_r_at | Day 3 Intra/Day 0 Intra | 0.505 | 3.3609E−03 |
| 123 | 1573 | Entrez Gene | cytochrome P450, family 2, subfamily J, | 501_g_t | Day 3 Intra/Day 3 Control | 0.529 | 4.2791E−03 |
| | | | polypeptide 2 | | | | |
| 124 | 22982 | Entrez Gene | KIAA0934 | 33408_at | Day 3 Intra/Day 0 Intra | 0.506 | 3.1621E−03 |
| 125 | 51710 | Entrez Gene | zinc finger protein 44 (KOX 7) | 35409_r_at | Day 3 Intra/Day 3 Peri | 0.507 | 5.1572E−03 |
| 126 | 2533 | Entrez Gene | FYN binding protein (FYB-120/130) | 41819_at | Day 3 Intra/Day 3 Peri | 0.507 | 3.0115E−03 |
| 127 | 9857 | Entrez Gene | centrosome-associated protein 350 | 33805_at | Day 3 Intra/Day 3 Peri | 0.508 | 4.3400E−05 |
| 128 | 9891 | Entrez Gene | NUAK family, SNF1-like kinase, 1 | 33787_at | Day 3 Intra/Day 0 Intra | 0.508 | 1.4528E−03 |
| 131 | 23051 | Entrez Gene | zinc fingers and homeoboxes 3 | 40461_at | Day 3 Intra/Day 0 Intra | 0.513 | 6.7006E−04 |
| | | | | 40461_at | Day 7 Intra/Day 0 Intra | 0.609 | 6.3269E−03 |
| 133 | 3422 | Entrez Gene | isopentenyl-diphosphate delta Isomerase 1 | 36985_at | Day 7 Intra/Day 7 Extra | 0.515 | 5.4407E−03 |
| 134 | 7728 | Entrez Gene | zinc finger protein 175 | 37500_at | Day 7 Intra/Day 7 Peri | 0.515 | 4.1955E−03 |
| 135 | 36 | Entrez Gene | acyl-Coenzyme A dehydrogenase, | 40673_at | Day 3 Intra/Day 3 Control | 0.515 | 1.3679E−03 |
| | | | short/branched chain | | | | |
| 139 | 10965 | Entrez Gene | acyl-CoA thioesterase 2 | 36625_at | Day 7 Intra/Day 7 Control | 0.518 | 7.3351E−04 |
| 140 | 10284 | Entrez Gene | Sin3-associated polypeptide, 18 kDa | 41277_at | Day 3 Intra/Day 0 Intra | 0.520 | 2.4318E−03 |
| 143 | 7073 | Entrez Gene | TIA1 cytotoxic granule-associated RNA | 41761_at | Day 3 Intra/Day 0 Intra | 0.521 | 4.5836E−03 |
| | | | binding protein-like 1 | | | | |
| 145 | 8853 | Entrez Gene | development and differentiation enhancing | 39410_at | Day 7 Intra/Day 0 Intra | 0.521 | 2.9990E−03 |
| | | | factor 2 | | | | |
| 146 | 4968 | Entrez Gene | 8-oxoguanine DNA glycosylase | 38335_at | Day 3 Intra/Day 0 Intra | 0.522 | 7.5868E−04 |
| | | | | 38335_at | Day 7 Intra/Day 0 Intra | 0.626 | 6.6349E−03 |
| 147 | 2110 | Entrez Gene | electron-transferring-flavoprotein | 33494_at | Day 7 Intra/Day 7 Extra | 0.523 | 1.1689E−03 |
| | | | dehydrogenase | | | | |
| 151 | 55187 | Entrez Gene | vacuolar protein sorting 13D (yeast) | 32743_at | Day 3 Intra/Day 0 Intra | 0.527 | 2.5585E−03 |
| 153 | 9140 | Entrez Gene | ATG12 autophagy related 12 homolog | 32720_at | Day 3 Intra/Day 3 Peri | 0.530 | 2.7861E−04 |
| | | | (S. cerevisiae) | | | | |
| 155 | 23506 | Entrez Gene | KIAA0240 | 38892_at | Day 3 Intra/Day 0 Intra | 0.531 | 5.3954E−03 |
| 158 | 6595 | Entrez Gene | SWI/SNF related, matrix associated, actin | 40961_at | Day 3 Intra/Day 0 Intra | 0.533 | 6.2437E−03 |
| | | | dependent regulator of chromatin, subfamily | | | | |
| | | | a, member 2 | | | | |
| 159 | 5110 | Entrez Gene | Protein-L-isoaspartate (D-aspartate) O- | 37737_at | Day 7 Intra/Day 7 Extra | 0.534 | 2.8151E−03 |
| | | | methyltransferase | | | | |
| 161 | 201229 | Entrez Gene | hypothetical protein LOC201229 | 39557_at | Day 3 Intra/Day 0 Intra | 0.535 | 2.3943E−03 |
| 162 | 22909 | Entrez Gene | KIAA1018 protein | 36458_at | Day 3 Intra/Day 0 Intra | 0.536 | 6.1234E−03 |
| 163 | 51099 | Entrez Gene | Abhydrolase domain containing 5 | 33309_at | Day 3 Intra/Day 3 Peri | 0.536 | 9.8735E−03 |
| 164 | 114882 | Entrez Gene | oxysterol binding protein-like 8 | 41438_at | Day 3 Intra/Day 3 Peri | 0.536 | 3.0621E−03 |
| 166 | 10653 | Entrez Gene | serine peptidase inhibitor, Kunitz type, 2 | 34348_at | Day 7 Intra/Day 7 Extra | 0.699 | 5.3002E−03 |
| 168 | 399563 | Entrez Gene | hypothetical protein FLJ43806 | 37151_at | Day 7 Intra/Day 0 Intra | 0.542 | 7.7014E−03 |
| 171 | 1662 | Entrez Gene | DEAD (Asp-Glu-Ala-Asp) box polypeptide 10 | 831_at | Day 7 Intra/Day 7 Extra | 0.544 | 5.2360E−03 |
| 172 | 5130 | Entrez Gene | phosphate cytidylyltransferase 1, choline, | 39285_at | Day 0 Intra/Day 0 Peri | 0.545 | 2.3355E−03 |
| | | | alpha | | | | |
| 174 | 26235 | Entrez Gene | F-box and leucine-rich repeat protein 4 | 39987_at | Day 7 Intra/Day 7 Peri | 0.546 | 3.2958E−03 |
| 176 | 202 | Entrez Gene | absent in melanoma 1 | 32112_s_at | Day 3 Intra/Day 3 Extra | 0.617 | 9.6499E−03 |
| 177 | 9774 | Entrez Gene | BCL2-associated transcription factor 1 | 38050_at | Day 7 Intra/Day 7 Extra | 0.547 | 4.7061E−03 |
| 181 | 4232 | Entrez Gene | mesoderm specific transcript homolog | 37749_at | Day 7 Intra/Day 7 Extra | 0.553 | 9.8929E−04 |
| | | | (mouse) | | | | |
| 182 | 2145 | Entrez Gene | enhancer of zeste homolog 1 (Drosophila) | 32259_at | Day 7 Intra/Day 0 Intra | 0.554 | 8.0450E−03 |
| 184 | U73737 | GenBank | — | 1017_at | Day 3 Intra/Day 3 Peri | 0.556 | 4.6650E−03 |
| 189 | 10300 | Entrez Gene | katanin p80 (WD repeat containing) subunit | 40976_at | Day 3 Intra/Day 3 Extra | 0.559 | 9.1699E−03 |
| | | | B1 | | | | |
| 251 | 8776 | Entrez Gene | myotubularin related protein 1 | 34654_at | Day 3 Intra/Day 3 Extra | 0.598 | 1.6699E−03 |
| | | | | 34654_at | Day 3 Intra/Day 3 Control | 0.631 | 5.7609E−03 |
| 253 | AL080234 | GenBank | Full-length cDNA clone CS0DM001YA04 of | 38070_at | Day 7 Intra/Day 0 Intra | 0.598 | 4.0826E−03 |
| | | | Fetal liver of Homo sapiens (human) | | | | |
| 255 | 23234 | Entrez Gene | DnaJ (Hsp40) homolog, subfamily C, | 41569_at | Day 7 Intra/Day 7 Extra | 0.615 | 8.2268E−03 |
| | | | member 9 | | | | |
| 256 | 94239 | Entrez Gene | H2A histone family, member V | 39046_at | Day 3 Intra/Day 0 Intra | 0.600 | 7.9321E−03 |
| | | | | 39046_at | Day 7 Intra/Day 0 Intra | 0.650 | 5.0612E−03 |
| 257 | AI557912 | GenBank | translocase of outer mitochondrial membrane | 39025_at | Day 7 Intra/Day 0 Intra | 0.601 | 3.8343E−03 |
| | | | 7 homolog (yeast) /// hypothetical protein | 39025_at | Day 7 Intra/Day 7 Control | 0.727 | 5.2071E−03 |
| | | | LOC201725 | | | | |
| 261 | 6651 | Entrez Gene | SON DNA binding protein | 39096_at | Day 7 Intra/Day 0 Intra | 0.607 | 3.4000E−03 |
| | | | | 39096_at | Day 7 Intra/Day 7 Peri | 0.641 | 4.2992E−03 |
| | | | | 39096_at | Day 7 Intra/Day 3 Intra | 0.710 | 4.2168E−03 |
| 263 | 6427 | Entrez Gene | splicing factor, arginine/serine-rich 2 | 36111_s_at | Day 7 Intra/Day 7 Extra | 0.665 | 1.5173E−04 |
| | | | | 36111_s_at | Day 7 Intra/Day 3 Intra | 0.709 | 6.0916E−03 |
| 264 | 221749 | Entrez Gene | chromosome 6 open reading frame 145 | 34246_at | Day 3 Intra/Day 0 Intra | 0.608 | 3.6829E−03 |
| 267 | 54665 | Entrez Gene | round spermatid basic protein 1 | 37828_at | Day 3 Intra/Day 0 Intra | 0.610 | 7.8572E−04 |
| 270 | 3301 | Entrez Gene | DnaJ (Hsp40) homolog, subfamily A, | 39118_at | Day 7 Intra/Day 7 Extra | 0.611 | 5.7425E−03 |
| | | | member 1 | | | | |
| 272 | 22826 | Entrez Gene | DnaJ (Hsp40) homolog, subfamily C, | 36156_at | Day 0 Intra/Day 0 Control | 0.612 | 5.7794E−03 |
| | | | member 8 | | | | |
| 273 | 23090 | Entrez Gene | zinc finger protein 423 | 34950_at | Day 7 Intra/Day 7 Peri | 0.623 | 7.6235E−03 |
| 274 | 64112 | Entrez Gene | modulator of apoptosis 1 | 34767_at | Day 3 Intra/Day 0 Intra | 0.614 | 4.4658E−03 |
| 275 | 10497 | Entrez Gene | unc-13 homolog B (C. elegans) | 40087_at | Day 0 Intra/Day 0 Control | 0.769 | 3.3634E−03 |
| 282 | 23392 | Entrez Gene | KIAA0368 | 34414_at | Day 7 Intra/Day 0 Intra | 0.616 | 1.2883E−03 |
| 283 | 5934 | Entrez Gene | retinoblastoma-like 2 (p130) | 32597_at | Day 3 Intra/Day 0 Intra | 0.616 | 8.7510E−03 |
| 284 | 4781 | Entrez Gene | Nuclear factor I/B | 41229_at | Day 3 Intra/Day 0 Intra | 0.616 | 3.0477E−03 |
| 288 | 3418 | Entrez Gene | isocitrate dehydrogenase 2 (NADP+), | 32332_at | Day 0 Intra/Day 0 Peri | 0.617 | 1.2980E−03 |
| | | | mitochondrial | | | | |
| 289 | 9 | Entrez Gene | N-acetyltransferase 1 (arylamine N- | 38187_at | Day 7 Intra/Day 7 Extra | 0.619 | 5.7524E−03 |
| | | | acetyltransferase) | | | | |
| 290 | 25797 | Entrez Gene | glutaminyl-peptide cyclotransferase | 35966_at | Day 3 Intra/Day 0 Intra | 0.619 | 4.9522E−03 |
| | | | (glutaminyl cyclase) | | | | |
| 297 | 8888 | Entrez Gene | MCM3 minichromosome maintenance | 40469_at | Day 7 Intra/Day 7 Extra | 0.624 | 4.8160E−03 |
| | | | deficient 3 (S. cerevisiae) associated protein | | | | |
| 298 | 10289 | Entrez Gene | translation factor sui1 homolog | 33351_at | Day 3 Intra/Day 0 Intra | 0.673 | 1.7268E−03 |
| 302 | 24139 | Entrez Gene | echinoderm microtubule associated protein | 41328_s_at | Day 0 Intra/Day 0 Peri | 0.627 | 4.4037E−03 |
| | | | like 2 | | | | |
| 305 | 23351 | Entrez Gene | KIAA0323 | 32592_at | Day 0 Intra/Day 0 Control | 0.679 | 8.2136E−03 |
| 310 | 22903 | Entrez Gene | BTB (POZ) domain containing 3 | 37755_at | Day 3 Intra/Day 0 Intra | 0.631 | 9.7703E−03 |
| 311 | 4681 | Entrez Gene | neuroblastoma, suppression of tumorigenicity 1 | 37005_at | Day 0 Intra/Day 0 Peri | 0.631 | 3.2813E−03 |
| 312 | 10301 | Entrez Gene | deleted in lymphocytic leukemia, 1 | 33791_at | Day 7 Intra/Day 0 Intra | 0.631 | 8.9965E−03 |
| | | | | 33791_at | Day 3 Intra/Day 0 Intra | 0.726 | 6.7457E−03 |
| 313 | AF052138 | GenBank | FLJ35348 /// Bromodomain containing 3 | 41841_at | Day 3 Intra/Day 0 Intra | 0.632 | 4.2490E−03 |
| 315 | 8930 | Entrez Gene | methyl-CpG binding domain protein 4 | 34386_at | Day 7 Intra/Day 7 Extra | 0.634 | 3.7388E−03 |
| 319 | 1428 | Entrez Gene | crystallin, mu | 38285_at | Day 3 Intra/Day 0 Intra | 0.636 | 1.2448E−03 |
| 320 | 10614 | Entrez Gene | Hexamethylene bis-acetamide inducible 1 | 40220_at | Day 3 Intra/Day 0 Intra | 0.636 | 3.7740E−03 |
| 324 | 1340 | Entrez Gene | cytochrome c oxidase subunit Vib | 40872_at | Day 3 Intra/Day 3 Extra | 0.698 | 6.3894E−03 |
| | | | polypeptide 1 (ubiquitous) | | | | |
| 329 | 4026 | Entrez Gene | LIM domain containing preferred | 41195_at | Day 3 Intra/Day 3 Peri | 0.639 | 3.3595E−03 |
| | | | translocation partner in lipoma | | | | |
| 334 | 6230 | Entrez Gene | ribosomal protein S25 | 31573_at | Day 7 Intra/Day 0 Intra | 0.643 | 9.4033E−03 |
| 337 | 27067 | Entrez Gene | staufen, RNA binding protein, homolog 2 | 38341_at | Day 3 Intra/Day 3 Extra | 0.645 | 8.2036E−03 |
| | | | (Drosophila) | | | | |
| 341 | 9521 | Entrez Gene | eukaryotic translation elongation factor 1 | 40587_s_at | Day 7 Intra/Day 3 Intra | 0.647 | 1.9928E−03 |
| | | | epsilon 1 | | | | |
| 342 | 1054 | Entrez Gene | CCAAT/enhancer binding protein (C/EBP), | 39219_at | Day 7 Intra/Day 7 Extra | 0.676 | 1.0916E−03 |
| | | | gamma | | | | |
| 352 | 8732 | Entrez Gene | RNA guanylyltransferase and 5′-phosphatase | 35202_at | Day 7 Intra/Day 7 Extra | 0.654 | 1.2688E−03 |
| 356 | 51567 | Entrez Gene | TRAF and TNF receptor associated protein | 34825_at | Day 3 Intra/Day 3 Extra | 0.655 | 3.0500E−05 |
| 360 | 3298 | Entrez Gene | heat shock transcription factor 2 | 202_at | Day 7 Intra/Day 0 Intra | 0.658 | 7.1609E−03 |
| 361 | 4677 | Entrez Gene | asparaginyl-tRNA synthetase | 41241_at | Day 0 Intra/Day 0 Control | 0.658 | 3.0519E−03 |
| 363 | 23384 | Entrez Gene | KIAA0376 protein | 34837_at | Day 3 Intra/Day 0 Intra | 0.659 | 2.3071E−03 |
| 365 | 6635 | Entrez Gene | small nuclear ribonucleoprotein polypeptide E | 38679_g_at | Day 7 Intra/Day 7 Extra | 0.661 | 3.7759E−04 |
| | | | | 38679_g_at | Day 7 Intra/Day 3 Intra | 0.697 | 1.8800E−05 |
| 369 | 7004 | Entrez Gene | TEA domain family member 4 | 41037_at | Day 7 Intra/Day 7 Extra | 0.710 | 7.7243E−03 |
| | | | | 41037_at | Day 0 Intra/Day 0 Peri | 0.755 | 4.0427E−03 |
| 371 | 4832 | Entrez Gene | non-metastatic cells 3, protein expressed in | 197_at | Day 3 Intra/Day 3 Control | 0.663 | 6.9663E−03 |
| 372 | 10557 | Entrez Gene | ribonuclease P/MRP 38 kDa subunit | 41040_at | Day 7 Intra/Day 7 Extra | 0.667 | 4.5174E−03 |
| 373 | 26156 | Entrez Gene | ribosomal L1 domain containing 1 | 39418_at | Day 7 Intra/Day 3 Intra | 0.691 | 5.6288E−03 |
| 376 | 26053 | Entrez Gene | autism susceptibility candidate 2 | 35648_at | Day 3 Intra/Day 0 Intra | 0.667 | 3.9622E−03 |
| 377 | 56339 | Entrez Gene | Methyltransferase like 3 | 32244_at | Day 3 Intra/Day 0 Intra | 0.667 | 8.9345E−04 |
| | | | | 32244_at | Day 3 Intra/Day 3 Extra | 0.726 | 7.6780E−03 |
| 382 | 10038 | Entrez Gene | poly (ADP-ribose) polymerase family, | 34756_g_at | Day 7 Intra/Day 7 Extra | 0.669 | 7.1429E−03 |
| | | | member 2 | | | | |
| 386 | 10904 | Entrez Gene | bladder cancer associated protein | 35267_g_at | Day 7 Intra/Day 0 Intra | 0.672 | 2.1451E−03 |
| 387 | 7411 | Entrez Gene | von Hippel-Lindau binding protein 1 | 171_at | Day 7 Intra/Day 7 Extra | 0.672 | 7.7370E−03 |
| 390 | 1349 | Entrez Gene | cytochrome c oxidase subunit VIIb | 36687_at | Day 7 Intra/Day 3 Intra | 0.673 | 1.8885E−03 |
| 391 | 54462 | Entrez Gene | KIAA1128 | 37617_at | Day 3 Intra/Day 0 Intra | 0.674 | 5.5416E−03 |
| | | | | 37617_at | Day 3 Intra/Day 3 Control | 0.696 | 9.3133E−03 |
| 392 | 80003 | Entrez Gene | pecanex-like 2 (Drosophila) | 39650_s_at | Day 3 Intra/Day 0 Intra | 0.674 | 6.1109E−03 |
| 393 | 25972 | Entrez Gene | unc-50 homolog (C. elegans) | 39442_at | Day 7 Intra/Day 7 Extra | 0.675 | 9.6835E−03 |
| 394 | 64795 | Entrez Gene | hypothetical protein FLJ13910 | 36580_at | Day 3 Intra/Day 0 Intra | 0.676 | 3.2610E−03 |
| 397 | 7743 | Entrez Gene | zinc finger protein 189 | 36072_at | Day 3 Intra/Day 0 Intra | 0.678 | 3.7246E−03 |
| 399 | 50813 | Entrez Gene | COP6 constitutive photomorphogenic | 34404_at | Day 0 Intra/Day 0 Peri | 0.681 | 4.7805E−03 |
| | | | homolog subunit 7A (Arabidopsis) | | | | |
| 400 | 3156 | Entrez Gene | 3-hydroxy-3-methylglutaryl-Coenzyme A | 39328_at | Day 3 Intra/Day 3 Extra | 0.681 | 6.1525E−03 |
| | | | reductase | | | | |
| 405 | 2961 | Entrez Gene | general transcription factor IIE, polypeptide 2, | 37295_at | Day 7 Intra/Day 7 Extra | 0.796 | 1.4862E−03 |
| | | | beta 34 kDa | | | | |
| 409 | 4170 | Entrez Gene | myeloid cell leukemia sequence 1 (BCL2- | 277_at | Day 3 Intra/Day 3 Extra | 0.684 | 9.6905E−03 |
| | | | related) | | | | |
| 414 | 23112 | Entrez Gene | trinucleotide repeat containing 6B | 37487_at | Day 3 Intra/Day 3 Peri | 0.688 | 3.9849E−03 |
| 417 | 9759 | Entrez Gene | histone deacetylase 4 | 38271_at | Day 3 Intra/Day 0 Intra | 0.689 | 4.9833E−03 |
| 418 | 4676 | Entrez Gene | nucleosome assembly protein 1-like 4 | 32575_at | Day 7 Intra/Day 7 Extra | 0.790 | 4.9890E−03 |
| 422 | 1749 | Entrez Gene | distal-less homeo box 5 | 41087_at | Day 3 Intra/Day 0 Intra | 0.689 | 3.6526E−03 |
| 427 | 22794 | Entrez Gene | cancer susceptibility candidate 3 | 38437_at | Day 7 Intra/Day 0 Intra | 0.692 | 2.7057E−03 |
| | | | | 38437_at | Day 3 Intra/Day 0 Intra | 0.742 | 4.1247E−03 |
| 428 | 322 | Entrez Gene | amyloid beta (A4) precursor protein binding, | 1101_at | Day 7 Intra/Day 0 Intra | 0.692 | 1.6594E−03 |
| | | | family B, member 1 (Fe65) | | | | |
| 430 | 6637 | Entrez Gene | small nuclear ribonucleoprotein polypeptide G | 37337_at | Day 7 Intra/Day 3 Intra | 0.693 | 3.9690E−03 |
| 435 | 889 | Entrez Gene | KRIT1, ankyrin repeat containing | 34031_i_at | Day 3 Intra/Day 3 Peri | 0.694 | 4.9207E−03 |
| 439 | 2549 | Entrez Gene | GRB2-associated binding protein 1 | 33997_at | Day 7 Intra/Day 0 Intra | 0.695 | 2.4091E−04 |
| | | | | 33997_at | Day 3 Intra/Day 0 Intra | 0.710 | 2.0825E−03 |
| 443 | 23125 | Entrez Gene | calmodulin binding transcription activator 2 | 41421_at | Day 7 Intra/Day 0 Intra | 0.696 | 8.6760E−03 |
| 444 | 2926 | Entrez Gene | G-rich RNA sequence binding factor 1 | 32595_at | Day 7 Intra/Day 7 Extra | 0.696 | 6.0979E−03 |
| 447 | 22849 | Entrez Gene | cytoplasmic polyadenylation element binding | 34931_at | Day 3 Intra/Day 0 Intra | 0.697 | 6.8691E−03 |
| | | | protein 3 | | | | |
| 448 | 10106 | Entrez Gene | CTD (carboxy-terminal domain, RNA | 41202_s_at | Day 3 Intra/Day 0 Intra | 0.698 | 3.5449E−03 |
| | | | polymerase II, polypeptide A) small | | | | |
| | | | phosphatase 2 | | | | |
| 450 | 10329 | Entrez Gene | transmembrane protein 5 | 37445_at | Day 3 Intra/Day 3 Extra | 0.700 | 2.4850E−03 |
| 451 | 26959 | Entrez Gene | HMG-box transcription factor 1 | 39809_at | Day 3 Intra/Day 0 Intra | 0.701 | 9.4669E−03 |
| 452 | 539 | Entrez Gene | ATP synthase, H+ transporting, mitochondrial | 37029_at | Day 7 Intra/Day 7 Control | 0.784 | 7.2352E−03 |
| | | | F1 complex, O subunit (oligomycin sensitivity | | | | |
| | | | conferring protein) | | | | |
| 453 | 10542 | Entrez Gene | hepatitis B virus x interacting protein | 38054_at | Day 7 Intra/Day 7 Extra | 0.782 | 3.3831E−03 |
| 454 | 8624 | Entrez Gene | Down syndrome critical region gene 2 | 36088_at | Day 7 Intra/Day 3 Intra | 0.704 | 8.0554E−03 |
| 456 | 5431 | Entrez Gene | polymerase (RNA) II (DNA directed) | 39746_at | Day 7 Intra/Day 7 Extra | 0.705 | 2.3347E−03 |
| | | | polypeptide B, 140 kDa | | | | |
| 464 | 22882 | Entrez Gene | zinc fingers and homeoboxes 2 | 41503_at | Day 7 Intra/Day 7 Peri | 0.750 | 6.4066E−03 |
| 467 | 1329 | Entrez Gene | cytochrome c oxidase subunit Vb | 39921_at | Day 7 Intra/Day 7 Control | 0.718 | 5.7364E−03 |
| 468 | 51020 | Entrez Gene | HD domain containing 2 | 34359_at | Day 7 Intra/Day 0 Intra | 0.712 | 2.4876E−03 |
| | | | | 34359_at | Day 7 Intra/Day 7 Extra | 0.777 | 2.8509E−03 |
| 473 | 3054 | Entrez Gene | host cell factor C1 (VP16-accessory protein) | 37910_at | Day 0 Intra/Day 0 Peri | 0.718 | 6.3341E−03 |
| 474 | 79026 | Entrez Gene | AHNAK nucleoprotein (desmoyokin) | 37027_at | Day 7 Intra/Day 0 Intra | 0.718 | 8.1471E−03 |
| 479 | 10611 | Entrez Gene | PDZ and LIM domain 5 | 37366_at | Day 3 Intra/Day 3 Extra | 0.722 | 2.3752E−03 |
| 482 | 9813 | Entrez Gene | KIAA0494 | 41830_at | Day 3 Intra/Day 0 Intra | 0.725 | 3.4757E−03 |
| 489 | 26100 | Entrez Gene | WIPI49-like protein 2 | 33392_at | Day 3 Intra/Day 3 Extra | 0.731 | 6.9435E−03 |
| 494 | 10075 | Entrez Gene | HECT, UBA and WWE domain containing 1 | 34374_g_at | Day 0 Intra/Day 0 Peri | 0.733 | 2.9867E−03 |
| 500 | 23259 | Entrez Gene | DDHD domain containing 2 | 35177_at | Day 7 Intra/Day 0 Intra | 0.736 | 6.5771E−03 |
| 509 | 51304 | Entrez Gene | zinc finger, DHHC-type containing 3 | 39751_at | Day 3 Intra/Day 3 Extra | 0.744 | 7.7468E−03 |
| 513 | 10541 | Entrez Gene | acidic (leucine-rich) nuclear phosphoprotein | 38479_at | Day 7 Intra/Day 7 Extra | 0.747 | 1.6161E−03 |
| | | | 32 family, member B | | | | |
| 515 | 23379 | Entrez Gene | KIAA0947 protein | 41595_at | Day 7 Intra/Day 7 Extra | 0.751 | 2.7370E−03 |
| 519 | 9215 | Entrez Gene | like-glycosyltransferase | 41346_at | Day 3 Intra/Day 0 Intra | 0.754 | 4.1376E−03 |
| 521 | 10241 | Entrez Gene | nuclear domain 10 protein | 40063_at | Day 3 Intra/Day 0 Intra | 0.755 | 7.6513E−03 |
| 526 | 1955 | Entrez Gene | EGF-like-domain, multiple 5 | 36488_at | Day 3 Intra/Day 0 Intra | 0.759 | 7.4685E−03 |
| 530 | 4601 | Entrez Gene | MAX interactor 1 | 39072_at | Day 3 Intra/Day 0 Intra | 0.762 | 4.0454E−03 |
| 532 | 1998 | Entrez Gene | E74-like factor 2 (ets domain transcription | 507_s_at | Day 7 Intra/Day 0 Intra | 0.763 | 4.9954E−03 |
| | | | factor) | | | | |
| 537 | 7693 | Entrez Gene | zinc finger protein 134 (clone pHZ-15) | 36295_at | Day 3 Intra/Day 0 Intra | 0.764 | 9.8397E−03 |
| 545 | 23294 | Entrez Gene | ankyrin repeat and sterile alpha motif domain | 40971_at | Day 3 Intra/Day 0 Intra | 0.769 | 7.4093E−03 |
| | | | containing 1 | | | | |
| 548 | 5636 | Entrez Gene | phosphoribosyl pyrophosphate synthetase- | 41853_at | Day 3 Intra/Day 3 Extra | 0.772 | 9.6928E−03 |
| | | | associated protein 2 | | | | |
| 550 | 9703 | Entrez Gene | KIAA0100 gene product | 39783_at | Day 0 Intra/Day 0 Peri | 0.773 | 4.2046E−03 |
| 551 | 10294 | Entrez Gene | DnaJ (Hsp40) homolog, subfamily A, | 34201_at | Day 3 Intra/Day 0 Intra | 0.775 | 2.1191E−03 |
| | | | member 2 | | | | |
| 552 | 9741 | Entrez Gene | lysosomal-associated protein | 39019_at | Day 3 Intra/Day 0 Intra | 0.776 | 6.0768E−03 |
| | | | transmembrane 4 alpha | 39019_at | Day 7 Intra/Day 0 Intra | 0.815 | 9.6992E−03 |
| 554 | 10556 | Entrez Gene | ribonuclease P/MRP 30 kDa subunit | 39702_at | Day 3 Intra/Day 3 Extra | 0.778 | 8.7433E−03 |
| 555 | 9562 | Entrez Gene | multiple inositol polyphosphate histidine | 38325_at | Day 3 Intra/Day 3 Extra | 0.779 | 8.9541E−03 |
| | | | phosphatase, 1 | | | | |
| 556 | 9528 | Entrez Gene | transmembrane protein 59 | 39033_at | Day 3 Intra/Day 3 Control | 0.779 | 6.9035E−03 |
| | | | | 39033_at | Day 3 Intra/Day 0 Intra | 0.787 | 2.6172E−04 |
| 557 | 5326 | Entrez Gene | pieiomorphic adenoma gene-like 2 | 40061_at | Day 3 Intra/Day 3 Peri | 0.779 | 7.8774E−03 |
| 561 | 670 | Entrez Gene | biphenyl hydrolase-like (serine hydrolase; | 40912_s_at | Day 0 Intra/Day 0 Control | 0.784 | 7.7779E−03 |
| | | | breast epithelial mucin-associated antigen) | | | | |
| 564 | 51780 | Entrez Gene | jumonji domain containing 1B | 33870_at | Day 3 Intra/Day 0 Intra | 0.789 | 6.0617E−03 |
| 568 | 56270 | Entrez Gene | WDR45-like | 39185_at | Day 3 Intra/Day 3 Control | 0.792 | 6.7923E−03 |
| 574 | 79073 | Entrez Gene | hypothetical protein MGC5508 | 39693_at | Day 3 Intra/Day 3 Extra | 0.797 | 7.6719E−03 |
| 587 | 369 | Entrez Gene | v-raf murine sarcoma 3611 viral oncogene | 1706_at | Day 7 Intra/Day 3 Intra | 0.832 | 4.3502E−04 |
| | | | homolog | | | | |
| 588 | 23 | Entrez Gene | ATP-binding cassette, sub-family F (GCN20), | 39141_at | Day 7 Intra/Day 3 Intra | 0.837 | 5.3611E−03 |
| | | | member 1 | | | | |
| 589 | 25966 | Entrez Gene | chromosome 21 open reading frame 25 | 32107_at | Day 7 Intra/Day 7 Extra | 0.845 | 5.8404E−03 |
| 590 | 23644 | Entrez Gene | autoantigen | 36670_at | Day 3 Intra/Day 0 Intra | 0.866 | 7.3351E−03 |

TABLE 21
Diagnostic Down

| Gene | Public | | | | | Fold | |
|---|---|---|---|---|---|---|---|
| ID | Identifier | Data Source | Gene Name | Probe_ID | Comparison | Change | P value |
| 1 | 4250 | Entrez Gene | secretoglobin, family 2A, member 2 | 36329_at | Day 0 Intra/Day 0 Control | 0.009 | 7.1701E−04 |
| | | | | 36329_at | Day 3 Intra/Day 3 Control | 0.017 | 3.3179E−04 |
| | | | | 36329_at | Day 7 Intra/Day 7 Control | 0.021 | 2.8985E−03 |
| 2 | 5304 | Entrez Gene | prolactin-induced protein | 41094_at | Day 0 Intra/Day 0 Control | 0.032 | 5.1676E−04 |
| | | | | 41094_at | Day 7 Intra/Day 7 Control | 0.076 | 9.5561E−03 |
| | | | | 41094_at | Day 3 Intra/Day 3 Control | 0.094 | 1.9791E−04 |
| 3 | 10647 | Entrez Gene | secretoglobin, family 1D, member 2 | 32880_at | Day 0 Intra/Day 0 Control | 0.033 | 4.4000E−05 |
| | | | | 32880_at | Day 7 Intra/Day 7 Control | 0.065 | 7.5456E−03 |
| | | | | 32880_at | Day 3 Intra/Day 3 Control | 0.075 | 8.2511E−04 |
| 4 | HG1763- | The Institute | — | 325_s_at | Day 7 Intra/Day 7 Control | 0.060 | 1.7598E−03 |
| | HT178 | for Genomic | | 325_s_at | Day 3 Intra/Day 3 Control | 0.073 | 2.6304E−04 |
| | | Research | | 325_s_at | Day 0 Intra/Day 0 Control | 0.091 | 6.8631E−04 |
| 5 | 2167 | Entrez Gene | fatty acid binding protein 4, adipocyte | 38430_at | Day 7 Intra/Day 7 Control | 0.075 | 4.2884E−03 |
| 27 | 3119 | Entrez Gene | major histocompatibility complex, class II, | 36878_f_at | Day 0 Intra/Day 0 Control | 0.350 | 9.9683E−03 |
| | | | DQ beta 1 | | | | |
| 41 | 3400 | Entrez Gene | Inhibitor of DNA binding 4, dominant | 41536_at | Day 3 Intra/Day 3 Control | 0.488 | 2.0560E−03 |
| | | | negative helix-loop-helix protein | | | | |
| 43 | 8710 | Entrez Gene | serpin peptidase inhibitor, clade B (ovalbumin), | 35577_at | Day 0 Intra/Day 0 Control | 0.489 | 6.7433E−03 |
| | | | member 7 | 35577_at | Day 3 Intra/Day 3 Control | 0.566 | 9.3574E−03 |
| 47 | 406 | Entrez Gene | aryl hydrocarbon receptor nuclear | 36896_s_at | Day 3 Intra/Day 3 Control | 0.391 | 9.1974E−03 |
| | | | translocator-like | | | | |
| 49 | 63928 | Entrez Gene | hepatocellular carcinoma antigen gene 520 | 33007_at | Day 3 Intra/Day 3 Control | 0.466 | 7.7206E−03 |
| 54 | 224 | Entrez Gene | aldehyde dehydrogenase 3 family, member A2 | 40409_at | Day 3 Intra/Day 3 Control | 0.410 | 5.0007E−03 |
| 55 | 90634 | Entrez Gene | hypothetical gene CG018 | 34239_at | Day 3 Intra/Day 3 Control | 0.472 | 5.9625E−03 |
| 57 | 23242 | Entrez Gene | cordon-bleu homolog (mouse) | 35669_at | Day 3 Intra/Day 3 Control | 0.417 | 3.3977E−03 |
| 79 | 56288 | Entrez Gene | par-3 partitioning defective 3 homolog | 40973_at | Day 3 Intra/Day 3 Control | 0.533 | 5.0405E−03 |
| | | | (C. elegans) | | | | |
| 88 | 10529 | Entrez Gene | nebulette | 34873_at | Day 3 Intra/Day 3 Control | 0.466 | 5.5136E−03 |
| 93 | 1152 | Entrez Gene | creatine kinase, brain | 40863_r_at | Day 7 Intra/Day 7 Control | 0.472 | 4.0142E−03 |
| 94 | 10450 | Entrez Gene | peptidylprolyl isomerase E (cyclophilin E) | 34365_at | Day 3 Intra/Day 3 Control | 0.518 | 4.1270E−03 |
| 99 | 10314 | Entrez Gene | LanC lantibiotic synthetase component C-like 1 | 39441_at | Day 0 Intra/Day 0 Control | 0.488 | 6.0119E−04 |
| | | | (bacterial) | | | | |
| 123 | 1573 | Entrez Gene | cytochrome P450, family 2, subfamily J, | 501_g_at | Day 3 Intra/Day 3 Control | 0.529 | 4.2791E−03 |
| | | | polypeptide 2 | | | | |
| 135 | 36 | Entrez Gene | acyl-Coenzyme A dehydrogenase, short/branched | 40673_at | Day 3 Intra/Day 3 Control | 0.515 | 1.3679E−03 |
| | | | chain | | | | |
| 139 | 10965 | Entrez Gene | acyl-CoA thioesterase 2 | 36625_at | Day 7 Intra/Day 7 Control | 0.518 | 7.3351E−04 |
| 200 | 8349 | Entrez Gene | histone 2, H2be | 33352_at | Day 3 Intra/Day 3 Control | 0.570 | 6.1256E−03 |
| 203 | 115817 | Entrez Gene | dehydrogenase/reductase (SDR family) member 1 | 39103_s_at | Day 0 Intra/Day 0 Control | 0.592 | 1.8258E−03 |
| 217 | 987 | Entrez Gene | LPS-responsive vesicle trafficking, beach and | 35371_at | Day 3 Intra/Day 3 Control | 0.652 | 2.1271E−03 |
| | | | anchor containing | | | | |
| 236 | HG3254- | The Institute | — | 1163_at | Day 3 Intra/Day 3 Control | 0.591 | 5.8755E−03 |
| | HT343 | for Genomic | | | | | |
| | | Research | | | | | |
| 251 | 8776 | Entrez Gene | myotubularin related protein 1 | 34654_at | Day 3 Intra/Day 3 Control | 0.631 | 5.7609E−03 |
| 257 | AI557912 | GenBank | translocase of outer mitochondrial membrane 7 | 39025_at | Day 7 Intra/Day 7 Control | 0.727 | 5.2071E−03 |
| | | | homolog (yeast) /// hypothetical protein | | | | |
| | | | LOC201725 | | | | |
| 272 | 22826 | Entrez Gene | DnaJ (Hsp40) homolog, subfamily C, member 8 | 36166_at | Day 0 Intra/Day 0 Control | 0.612 | 5.7794E−03 |
| 275 | 10497 | Entrez Gene | unc-13 homolog B (C. elegans) | 40087_at | Day 0 Intra/Day 0 Control | 0.769 | 3.3634E−03 |
| 305 | 23351 | Entrez Gene | KIAA0323 | 32592_at | Day 0 Intra/Day 0 Control | 0.679 | 8.2136E−03 |
| 361 | 4677 | Entrez Gene | asparaginyl-tRNA synthetase | 41241_at | Day 0 Intra/Day 0 Control | 0.658 | 3.0519E−03 |
| 371 | 4832 | Entrez Gene | non-metastatic cells 3, protein expressed in | 197_at | Day 3 Intra/Day 3 Control | 0.663 | 6.9663E−03 |
| 391 | 54462 | Entrez Gene | KIAA1128 | 37617_at | Day 3 Intra/Day 3 Control | 0.696 | 9.3133E−03 |
| 452 | 539 | Entrez Gene | ATP synthase, H+ transporting, mitochondrial F1 | 37029_at | Day 7 Intra/Day 7 Control | 0.784 | 7.2352E−03 |
| | | | complex, O subunit (oligomycin sensitivity | | | | |
| | | | conferring protein) | | | | |
| 467 | 1329 | Entrez Gene | cytochrome c oxidase subunit Vb | 39921_at | Day 7 Intra/Day 7 Control | 0.718 | 5.7364E−03 |
| 556 | 9528 | Entrez Gene | transmembrane protein 59 | 39033_at | Day 3 Intra/Day 3 Control | 0.779 | 6.9035E−03 |
| 561 | 670 | Entrez Gene | biphenyl hydrolase-like (serine hydrolase; breast | 40912_s_at | Day 0 Intra/Day 0 Control | 0.784 | 7.7779E−03 |
| | | | epithelial mucin-associated antigen) | | | | |
| 568 | 56270 | Entrez Gene | WDR45-like | 39185_at | Day 3 Intra/Day 3 Control | 0.792 | 6.7923E−03 |

TABLE 22
Diagnostic Down

| Gene | Public | | | | | Fold | |
|---|---|---|---|---|---|---|---|
| ID | Identifier | Data Source | Gene Name | Probe_ID | Comparison | Change | P value |
| 1 | 4250 | Entrez Gene | secretoglobin, family 2A, member 2 | 36329_at | Day 3 Intra/Day 3 Extra | 0.023 | 2.7212E−03 |
| 3 | 10647 | Entrez Gene | secretoglobin, family 1D, member 2 | 32880_at | Day 3 Intra/Day 3 Extra | 0.102 | 5.4085E−03 |
| 4 | HG1763- | The Institute | — | 325_s_at | Day 3 Intra/Day 3 Extra | 0.038 | 6.4653E−03 |
| | HT178 | for Genomic | | | | | |
| | | Research | | | | | |
| 5 | 2167 | Entrez Gene | fatty acid binding protein 4, adipocyte | 38430_at | Day 7 Intra/Day 7 Extra | 0.053 | 1.3371E−03 |
| 7 | 6288 | Entrez Gene | serum amyloid A1 | 33272_at | Day 3 Intra/Day 3 Extra | 0.069 | 6.8147E−03 |
| | | | | 33272_at | Day 7 Intra/Day 7 Extra | 0.117 | 4.3324E−03 |
| 10 | 1690 | Entrez Gene | coagulation factor C homolog, cochlin (Limulus | 34190_at | Day 3 Intra/Day 3 Extra | 0.129 | 1.6089E−03 |
| | | | polyphemus) | | | | |
| 26 | 5271 | Entrez Gene | serpin peptidase inhibitor, clade B (ovalbumin), | 36312_at | Day 7 Intra/Day 7 Extra | 0.482 | 4.6083E−03 |
| | | | member 8 | | | | |
| 57 | 23242 | Entrez Gene | cordon-bleu homolog (mouse) | 35669_at | Day 3 Intra/Day 3 Extra | 0.522 | 4.0595E−04 |
| 69 | 13 | Entrez Gene | arylacetamide deacetylase (esterase) | 36512_at | Day 3 Intra/Day 3 Extra | 0.434 | 1.5905E−04 |
| 75 | 26 | Entrez Gene | amiloride binding protein 1 (amine oxidase | 37186_s_at | Day 3 Intra/Day 3 Extra | 0.443 | 5.1668E−03 |
| | | | (copper-containing)) | | | | |
| 80 | 26154 | Entrez Gene | ATP-binding cassette, sub-family A | 31754_at | Day 3 Intra/Day 3 Extra | 0.557 | 5.7474E−03 |
| | | | (ABC1), member 12 | | | | |
| 82 | 1410 | Entrez Gene | crystallin, alpha B | 32243_g_at | Day 3 Intra/Day 3 Extra | 0.468 | 5.9480E−03 |
| 84 | 10733 | Entrez Gene | polo-like kinase 4 (Drosophila) | 975_at | Day 7 Intra/Day 7 Extra | 0.455 | 5.4588E−03 |
| 133 | 3422 | Entrez Gene | isopentenyl-diphosphate delta isomerase 1 | 36985_at | Day 7 Intra/Day 7 Extra | 0.515 | 5.4407E−03 |
| 147 | 2110 | Entrez Gene | electron-transferring-flavoprotein dehydrogenase | 33494_at | Day 7 Intra/Day 7 Extra | 0.523 | 1.1689E−03 |
| 159 | 5110 | Entrez Gene | Protein-L-isoaspartate (D-aspartate) O- | 37737_at | Day 7 Intra/Day 7 Extra | 0.534 | 2.8151E−03 |
| | | | methyltransferase | | | | |
| 166 | 10653 | Entrez Gene | serine peptidase inhibitor, Kunitz type, 2 | 34348_at | Day 7 Intra/Day 7 Extra | 0.699 | 5.3002E−03 |
| 171 | 1662 | Entrez Gene | DEAD (Asp-Glu-Ala-Asp) box polypeptide 10 | 831_at | Day 7 Intra/Day 7 Extra | 0.544 | 5.2360E−03 |
| 176 | 202 | Entrez Gene | absent in melanoma 1 | 32112_s_at | Day 3 Intra/Day 3 Extra | 0.617 | 9.6499E−03 |
| 177 | 9774 | Entrez Gene | BCL2-associated transcription factor 1 | 38050_at | Day 7 Intra/Day 7 Extra | 0.547 | 4.7061E−03 |
| 181 | 4232 | Entrez Gene | mesoderm specific transcript homolog (mouse) | 37749_at | Day 7 Intra/Day 7 Extra | 0.553 | 9.8929E−04 |
| 189 | 10300 | Entrez Gene | katanin p80 (WD repeat containing) subunit B1 | 40976_at | Day 3 Intra/Day 3 Extra | 0.559 | 9.1699E−03 |
| 190 | 55651 | Entrez Gene | nucleolar protein family A, member 2 | 41322_s_at | Day 3 Intra/Day 3 Extra | 0.675 | 9.6475E−03 |
| | | | (H/ACA small nucleolar RNPs) | | | | |
| 200 | 8349 | Entrez Gene | histone 2, H2be | 33352_at | Day 3 Intra/Day 3 Extra | 0.617 | 5.0561E−03 |
| 201 | 171546 | Entrez Gene | chromosome 14 open reading frame 147 | 33399_at | Day 3 Intra/Day 3 Extra | 0.656 | 8.8383E−03 |
| 214 | 11066 | Entrez Gene | U11/U12 snRNP 35K | 41029_at | Day 3 Intra/Day 3 Extra | 0.589 | 7.2521E−03 |
| 221 | 5917 | Entrez Gene | arginyl-tRNA synthetase | 549_at | Day 7 Intra/Day 7 Extra | 0.584 | 6.5983E−03 |
| 249 | 545 | Entrez Gene | ataxia telangiectasia and Rad3 related | 37229_at | Day 7 Intra/Day 7 Extra | 0.597 | 5.6822E−03 |
| 251 | 8776 | Entrez Gene | myotubularin related protein 1 | 34654_at | Day 3 Intra/Day 3 Extra | 0.598 | 1.6699E−03 |
| 255 | 23234 | Entrez Gene | DnaJ (Hsp40) homolog, subfamily C, member 9 | 41569_at | Day 7 Intra/Day 7 Extra | 0.615 | 8.2268E−03 |
| 263 | 6427 | Entrez Gene | splicing factor, arginine/serine-rich 2 | 36111_s_at | Day 7 Intra/Day 7 Extra | 0.665 | 1.5173E−04 |
| 270 | 3301 | Entrez Gene | DnaJ (Hsp40) homolog, subfamily A, member 1 | 39118_at | Day 7 Intra/Day 7 Extra | 0.611 | 5.7425E−03 |
| 289 | 9 | Entrez Gene | N-acetyltransferase 1 (arylamine | 38187_at | Day 7 Intra/Day 7 Extra | 0.619 | 5.7524E−03 |
| | | | N-acetyltransferase) | | | | |
| 297 | 8888 | Entrez Gene | MCM3 minichromosome maintenance | 40469_at | Day 7 Intra/Day 7 Extra | 0.624 | 4.8160E−03 |
| | | | deficient 3 (S. cerevisiae) associated protein | | | | |
| 315 | 8930 | Entrez Gene | methyl-CpG binding domain protein 4 | 34386_at | Day 7 Intra/Day 7 Extra | 0.634 | 3.7388E−03 |
| 324 | 1340 | Entrez Gene | cytochrome c oxidase subunit Vib polypeptide 1 | 40872_at | Day 3 Intra/Day 3 Extra | 0.698 | 6.3894E−03 |
| | | | (ubiquitous) | | | | |
| 337 | 27067 | Entrez Gene | staufen, RNA binding protein, homolog 2 | 38341_at | Day 3 Intra/Day 3 Extra | 0.645 | 8.2036E−03 |
| | | | (Drosophila) | | | | |
| 342 | 1054 | Entrez Gene | CCAAT/enhancer binding protein (C/EBP), | 39219_at | Day 7 Intra/Day 7 Extra | 0.676 | 1.0916E−03 |
| | | | gamma | | | | |
| 352 | 8732 | Entrez Gene | RNA guanylyltransferase and 5′-phosphatase | 35202_at | Day 7 Intra/Day 7 Extra | 0.654 | 1.2688E−03 |
| 356 | 51567 | Entrez Gene | TRAF and TNF receptor associated protein | 34825_at | Day 3 Intra/Day 3 Extra | 0.655 | 3.0500E−05 |
| 365 | 6635 | Entrez Gene | small nuclear ribonucleoprotein polypeptide E | 38679_g_at | Day 7 Intra/Day 7 Extra | 0.661 | 3.7759E−04 |
| 369 | 7004 | Entrez Gene | TEA domain family member 4 | 41037_at | Day 7 Intra/Day 7 Extra | 0.710 | 7.7243E−03 |
| 372 | 10557 | Entrez Gene | ribonuclease P/MRP 38 kDa subunit | 41040_at | Day 7 Intra/Day 7 Extra | 0.667 | 4.5174E−03 |
| 377 | 56339 | Entrez Gene | Methyltransferase like 3 | 32244_at | Day 3 Intra/Day 3 Extra | 0.726 | 7.6780E−03 |
| 382 | 10038 | Entrez Gene | poly (ADP-ribose) polymerase family, member 2 | 34756_g_at | Day 7 Intra/Day 7 Extra | 0.669 | 7.1429E−03 |
| 387 | 7411 | Entrez Gene | von Hippel-Lindau binding protein 1 | 171_at | Day 7 Intra/Day 7 Extra | 0.672 | 7.7370E−03 |
| 393 | 25972 | Entrez Gene | unc-50 homolog (C. elegans) | 39442_at | Day 7 Intra/Day 7 Extra | 0.675 | 9.6835E−03 |
| 400 | 3156 | Entrez Gene | 3-hydroxy-3-methylglutaryl-Coenzyme A reductase | 39328_at | Day 3 Intra/Day 3 Extra | 0.681 | 6.1525E−03 |
| 405 | 2961 | Entrez Gene | general transcription factor IIE, polypeptide 2, beta | 37295_at | Day 7 Intra/Day 7 Extra | 0.796 | 1.4862E−03 |
| | | | 34 kDa | | | | |
| 409 | 4170 | Entrez Gene | myeloid cell leukemia sequence 1 (BCL2-related) | 277_at | Day 3 Intra/Day 3 Extra | 0.684 | 9.6905E−03 |
| 418 | 4676 | Entrez Gene | nucleosome assembly protein 1-like 4 | 32575_at | Day 7 Intra/Day 7 Extra | 0.790 | 4.9890E−03 |
| 444 | 2926 | Entrez Gene | G-rich RNA sequence binding factor 1 | 32595_at | Day 7 Intra/Day 7 Extra | 0.696 | 6.0979E−03 |
| 450 | 10329 | Entrez Gene | transmembrane protein 5 | 37445_at | Day 3 Intra/Day 3 Extra | 0.700 | 2.4850E−03 |
| 453 | 10542 | Entrez Gene | hepatitis B virus x interacting protein | 38054_at | Day 7 Intra/Day 7 Extra | 0.782 | 3.3831E−03 |
| 456 | 5431 | Entrez Gene | polymerase (RNA) II (DNA directed) | 39746_at | Day 7 Intra/Day 7 Extra | 0.705 | 2.3347E−03 |
| | | | polypeptide B, 140 kDa | | | | |
| 468 | 51020 | Entrez Gene | HD domain containing 2 | 34359_at | Day 7 Intra/Day 7 Extra | 0.777 | 2.8509E−03 |
| 479 | 10611 | Entrez Gene | PDZ and LIM domain 5 | 37366_at | Day 3 Intra/Day 3 Extra | 0.722 | 2.3752E−03 |
| 489 | 26100 | Entrez Gene | WIPI49-like protein 2 | 33392_at | Day 3 Intra/Day 3 Extra | 0.731 | 6.9435E−03 |
| 509 | 51304 | Entrez Gene | zinc finger, DHHC-type containing 3 | 39751_at | Day 3 Intra/Day 3 Extra | 0.744 | 7.7468E−03 |
| 513 | 10541 | Entrez Gene | acidic (leucine-rich) nuclear phosphoprotein | 38479_at | Day 7 Intra/Day 7 Extra | 0.747 | 1.6161E−03 |
| | | | 32 family, member B | | | | |
| 515 | 23379 | Entrez Gene | KIAA0947 protein | 41595_at | Day 7 Intra/Day 7 Extra | 0.751 | 2.7370E−03 |
| 548 | 5636 | Entrez Gene | phosphoribosyl pyrophosphate synthetase- | 41853_at | Day 3 Intra/Day 3 Extra | 0.772 | 9.6928E−03 |
| | | | associated protein 2 | | | | |
| 554 | 10556 | Entrez Gene | ribonuclease P/MRP 30 kDa subunit | 39702_at | Day 3 Intra/Day 3 Extra | 0.778 | 8.7433E−03 |
| 555 | 9562 | Entrez Gene | multiple inositol polyphosphate histidine | 38325_at | Day 3 Intra/Day 3 Extra | 0.779 | 8.9541E−03 |
| | | | phosphatase, 1 | | | | |
| 574 | 79073 | Entrez Gene | hypothetical protein MGC5508 | 39693_at | Day 3 Intra/Day 3 Extra | 0.797 | 7.6719E−03 |
| 589 | 25966 | Entrez Gene | chromosome 21 open reading frame 25 | 32107_at | Day 7 Intra/Day 7 Extra | 0.845 | 5.8404E−03 |

TABLE 23
Diagnostic Down

| Gene | Public | | | | | Fold | |
|---|---|---|---|---|---|---|---|
| ID | Identifier | Data Source | Gene Name | Probe_ID | Comparison | Change | P value |
| 166 | 10653 | Entrez Gene | serine peptidase inhibitor, Kunitz type, 2 | 34348_at | Day 7 Intra/Day 7 Extra | 0.699 | 5.3002E−03 |

TABLE 24
Diagnostic Down

| Gene | Public | | | | | Fold | |
|---|---|---|---|---|---|---|---|
| ID | Identifier | Data Source | Gene Name | Probe_ID | Comparison | Change | P value |
| 6 | 3283 | Entrez Gene | hydroxy-delta-5-steroid dehydrogenase, 3 beta- and | 35721_at | Day 0 Intra/Day 0 Peri | 0.062 | 4.4497E−03 |
| | | | steroid delta-isomerase 1 | 32190_at | Day 0 Intra/Day 0 Peri | 0.081 | 5.6381E−04 |
| | | | | 32190_at | Day 7 Intra/Day 7 Peri | 0.201 | 7.6424E−03 |
| 9 | 247 | Entrez Gene | arachidonate 15-lipoxygenase, second type | 37430_at | Day 0 Intra/Day 0 Peri | 0.096 | 4.5492E−03 |
| 12 | HG371- | The Institute | — | 700_s_at | Day 0 Intra/Day 0 Peri | 0.143 | 4.4849E−03 |
| | HT2638 | for Genomic | | | | | |
| | | Research | | | | | |
| 16 | L09190 | GenBank | Full-length cDNA clone CS0DI027YJ05 of Placenta | 37635_at | Day 3 Intra/Day 3 Peri | 0.206 | 2.8404E−03 |
| | | | Cot 25-normalized of Homo sapiens (human) | | | | |
| 19 | 10351 | Entrez Gene | ATP-binding cassette, sub-family A (ABC1), | 35717_at | Day 3 Intra/Day 3 Peri | 0.305 | 5.8783E−04 |
| | | | member 8 | | | | |
| 30 | 3885 | Entrez Gene | keratin, hair, acidic, 4 | 34012_at | Day 0 Intra/Day 0 Peri | 0.352 | 4.5590E−03 |
| 44 | 2194 | Entrez Gene | fatty acid synthase | 38429_at | Day 0 Intra/Day 0 Peri | 0.386 | 9.3714E−03 |
| 68 | 1117 | Entrez Gene | chitinase 3-like 2 | 31891_at | Day 0 Intra/Day 0 Pari | 0.432 | 5.3219E−04 |
| 73 | 57798 | Entrez Gene | GATA zinc finger domain containing 1 | 34195_at | Day 3 Intra/Day 3 Peri | 0.441 | 6.2766E−04 |
| 89 | 60481 | Entrez Gene | ELOVL family member 5, elongation of long chain | 33821_at | Day 0 Intra/Day 0 Peri | 0.468 | 8.7794E−03 |
| | | | fatty acids (FEN1/Elo2, SUR4/Elo3-like, yeast) | | | | |
| 92 | 1545 | Entrez Gene | cytochrome P450, family 1, subfamily B, | 40071_at | Day 7 Intra/Day 7 Peri | 0.471 | 9.8480E−03 |
| | | | polypeptide 1 | | | | |
| 105 | 9987 | Entrez Gene | Heterogeneous nuclear ribonucleoprotein D-like | 32393_s_at | Day 3 Intra/Day 3 Peri | 0.489 | 1.9407E−03 |
| 107 | 4163 | Entrez Gene | mutated in colorectal cancers | 35561_at | Day 0 Intra/Day 0 Peri | 0.489 | 6.2800E−05 |
| 119 | 6662 | Entrez Gene | SRY (sex determining region Y)-box 9 (campomelic | 33436_at | Day 0 Intra/Day 0 Peri | 0.521 | 8.5777E−03 |
| | | | dysplasia, autosomal sex-reversal) | | | | |
| 125 | 51710 | Entrez Gene | zinc finger protein 44 (KOX 7) | 35409_r_at | Day 3 Intra/Day 3 Peri | 0.507 | 5.1572E−03 |
| 126 | 2533 | Entrez Gene | FYN binding protein (FYB-120/130) | 41819_at | Day 3 Intra/Day 3 Peri | 0.507 | 3.0115E−03 |
| 127 | 9857 | Entrez Gene | centrosome-associated protein 350 | 33805_at | Day 3 Intra/Day 3 Peri | 0.508 | 4.3400E−05 |
| 134 | 7728 | Entrez Gene | zinc finger protein 175 | 37500_at | Day 7 Intra/Day 7 Peri | 0.515 | 4.1955E−03 |
| 153 | 9140 | Entrez Gene | ATG12 autophagy related 12 homolog (S. cerevisiae) | 32720_at | Day 3 Intra/Day 3 Peri | 0.530 | 2.7861E−04 |
| 163 | 51099 | Entrez Gene | Abhydrolase domain containing 5 | 33309_at | Day 3 Intra/Day 3 Peri | 0.536 | 9.8735E−03 |
| 164 | 114882 | Entrez Gene | oxysterol binding protein-like 8 | 41438_at | Day 3 Intra/Day 3 Peri | 0.536 | 3.0621E−03 |
| 172 | 5130 | Entrez Gene | phosphate cytidylyltransferase 1, choline, alpha | 39285_at | Day 0 Intra/Day 0 Peri | 0.545 | 2.3355E−03 |
| 174 | 26235 | Entrez Gene | F-box and leucine-rich repeat protein 4 | 39987_at | Day 7 Intra/Day 7 Peri | 0.546 | 3.2958E−03 |
| 184 | U73737 | GenBank | — | 1017_at | Day 3 Intra/Day 3 Peri | 0.556 | 4.6650E−03 |
| 211 | 8315 | Entrez Gene | BRCA1 associated protein | 41512_at | Day 3 Intra/Day 3 Peri | 0.677 | 7.0812E−04 |
| 219 | 65055 | Entrez Gene | chromosome 2 open reading frame 23 | 36096_at | Day 3 Intra/Day 3 Peri | 0.583 | 2.9762E−03 |
| 225 | 5927 | Entrez Gene | Jumonji, AT rich interactive domain 1A | 1785_at | Day 3 Intra/Day 3 Peri | 0.585 | 3.1353E−03 |
| | | | (RBBP2-like) | | | | |
| 226 | 2824 | Entrez Gene | glycoprotein M6B | 37251_s_at | Day 3 Intra/Day 3 Peri | 0.586 | 8.6602E−03 |
| 247 | 2355 | Entrez Gene | FOS-like antigen 2 | 40268_at | Day 0 Intra/Day 0 Peri | 0.596 | 5.8255E−03 |
| 261 | 6651 | Entrez Gene | SON DNA binding protein | 39096_at | Day 7 Intra/Day 7 Peri | 0.641 | 4.2992E−03 |
| 273 | 23090 | Entrez Gene | zinc finger protein 423 | 34950_at | Day 7 Intra/Day 7 Peri | 0.623 | 7.6235E−03 |
| 288 | 3418 | Entrez Gene | isocitrate dehydrogenase 2 (NADP+), mitochondrial | 32332_at | Day 0 Intra/Day 0 Peri | 0.617 | 1.2980E−03 |
| 302 | 24139 | Entrez Gene | echinoderm microtubule associated protein like 2 | 41328_s_at | Day 0 Intra/Day 0 Peri | 0.627 | 4.4037E−03 |
| 311 | 4681 | Entrez Gene | neuroblastoma, suppression of tumorigenicity 1 | 37005_at | Day 0 Intra/Day 0 Peri | 0.631 | 3.2813E−03 |
| 329 | 4026 | Entrez Gene | LIM domain containing preferred translocation | 41195_at | Day 3 Intra/Day 3 Peri | 0.639 | 3.3595E−03 |
| | | | partner in lipoma | | | | |
| 369 | 7004 | Entrez Gene | TEA domain family member 4 | 41037_at | Day 0 Intra/Day 0 Peri | 0.755 | 4.0427E−03 |
| 399 | 50813 | Entrez Gene | COP9 constitutive photomorphogenic homolog | 34404_at | Day 0 Intra/Day 0 Peri | 0.681 | 4.7805E−03 |
| | | | subunit 7A (Arabidopsis) | | | | |
| 414 | 23112 | Entrez Gene | trinucleotide repeat containing 6B | 37487_at | Day 3 Intra/Day 3 Peri | 0.688 | 3.9849E−03 |
| 435 | 889 | Entrez Gene | KRIT1, ankyrin repeat containing | 34031_i_at | Day 3 Intra/Day 3 Peri | 0.694 | 4.9207E−03 |
| 464 | 22882 | Entrez Gene | zinc fingers and homeoboxes 2 | 41503_at | Day 7 Intra/Day 7 Peri | 0.750 | 6.4066E−03 |
| 473 | 3054 | Entrez Gene | host cell factor C1 (VP16-accessory protein) | 37910_at | Day 0 Intra/Day 0 Peri | 0.718 | 6.3341E−03 |
| 494 | 10075 | Entrez Gene | HECT, UBA and WWE domain containing 1 | 34374_g_at | Day 0 Intra/Day 0 Peri | 0.733 | 2.9867E−03 |
| 550 | 9703 | Entrez Gene | KIAA0100 gene product | 39783_at | Day 0 Intra/Day 0 Peri | 0.773 | 4.2046E−03 |
| 557 | 5326 | Entrez Gene | pleiomorphic adenoma gene-like 2 | 40061_at | Day 3 Intra/Day 3 Peri | 0.779 | 7.8774E−03 |

TABLE 25
Diagnostic Down

| | Public | | | | | Fold | |
|---|---|---|---|---|---|---|---|
| Gene ID | Identifier | Data Source | Gene Name | Probe_ID | Comparison | Change | P value |
| 329 | 4026 | Entrez Gene | LIM domain containing preferred | 41195_at | Day 3 Intra/Day 3 Peri | 0.639 | 3.3595E−03 |
| | | | translocation partner in lipoma | | | | |

TABLE 26
Diagnostic Down

| Gene | Public | | | | | Fold | |
|---|---|---|---|---|---|---|---|
| ID | Identifier | Data Source | Gene Name | Probe_ID | Comparison | Change | P value |
| 13 | 125 | Entrez Gene | alcohol dehydrogenase IB (class I), beta | 35730_at | Day 3 Intra/Day 0 Intra | 0.162 | 9.2302E−03 |
| | | | polypeptide | | | | |
| 18 | 7123 | Entrez Gene | C-type lectin domain family 3, member B | 36569_at | Day 3 Intra/Day 0 Intra | 0.243 | 7.7786E−03 |
| 19 | 10351 | Entrez Gene | ATP-binding cassette, sub-family A (ABC1), | 35717_at | Day 3 Intra/Day 0 Intra | 0.279 | 3.1166E−03 |
| | | | member 8 | | | | |
| 20 | AB011538 | GenBank | CDNA clone IMAGE: 5922621 | 35324_at | Day 3 Intra/Day 0 Intra | 0.280 | 3.8006E−03 |
| 21 | 3131 | Entrez Gene | Hepatic leukemia factor | 38627_at | Day 3 Intra/Day 0 Intra | 0.289 | 1.1838E−03 |
| | | | | 38627_at | Day 7 Intra/Day 0 Intra | 0.325 | 6.7101E−03 |
| 23 | 4211 | Entrez Gene | Meis1, myeloid ecotropic viral integration site 1 | 40763_at | Day 3 Intra/Day 0 Intra | 0.324 | 4.5206E−03 |
| | | | homolog (mouse) | | | | |
| 24 | 2053 | Entrez Gene | epoxide hydrolase 2, cytoplasmic | 41473_at | Day 3 Intra/Day 0 Intra | 0.327 | 3.5922E−03 |
| 25 | 4253 | Entrez Gene | CTAGE family, member 5 | 41615_at | Day 3 Intra/Day 0 Intra | 0.330 | 3.8948E−03 |
| 32 | 11075 | Entrez Gene | stathmin-like 2 | 38800_at | Day 3 Intra/Day 0 Intra | 0.356 | 5.6882E−03 |
| 33 | 3075 | Entrez Gene | complement factor H | 32250_at | Day 7 Intra/Day 0 Intra | 0.356 | 2.4648E−03 |
| 34 | 10458 | Entrez Gene | BAI1-associated protein 2 | 37760_at | Day 7 Intra/Day 3 Intra | 0.799 | 8.1747E−03 |
| 35 | 9086 | Entrez Gene | eukaryotic translation initiation factor 1A, Y-linked | 40097_at | Day 3 Intra/Day 0 Intra | 0.358 | 8.6154E−03 |
| 36 | 2273 | Entrez Gene | four and a half LIM domains 1 | 32542_at | Day 3 Intra/Day 0 Intra | 0.367 | 5.7500E−03 |
| 40 | 4223 | Entrez Gene | mesenchyme homeo box 2 (growth arrest-specific | 40398_s_at | Day 3 Intra/Day 0 Intra | 0.376 | 7.2046E−03 |
| | | | homeo box) | | | | |
| 41 | 3400 | Entrez Gene | inhibitor of DNA binding 4, dominant negative | 41536_at | Day 3 Intra/Day 0 Intra | 0.381 | 8.0900E−05 |
| | | | helix-loop-helix protein | 41536_at | Day 7 Intra/Day 0 Intra | 0.410 | 2.1354E−03 |
| 48 | 9452 | Entrez Gene | integral membrane protein 2A | 40775_at | Day 3 Intra/Day 0 Intra | 0.392 | 3.3491E−03 |
| | | | | 40775_at | Day 7 Intra/Day 0 Intra | 0.400 | 2.0273E−03 |
| 51 | 5354 | Entrez Gene | proteolipid protein 1 (Pelizaeus-Merzbacher | 41158_at | Day 3 Intra/Day 0 Intra | 0.403 | 1.1357E−04 |
| | | | disease, spastic paraplegia 2, uncomplicated) | 41158_at | Day 7 Intra/Day 0 Intra | 0.503 | 1.7500E−03 |
| 54 | 224 | Entrez Gene | aldehyde dehydrogenase 3 family, member A2 | 40409_at | Day 3 Intra/Day 0 Intra | 0.466 | 8.5061E−04 |
| 57 | 23242 | Entrez Gene | cordon-bleu homolog (mouse) | 35669_at | Day 3 Intra/Day 0 Intra | 0.414 | 5.3389E−03 |
| 61 | M11119 | GenBank | — | 38850_at | Day 3 Intra/Day 0 Intra | 0.428 | 8.8745E−03 |
| 62 | 9687 | Entrez Gene | GREB1 protein | 38875_r_at | Day 7 Intra/Day 0 Intra | 0.428 | 1.0961E−04 |
| 63 | 51097 | Entrez Gene | saccharopine dehydrogenase (putative) | 34862_at | Day 7 Intra/Day 0 Intra | 0.680 | 6.5823E−03 |
| 67 | 9145 | Entrez Gene | synaptogyrin 1 | 35354_at | Day 7 Intra/Day 0 Intra | 0.432 | 1.9735E−03 |
| | | | | 35354_at | Day 3 Intra/Day 0 Intra | 0.469 | 5.7597E−03 |
| 76 | AL080082 | GenBank | MRNA; cDNA DKFZp564G1162 (from clone | 35581_at | Day 3 Intra/Day 0 Intra | 0.444 | 9.2254E−03 |
| | | | DKFZp564G1162) | | | | |
| 85 | 8848 | Entrez Gene | TSC22 domain family, member 1 | 39032_at | Day 3 Intra/Day 0 Intra | 0.455 | 1.8273E−03 |
| 87 | 5264 | Entrez Gene | phytanoyl-CoA hydroxylase (Refsum disease) | 32724_at | Day 3 Intra/Day 0 Intra | 0.465 | 4.1244E−03 |
| 91 | 10248 | Entrez Gene | processing of precursor 7, ribonuclease P subunit | 32213_at | Day 7 Intra/Day 3 Intra | 0.470 | 9.0083E−03 |
| | | | (S. cerevisiae) | | | | |
| 96 | 10090 | Entrez Gene | uronyl-2-sulfotransferase | 41859_at | Day 3 Intra/Day 0 Intra | 0.474 | 1.8927E−03 |
| | | | | 41859_at | Day 7 Intra/Day 0 Intra | 0.521 | 1.4198E−04 |
| 98 | 352961 | Entrez Gene | MHC class I mRNA fragment 3.8-1 | 34934_at | Day 3 Intra/Day 0 Intra | 0.479 | 6.5297E−03 |
| 100 | 10628 | Entrez Gene | thioredoxin interacting protein | 31508_at | Day 3 Intra/Day 0 Intra | 0.482 | 9.0159E−03 |
| 102 | 6038 | Entrez Gene | ribonuclease, RNase A family, 4 | 32664_at | Day 3 Intra/Day 0 Intra | 0.483 | 2.7472E−03 |
| | | | | 32664_at | Day 7 Intra/Day 0 Intra | 0.569 | 4.9291E−04 |
| 104 | 6414 | Entrez Gene | selenoprotein P, plasma, 1 | 34363_at | Day 3 Intra/Day 0 Intra | 0.488 | 6.0330E−03 |
| 111 | 2947 | Entrez Gene | glutathione S-transferase M3 (brain) | 1120_at | Day 7 Intra/Day 0 Intra | 0.494 | 7.1503E−03 |
| 114 | 8864 | Entrez Gene | period homolog 2 (Drosophila) | 35008_at | Day 3 Intra/Day 0 Intra | 0.497 | 4.3990E−03 |
| 117 | 104 | Entrez Gene | adenosine deaminase, RNA-specific, B1 (RED1 | 38748_at | Day 3 Intra/Day 0 Intra | 0.499 | 8.5077E−04 |
| | | | homolog rat) | 38748_at | Day 7 Intra/Day 0 Intra | 0.541 | 1.9004E−03 |
| 122 | 9831 | Entrez Gene | zinc finger protein 623 | 39954_r_at | Day 3 Intra/Day 0 Intra | 0.505 | 3.3609E−03 |
| 124 | 22982 | Entrez Gene | KIAA0934 | 33408_at | Day 3 Intra/Day 0 Intra | 0.506 | 3.1621E−03 |
| 128 | 9891 | Entrez Gene | NUAK family, SNF1-like kinase, 1 | 33787_at | Day 3 Intra/Day 0 Intra | 0.508 | 1.4528E−03 |
| 131 | 23051 | Entrez Gene | zinc fingers and homeoboxes 3 | 40461_at | Day 3 Intra/Day 0 Intra | 0.513 | 6.7006E−04 |
| | | | | 40461_at | Day 7 Intra/Day 0 Intra | 0.609 | 6.3269E−03 |
| 140 | 10284 | Entrez Gene | Sin3-associated polypeptide, 18 kDa | 41217_at | Day 3 Intra/Day 0 Intra | 0.520 | 2.4318E−03 |
| 143 | 7073 | Entrez Gene | TIA1 cytotoxic granule-associated RNA binding | 41761_at | Day 3 Intra/Day 0 Intra | 0.521 | 4.5836E−03 |
| | | | protein-like 1 | | | | |
| 145 | 8853 | Entrez Gene | development and differentiation enhancing factor 2 | 39410_at | Day 7 Intra/Day 0 Intra | 0.521 | 2.9990E−03 |
| 146 | 4968 | Entrez Gene | 8-oxoguanine DNA glycosylase | 38335_at | Day 3 Intra/Day 0 Intra | 0.522 | 7.5868E−04 |
| | | | | 38335_at | Day 7 Intra/Day 0 Intra | 0.626 | 6.6349E−03 |
| 151 | 55187 | Entrez Gene | vacuolar protein sorting 13D (yeast) | 32743_at | Day 3 Intra/Day 0 Intra | 0.527 | 2.5585E−03 |
| 155 | 23506 | Entrez Gene | KIAA0240 | 38892_at | Day 3 Intra/Day 0 Intra | 0.531 | 5.3954E−03 |
| 158 | 6595 | Entrez Gene | SWI/SNF related, matrix associated, actin | 40961_at | Day 3 Intra/Day 0 Intra | 0.533 | 6.2437E−03 |
| | | | dependent regulator of chromatin, subfamily a, | | | | |
| | | | member 2 | | | | |
| 161 | 201229 | Entrez Gene | hypothetical protein LOC201229 | 39557_at | Day 3 Intra/Day 0 Intra | 0.535 | 2.3943E−03 |
| 162 | 22909 | Entrez Gene | KIAA1018 protein | 36458_at | Day 3 Intra/Day 0 Intra | 0.536 | 6.1234E−03 |
| 168 | 399563 | Entrez Gene | hypothetical protein FLJ43806 | 37151_at | Day 7 Intra/Day 0 Intra | 0.542 | 7.7014E−03 |
| 182 | 2145 | Entrez Gene | enhancer of zeste homolog 1 (Drosophila) | 32259_at | Day 7 Intra/Day 0 Intra | 0.554 | 8.0450E−03 |
| 196 | 57209 | Entrez Gene | zinc finger protein 248 | 35139_at | Day 3 Intra/Day 0 Intra | 0.565 | 2.1978E−03 |
| 211 | 8315 | Entrez Gene | BRCA1 associated protein | 41512_at | Day 3 Intra/Day 0 Intra | 0.577 | 1.1128E−03 |
| 212 | 8623 | Entrez Gene | acetylserotonin O-methyltransferase-like | 36553_at | Day 3 Intra/Day 0 Intra | 0.578 | 7.1773E−04 |
| 213 | 169611 | Entrez Gene | olfactomedin-like 2A | 38312_at | Day 3 Intra/Day 0 Intra | 0.579 | 3.6248E−03 |
| 218 | 128 | Entrez Gene | alcohol dehydrogenase 5 (class III), chi polypeptide | 37708_r_at | Day 3 Intra/Day 0 Intra | 0.582 | 8.9780E−03 |
| 223 | 2621 | Entrez Gene | growth arrest-specific 6 | 1597_at | Day 3 Intra/Day 0 Intra | 0.585 | 6.6299E−03 |
| 224 | 831 | Entrez Gene | calpastatin | 41257_at | Day 3 Intra/Day 0 Intra | 0.585 | 5.3045E−04 |
| 227 | 1153 | Entrez Gene | cold inducible RNA binding protein | 39864_at | Day 3 Intra/Day 0 Intra | 0.587 | 2.1268E−03 |
| | | | | 39864_at | Day 7 Intra/Day 0 Intra | 0.611 | 6.9554E−03 |
| 230 | AB006780 | GenBank | lectin, galactoside-binding, soluble, 3 (galectin 3) /// | 35367_at | Day 3 Intra/Day 0 Intra | 0.590 | 8.4864E−03 |
| | | | galectin-3 internal gene | | | | |
| 231 | 6929 | Entrez Gene | transcription factor 3 (E2A immunoglobulin | 1373_at | Day 3 Intra/Day 0 Intra | 0.590 | 2.7482E−03 |
| | | | enhancer binding factors E12/E47) | | | | |
| 242 | 23353 | Entrez Gene | unc-84 homolog A (C. elegans) | 36588_at | Day 7 Intra/Day 0 Intra | 0.594 | 2.9354E−03 |
| 244 | 6670 | Entrez Gene | Sp3 transcription factor | 41573_at | Day 3 Intra/Day 0 Intra | 0.594 | 6.5653E−03 |
| 253 | AL080234 | GenBank | Full-length cDNA clone CS0DM001YA04 of Fetal | 38070_at | Day 7 Intra/Day 0 Intra | 0.598 | 4.0826E−03 |
| | | | liver of Homo sapiens (human) | | | | |
| 256 | 94239 | Entrez Gene | H2A histone family, member V | 39046_at | Day 3 Intra/Day 0 Intra | 0.600 | 7.9321E−03 |
| | | | | 39046_at | Day 7 Intra/Day 0 Intra | 0.650 | 5.0612E−03 |
| 257 | AI557912 | GenBank | translocase of outer mitochondrial membrane 7 | 39025_at | Day 7 Intra/Day 0 Intra | 0.601 | 3.8343E−03 |
| | | | homolog (yeast) /// hypothetical protein LOC201725 | | | | |
| 261 | 6651 | Entrez Gene | SON DNA binding protein | 39096_at | Day 7 Intra/Day 0 Intra | 0.607 | 3.4000E−03 |
| | | | | 39096_at | Day 7 Intra/Day 3 Intra | 0.710 | 4.2168E−03 |
| 263 | 6427 | Entrez Gene | splicing factor, arginine/serine-rich 2 | 36111_s_at | Day 7 Intra/Day 3 Intra | 0.709 | 6.0916E−03 |
| 264 | 221749 | Entrez Gene | chromosome 6 open reading frame 145 | 34246_at | Day 3 Intra/Day 0 Intra | 0.608 | 3.6829E−03 |
| 267 | 54665 | Entrez Gene | round spermatid basic protein 1 | 37828_at | Day 3 Intra/Day 0 Intra | 0.610 | 7.8572E−04 |
| 274 | 64112 | Entrez Gene | modulator of apoptosis 1 | 34767_at | Day 3 Intra/Day 0 Intra | 0.614 | 4.4658E−03 |
| 282 | 23392 | Entrez Gene | KIAA0368 | 34414_at | Day 7 Intra/Day 0 Intra | 0.616 | 1.2883E−03 |
| 283 | 5934 | Entrez Gene | retinoblastoma-like 2 (p130) | 32597_at | Day 3 Intra/Day 0 Intra | 0.616 | 8.7510E−03 |
| 284 | 4781 | Entrez Gene | Nuclear factor I/B | 41229_at | Day 3 Intra/Day 0 Intra | 0.616 | 3.0477E−03 |
| 290 | 25797 | Entrez Gene | glutaminyl-peptide cyclotransferase (glutaminyl | 35966_at | Day 3 Intra/Day 0 Intra | 0.619 | 4.9522E−03 |
| | | | cyclase) | | | | |
| 298 | 10289 | Entrez Gene | translation factor sul1 homolog | 33351_at | Day 3 Intra/Day 0 Intra | 0.673 | 1.7268E−03 |
| 310 | 22903 | Entrez Gene | BTB (POZ) domain containing 3 | 37755_at | Day 3 Intra/Day 0 Intra | 0.631 | 9.7703E−03 |
| 312 | 10301 | Entrez Gene | deleted in lymphocytic leukemia, 1 | 33791_at | Day 7 Intra/Day 0 Intra | 0.631 | 8.9965E−03 |
| | | | | 33791_at | Day 3 Intra/Day 0 Intra | 0.726 | 6.7457E−03 |
| 313 | AF052138 | GenBank | FLJ35348 /// Bromodomain containing 3 | 41841_at | Day 3 Intra/Day 0 Intra | 0.632 | 4.2490E−03 |
| 319 | 1428 | Entrez Gene | crystallin, mu | 38285_at | Day 3 Intra/Day 0 Intra | 0.636 | 1.2448E−03 |
| 320 | 10614 | Entrez Gene | Hexamethylene bis-acetamide inducible 1 | 40220_at | Day 3 Intra/Day 0 Intra | 0.636 | 3.7740E−03 |
| 334 | 6230 | Entrez Gene | ribosomal protein S25 | 31573_at | Day 7 Intra/Day 0 Intra | 0.643 | 9.4033E−03 |
| 341 | 9521 | Entrez Gene | eukaryotic translation elongation factor 1 epsilon 1 | 40587_s_at | Day 7 Intra/Day 3 Intra | 0.647 | 1.9928E−03 |
| 360 | 3298 | Entrez Gene | heat shock transcription factor 2 | 202_at | Day 7 Intra/Day 0 Intra | 0.658 | 7.1609E−03 |
| 363 | 23384 | Entrez Gene | KIAA0376 protein | 34837_at | Day 3 Intra/Day 0 Intra | 0.659 | 2.3071E−03 |
| 365 | 6635 | Entrez Gene | small nuclear ribonucleoprotein polypeptide E | 38679_g_at | Day 7 Intra/Day 3 Intra | 0.697 | 1.8800E−05 |
| 373 | 26156 | Entrez Gene | ribosomal L1 domain containing 1 | 39418_at | Day 7 Intra/Day 3 Intra | 0.691 | 5.6288E−03 |
| 376 | 26053 | Entrez Gene | autism susceptibility candidate 2 | 35648_at | Day 3 Intra/Day 0 Intra | 0.667 | 3.9622E−03 |
| 377 | 56339 | Entrez Gene | Methyltransferase like 3 | 32244_at | Day 3 Intra/Day 0 Intra | 0.667 | 8.9345E−04 |
| 386 | 10904 | Entrez Gene | bladder cancer associated protein | 35267_g_at | Day 7 Intra/Day 0 Intra | 0.672 | 2.1451E−03 |
| 390 | 1349 | Entrez Gene | cytochrome c oxidase subunit VIIb | 36687_at | Day 7 Intra/Day 3 Intra | 0.673 | 1.8885E−03 |
| 391 | 54462 | Entrez Gene | KIAA1128 | 37617_at | Day 3 Intra/Day 0 Intra | 0.674 | 5.5416E−03 |
| 392 | 80003 | Entrez Gene | pecanex-like 2 (Drosophila) | 39650_s_at | Day 3 Intra/Day 0 Intra | 0.674 | 6.1109E−03 |
| 394 | 64795 | Entrez Gene | hypothetical protein FLJ13910 | 36580_at | Day 3 Intra/Day 0 Intra | 0.676 | 3.2610E−03 |
| 397 | 7743 | Entrez Gene | zinc finger protein 189 | 36072_at | Day 3 Intra/Day 0 Intra | 0.678 | 3.7246E−03 |
| 417 | 9759 | Entrez Gene | histone deacetylase 4 | 38271_at | Day 3 Intra/Day 0 Intra | 0.689 | 4.9833E−03 |
| 422 | 1749 | Entrez Gene | distal-less homeo box 5 | 41087_at | Day 3 Intra/Day 0 Intra | 0.689 | 3.6526E−03 |
| 427 | 22794 | Entrez Gene | cancer susceptibility candidate 3 | 38437_at | Day 7 Intra/Day 0 Intra | 0.692 | 2.7057E−03 |
| | | | | 38437_at | Day 3 Intra/Day 0 Intra | 0.742 | 4.1247E−03 |
| 428 | 322 | Entrez Gene | amyloid beta (A4) precursor protein-binding, family | 1101_at | Day 7 Intra/Day 0 Intra | 0.692 | 1.6594E−03 |
| | | | B, member 1 (Fe65) | | | | |
| 430 | 6637 | Entrez Gene | small nuclear ribonucleoprotein polypeptide G | 37337_at | Day 7 Intra/Day 3 Intra | 0.693 | 3.9690E−03 |
| 439 | 2549 | Entrez Gene | GRB2-associated binding protein 1 | 33997_at | Day 7 Intra/Day 0 Intra | 0.695 | 2.4091E−04 |
| | | | | 33997_at | Day 3 Intra/Day 0 Intra | 0.710 | 2.0825E−03 |
| 443 | 23125 | Entrez Gene | calmodulin binding transcription activator 2 | 41421_at | Day 7 Intra/Day 0 Intra | 0.696 | 8.6760E−03 |
| 447 | 22849 | Entrez Gene | cytoplasmic polyadenylation element binding | 34931_at | Day 3 Intra/Day 0 Intra | 0.697 | 6.8691E−03 |
| | | | protein 3 | | | | |
| 448 | 10106 | Entrez Gene | CTD (carboxy-terminal domain, RNA polymerase | 41202_s_at | Day 3 Intra/Day 0 Intra | 0.698 | 3.5449E−03 |
| | | | II, polypeptide A) small phosphatase 2 | | | | |
| 451 | 26959 | Entrez Gene | HMG-box transcription factor 1 | 39809_at | Day 3 Intra/Day 0 Intra | 0.701 | 9.4669E−03 |
| 454 | 8624 | Entrez Gene | Down syndrome critical region gene 2 | 36088_at | Day 7 Intra/Day 3 Intra | 0.704 | 8.0554E−03 |
| 468 | 51020 | Entrez Gene | HD domain containing 2 | 34359_at | Day 7 Intra/Day 0 Intra | 0.712 | 2.4876E−03 |
| 474 | 79026 | Entrez Gene | AHNAK nucleoprotein (desmoyokin) | 37027_at | Day 7 Intra/Day 0 Intra | 0.718 | 8.1471E−03 |
| 482 | 9813 | Entrez Gene | KIAA0494 | 41830_at | Day 3 Intra/Day 0 Intra | 0.725 | 3.4757E−03 |
| 500 | 23259 | Entrez Gene | DDHD domain containing 2 | 35177_at | Day 7 Intra/Day 0 Intra | 0.736 | 6.5771E−03 |
| 519 | 9215 | Entrez Gene | like-glycosyltransferase | 41346_at | Day 3 Intra/Day 0 Intra | 0.754 | 4.1376E−03 |
| 521 | 10241 | Entrez Gene | nuclear domain 10 protein | 40063_at | Day 3 Intra/Day 0 Intra | 0.755 | 7.6513E−03 |
| 526 | 1955 | Entrez Gene | EGF-like-domain, multiple 5 | 36488_at | Day 3 Intra/Day 0 Intra | 0.759 | 7.4685E−03 |
| 530 | 4601 | Entrez Gene | MAX interactor 1 | 39072_at | Day 3 Intra/Day 0 Intra | 0.762 | 4.0454E−03 |
| 532 | 1998 | Entrez Gene | E74-like factor 2 (ets domain transcription factor) | 507_s_at | Day 7 Intra/Day 0 Intra | 0.763 | 4.9954E−03 |
| 537 | 7693 | Entrez Gene | zinc finger protein 134 (clone pHZ-15) | 36295_at | Day 3 Intra/Day 0 Intra | 0.764 | 9.8397E−03 |
| 545 | 23294 | Entrez Gene | ankyrin repeat and sterile alpha motif domain | 40971_at | Day 3 Intra/Day 0 Intra | 0.769 | 7.4093E−03 |
| | | | containing 1 | | | | |
| 551 | 10294 | Entrez Gene | DnaJ (Hsp40) homolog, subfamily A, member 2 | 34201_at | Day 3 Intra/Day 0 Intra | 0.775 | 2.1191E−03 |
| 552 | 9741 | Entrez Gene | lysosomal-associated protein transmembrane 4 | 39019_at | Day 3 Intra/Day 0 Intra | 0.776 | 6.0768E−03 |
| | | | alpha | 39019_at | Day 7 Intra/Day 0 Intra | 0.815 | 9.6992E−03 |
| 556 | 9528 | Entrez Gene | transmembrane protein 59 | 39033_at | Day 3 Intra/Day 0 Intra | 0.787 | 2.6172E−04 |
| 564 | 51780 | Entrez Gene | Jumonji domain containing 1B | 33870_at | Day 3 Intra/Day 0 Intra | 0.789 | 6.0617E−03 |
| 587 | 369 | Entrez Gene | v-raf murine sarcoma 3611 viral oncogene homolog | 1706_at | Day 7 Intra/Day 3 Intra | 0.832 | 4.3502E−04 |
| 588 | 23 | Entrez Gene | ATP-binding cassette, sub-family F (GCN20), | 39141_at | Day 7 Intra/Day 3 Intra | 0.837 | 5.3611E−03 |
| | | | member 1 | | | | |
| 590 | 23644 | Entrez Gene | autoantigen | 36670_at | Day 3 Intra/Day 0 Intra | 0.866 | 7.3351E−03 |

TABLE 27
Diagnostic Down

| Gene | Public | | | | | Fold | |
|---|---|---|---|---|---|---|---|
| ID | Identifier | Data Source | Gene Name | Probe_ID | Comparison | Change | P value |
| 24 | 2053 | Entrez Gene | epoxide hydrolase 2, cytoplasmic | 41473_at | Day 3 Intra/Day 0 Intra | 0.327 | 3.5922E−03 |
| 417 | 9759 | Entrez Gene | histone deacetylase 4 | 38271_at | Day 3 Intra/Day 0 Intra | 0.689 | 4.9833E−03 |
| 588 | 23 | Entrez Gene | ATP-binding cassette, sub-family F | 39141_at | Day 7 Intra/Day 3 Intra | 0.837 | 5.3611E−03 |
| | | | (GCN20), member 1 | | | | |

TABLE 28
Diagnostic Down

| | Public | | | | | | |
|---|---|---|---|---|---|---|---|
| Gene ID | Identifier | Data Source | Gene Name | Probe_ID | Comparison | Fold Change | P value |
| 102 | 6038 | Entrez Gene | ribonuclease, RNase A family, 4 | 32664_at | Day 3 Intra/Day 0 Intra | 0.483 | 2.7472E−03 |
| | | | | 32664_at | Day 7 Intra/Day 0 Intra | 0.569 | 4.9291E−04 |

1. A method for diagnosing a scar of interest as keloid or non-keloid, the method comprising: comparing expression in a sample representative of gene expression in the scar of interest, of at least one gene, selected from the group of genes set out in Table 1, with expression of the said at least one gene in a comparator tissue; wherein decreased expression of said at least one gene in the scar of interest compared to expression of said at least one gene in the comparator tissue indicates that the scar of interest comprises a keloid.

2. A method according to claim 1, wherein the method is an in vitro method.

3. A method according to claim 1, comprising comparing the expression of at least one gene selected from the group of genes set out in Table 2.

4. A method according to claim 1, comprising comparing the expression of at least one gene selected from the group of genes set out in Table 3.

5. A method according to claim 1, comprising comparing the expression of at least one gene selected from the group of genes set out in Table 8.

6. A method according to claim 1 comprising comparing the expression of at least one gene selected from the group of genes set out in Table 13.

7. A method according to claim 1, comprising comparing the expression of at least one gene selected from the group of genes set out in Table 17.

8. A method according to claim 1, comprising comparing the expression of at least one gene selected from the group of genes set out in Table 20.

9. A method according to claim 1, comprising comparing the expression of at least one gene selected from the group of genes set out in Table 21.

10. A method according to claim 1 comprising comparing the expression of at least one gene selected from the group of genes set out in Table 22.

11. A method according to claim 1, comprising comparing the expression of at least one gene selected from the group of genes set out in Table 24.

12. A method according to claim 1, comprising comparing the expression of at least one gene selected from the group of genes set out in Table 26.

13. A method according to claim 1, wherein the sample representative of gene expression in the scar of interest comprises a nucleic acid target molecule.

14. A method according to claim 13, wherein the nucleic acid target molecule comprises an RNA oligonucleotide.

15. A method according to claim 13, wherein the nucleic acid target molecule comprises a DNA oligonucleotide.

16. A method according to claim 1, wherein the sample representative of gene expression in the scar of interest comprises a protein target molecule.

17. A method according to claim 13, wherein the comparison of gene expression is effected using a probe molecule capable of binding specifically to the target molecule.

18. A method according to claim 17, wherein the probe molecule is selected from the group comprising oligonucleotide probes, antibodies and aptamers.

19. A method according to claim 1, wherein expression. in the sample and expression in the comparator tissue is compared for at least 5 genes.

20. A method according to claim 1, wherein expression in the sample and expression in the comparator tissue is compared for between 5 and 10 genes.

21. A kit for diagnosing a scar of interest as keloid or non-keloid, the kit comprising: i) at least one probe capable of binding specifically to a target molecule representative of expression in the scar of interest of at least one gene selected from the group set out in Table 1; and ii) reference material able to indicate the level of expression of said at least one gene in comparator tissue.

22. A kit according to claim 21, wherein the probe comprises an oligonucleotide probe.

23. A kit according to claim 21, wherein the probe comprises an antibody.

24. A kit according to claim 21, wherein the probe comprises an aptamer.

25. A kit according to claim 21, wherein the probe is a labelled probe.

26. A kit according to claim 25, wherein the probe is a fluorescent-labelled probe.

27. A kit according to claim 25, wherein the probe is an enzyme-labelled probe.

28. A kit according to claim 25, wherein the probe is a radioactive-labelled probe.

29. A kit according to claim 21, comprising probes capable of binding specifically to target molecules representative of expression of at least 5 genes selected from the group set out in Table 1.

30. A kit according to claim 21, comprising probes capable of binding specifically to target molecules representative of expression of between 5 and 10 genes selected from the group set out in Table 1.

31. A kit according to claim 21, wherein the kit comprises probes capable of binding specifically to target molecules representative of gene expression of at least one gene selected from those set out in Table 2; and/or those set out in Table 3; and/or those set out in Table 8; and/or those set out in Table 13; and/or those set out in Table 17 and/or those set out in Table 20; and/or those set out in Table 21; and/or those set out in Table 22; and/or those set out in Table 24; and/or those set out in Table 26.

32. A kit according to claim 21, wherein the reference material comprises a library of nucleic acid targets representative of expression of said at least one gene selected from the group of genes set out in Table 1.

33. A kit according to claim 21, wherein the reference material comprises a library of protein targets representative of expression of said at least one gene selected from the group of genes set out in Table 1.

34. A kit according to claim 21, wherein the reference material comprises data as to the expression of said at least one gene selected from the group of genes set out in Table 1.

35. A kit according to claim 21, further comprising a diagnostic algorithm.

36. A kit according to claim 21, further comprising assay control material able to indicate that an assay has been performed correctly.

37. A kit according to claim 21, further comprising materials for the preparation of a population of target molecules representative of gene expression in a scar of interest.

38. An array of oligonucleotide probes, characterised in that at least 7.0% of the oligonucleotides probes present in the array are selected from the group of genes set out in Table l.

39. An array comprising a nylon substrate to which are adhered nucleic acid probes representative of genes selected from the group of genes set out in Table 1.

40. An array comprising immobilized antibody probes capable of binding specifically to molecules representative of expression of one or more of the group of genes set out in Table l.

41. An array according to claim 38, wherein the array comprises probes capable of binding specifically to target molecules representative of gene expression of at least one gene selected from those set out in Table 2; and/or those set out in Table 3; and/or those set out in Table 8; and/or those set out in Table 13; and/or those set out in Table 17 and/or those set out in Table 20; and/or those set out in Table 21; and/or those set out in Table 22; and/or those set out in Table 24; and/or those set out in Table 26.

42. An array according to claim 39, wherein the array comprises probes capable of binding specifically to target molecules representative of gene expression of at least one gene selected from those set out in Table 2; and/or those set out in Table 3; and/or those set out in Table 8; and/or those set out in Table 13; and/or those set out in Table 17 and/or those set out in Table 20; and/or those set out in Table 21; and/or those set out in Table 22; and/or those set out in Table 24; and/or those set out in Table 26.

43. An array according to claim 40, wherein the array comprises probes capable of binding specifically to target molecules representative of gene expression of at least one gene selected from those set out in Table 2; and/or those set out in Table 3; and/or those set out in Table 8; and/or those set out in Table 13; and/or those set out in Table 17 and/or those set out in Table 20; and/or those set out in Table 21; and/or those set out in Table 22; and/or those set out in Table 24; and/or those set out in Table 26.

44. A method according to claim 16, wherein the comparison of gene expression is effected using a probe molecule capable of binding specifically to the target molecule.
